(19) United States
(12) Patent Application Publication
(10) Pub. No.: US 2023/0233671 A1
(43) Pub. Date: Jul. 27, 2023

(54) RNA MOLECULES

(71) Applicant: Pfizer Inc., New York, NY (US)

(72) Inventors: Eric Matthew Bennett, Arlington, MA (US); Fernando Martin Diaz, New York, NY (US); Philip Ralph Dormitzer, Sherborn, MA (US); Kathrin Ute Jansen, New York, NY (US); Raquel Munoz-Moreno, Edgewater, NJ (US); Alicia Solorzano Quijano, New York, NY (US)

(21) Appl. No.: 18/045,967

(22) Filed: Oct. 12, 2022

Related U.S. Application Data
(60) Provisional application No. 63/373,539, filed on Aug. 25, 2022.
(60) Provisional application No. 63/293,220, filed on Dec. 23, 2021.
(60) Provisional application No. 63/256,283, filed on Oct. 15, 2021.

(51) Int. Cl. A61K 39/25; A61K 47/69; A61K 47/10; A61K 47/28; A61P 37/04

(52) U.S. Cl. CPC A61K 39/25; A61K 47/6929; A61K 47/10; A61K 47/28; A61P 37/04

(57) ABSTRACT

The present disclosure relates to RNA molecules encoding a varicella zoster virus (VZV). The present disclosure further relates to compositions comprising the RNA molecules formulated in a lipid nanoparticle (RNA-LNP). The present disclosure further relates to the use of the RNA molecules, RNA-LNPs and compositions for the treatment or prevention of herpes zoster or shingles.

### REFERENCE TO SEQUENCE LISTING

The instant application contains a sequence listing which has been submitted electronically in .xml format and is hereby incorporated by reference in its entirety. The .xml file, named “PC072772A_SequenceListing-v2.xml”, was created on Feb. 28, 2023, and is 791 KB in size.

### BACKGROUND

Varicella-zoster virus (VZV), also known as human herpesvirus 3 (HHV-3), is a human pathogen that causes varicella or chicken pox in children and re-emerges later as herpes zoster or shingles. Two vaccines have been developed and licensed in various countries to prevent herpes zoster. First is ZOSTAVAX® (Merck & Co., Inc., Kenilworth, N.J., USA), a live attenuated VZV vaccine. The US FDA approved ZOSTAVAX® in 2006, however as of November 2020 ZOSTAVAX® is no longer available in the US. Second is SHINGRIX® (GlaxoSmithKline, Rockville, Md., USA), an AS01_{B} adjuvanted VZV gE subunit protein vaccine. The US FDA approved SHINGRIX® in 2017.

In the US, approximately 1 million cases of shingles occur annually and approximately 30% of all people who have been infected with chickenpox will later develop shingles. The incidence of shingles continues to increase globally. Thus, in view of the high prevalence of shingles, there remains a need for improved vaccines for the prevention of shingles.

### SUMMARY

The present disclosure provides immunogenic compositions and methods for treating a subject comprising the administration of RNA molecules, e.g., immunogenic RNA polynucleotide encoding an amino acid sequence, e.g., an immunogenic antigen, comprising a varicella-zoster virus (VZV) protein, an immunogenic variant thereof, or an immunogenic fragment of the VZV protein or the immunogenic variant thereof, e.g., an antigenic peptide or protein. Thus, the immunogenic antigen comprises an epitope of a VZV protein for inducing an immune response against VZV, in the subject. RNA polynucleotide encoding an immunogenic antigen is administered to provide (following expression of the polynucleotide by appropriate target cells) antigen for induction, e.g., stimulation, priming, and/or expansion, of an immune response, e.g., antibodies and/or immune effector cells. In one aspect, the immune response to be induced according to the present disclosure is a B cell-mediated immune response, e.g., an antibody-mediated immune response. Additionally or alternatively, the immune response to be induced according to the present disclosure may be a T cell-mediated immune response. In one aspect, the immune response is an anti-VZV immune response.

The immunogenic compositions described herein comprise RNA molecules comprising RNA (as the active principle) that may be translated into a protein in a recipient's cells. In addition to wild type or codon-optimized sequences encoding the antigen sequence, the RNA molecules may contain one or more structural elements optimized for maximal efficacy of the RNA with respect to stability and translational efficiency (5′ cap, 5′ UTR, 3′ UTR, poly-A-tail). In one aspect, the RNA molecules contain all of these elements. In some aspects, each uridine of the RNA molecule is replaced by N1-methylpseudouridine (ψ) (e.g., modified RNA; modRNA). The RNA molecules described herein may be formulated with, encapsulated in, or complexed with lipids and/or proteins to generate RNA-particles (e.g., lipid nanoparticles (LNPs)) for administration. In one aspect, the RNA molecules described herein are formulated with, encapsulated in, or complexed with lipids to generate RNA-lipid nanoparticles (e.g. RNA-LNPs) for administration. In one aspect, the RNA molecules described herein are formulated with, encapsulated in, or complexed with proteins for administration. In one aspect, the RNA molecules described herein are formulated with, encapsulated in, complexed with lipids and proteins for administration. If a combination of different RNA molecules is used, the RNA molecules may be formulated together or formulated separately with lipids and/or proteins to generate RNA-particles for administration.

The present disclosure provides for RNA molecules and RNA-LNPs that include at least one open reading frame (ORF) encoding a VZV antigen. In some aspects, the VZV antigen is a VZV polypeptide. In some aspects, the VZV polypeptide is a VZV glycoprotein. In some aspects, the VZV glycoprotein is VZV gK, gN, gC, gB, gH, gM, gL gI or gE. In some aspects, the VZV glycoprotein is VZV gE. In some aspects, the VZV polypeptide is a full-length, truncated, fragment or variant thereof. In some aspects, the VZV polypeptide comprises at least one mutation.

The present disclosure provides for RNA molecules and RNA-LNPs that include at least one ORF encoding a VZV polypeptide of Table 1. In some aspects, the VZV polypeptide has, has at least, or has at most 90%, 91%, 92%, 93%, 94%, 95, 96%, 97%, 98% or 99% or higher identity to any of the amino acid sequences of Table 1, for example, any of SEQ ID NO: 1 to 11. In some aspects, the VZV polypeptide comprises an amino acid sequence selected from SEQ ID NO: 1 to 11. In some aspects, the VZV polypeptide consists of any of the amino acid sequences of Table 1, for example, any of SEQ ID NO: 1 to 11.

The present disclosure provides for RNA molecules and RNA-LNPs comprising at least one ORF transcribed from at least one DNA nucleic acid of Table 2. In some aspects, the RNA molecule comprises an ORF transcribed from a nucleic acid sequence that has, has at least, or has at most 90%, 91%, 92%, 93%, 94%, 95, 96%, 97%, 98% or 99% or higher identity to any of the nucleic acid sequences of Table 2, for example, any of SEQ ID NO: 12 to 145. In some aspects, the RNA molecule is transcribed from a nucleic acid sequence selected from SEQ ID NO: 12 to 145. In some aspects, the RNA molecule comprises an ORF transcribed from a nucleic acid sequence that consists of any of the nucleic acid sequences of Table 2, for example, any of SEQ ID NO: 12 to 145.

The present disclosure further provides for RNA molecules and RNA-LNPs comprising at least one ORF comprising an RNA nucleic acid sequence of Table 3. In some aspects, the RNA molecule comprises a nucleic acid sequence that has, has at least, or has at most 90%, 91%, 92%, 93%, 94%, 95, 96%, 97%, 98% or 99% or higher identity to any of the nucleic acid sequences of Table 3, for example, any of SEQ ID NO: 146 to 279. In some aspects, the RNA molecule comprises a nucleic acid sequence selected from SEQ ID NO: 146 to 279. In some aspects, the RNA molecule comprises a nucleic acid sequence that consists of any of the nucleic acid sequences of Table 3, for example, any of SEQ ID NO: 146 to 279. In some aspects, each uridine of any of SEQ ID NO: 146 to 279 is replaced by N1-methylpseudouridine (w) (e.g., modified RNA; modRNA).

The present disclosure further provides for RNA molecules and RNA-LNPs that include a 5′ untranslated region (5′-UTR) and/or a 3′ untranslated region (3′-UTR). In some aspects, the RNA molecule includes a 5′ untranslated region (5′-UTR). In some aspects, the 5′ UTR comprises a sequence selected from any of SEQ ID NO: 281 (SEQ ID NO: 280—DNA; SEQ ID NO: 282—RNA with ψ) and 312 to 313. In some aspects, the 5′ UTR comprises a sequence having at least 90%, 91%, 92%, 93%, 94%, 95, 96%, 97%, 98% or 99% or higher identity to any of SEQ ID NO: 281 and 312 to 313. In some aspects, the 5′ UTR comprises a sequence selected from any of SEQ ID NO: 281 and 312 to 313. In some aspects, the 5′ UTR comprises a sequence consisting of any of SEQ ID NO: 281 and 312 to 313.

In some aspects, the RNA molecules and RNA-LNPs include a 3′ untranslated region (3′-UTR). In some aspects, the 3′ UTR comprises a sequence selected from any of SEQ ID NO: 284 (SEQ ID NO: 283—DNA; SEQ ID NO: 285—RNA with ψ), 314 and 317 (SEQ ID NO: 318—RNA with ψ). In some aspects, the 3′ UTR comprises a sequence having at least 90%, 91%, 92%, 93%, 94%, 95, 96%, 97%, 98% or 99% or higher identity to any of SEQ ID NO: 284, 314 and 317. In some aspects, the 3′ UTR comprises a sequence selected from any of SEQ ID NO: 284, 314 and 317. In some aspects, the 3′ UTR comprises a sequence consisting of any of SEQ ID NO: 284, 314 and 317.

The present disclosure further provides for RNA molecules and RNA-LNPs that include a 5′ cap moiety. In some aspects, the 5′ cap moiety is (3′OMe)-m₂^{7,3′-O}Gppp (m₁^{2′-O})ApG. The present disclosure further provides for RNA molecules and RNA-LNPs that include a 3′ poly-A tail. In some aspects, the poly-A tail comprises a sequence selected from any of SEQ ID NO: 287 (SEQ ID NO: 286—DNA; SEQ ID NOs: 288—RNA with ψ) and 315 (SEQ ID NO: 316—RNA with ψ). In some aspects, the poly-A tail comprises a sequence selected from any of SEQ ID NO: 287 and 315+/−1 adenosine (A) or +/−2 adenosine (A).

In some aspects, the RNA molecule includes a 5′ UTR and 3′ UTR. In some aspects, the RNA molecule includes a 5′ cap, 5′ UTR, and 3′ UTR. In some aspects, the RNA molecule includes a 5′ cap, 5′ UTR, 3′ UTR, and poly-A tail. In some aspects, the RNA molecule includes a 5′ UTR, 3′ UTR, and poly-A tail. In some aspects, each uridine of any of the 5′ UTR, 3′ UTR, and poly-A tail is replaced by N1-methylpseudouridine (ψ) (e.g., modified RNA; modRNA).

The present disclosure provides for RNA molecules as described in Table 5. In some aspects, the RNA molecule comprises a 5′ UTR of SEQ ID NO: 281 or 312, a VZV ORF of SEQ ID NO: 146, a 3′ UTR of SEQ ID NO: 284 or 317 and/or a poly-A tail of SEQ ID NO: 287 or 315 (gE WT). In some aspects, the RNA molecule comprises a 5′ UTR of SEQ ID NO: 281 or 312, a VZV ORF of SEQ ID NO: 147, a 3′ UTR of SEQ ID NO: 284 or 317 and/or a poly-A tail of SEQ ID NO: 287 or 315 (gE WT CO1). In some aspects, the RNA molecule comprises a 5′ UTR of SEQ ID NO: 281 or 312, a VZV ORF of SEQ ID NO: 148, a 3′ UTR of SEQ ID NO: 284 or 317 and/or a poly-A tail of SEQ ID NO: 287 or 315 (gE WT CO2). In some aspects, the RNA molecule comprises a 5′ UTR of SEQ ID NO: 281 or 312, a VZV ORF of SEQ ID NO: 149, a 3′ UTR of SEQ ID NO: 284 or 317 and/or a poly-A tail of SEQ ID NO: 287 or 315 (ms3 CO1). In some aspects, the RNA molecule comprises a 5′ UTR of SEQ ID NO: 281 or 312, a VZV ORF of SEQ ID NO: 150, a 3′ UTR of SEQ ID NO: 284 or 317 and/or a poly-A tail of SEQ ID NO: 287 or 315 (ms3 CO2). In some aspects, the RNA molecule comprises a 5′ UTR of SEQ ID NO: 281 or 312, a VZV ORF of SEQ ID NO: 151, a 3′ UTR of SEQ ID NO: 284 or 317 and/or a poly-A tail of SEQ ID NO: 287 or 315 (ms4 CO1). In some aspects, the RNA molecule comprises a 5′ UTR of SEQ ID NO: 281 or 312, a VZV ORF of SEQ ID NO: 152, a 3′ UTR of SEQ ID NO: 284 or 317 and/or a poly-A tail of SEQ ID NO: 287 or 315 (ms4 CO2). In some aspects, the RNA molecule comprises a 5′ UTR of SEQ ID NO: 281 or 312, a VZV ORF of SEQ ID NO: 153, a 3′ UTR of SEQ ID NO: 284 or 317 and/or a poly-A tail of SEQ ID NO: 287 or 315 (ms5 CO1). In some aspects, the RNA molecule comprises a 5′ UTR of SEQ ID NO: 281 or 312, a VZV ORF of SEQ ID NO: 154, a 3′ UTR of SEQ ID NO: 284 or 317 and/or a poly-A tail of SEQ ID NO: 287 or 315 (ms5 CO2). In some aspects, the RNA molecule comprises a 5′ UTR of SEQ ID NO: 281 or 312, a VZV ORF of SEQ ID NO: 155, a 3′ UTR of SEQ ID NO: 284 or 317 and/or a poly-A tail of SEQ ID NO: 287 or 315 (ms5 CO2 v2). In some aspects, the RNA molecule comprises a 5′ UTR of SEQ ID NO: 281 or 312, a VZV ORF of SEQ ID NO: 156, a 3′ UTR of SEQ ID NO: 284 or 317 and/or a poly-A tail of SEQ ID NO: 287 or 315 (ms6 CO1). In some aspects, the RNA molecule comprises a 5′ UTR of SEQ ID NO: 281 or 312, a VZV ORF of SEQ ID NO: 157, a 3′ UTR of SEQ ID NO: 284 or 317 and/or a poly-A tail of SEQ ID NO: 287 or 315 (ms6 CO2). In some aspects, the RNA molecule comprises a 5′ UTR of SEQ ID NO: 281 or 312, a VZV ORF of SEQ ID NO: 158, a 3′ UTR of SEQ ID NO: 284 or 317 and/or a poly-A tail of SEQ ID NO: 287 or 315 (ms8 CO1). In some aspects, the RNA molecule comprises a 5′ UTR of SEQ ID NO: 281 or 312, a VZV ORF of SEQ ID NO: 159, a 3′ UTR of SEQ ID NO: 284 or 317 and/or a poly-A tail of SEQ ID NO: 287 or 315 (ms9 CO1). In some aspects, the RNA molecule comprises a 5′ UTR of SEQ ID NO: 281 or 312, a VZV ORF of SEQ ID NO: 160, a 3′ UTR of SEQ ID NO: 284 or 317 and/or a poly-A tail of SEQ ID NO: 287 or 315 (ms9 CO2). In some aspects, the RNA molecule comprises a 5′ UTR of SEQ ID NO: 281 or 312, a VZV ORF of SEQ ID NO: 161, a 3′ UTR of SEQ ID NO: 284 or 317 and/or a poly-A tail of SEQ ID NO: 287 or 315 (ms10 CO1). In some aspects, the RNA molecule comprises a 5′ UTR of SEQ ID NO: 281 or 312, a VZV ORF of SEQ ID NO: 162, a 3′ UTR of SEQ ID NO: 284 or 317 and/or a poly-A tail of SEQ ID NO: 287 or 315 (ms10 CO2). In some aspects, the RNA molecule comprises a 5′ UTR of SEQ ID NO: 281 or 312, a VZV ORF of SEQ ID NO: 163, a 3′ UTR of SEQ ID NO: 284 or 317 and/or a poly-A tail of SEQ ID NO: 287 or 315 (ms10 CO3). In some aspects, the RNA molecule comprises a 5′ UTR of SEQ ID NO: 281 or 312, a VZV ORF of SEQ ID NO: 164, a 3′ UTR of SEQ ID NO: 284 or 317 and/or a poly-A tail of SEQ ID NO: 287 or 315 (ms11 CO1). In some aspects, the RNA molecule comprises a 5′ UTR of SEQ ID NO: 281 or 312, a VZV ORF of SEQ ID NO: 165, a 3′ UTR of SEQ ID NO: 284 or 317 and/or a poly-A tail of SEQ ID NO: 287 or 315 (ms11 CO2). In some aspects, the RNA molecule comprises a 5′ UTR of SEQ ID NO: 281 or 312, a VZV ORF of SEQ ID NO: 166, a 3′ UTR of SEQ ID NO: 284 or 317 and/or a poly-A tail of SEQ ID NO: 287 or 315 (ms12 CO1). In some aspects, the RNA molecule comprises a 5′ UTR of SEQ ID NO: 281 or 312, a VZV ORF of SEQ ID NO: 167, a 3′ UTR of SEQ ID NO: 284 or 317 and/or a poly-A tail of SEQ ID NO: 287 or 315 (ms12 CO2). In some aspects, the RNA molecule comprises a 5′ UTR of SEQ ID NO: 281 or 312, a VZV ORF of SEQ ID NO: 168, a 3′ UTR of SEQ ID NO: 284 or 317 and/or a poly-A tail of SEQ ID NO: 287 or 315. In some aspects, the RNA molecule comprises a 5′ UTR of SEQ ID NO: 281 or 312, a VZV ORF of SEQ ID NO: 169, a 3′ UTR of SEQ ID NO: 284 or 317 and/or a poly-A tail of SEQ ID NO: 287 or 315. In some aspects, the RNA molecule comprises a 5′ UTR of SEQ ID NO: 281 or 312, a VZV ORF of SEQ ID NO: 170, a 3′ UTR of SEQ ID NO: 284 or 317 and/or a poly-A tail of SEQ ID NO: 287 or 315. In some aspects, the RNA molecule comprises a 5′ UTR of SEQ ID NO: 281 or 312, a VZV ORF of SEQ ID NO: 171, a 3′ UTR of SEQ ID NO: 284 or 317 and/or a poly-A tail of SEQ ID NO: 287 or 315. In some aspects, the RNA molecule comprises a 5′ UTR of SEQ ID NO: 281 or 312, a VZV ORF of SEQ ID NO: 172, a 3′ UTR of SEQ ID NO: 284 or 317 and/or a poly-A tail of SEQ ID NO: 287 or 315. In some aspects, the RNA molecule comprises a 5′ UTR of SEQ ID NO: 281 or 312, a VZV ORF of SEQ ID NO: 173, a 3′ UTR of SEQ ID NO: 284 or 317 and/or a poly-A tail of SEQ ID NO: 287 or 315. In some aspects, the RNA molecule comprises a 5′ UTR of SEQ ID NO: 281 or 312, a VZV ORF of SEQ ID NO: 174, a 3′ UTR of SEQ ID NO: 284 or 317 and/or a poly-A tail of SEQ ID NO: 287 or 315. In some aspects, the RNA molecule comprises a 5′ UTR of SEQ ID NO: 281 or 312, a VZV ORF any one of SEQ ID NO: 175 to 238, a 3′ UTR of SEQ ID NO: 284 or 317 and/or a poly-A tail of SEQ ID NO: 287 or 315. In some aspects, the RNA molecule comprises a 5′ UTR of SEQ ID NO: 281 or 312, a VZV ORF of SEQ ID NO: 239, a 3′ UTR of SEQ ID NO: 284 or 317 and/or a poly-A tail of SEQ ID NO: 287 or 315. In some aspects, the RNA molecule comprises a 5′ UTR of SEQ ID NO: 281 or 312, a VZV ORF any one of SEQ ID NO: 240 to 254, a 3′ UTR of SEQ ID NO: 284 or 317 and/or a poly-A tail of SEQ ID NO: 287 or 315. In some aspects, the RNA molecule comprises a 5′ UTR of SEQ ID NO: 281 or 312, a VZV ORF any one of SEQ ID NO: 255 to 267, a 3′ UTR of SEQ ID NO: 284 or 317 and/or a poly-A tail of SEQ ID NO: 287 or 315. In some aspects, the RNA molecule comprises a 5′ UTR of SEQ ID NO: 281 or 312, a VZV ORF any one of SEQ ID NO: 268 to 279, a 3′ UTR of SEQ ID NO: 284 or 317 and/or a poly-A tail of SEQ ID NO: 287 or 315. In some aspects, the VZV ORF further comprises a stop codon described herein. In some aspects, the poly-A tail length may contain +1/−1 A or +2/−2 A. In some aspects, each uridine of the RNA molecule is replaced by N1-methylpseudouridine (ψ) (e.g., modified RNA; modRNA).

The present disclosure further provides for RNA molecules that include at least one open reading frame that was generated from codon-optimized DNA. In some aspects, the open reading frame comprises a G/C content of at least 55%, at least 60%, at least 65%, at least 70%, at least 75%, about 50% to 75%, or about 55% to 70%. In some aspects, the G/C content is about 58%, is about 66%, or about 62%. The present disclosure further provides for RNA molecules that encode VZV polypeptides that localizes in the cellular membrane, localizes in the Golgi and/or are anchored in the membrane and are secreted. The present disclosure further provides RNA molecules comprising stabilized RNA. The present disclosure further provides for RNA molecules that include RNA having at least one modified nucleotide (e.g., modified RNA; modRNA). In some aspects, the modified nucleotide is pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine or 2′-O-methyl uridine. In some aspects, the modified nucleotide is N1-methylpseudouridine (w).

The present disclosure further provides for RNA molecules that are messenger-RNA (mRNA) or self-replicating RNA. In some aspects, the RNA is a mRNA.

The present disclosure further provides for immunogenic compositions including the RNA molecules described herein. The RNA molecules may be formulated in, encapsulated in, complex with, bound to or adsorbed on a lipid nanoparticle (LNP) (e.g., VZV RNA-LNPs) in such immunogenic compositions. In some aspects, lipid nanoparticle includes at least one of a cationic lipid, a PEGylated lipid, and at least one structural lipid (e.g., a neutral lipid and a steroid or steroid analog).

In some aspects, the lipid nanoparticle includes a cationic lipid. In some aspects, the cationic lipid is (4-hydroxybutyl)azanediyl)bis(hexane-6,1-diyl)bis(2-hexyldecanoate) (ALC-0315).

In some aspects, lipid nanoparticle includes a polymer conjugated lipid. In some aspects, lipid nanoparticle includes a PEGylated lipid, also referred to PEG-lipid. In some aspects, the PEGylated lipid is PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols, 2-[(polyethylene glycol)-2000]-N,N-ditetradecylacetamide, glycol-lipids including PEG-c-DOMG, PEG-c-DMA, PEG-s-DMG, N-[(methoxy polyethylene glycol)2000)carbamyl]-1,2-dimyristyloxlpropyl-3-amine (PEG-c-DMA), and PEG-2000-DMG, PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-((o-methoxy(polyethoxy)ethyl)butanedioate (PEG-S-DMG), a PEGylated ceramide (PEG-cer), or a PEG dialkoxypropylcarbamate such as co-methoxy(polyethoxy)ethyl-N-(2,3di(tetradecanoxy)propyl)carbamate or 2,3-di(tetradecanoxy)propyl-N-(u>-methoxy(polyethoxy)ethyl)carbamate. In some aspects, the PEGylated lipid is 2-[(polyethylene glycol)-2000]-N,N-ditetradecylacetamide (ALC-0159).

In some aspects, lipid nanoparticle includes at least one structural lipid, such as a neutral lipid. In some aspects, the neutral lipid is selected from 1,2-distearoyl-sn-glycero-3-phosphocholine (DSPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1 carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and/or 1,2-dielaidoyl-sn-glycero-3-phophoethanolamine (transDOPE). In some aspects, the neutral lipid is 1,2-distearoyl-sn-glycero-3-phosphocholine (DSPC).

In some aspects, the lipid nanoparticle includes a second structural lipid, such as a steroid or steroid analog. In some aspects, the steroid or steroid analog is cholesterol.

In some aspects, the lipid nanoparticle has a mean diameter of about 1 to about 500 nm.

In some aspects, the RNA-LNP immunogenic composition is a liquid RNA-LNP composition comprising a RNA molecule/polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, encapsulated in LNPs with a lipid composition comprising a cationic lipid at a concentration of about 0.8 to 0.95 mg/mL, a PEGylated lipid at a concentration of about 0.05 to 0.15 mg/mL, a first structural lipid at a concentration of about 0.1 to 0.25 mg/mL, and a second structural lipid at a concentration of about 0.3 to 0.45 mg/mL. In some aspects, the liquid composition further comprises a buffer composition comprising a first buffer at a concentration of about 0.15 to 0.3 mg/mL, a second buffer at a concentration of about 1.25 to 1.4 mg/mL, and a stabilizing agent at a concentration of about 95 to 110 mg/mL.

In specific aspects, the liquid RNA-LNP immunogenic composition comprises a RNA molecule/polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, encapsulated in LNPs with a lipid composition comprising ((4-hydroxybutyl)azanediyl)bis(hexane-6,1-diyl)bis(2-hexyldecanoate) (ALC-0315) at a concentration of about 0.8 to 0.95 mg/mL, 2-[(polyethylene glycol)-2000]-N,N-ditetradecylacetamide (ALC-0159) at a concentration of about 0.05 to 0.15 mg/mL, 1,2-Distearoyl-sn-glycero-3-phosphocholine (DSPC) at a concentration of about 0.1 to 0.25 mg/mL, and cholesterol at a concentration of about 0.3 to 0.45 mg/mL. In some aspects, the liquid composition further comprises a Tris buffer composition comprising tromethamine at a concentration of about 0.1 to 0.3 mg/mL and Tris hydrochloride (HCl) at a concentration of about 1.25 to 1.4 mg/mL, and sucrose at a concentration of about 95 to 110 mg/mL.

In some aspects, the liquid RNA-LNP immunogenic composition comprises a RNA molecule/polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, encapsulated in a LNP, and further comprising about 5 to 15 mM Tris buffer, 200 to 400 mM sucrose at a pH of about 7.0 to 8.0.

In some aspects, the RNA-LNP immunogenic composition is a lyophilized (reconstituted) RNA-LNP composition comprising a RNA molecule/polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, encapsulated in LNPs with a lipid composition comprising a cationic lipid at a concentration of about 0.8 to 0.95 mg/mL, a PEGylated lipid at a concentration of about 0.05 to 0.15 mg/mL, a first structural lipid at a concentration of about 0.1 to 0.25 mg/mL, and a second structural lipid at a concentration of about 0.3 to 0.45 mg/mL. In some aspects, the lyophilized composition further comprises a first buffer at a concentration of about 0.01 and 0.15 mg/mL, a second buffer at a concentration of about 0.5 and 0.65 mg/mL, a stabilizing agent at a concentration of about 35 to 50 mg/mL, and a salt diluent at a concentration of about 5 to 15 mg/mL for reconstitution. In specific aspects, the lyophilized compositions are reconstituted in about 0.6 to 0.75 mL of the salt diluent. Concentrations in the lyophilized RNA-LNP composition are determined post-reconstitution.

In specific aspects, a lyophilized (reconstituted) RNA-LNP composition comprises a RNA polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, encapsulated in LNPs with a lipid composition of ALC-0315 at a concentration of about 0.8 to 0.95 mg/mL, ALC-0159 at a concentration of about 0.05 to 0.15 mg/mL, DSPC at a concentration of about 0.1 to 0.25 mg/mL, and cholesterol at a concentration of about 0.3 to 0.45 mg/mL, and further comprises a Tris buffer composition comprising tromethamine at a concentration of about 0.01 and 0.15 mg/mL and Tris HCl at a concentration of about 0.5 and 0.65 mg/mL, sucrose at a concentration of about 35 to 50 mg/mL, and sodium chloride (NaCl) diluent at a concentration of about 5 to 15 mg/mL for reconstitution. In specific aspects, the lyophilized compositions are reconstituted in about 0.6 to 0.75 mL of sodium chloride. Concentrations in the lyophilized RNA-LNP composition are determined post-reconstitution.

The present disclosure provides for RNA molecules, RNA-LNPs and immunogenic compositions that may be administered to a subject at a dose of at least, at most, exactly, or between any two of 1 μg, 15 μg, 30 μg, 45 μg, 60 μg, 75 μg, 90 μg, 100 μg or higher per administration of VZV RNA encapsulated in LNP.

The present disclosure provides for RNA molecules, RNA-LNPs and immunogenic compositions that may be administered in a single dose. The present disclosure further provides for RNA molecules, RNA-LNPs and immunogenic compositions that may be administered twice (e.g., Day 0 and about Day 7, Day 0 and about Day 14, Day 0 and about Day 21, Day 0 and about Day 28, Day 0 and about Day 60, Day 0 and about Day 90, Day 0 and about Day 120, Day 0 and about Day 150, Day 0 and about Day 180, Day 0 and about 1 month later, Day 0 and about 2 months later, Day 0 and about 3 months later, Day 0 and about 6 months later, Day 0 and about 9 months later, Day 0 and about 12 months later, Day 0 and about 18 months later, Day 0 and about 2 years later, Day 0 and about 5 years later, or Day 0 and about 10 years later). The present disclosure further provides for RNA molecules, RNA-LNPs and immunogenic compositions that may be administered twice at Day 0 and about 2 months later. The present disclosure further provides for RNA molecules, RNA-LNPs and immunogenic compositions that may be administered twice at Day 0 and about 6 months later. The present disclosure further provides for RNA molecules, RNA-LNPs and immunogenic compositions that may be administered three, four, five, six, seven, eight, nine, ten, eleven, twelve, thirteen, fourteen, or more administrations. In some aspects, periodic boosters at intervals of 1-5 years may be desirable to maintain protective levels of the antibodies. The present disclosure further provides for administration of a at least one booster dose.

The present disclosure provides for a method of inducing an immune response against VZV in a subject, including administering to the subject an effective amount of an RNA molecule, RNA-LNP and/or immunogenic composition described herein. The present disclosure further provides for the use of an RNA molecule, RNA-LNP and/or immunogenic composition described herein in the manufacture of a medicament for use in inducing an immune response against VZV in a subject.

The present disclosure provides for a method of inducing an immune response against VZV in a subject, including administering to the subject an effective amount of an RNA molecule and/or RNA-LNP that includes at least one open reading frame encoding a VZV polypeptide or composition described herein. The present disclosure further provides for the use of an RNA molecule and/or RNA-LNP that includes at least one open reading frame encoding a VZV polypeptide or composition described herein in the manufacture of a medicament for use in inducing an immune response against VZV in a subject.

The present disclosure provides for a method of inducing an immune response against VZV in a subject, including administering to the subject an effective amount of an RNA molecule and/or RNA-LNP that includes at least one open reading frame encoding a polypeptide of a gene of interest or composition described herein. The present disclosure further provides for the use of an RNA molecule and/or RNA-LNP that includes at least one open reading frame encoding a polypeptide of a gene of interest or composition described herein in the manufacture of a medicament for use in inducing an immune response against VZV in a subject.

The present disclosure provides for a method of preventing, treating or ameliorating an infection, disease or condition in a subject, including administering to a subject an effective amount of an RNA molecule, RNA-LNP and/or immunogenic composition described herein. The present disclosure further provides for the use of an RNA molecule RNA-LNP and/or immunogenic composition described herein in the manufacture of a medicament for use in preventing, treating or ameliorating an infection, disease or condition in a subject. In some aspects, the infection, disease or condition is associated with VZV. In some aspects, the infection, disease or condition is herpes zoster (shingles). In some aspects, the infection, disease or condition is postherpetic neuralgia.

The present disclosure provides for a method of preventing, treating or ameliorating an infection, disease or condition in a subject, including administering to a subject an effective amount of an RNA molecule and/or RNA-LNP that includes at least one open reading frame encoding a VZV polypeptide or immunogenic composition described herein. The present disclosure further provides for the use of an RNA molecule and/or RNA-LNP that includes at least one open reading frame encoding a VZV polypeptide or immunogenic composition described herein in the manufacture of a medicament for use in preventing, treating or ameliorating an infection, disease or condition in a subject. In some aspects, the infection, disease or condition is associated with VZV. In some aspects, the infection, disease or condition is herpes zoster or shingles. In some aspects, the infection, disease or condition is postherpetic neuralgia.

The present disclosure further provides for a method of preventing, treating or ameliorating an infection, disease or condition in a subject, including administering to a subject an effective amount of RNA molecules and/or RNA-LNPs that include at least one open reading frame encoding a polypeptide of a gene of interest or immunogenic compositions described herein. The present disclosure further provides for the use of RNA molecules and/or RNA-LNPs that include at least one open reading frame encoding a polypeptide of a gene of interest or immunogenic compositions described herein in the manufacture of a medicament for use in preventing, treating or ameliorating an infection, disease or condition in a subject. In some aspects, the infection, disease or condition is associated with the gene of interest.

In some aspects, the subject is, is at least, or is at most less than about 1 year of age, about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older. In some aspects, the subject the subject is about 50 years of age or older.

In some aspects, the subject is immunocompetent. In some aspects, the subject is immunocompromised.

The present disclosure provides for a method or use described herein, wherein the RNA molecule, RNA-LNP and/or immunogenic composition is administered as a vaccine.

The present disclosure provides a method or use described herein, wherein the RNA molecule, RNA-LNP and/or immunogenic composition is administered by intradermal or intramuscular injection.

It is contemplated that any aspect discussed in this specification may be implemented with respect to any method or composition of the disclosure, and vice versa. Furthermore, compositions of the disclosure may be used to achieve methods of the disclosure.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

FIG. 1 schematically illustrates wild-type (WT) varicella-zoster virus (VZV) gE protein (gE WT) and variant VZV gE proteins, where SP refers to a signal peptide sequence, ectodomain refers to a peptide sequence corresponding to the portion of the protein that extends into the extracellular space, TM refers to a transmembrane peptide sequence corresponding to the portion of the protein that spans the cell membrane, and CT refers to a cytoplasmic tail peptide sequence corresponding to the portion of the protein that extends into the cell cytoplasm. Variant VZV gE proteins having cytoplasmic tail modifications are designated ms4, ms5, ms8, ms9, ms10, ms11, and ms12. Secreted variant VZV gE proteins having TM modifications are designated ms3 and ms6. VZV gE RNA constructs encoding the VZV gE proteins were generated from codon-optimized (CO) DNA, where CO1 indicates CO constructs with about 58% G/C content, CO2 indicates CO constructs with about 66% G/C content, and CO3 indicates CO constructs with about 62% G/C content.

FIG. 2 shows VZV gE expression by Vero cells transfected with 10 ng, 25 ng, or 50 ng of VZV RNA constructs, including WT constructs (gE_WT CO1 and gE_WT CO2) and constructs having modifications in the cytoplasmic tail (CT) (ms4, ms5, ms8, ms9, ms10, ms11, and ms12; CO1 and CO2 for each). Cells were imaged for VZV gE expression, and the percentage of VZV gE⁺ transfected Vero cells is shown for each VZV RNA construct.

FIG. 3 shows VZV gE expression by Vero cells transfected with 10 ng, 25 ng, or 50 ng of the VZV RNA constructs having modifications in the transmembrane (TM) (secreted) (ms3 and ms6; CO1 and CO2 for each). Cells were imaged for VZV gE expression, and the percentage of VZV gE⁺ transfected Vero cells is shown for each VZV RNA construct.

FIG. 4 shows the mean fluorescence intensity (MFI) of Vero cells transfected with 10 ng, 25 ng, or 50 ng of the VZV RNA constructs, including WT constructs (gE_WT CO1 and gE_WT CO2) and constructs having modifications in the cytoplasmic tail (CT) (ms4, ms5, ms8, ms9, ms10, ms11, and ms12; CO1 and CO2 for each).

FIG. 5 shows the mean fluorescence intensity (MFI) of Vero cells transfected with 10 ng, 25 ng, or 50 ng of the VZV RNA constructs having modifications in the transmembrane (TM) (secreted) (ms3 and ms6; CO1 and CO2 for each).

FIG. 6 shows the subcellular localization at 63× magnification of VZV gE in Vero cells transfected with 50 ng of gE WT VZV RNA constructs and variant VZV gE RNA constructs (ms4, ms5, ms8) having cytoplasmic tail modifications.

FIG. 7 shows the subcellular localization at 63× magnification of VZV gE in Vero cells transfected with 50 ng of variant VZV gE RNA constructs (ms9, ms11, and ms12) having cytoplasmic tail modifications.

FIG. 8 shows the subcellular localization at 63× magnification of VZV gE in Vero cells transfected with 50 ng of variant VZV gE RNA constructs (ms10) having cytoplasmic tail modifications.

FIG. 9 shows the subcellular localization at 63× magnification of VZV gE in Vero cells transfected with 50 ng variant VZV gE RNA constructs (ms3 and ms6) having TM modifications (secreted).

FIG. 10 shows the subcellular localization at 10× magnification of VZV gE in Vero cells transfected with 25 ng of gE WT VZV RNA constructs and variant VZV gE RNA (ms4, ms5, ms8) having cytoplasmic tail modifications.

FIG. 11 shows the subcellular localization at 10× magnification of VZV gE in Vero cells transfected with 25 ng of variant VZV gE RNA constructs (ms9, ms11, ms12) having cytoplasmic tail modifications.

FIG. 12 shows the subcellular localization at 10× magnification of VZV gE in Vero cells transfected with 25 ng of variant VZV gE RNA constructs (ms10) having cytoplasmic tail modifications.

FIG. 13 shows the subcellular localization at 10× magnification of VZV gE in Vero cells transfected with 25 ng of variant VZV gE RNA constructs (ms3 and ms6) having TM modifications (secreted).

FIG. 14 shows a titration curve created by plotting input RNA quantities against the percentage of VZV gE-expressing human embryonic kidney (HEK) 293T cells, where the titration curve is used to determine the EC₅₀ of VZV RNA-LNP vaccines formulated with a VZV gE WT RNA construct (gE WT CO1 and gE WT CO2), a variant VZV gE RNA construct having cytoplasmic tail modifications (ms4 CO1) and a variant VZV gE RNA construct having transmembrane (TM) modifications (ms3 CO1) (secreted).

FIG. 15 shows a titration curve created by plotting input RNA quantities against the percentage of VZV gE-expressing human embryonic kidney (HEK) 293T cells, where the titration curve is used to determine the EC₅₀ of VZV RNA-LNP vaccines formulated with a VZV gE WT RNA construct (gE WT CO1 and gE WT CO2), a variant VZV gE RNA construct having cytoplasmic tail modifications (ms5 CO1) and a variant VZV gE RNA construct having TM modifications (ms6 CO2) (secreted).

FIG. 16 shows serum IgG levels in mice on Day 28 after immunization on Day 0 with a priming dose of: 1 μg, 2.5 μg, or 5 μg of SHINGRIX®; 0.5 μg or 1 μg of a VZV RNA-LNP vaccine formulated with gE_WT CO1 RNA construct; 0.5 μg of a VZV RNA-LNP vaccine formulated with ms3 CO1 RNA construct having TM modifications (secreted); 0.5 μg of a VZV RNA-LNP vaccine formulated with ms4 CO1 RNA construct having cytoplasmic tail modifications; 0.5 μg of a VZV RNA-LNP vaccine formulated with gE_WT CO2 RNA construct; or 0.5 μg or 1 μg of a lyophilized VZV RNA-LNP vaccine formulated with gE_WT CO1 RNA construct (gE_WT CO1 LYO).

FIG. 17 shows serum IgG levels in mice on Day 34 after immunization on Day 0 with a priming dose and immunization on Day 28 with a booster dose of: 1 μg, 2.5 μg, or 5 μg of SHINGRIX®; 0.5 μg or 1 μg of a VZV RNA-LNP vaccine formulated with gE_WT CO1 RNA construct; 0.5 μg of a VZV RNA-LNP vaccine formulated with ms3 CO1 RNA construct having TM modifications (secreted); 0.5 μg of a VZV RNA-LNP vaccine formulated with ms4 CO1 RNA construct having cytoplasmic tail modifications; 0.5 μg of a VZV RNA-LNP vaccine formulated with gE_WT CO2 RNA construct; or 0.5 μg or 1 μg of a lyophilized VZV RNA-LNP vaccine formulated with gE_WT CO1 RNA construct (gE_WT CO1 LYO).

FIG. 18 shows serum IgG levels in mice on Day 42 after immunization on Day 0 with a priming dose and immunization on Day 28 with a booster dose of: 1 μg, 2.5 μg, or 5 μg of SHINGRIX®; 0.5 μg or 1 μg of a VZV RNA-LNP vaccine formulated with gE_WT CO1 RNA construct; 0.5 μg of a VZV RNA-LNP vaccine formulated with ms3 CO1 RNA construct having TM modifications (secreted); 0.5 μg of a VZV RNA-LNP vaccine formulated with ms4 CO1 RNA construct having cytoplasmic tail modifications; 0.5 μg of a VZV RNA-LNP vaccine formulated with gE_WT CO2 RNA construct; or 0.5 μg or 1 μg of a lyophilized VZV RNA-LNP vaccine formulated with gE_WT CO1 RNA construct (gE_WT CO1 LYO).

FIG. 19 shows a comparison of serum IgG levels in mice on Day 28 (priming dose only), Day 34 (six days after booster dose), and Day 42 (two weeks after booster dose) after immunization on Day 0 with a priming dose and immunization on Day 28 with a booster dose of SHINGRIX® (1 μg) or a VZV RNA-LNP vaccine (0.5 μg).

FIG. 20 shows the percentage of CD4⁺ IFN-γ⁺-staining T cells following ex vivo stimulation of splenocytes harvested from mice on Day 34 after immunization on Day 0 with a priming dose and immunization on Day 28 with a booster dose of SHINGRIX® (1 μg, 2.5 μg, or 5 μg) or a VZV RNA-LNP vaccine (0.5 μg and/or 1 μg).

FIG. 21 shows the percentage of CD8⁺ IFN-γ⁺-staining T cells following ex vivo stimulation of splenocytes harvested from mice on Day 34 after immunization on Day 0 with a priming dose and immunization on Day 28 with a booster dose of SHINGRIX® (1 μg, 2.5 μg, or 5 μg) or a VZV RNA-LNP vaccine (0.5 μg and/or 1 μg).

FIG. 22 shows serum IgG levels in mice on Day 35 after a subcutaneous vaccination with 1350 pfu live-attenuated varicella (LAV) on Day 0.

FIG. 23 shows serum IgG levels in mice on Day 63 (1 month post dose 1) after vaccination with LAV vaccine on Day 0 and immunization on Day 35 with a dose of SHINGRIX® (5 μg, 2.5 μg or 1 μg); VZV RNA-LNP vaccine formulated with gE_WT CO2 RNA construct (1 μg or 0.5 μg); VZV RNA-LNP vaccine formulated with ms5 CO1 RNA construct having cytoplasmic tail modifications (1 μg or 0.5 μg); or VZV RNA-LNP vaccine formulated with ms6 CO2 RNA construct having TM modifications (secreted) (1 μg or 0.5 μg).

FIG. 24 shows serum IgG levels in mice on Day 76 (13 days post dose 2/boost) after vaccination with LAV vaccine on Day 0 and immunization on Day 35 and Day 63 with a dose of SHINGRIX® (5 μg, 2.5 μg or 1 μg); VZV RNA-LNP vaccine formulated with gE_WT CO2 RNA construct (1 μg or 0.5 μg); VZV RNA-LNP vaccine formulated with ms5 CO1 RNA construct having cytoplasmic tail modifications (1 μg or 0.5 μg); or VZV RNA-LNP vaccine formulated with ms6 CO2 RNA construct having TM modifications (secreted) (1 μg or 0.5 μg).

FIG. 25 shows serum IgG levels in mice on Day 63 (1 month post dose 1) after vaccination with LAV vaccine on Day 0 and immunization on Day 35 with a dose of VZV RNA-LNP vaccine formulated with gE_WT CO2 RNA construct (1 μg or 0.5 μg) or lyophilized VZV RNA-LNP vaccine formulated with gE_WT CO2 RNA construct (gE_WT CO2 lyo) (1 μg or 0.5 μg); and on Day 76 (13 days post dose 2/boost) after vaccination with LAV vaccine on Day 0 and immunization on Day 35 and Day 63 with a dose of VZV RNA-LNP vaccine formulated with gE_WT CO2 (1 μg or 0.5 μg) or lyophilized VZV RNA-LNP vaccine formulated with gE_WT CO2 RNA construct (gE_WT CO2 lyo) (1 μg or 0.5 μg).

FIGS. 26A-26D show the VZV gE-specific T cell and B cell responses induced by vaccines in splenocytes collected on Day 48 (13 days post dose 1) in LAV-experienced mice immunized at Day 35 with a dose of SHINGRIX® (5 μg or 1 μg); VZV RNA-LNP vaccine formulated with gE_WT CO2 RNA construct (1 μg or 0.5 μg); VZV RNA-LNP vaccine formulated with ms5 CO1 RNA construct having cytoplasmic tail modifications (1 μg); VZV RNA-LNP vaccine formulated with ms6 CO2 RNA construct having TM modifications (1 μg); or lyophilized VZV RNA-LNP vaccine formulated with gE_WT CO2 (gE_WT CO2 Lyo) (1 μg). FIG. 26A shows ELISpot measured number of VZV gE-specific cells secreting IFN-γ and results are expressed as spot forming cells (SFC) per million cells. FIG. 26B shows ICS assay measured IFN-γ-expressing cells within CD4⁺ T cells expressed as percentage of IFN-γ⁺ cells within CD4⁺ T cells. FIG. 26C shows ICS assay measured IFN-γ-expressing cells within CD8⁺ T cells expressed as percentage of IFN-γ⁺ cells within CD8⁺ T cells. FIG. 26D shows B-cell response evaluated in splenocytes by measuring the frequency of gE-specific IgG⁺ B cells by flow cytometry.

FIG. 27A-27C show the VZV gE-specific T cell and B cell responses induced by vaccines in splenocytes collected on Day 76 (13 days post dose 2/boost) in LAV-experienced mice immunized at Day 35 and Day 63 with a dose of SHINGRIX® (5 μg or 1 μg); VZV RNA-LNP vaccine formulated with gE_WT CO2 RNA construct (1 μg or 0.5 μg); VZV RNA-LNP vaccine formulated with ms5 CO1 RNA construct having cytoplasmic tail modifications (1 μg); VZV RNA-LNP vaccine formulated with ms6 CO2 RNA construct having TM modifications (1 μg); or lyophilized VZV RNA-LNP vaccine formulated with gE_WT CO2 (gE_WT CO2 Lyo) (1 μg). FIG. 27A shows ICS assay measured IFN-γ-expressing cells within CD4⁺ T cells expressed as percentage of IFN-γ⁺ cells within CD4⁺ T cells. FIG. 27B shows ICS assay measured IFN-γ-expressing cells within CD8⁺ T cells expressed as percentage of IFN-γ⁺ cells within CD8⁺ T cells. FIG. 27C shows B-cell response evaluated in splenocytes by measuring the frequency of gE-specific IgG⁺ B cells by flow cytometry.

### DETAILED DESCRIPTION

The present disclosure provides for an RNA molecule (e.g., RNA polynucleotide) comprising at least one open reading frame (ORF) encoding a varicella-zoster virus (VZV) antigen. In some aspects, the VZV antigen is a VZV polypeptide. In some aspects, the VZV polypeptide is a VZV gE polypeptide. In some aspects, the VZV polypeptide comprises an amino acid sequence of Table 1. In some aspects, the RNA molecules comprise an ORF transcribed from at least one DNA nucleic acid sequence of Table 2. In some aspects, the RNA molecules comprise an ORF comprising an RNA nucleic acid sequence of Table 3. In some aspects the RNA molecule comprises at least one of a 5′ cap, 5′ UTR, 3′ UTR and poly-A tail. The present disclosure provides for an RNA molecule comprising modified nucleotides (e.g., modified RNA; modRNA). The present disclosure provides for an immunogenic composition comprising any one of the RNA molecules encoding a VZV polypeptide described herein complexed with, encapsulated in, or formulated with one or more lipids, and forming lipid nanoparticles (RNA-LNPs). The present disclosure further provides for an immunogenic composition comprising any one of the RNA molecules comprising at least one RNA nucleic acid described herein complexed with, encapsulated in, or formulated with one or more lipids, and forming RNA-LNPs. The present disclosure further provides for a method of preventing, treating or ameliorating an infection, disease or condition (e.g., herpes zoster or shingles) in a subject via administering to a subject an effective amount of an RNA molecule, RNA-LNP or an immunogenic composition described herein. The present disclosure further provides for the use of the RNA molecule, RNA-LNP and/or an immunogenic compositions described herein as a vaccine.

### I. Examples of Definitions

Throughout this application, the term “about” is used according to its plain and ordinary meaning in the area of cell and molecular biology to indicate a deviation of ±10% of the value(s) to which it is attached.

Recitation of ranges of values herein is merely intended to serve as a shorthand method of referring individually to each separate value falling within the range. Unless otherwise indicated herein, each individual value is incorporated into the specification as if it was individually recited herein.

The use of the word “a” or “an” when used in conjunction with the term “comprising” may mean “one,” but it is also consistent with the meaning of “one or more,” “at least one,” and “one or more than one.”

The phrase “and/or” means “and” or “or.” To illustrate, A, B, and/or C includes: A alone, B alone, C alone, a combination of A and B, a combination of A and C, a combination of B and C, or a combination of A, B, and C. In other words, “and/or” operates as an inclusive or.

The phrase “essentially all” is defined as “at least 95%”; if essentially all members of a group have a certain property, then at least 95% of members of the group have that property. In some aspects, essentially all means equal to any one of, at least any one of, or between any two of 95, 96, 97, 98, 99, or 100% of members of the group have that property.

The compositions and methods for their use may “comprise,” “consist essentially of,” or “consist of” any of the ingredients or steps disclosed throughout the specification. Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and will be understood to imply the inclusion of a stated step or element or group of steps or elements but not the exclusion of any other step or element or group of steps or elements. It is contemplated that aspects described herein in the context of the term “comprising” may also be implemented in the context of the term “consisting of” or “consisting essentially of.” Compositions and methods “consisting essentially of” any of the ingredients or steps disclosed limits the scope of the claim to the specified materials or steps which do not materially affect the basic and novel characteristic of the claimed disclosure. The words “consisting of” (and any form of consisting of, such as “consist of” and “consists of”) means including, and limited to, whatever follows the phrase “consisting of.” Thus, the phrase “consisting of” indicates that the listed elements are required or mandatory, and that no other elements may be present.

Reference throughout this specification to “one aspect,” “an aspect,” “a particular aspect,” “a related aspect,” “a certain aspect,” “an additional aspect,” or “a further aspect” or combinations thereof means that a particular feature, structure or characteristic described in connection with the aspect is included in at least one aspect of the present disclosure. Thus, the appearances of the foregoing phrases in various places throughout this specification are not necessarily all referring to the same aspect. Furthermore, the particular features, structures, or characteristics may be combined in any suitable manner in one or more aspects.

The terms “inhibiting,” “decreasing,” or “reducing” or any variation of these terms includes any measurable decrease (e.g., a 5%, 10%, 15%, 20%, 25%, 30%, 35%, 40%, 45%, 50%, 55%, 60%, 65%, 70%, 75%, 80%, 85%, 90%, 95%, or 99% decrease) or complete inhibition to achieve a desired result. The terms “improve,” “promote,” or “increase” or any variation of these terms includes any measurable increase (e.g., a 5%, 10%, 15%, 20%, 25%, 30%, 35%, 40%, 45%, 50%, 55%, 60%, 65%, 70%, 75%, 80%, 85%, 90%, 95%, or 99% increase) to achieve a desired result or production of a protein or molecule.

As used herein, the terms “reference,” “standard,” or “control” describe a value relative to which a comparison is performed. For example, an agent, subject, population, sample, or value of interest is compared with a reference, standard, or control agent, subject, population, sample, or value of interest. A reference, standard, or control may be tested and/or determined substantially simultaneously and/or with the testing or determination of interest for an agent, subject, population, sample, or value of interest and/or may be determined or characterized under comparable conditions or circumstances to the agent, subject, population, sample, or value of interest under assessment.

The term “isolated” may refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that may be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state and/or that is altered or removed from the natural state through human intervention. For example, a DNA naturally present in a living animal is not “isolated,” but a synthetic DNA, or a DNA partially or completely separated from the coexisting materials of its natural state is “isolated.” An isolated nucleic acid may exist in substantially purified form, or may exist in a non-native environment such as, for example, a cell into which the nucleic acid has been delivered.

A “nucleic acid,” as used herein, is a molecule comprising nucleic acid components and refers to DNA or RNA molecules. It may be used interchangeably with the term “polynucleotide.” A nucleic acid molecule is a polymer comprising or consisting of nucleotide monomers, which are covalently linked to each other by phosphodiester-bonds of a sugar/phosphate-backbone. Nucleic acids may also encompass modified nucleic acid molecules, such as base-modified, sugar-modified or backbone-modified etc. DNA or RNA molecules. Nucleic acids may exist in a variety of forms such as: isolated segments and recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, mRNA, saRNA, and complementary sequences of the foregoing described herein. Nucleic acids may encode an epitope to which antibodies may bind.

The term “epitope” refers to a moiety that is specifically recognized by an immunoglobulin (e.g., antibody or receptor) binding component. In some aspects, an epitope is comprised of a plurality of chemical atoms or groups on an antigen. In some aspects, such chemical atoms or groups are surface-exposed when the antigen adopts a relevant three-dimensional conformation. In some aspects, such chemical atoms or groups are physically near to each other in space when the antigen adopts such a conformation. In some aspects, at least some such chemical atoms are groups are physically separated from one another when the antigen adopts an alternative conformation (e.g., is linearized).

Nucleic acids may be single-stranded or double-stranded and may comprise RNA and/or DNA nucleotides and artificial variants thereof (e.g., peptide nucleic acids). In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. A tag or other heterologous polypeptide may be added to the modified polypeptide-encoding sequence, wherein “heterologous” refers to a polypeptide that is not the same as the modified polypeptide.

The term “polynucleotide” refers to a nucleic acid molecule that may be recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA, or synthetic), analogs thereof, or a combination thereof. Additional coding or non-coding sequences may, but need not, be present within a polynucleotide.

In certain aspects, there are polynucleotide variants having substantial identity to the sequences disclosed herein; those comprising equal to any one of, at least any one of, at most any one of, or between any two of 70%, 75%, 80%, 85%, 90%, 95%, 96%, 97%, 98%, or 99% or higher sequence identity, compared to a polynucleotide sequence provided herein using the methods described herein (e.g., BLAST analysis using standard parameters). In certain aspects, the isolated polynucleotide will comprise a nucleotide sequence encoding a polypeptide that has at least 90% identity to an amino acid sequence described herein, over the entire length of the sequence; or a nucleotide sequence complementary to said isolated polynucleotide. In some aspects, the isolated polynucleotide will comprise a nucleotide sequence encoding a polypeptide that has at least 95% identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids may be any length. They may be, for example, equal to any one of, at least any one of, at most any one of, or between any two of 5, 10, 15, 20, 25, 30, 35, 40, 45, 50, 75, 100, 125, 175, 200, 250, 300, 350, 400, 450, 500, 750, 1000, 1500, 3000, 5000, 6000, 7000, 8000, 9000, 10000, 11000, 12000, 13000, 14000, 15000 or more nucleotides in length, and/or may comprise one or more additional sequences, for example, regulatory sequences, and/or be a part of a larger nucleic acid, for example, a vector. It is therefore contemplated that a nucleic acid fragment of almost any length may be employed, with the total length being limited by the ease of preparation and use in the intended recombinant nucleic acid protocol.

In this respect, the term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

As used herein, the term “expression” of a nucleic acid sequence refers to the generation of any gene product from the nucleic acid sequence. In some aspects, a gene product may be a transcript. In some aspects, a gene product may be a polypeptide. In some aspects, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

The term “DNA,” as used herein, means a nucleic acid molecule comprising nucleotides such as deoxy-adenosine-monophosphate, deoxy-thymidine-monophosphate, deoxy-guanosine-monophosphate and deoxy-cytidine-monophosphate monomers which are composed of a sugar moiety (deoxyribose), a base moiety and a phosphate moiety, and polymerize by a characteristic backbone structure. The backbone structure is, typically, formed by phosphodiester bonds between the sugar moiety of the nucleotide, e.g., deoxyribose, of a first and a phosphate moiety of a second, adjacent monomer. The specific order of the monomers, e.g., the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by A/T-base-pairing and G/C-base-pairing. DNA may contain all, or a majority of, deoxyribonucleotide residues. As used herein, the term “deoxyribonucleotide” means a nucleotide lacking a hydroxyl group at the 2′ position of a β-D-ribofuranosyl group. Without any limitation, DNA may encompass double stranded DNA, antisense DNA, single stranded DNA, isolated DNA, synthetic DNA, DNA that is recombinantly produced, and modified DNA.

The term “RNA,” as used herein, means a nucleic acid molecule comprising nucleotides such as adenosine-monophosphate, uridine-monophosphate, guanosine-monophosphate and cytidine-monophosphate monomers which are connected to each other along a so-called backbone. The backbone is formed by phosphodiester bonds between the sugar, e.g., ribose, of a first and a phosphate moiety of a second, adjacent monomer. RNA may be obtainable by transcription of a DNA-sequence, e.g., inside a cell. In eukaryotic cells, transcription is typically performed inside the nucleus or the mitochondria. In vivo, transcription of DNA may result in premature RNA which is processed into messenger-RNA (mRNA). Processing of the premature RNA, e.g. in eukaryotic organisms, comprises various posttranscriptional modifications such as splicing, 5′ capping, polyadenylation, export from the nucleus or the mitochondria. Mature messenger RNA is processed and provides the nucleotide sequence that may be translated into an amino acid sequence of a peptide or protein. A mature mRNA may comprise a 5′ cap, a 5′ UTR, an open reading frame, a 3′ UTR and a poly-A tail sequence. RNA may contain all, or a majority of, ribonucleotide residues. As used herein, the term “ribonucleotide” means a nucleotide with a hydroxyl group at the 2′ position of a β-D-ribofuranosyl group. In one aspect, RNA may be messenger RNA (mRNA) that relates to a RNA transcript which encodes a peptide or protein. As known to those of skill in the art, mRNA generally contains a 5′ untranslated region (5′ UTR), a polypeptide coding region, and a 3′ untranslated region (3′ UTR). Without any limitation, RNA may encompass double stranded RNA, antisense RNA, single stranded RNA, isolated RNA, synthetic RNA, RNA that is recombinantly produced, and modified RNA (modRNA).

An “isolated RNA” is defined as an RNA molecule that may be recombinant or has been isolated from total genomic nucleic acid. An isolated RNA molecule or protein may exist in substantially purified form, or may exist in a non-native environment such as, for example, a host cell.

A “modified RNA” or “modRNA” refers to an RNA molecule having at least one addition, deletion, substitution, and/or alteration of one or more nucleotides as compared to naturally occurring RNA. Such alterations may refer to the addition of non-nucleotide material to internal RNA nucleotides, or to the 5′ and/or 3′ end(s) of RNA. In one aspect, such modRNA contains at least one modified nucleotide, such as an alteration to the base of the nucleotide. For example, a modified nucleotide may replace one or more uridine and/or cytidine nucleotides. For example, these replacements may occur for every instance of uridine and/or cytidine in the RNA sequence, or may occur for only select uridine and/or cytidine nucleotides. Such alterations to the standard nucleotides in RNA may include non-standard nucleotides, such as chemically synthesized nucleotides or deoxynucleotides. For example, at least one uridine nucleotide may be replaced with N1-methylpseudouridine in an RNA sequence. Other such altered nucleotides are known to those of skill in the art. Such altered RNA molecules are considered analogs of naturally-occurring RNA. In some aspects, the RNA is produced by in vitro transcription using a DNA template, where DNA refers to a nucleic acid that contains deoxyribonucleotides. In some aspects, the RNA may be replicon RNA (replicon), in particular self-replicating RNA, or self-amplifying RNA (saRNA).

As contemplated herein, without any limitations, RNA may be used as a therapeutic modality to treat and/or prevent a number of conditions in mammals, including humans. Methods described herein comprise administration of the RNA described herein to a mammal, such as a human. For example, in one aspect such methods of use for RNA include an antigen-coding RNA vaccine to induce robust neutralizing antibodies and accompanying/concomitant T-cell response to achieve protective immunization. In some aspects, minimal vaccine doses are administered to induce robust neutralizing antibodies and accompanying/concomitant T-cell response to achieve protective immunization. In one aspect, the RNA administered is in vitro transcribed RNA. For example, such RNA may be used to encode at least one antigen intended to generate an immune response in said mammal. Pathogenic antigens are peptide or protein antigens derived from a pathogen associated with infectious disease. In specific aspects, the pathogenic are peptide or protein antigens derived from VZV. Conditions and/or diseases that may be treated with RNA disclosed herein include, but are not limited to, those caused and/or impacted by viral infection. Such viruses include, but are not limited to, VZV.

“Prevent” or “prevention,” as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder, or condition has been delayed for a predefined period of time.

As will be understood from context, “risk” of a disease, disorder, and/or condition refers to a likelihood that a particular individual will develop the disease, disorder, and/or condition. In some aspects, risk is expressed as a percentage. In some aspects, risk is, is at least, or is at most from 0, 1, 2, 3, 4, 5, 6, 7, 8, 9, 10, 20, 30, 40, 50, 60, 70, 80, 90 up to 100%. In some aspects risk is expressed as a risk relative to a risk associated with a reference sample or group of reference samples. In some aspects, a reference sample or group of reference samples have a known risk of a disease, disorder, condition and/or event. In some aspects a reference sample or group of reference samples are from individuals comparable to a particular individual. In some aspects, risk may reflect one or more genetic attributes, e.g., which may predispose an individual toward development (or not) of a particular disease, disorder and/or condition. In some aspects, risk may reflect one or more epigenetic events or attributes and/or one or more lifestyle or environmental events or attributes. Susceptible to: An individual who is “susceptible to” a disease, disorder, and/or condition is one who has a higher risk of developing the disease, disorder, and/or condition than does a member of the general public. In some aspects, an individual who is susceptible to a disease, disorder and/or condition may not have been diagnosed with the disease, disorder, and/or condition. In some aspects, an individual who is susceptible to a disease, disorder, and/or condition may exhibit symptoms of the disease, disorder, and/or condition. In some aspects, an individual who is susceptible to a disease, disorder, and/or condition may not exhibit symptoms of the disease, disorder, and/or condition. In some aspects, an individual who is susceptible to a disease, disorder, and/or condition will develop the disease, disorder, and/or condition. In some aspects, an individual who is susceptible to a disease, disorder, and/or condition will not develop the disease, disorder, and/or condition.

The terms “protein,” “polypeptide,” or “peptide” are used herein as synonyms and refer to a polymer of amino acid monomers, e.g., a molecule comprising at least two amino acid residues. Polypeptides may include gene products, naturally occurring polypeptides, synthetic polypeptides, homologs, orthologs, paralogs, fragments and other equivalents, variants, and analogs of the foregoing. Polypeptides may be a single molecule or may be a multi-molecular complex such as a dimer, trimer or tetramer. A protein comprises one or more peptides or polypeptides, and may be folded into a 3-dimensional form, which may be required for the protein to exert its biological function.

As used herein, the term “wild type” or “WT” or “native” refers to the endogenous version of a molecule that occurs naturally in an organism. In some aspects, wild type versions of a protein or polypeptide are employed, however, in other aspects of the disclosure, a modified protein or polypeptide is employed to generate an immune response. The terms described above may be used interchangeably.

A “modified protein” or “modified polypeptide” or a “variant” refers to a protein or polypeptide whose chemical structure, particularly its amino acid sequence, is altered with respect to the wild type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild type activity or function in other respects, such as immunogenicity. Where a protein is specifically mentioned herein, it is in general a reference to a native (wild type) or recombinant (modified) protein. The protein may be isolated directly from the organism of which it is native, produced by recombinant DNA/exogenous expression methods, produced by solid-phase peptide synthesis (SPPS), or other in vitro methods. In particular aspects, there are isolated nucleic acid segments and recombinant vectors incorporating nucleic acid sequences that encode a polypeptide (e.g., an antigen or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

The term “fragment,” with reference to an amino acid sequence (peptide or protein), relates to a part of an amino acid sequence, e.g., a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90%, or at least 99% of the amino acid residues from an amino acid sequence. In the present disclosure, a fragment of a polypeptide, DNA nucleic acid or RNA nucleic acid sequence refers to a sequence having sequence identity of at least, at most, exactly, or between any two of 5%, 10%, 20%, 30%, 40%, 50%, 60%, 70%, 80%, 85%, 86%, 87%, 88%, 89%, 90%, 91%, 92%, 93%, 94%, 95%, 96%, 97%, 98%, or 99% with a polypeptide, DNA nucleic acid or RNA nucleic acid sequence, from which it is derived.

In one aspect, a fragment of a polypeptide, DNA nucleic acid or RNA nucleic acid sequence refers to a sequence having sequence identity of at least 70% with a polypeptide, DNA nucleic acid or RNA nucleic acid sequence, from which it is derived.

In one aspect, a fragment of a polypeptide, DNA nucleic acid or RNA nucleic acid sequence refers to a sequence having sequence identity of at least 80% with a polypeptide, DNA nucleic acid or RNA nucleic acid sequence, from which it is derived.

In one aspect, a fragment of a polypeptide, DNA nucleic acid or RNA nucleic acid sequence refers to a sequence having sequence identity of at least 85% with a polypeptide, DNA nucleic acid or RNA nucleic acid sequence, from which it is derived.

In one aspect, a fragment of a polypeptide, DNA nucleic acid or RNA nucleic acid sequence refers to a sequence having sequence identity of at least 90% with a polypeptide, DNA nucleic acid or RNA nucleic acid sequence, from which it is derived.

In one aspect, a fragment of a polypeptide, DNA nucleic acid or RNA nucleic acid sequence refers to a sequence having sequence identity of at least 95% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 97% with a polypeptide, DNA nucleic acid or RNA nucleic acid sequence, from which it is derived. In one aspect, a fragment of a polypeptide, DNA nucleic acid or RNA nucleic acid sequence refers to a sequence having sequence identity of at least 99% with a polypeptide, DNA nucleic acid or RNA nucleic acid sequence, from which it is derived.

As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some aspects, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. In some aspects, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some aspects, a variant polypeptide or nucleic acid shows an overall sequence identity with a reference polypeptide or nucleic acid that is at least, at most, exactly, or between any two of 85%, 86%, 87%, 88%, 89%, 90%, 91%, 92%, 93%, 94%, 95%, 96%, 97%, or 99%. In some aspects, a variant polypeptide or nucleic acid does not share at least one characteristic sequence element with a reference polypeptide or nucleic acid. In some aspects, a reference polypeptide or nucleic acid has one or more biological activities. In some aspects, a variant polypeptide or nucleic acid shares one or more of the biological activities of the reference polypeptide or nucleic acid. In some aspects, a variant polypeptide or nucleic acid lacks one or more of the biological activities of the reference polypeptide or nucleic acid. In some aspects, a variant polypeptide or nucleic acid shows a reduced level of one or more biological activities as compared to the reference polypeptide or nucleic acid. In some aspects, a polypeptide or nucleic acid of interest is considered to be a “variant” of a reference polypeptide or nucleic acid if it has an amino acid or nucleotide sequence that is identical to that of the reference but for a small number of sequence alterations at particular positions. Preferably, the variant polypeptide or nucleic acid sequence has at least one modification compared to the reference polypeptide or nucleic acid sequence, e.g., from 1 to about 20 modifications. In one aspect, the variant polypeptide or nucleic acid sequence has from 1 to about 10 modifications compared to the reference polypeptide or nucleic acid sequence. In one aspect, the variant polypeptide or nucleic acid sequence has from 1 to about 5 modifications compared to the reference polypeptide or nucleic acid sequence. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. Often, a variant polypeptide or nucleic acid comprises a very small number (e.g., fewer than about 5, about 4, about 3, about 2, or about 1) number of substituted, inserted, or deleted, functional residues (e.g., residues that participate in a particular biological activity) relative to the reference. In some aspects, a variant polypeptide or nucleic acid comprises about 10, about 9, about 8, about 7, about 6, about 5, about 4, about 3, about 2, or about 1 substituted residues as compared to a reference. In some aspects, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some aspects, a variant polypeptide or nucleic acid comprises not more than about 5, about 4, about 3, about 2, or about 1 addition or deletion, and, in some aspects, comprises no additions or deletions, as compared to the reference.

In some aspects, a reference polypeptide or nucleic acid is a “wild type” or “WT” or “native” sequence found in nature, including allelic variations. A wild type polypeptide or nucleic acid sequence has a sequence that has not been intentionally modified. For the purposes of the present disclosure, “variants” of an amino acid sequence (peptide, protein, or polypeptide) comprise amino acid insertion variants, amino acid addition variants, amino acid deletion variants and/or amino acid substitution variants. “Variants” of a nucleotide sequence comprise nucleotide insertion variants, nucleotide addition variants, nucleotide deletion variants and/or nucleotide substitution variants. The term “variant” includes all mutants, splice variants, post-translationally modified variants, conformations, isoforms, allelic variants, species variants, and species homologs, in particular those which are naturally occurring. The term “variant” includes, in particular, fragments of an amino acid or nucleic acid sequence.

Changes may be introduced by mutation into a nucleic acid, thereby leading to changes in the amino acid sequence of a polypeptide (e.g., an antigen or antibody or antibody derivative) that it encodes. Mutations may be introduced using any technique known in the art. In one aspect, one or more particular amino acid residues are changed using, for example, a site-directed mutagenesis protocol. In another aspect, one or more randomly selected residues are changed using, for example, a random mutagenesis protocol. In some aspects, however it is made, a mutant polypeptide may be expressed and screened for a desired property.

Mutations may be introduced into a nucleic acid without significantly altering the biological activity of a polypeptide that it encodes. For example, one may make nucleotide substitutions leading to amino acid substitutions at non-essential amino acid residues. Alternatively, one or more mutations may be introduced into a nucleic acid that selectively changes the biological activity of a polypeptide that it encodes. For example, the mutation may quantitatively or qualitatively change the biological activity. Examples of quantitative changes include increasing, reducing or eliminating the activity. Examples of qualitative changes include altering the antigen specificity of an antibody.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group). In some aspects, percent identity of two sequences is determined using the BLASTN or BLASTP algorithm, as available on the United States National Center for Biotechnology Information (NCBI) website.

Percentage identity is obtained by determining the number of identical positions at which the sequences to be compared correspond, dividing this number by the number of positions compared (e.g., the number of positions in the reference sequence) and multiplying this result by 100.

In some aspects, the degree of similarity or identity is given for a region that is at least, at most, exactly, or between any two of about 50%, about 60%, about 70%, about 80%, about 90%, or about 100% of the entire length of the reference sequence. For example, if the reference nucleic acid sequence consists of 200 nucleotides, the degree of identity is given for at least, at most, exactly, or between any two of about 100, about 120, about 140, about 160, about 180, or about 200 nucleotides, in some aspects, continuous nucleotides. In some aspects, the degree of similarity or identity is given for the entire length of the reference sequence.

Homologous amino acid sequences may exhibit at least, at most, exactly, or between any two of 40%, 50%, 60%, 70%, 80%, 90%, 95%, 98%, or 99% identity of the amino acid residues. In one aspect, homologous amino acid sequences exhibit at least 95% identity of the amino acid residues. In one aspect, homologous amino acid sequences exhibit at least 98% identity of the amino acid residues. In one aspect, homologous amino acid sequences exhibit at least 99% identity of the amino acid residues.

A fragment or variant of an amino acid sequence (peptide or protein) may be a “functional fragment” or “functional variant.” The term “functional fragment” or “functional variant” of an amino acid sequence relates to any fragment or variant exhibiting one or more functional properties identical or similar to those of the amino acid sequence from which it is derived, e.g., it is functionally equivalent. With respect to antigens or antigenic sequences, one particular function is one or more immunogenic activities displayed by the amino acid sequence from which the fragment or variant is derived. The term “functional fragment” or “functional variant,” as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In one aspect, the modifications in the amino acid sequence of the parent molecule or sequence do not significantly affect or alter the characteristics of the molecule or sequence.

An amino acid sequence (peptide, protein, or polypeptide) “derived from” a designated amino acid sequence (peptide, protein, or polypeptide) refers to the origin of the first amino acid sequence. Preferably, the amino acid sequence which is derived from a particular amino acid sequence has an amino acid sequence that is identical, essentially identical, or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

In the present disclosure, a vector refers to a nucleic acid molecule, such as an artificial nucleic acid molecule. A vector may be used to incorporate a nucleic acid sequence, such as a nucleic acid sequence comprising an open reading frame. Vectors include, but are not limited to, storage vectors, expression vectors, cloning vectors, transfer vectors. A vector may be an RNA vector or a DNA vector. In some aspects the vector is a DNA molecule. In some aspects, the vector is a plasmid vector. In some aspects, the vector is a viral vector. Typically, an expression vector will contain a desired coding sequence and appropriate other sequences necessary for the expression of the operably linked coding sequence in a particular host organism (e.g., bacteria, yeast, plant, insect, or mammal) or in in vitro expression systems. Cloning vectors are generally used to engineer and amplify a certain desired fragment (typically a DNA fragment), and may lack functional sequences needed for expression of the desired fragment(s).

As used herein, the term “pharmaceutical composition” refers to an active agent, formulated together with one or more pharmaceutically acceptable carriers. Pharmaceutical compositions may be immunogenic compositions. In some aspects, active agent is present in unit dose amount appropriate for administration in a therapeutic regimen that shows a statistically significant probability of achieving a predetermined therapeutic effect when administered to a relevant population. In some aspects, pharmaceutical compositions may be specially formulated for parenteral administration, for example, by subcutaneous, intramuscular, intravenous or epidural injection as, for example, a sterile solution or suspension, or sustained-release formulation.

As used herein, the term “vaccination” refers to the administration of an immunogenic composition intended to generate an immune response, for example to a disease-associated (e.g., disease-causing) agent (e.g., a virus). In some aspects, vaccination may be administered before, during, and/or after exposure to a disease-associated agent, and in certain aspects, before, during, and/or shortly after exposure to the agent. In some aspects, vaccination includes multiple administrations, appropriately spaced in time, of a vaccine composition. In some aspects, vaccination generates an immune response to an infectious agent. In some aspects, vaccination generates an immune response to a tumor; in some such aspects, vaccination is “personalized” in that it is partly or wholly directed to epitope(s) (e.g., which may be or include one or more neoepitopes) determined to be present in a particular individual's tumors.

An immune response refers to a humoral response, a cellular response, or both a humoral and cellular response in an organism. An immune response may be measured by assays that include, but are not limited to, assays measuring the presence or amount of antibodies that specifically recognize a protein or cell surface protein, assays measuring T-cell activation or proliferation, and/or assays that measure modulation in terms of activity or expression of one or more cytokines.

As used herein, the term “combination therapy” refers to those situations in which a subject is simultaneously exposed to two or more therapeutic regimens (e.g., two or more therapeutic agents). In some aspects, the two or more regimens may be administered simultaneously; in some aspects, such regimens may be administered sequentially (e.g., all “doses” of a first regimen are administered prior to administration of any doses of a second regimen); in some aspects, such agents are administered in overlapping dosing regimens. In some aspects, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some aspects, two or more agents, or active moieties thereof, may be administered together in a combination composition, or even in a combination compound (e.g., as part of a single chemical complex or covalent entity).

Those skilled in the art will appreciate that the term “dosing regimen” may be used to refer to a set of unit doses (typically more than one) that are administered individually to a subject, typically separated by periods of time. In some aspects, a given therapeutic agent has a recommended dosing regimen, which may involve one or more doses. In some aspects, a dosing regimen comprises a plurality of doses each of which is separated in time from other doses. In some aspects, individual doses are separated from one another by a time period of the same length; in some aspects, a dosing regimen comprises a plurality of doses and at least two different time periods separating individual doses. In some aspects, all doses within a dosing regimen are of the same unit dose amount. In some aspects, different doses within a dosing regimen are of different amounts. In some aspects, a dosing regimen comprises a first dose in a first dose amount, followed by one or more additional doses in a second dose amount different from the first dose amount. In some aspects, a dosing regimen comprises a first dose in a first dose amount, followed by one or more additional doses in a second dose amount same as the first dose amount. In some aspects, a dosing regimen is correlated with a desired or beneficial outcome when administered across a relevant population (e.g., is a therapeutic dosing regimen).

### II. Varicella Zoster Virus (VZV)

The present disclosure provides for RNA molecules (e.g., RNA polynucleotides) comprising at least one open reading frame encoding a varicella-zoster virus (VZV) polypeptide. The present disclosure further provides for an immunogenic composition comprising at least one RNA molecule encoding a VZV polypeptide complexed with, encapsulated in, or formulated with one or more lipids, and forming lipid nanoparticles (LNPs).

Varicella-zoster virus (VZV), also known as human herpesvirus 3 (HHV-3), is a human pathogen that causes varicella or chicken pox in children and re-emerges later as herpes zoster or shingles. VZV has an inner capsid that surrounds a linear double stranded DNA genome. Surrounding the capsid is a tegument layer with glycoproteins, and the outermost layer is a lipid-rich envelope with glycoproteins. Glycoproteins have a variety of functions, from DNA replication or capsid assembly to interacting with cell surface molecules and assisting with fusion into the plasma membrane. For example, glycoprotein E is an integral membrane protein thought to be important for T-cell infection and cell-to-cell spread of the virus. VZV shows tropism for neurons and T cells.

Upon primary infection with VZV (e.g., varicella or “chickenpox”), VZV establishes latency in sensory ganglia. VZV-specific T cells are needed to clear the primary infection and prevent reactivation. The mechanisms of reactivation are unknown, but VZV cell-mediated immunity is thought to play a role. Deficiencies in cell-mediate immunity (e.g., advanced age, immunocompromising conditions) are risk factors for reactivation. Reactivation allows for VZV replication and transport to the skin, possibly manifesting as herpes zoster (HZ).

Herpes zoster most commonly manifests as a unilateral vesicular rash with pain that is typically restricted to one dermatome or to several contiguous dermatomes. Within days of onset of the rash, grouped vesicles, bullae, or pustules may develop; these lesions contain VZV and are considered to be infectious. Characteristic pain of herpes zoster includes sensations of burning or numbness, pruritis, or allodynia. Many people develop prodromal pain 2-3 days before the rash appears. Among immunocompetent persons, lesions crust in 7-10 days and are no longer infectious once crusted.

The most common complication of herpes zoster is postherpetic neuralgia (PHN), and up to 15% of persons with herpes zoster develop PHN. PHN is significant pain in the area affected by herpes zoster after crusting of the rash. Older age and prodromal symptoms are considered to be risk factors for PHN. Other complications of herpes zoster include ocular complications (herpes zoster opthalmicus or keratitis, acute retinal necrosis), neurologic complications (herpes zoster oticus, meningitis, encephalitis, myelitis, peripheral motor neuropathy, Guillan-Barré syndrome, and stroke), and secondary bacterial skin and soft tissue infections.

The VZV genome encodes at least 71 unique proteins (ORF0-ORF68) with three more opening reading frames (ORF69-ORF71) that duplicate earlier open reading frames (ORF64-62, respectively). Encoded proteins form the structure of the virus particle, including nine glycoproteins: ORF5 (gK), ORF9A (gN), ORF14 (gC), ORF31 (gB), ORF37 (gH), ORF50 (gM), ORF60 (gL), ORF67 (gI), and ORF68 (gE). The encoded glycoproteins gE, gI, gB, gH, gK, gL, gC, gN, and gM function in different steps of the viral replication cycle. The most abundant glycoprotein found in infected cells, as well as in the mature virion, is glycoprotein E (gE, ORF 68), which is a major component of the virion envelope and is essential for viral replication. Glycoprotein I (gI, ORG 67) forms a complex with gE in infected cells, which facilitates the endocytosis of both glycoproteins and directs them to the trans-Golgi network (TGN) where the final viral envelope is acquired. VZV gE is a 623-amino-acid type I membrane protein encoded by open reading frame 68 (ORF68) and is the most abundant viral glycoprotein expressed on the surface of VZV-infected cells. Glycoprotein I (gI) is required within the TGN for VZV envelopment and for efficient membrane fusion during VZV replication. VZV gE and gI are found complexed together on the infected host cell surface. Glycoprotein B (ORF 31), which is the second most prevalent glycoprotein and thought to play a role in virus entry, binds to neutralizing antibodies. Glycoprotein H is thought to have a fusion function facilitating cell to cell spread of the virus. Antibodies to gE, gB, and gH are prevalent after natural infection and following vaccination and have been shown to neutralize viral activity in vitro. As used herein, the term “varicella-zoster virus” or “VZV” is not limited to any particular strain or variant.

In some aspects, the RNA molecule comprises an open reading frame encoding a VZV antigen. In some aspects, the VZV antigen is a VZV polypeptide. In some aspects, the VZV polypeptide is a VZV glycoprotein (e.g. gK, gN, gC, gB, gH, gM, gL gI and gE) or a fragment or a variant thereof. In some aspects, the RNA molecule encodes a VZV gK polypeptide, the RNA molecule encodes a VZV gN polypeptide, the RNA molecule encodes a VZV gC polypeptide, the RNA molecule encodes a VZV gB polypeptide, the RNA molecule encodes a VZV gH polypeptide, the RNA molecule encodes a VZV gM polypeptide, the RNA molecule encodes a VZV gL polypeptide, the RNA molecule encodes a VZV gI polypeptide, and/or the RNA molecule encodes a VZV gE polypeptide. In a one aspect, the RNA molecule encodes a VZV gE polypeptide. In some aspects, the VZV polypeptide comprises two or more (e.g., 2, 3, 4, 5, 6, 7, 8, 9, or more) VZV polypeptides.

In some aspects, the VZV polypeptide is a full-length VZV polypeptide. In some aspects, the VZV polypeptide is a truncated VZV polypeptide. In some aspects, the VZV polypeptide is a variant of a VZV polypeptide. In some aspects, the VZV polypeptide is a fragment of a VZV polypeptide.

In some aspects, the VZV polypeptide is a full-length gK polypeptide. In some aspects, the VZV polypeptide is a truncated VZV gK polypeptide. In some aspects, the VZV polypeptide is a variant of a VZV gK polypeptide. In some aspects, the VZV polypeptide is a fragment of a VZV gK polypeptide.

In some aspects, the VZV polypeptide is a full-length gN polypeptide. In some aspects, the VZV polypeptide is a truncated VZV gN polypeptide. In some aspects, the VZV polypeptide is a variant of a VZV gN polypeptide. In some aspects, the VZV polypeptide is a fragment of a VZV gN polypeptide.

In some aspects, the VZV polypeptide is a full-length gC polypeptide. In some aspects, the VZV polypeptide is a truncated VZV gC polypeptide. In some aspects, the VZV polypeptide is a variant of a VZV gC polypeptide. In some aspects, the VZV polypeptide is a fragment of a VZV gC polypeptide.

In some aspects, the VZV polypeptide is a full-length gB polypeptide. In some aspects, the VZV polypeptide is a truncated VZV gB polypeptide. In some aspects, the VZV polypeptide is a variant of a VZV gB polypeptide. In some aspects, the VZV polypeptide is a fragment of a VZV gB polypeptide.

In some aspects, the VZV polypeptide is a full-length gH polypeptide. In some aspects, the VZV polypeptide is a truncated VZV gH polypeptide. In some aspects, the VZV polypeptide is a variant of a VZV gH polypeptide. In some aspects, the VZV polypeptide is a fragment of a VZV gH polypeptide.

In some aspects, the VZV polypeptide is a full-length gM polypeptide. In some aspects, the VZV polypeptide is a truncated VZV gM polypeptide. In some aspects, the VZV polypeptide is a variant of a VZV gM polypeptide. In some aspects, the VZV polypeptide is a fragment of a VZV gM polypeptide.

In some aspects, the VZV polypeptide is a full-length gL polypeptide. In some aspects, the VZV polypeptide is a truncated VZV gL polypeptide. In some aspects, the VZV polypeptide is a variant of a VZV gL polypeptide. In some aspects, the VZV polypeptide is a fragment of a VZV gL polypeptide.

In some aspects, the VZV polypeptide is a full-length gI polypeptide. In some aspects, the VZV polypeptide is a truncated VZV gI polypeptide. In some aspects, the VZV polypeptide is a variant of a VZV gI polypeptide. In some aspects, the VZV polypeptide is a fragment of a VZV gI polypeptide.

In some aspects, the VZV polypeptide is a full-length gE polypeptide. In some aspects, the VZV polypeptide is a truncated VZV gE polypeptide. In some aspects, the VZV polypeptide is a variant of a VZV gE polypeptide. In some aspects, the VZV polypeptide is a fragment of a VZV gE polypeptide.

In some aspects, the VZV polypeptide comprises at least one mutation. In some aspects, the VZV polypeptide is a VZV gK polypeptide comprising at least one mutation. In some aspects, the VZV polypeptide is a VZV gN polypeptide comprising at least one mutation. In some aspects, the VZV polypeptide is a VZV gC polypeptide comprising at least one mutation. In some aspects, the VZV polypeptide is a VZV gB polypeptide comprising at least one mutation. In some aspects, the VZV polypeptide is a VZV gH polypeptide comprising at least one mutation. In some aspects, the VZV polypeptide is a VZV gM polypeptide comprising at least one mutation. In some aspects, the VZV polypeptide is a VZV gL polypeptide comprising at least one mutation. In some aspects, the VZV polypeptide is a VZV gI polypeptide comprising at least one mutation. In some aspects, the VZV polypeptide is a VZV gE polypeptide comprising at least one mutation.

In some aspects, the RNA molecule encodes a VZV gE polypeptide comprising the amino acid sequence according to any one of GENBANK® Accession No.: AAG32558.1, ABE03086.1, AAK01047.1, Q9J3M8.1, AEW88548.1, AGY33616.1, AEW89124.1. AIT53150.1, CAA25033.1, NP_040190.1, AKG56356.1, AEW89412.1, ABF21714.1, ABF21714.1, AAT07749.1, AEW88764.1, AAG48520.1, and/or AEW88980.1, or fragment or variant thereof, the respective sequences of which are herein incorporated by reference. In some aspects, the RNA molecule encodes a VZV gE polypeptide comprising the amino acid sequence according to GENBANK® Accession No. AH009994.2 (ORF68), or fragment or variant thereof, the sequence of which is herein incorporated by reference.

In some aspects, the RNA molecule encodes a VZV polypeptide of Table 1 (see Example 7). In some aspects, the RNA molecule encodes a VZV gE polypeptide comprising an amino acid sequence of any of SEQ ID NO: 1 to 11, or fragment or variant thereof. In some aspects, VZV gE polypeptide may have at least, at most, exactly, or between any two of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to any of the amino acid sequences of Table 1, for example, any of SEQ ID NO: 1 to 11. In some aspects, VZV gE polypeptide consists of any of the amino acid sequences of Table 1, for example, any of SEQ ID NO: 1 to 11.

In some aspects, the RNA molecule sequence is transcribed from a DNA nucleic acid sequence (DNA polynucleotide) of Table 2 (see Example 7). In some aspects, the RNA molecule comprises an ORF transcribed from a nucleic acid sequence of any of SEQ ID NO: 12 to 145, or fragment or variant thereof. In some aspects, the RNA molecule comprises an ORF transcribed from a nucleic acid sequence that may have at least, at most, exactly, or between any two of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to any of the nucleic sequences of Table 2, for example, any of SEQ ID NO: 12 to 145. In some aspects, the RNA molecule comprises an ORF transcribed from a nucleic acid sequence that consists of any of the nucleic sequences of Table 2, for example, any of SEQ ID NO: 12 to 145.

In some aspects, the RNA molecule comprises an ORF comprising an RNA nucleic acid sequence (RNA polynucleotide) of Table 3 (see Example 7). In some aspects, the RNA molecule comprises an ORF comprising a nucleic acid sequence of any of SEQ ID NO: 146 to 279, or fragment or variant thereof. In some aspects, the RNA molecule comprises an ORF comprising a nucleic acid sequence that may have at least, at most, exactly, or between any two of 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to any of the RNA nucleic acid sequences of Table 3, for example, any of SEQ ID NO: 146 to 279. In some aspects, the RNA molecule comprises an ORF comprising a nucleic acid sequence that consists of any of the RNA nucleic acid sequences of Table 3, for example, any of SEQ ID NO: 146 to 279.

In some aspects, the RNA molecule comprises stabilized RNA. In some aspects, the RNA molecule comprises a nucleic acid sequence having at least one uridine replaced by N1-methylpseudouridine. In some aspects, the RNA molecule comprises a sequence having all uridines replaced by N1-methylpseudouridine (designated as “ψ”). In some aspects, the RNA molecule comprises an ORF comprising a nucleic acid sequence of any of SEQ ID NO: 146 to 279, wherein all uridines have been replaced by N1-methylpseudouridine (designated as “ψ”).

In some aspects, the RNA molecule comprises an open reading frame encoding a VZV polypeptide amino acid sequence that may be at least, at most, exactly, or between any two of 70%, 80%, 85%, 86%, 87%, 88%, 89%, 90%, 91%, 92%, 93%, 94%, 95%, 96%, 97%, 98%, or 99% identical to any of the VZV polypeptide sequences of SEQ ID NO: 1 to 11 (Table 1) or other VZV polypeptide described herein. In some aspects, the RNA molecule comprises an open reading frame encoding a VZV polypeptide amino acid sequence that consists of any of the VZV polypeptide sequences of SEQ ID NO: 1 to 11 (Table 1) or other VZV polypeptide described herein.

In some aspects, the RNA molecule comprises an open reading frame transcribed from a DNA nucleic acid sequence that may be at least, at most, exactly, or between any two of 70%, 80%, 85%, 86%, 87%, 88%, 89%, 90%, 91%, 92%, 93%, 94%, 95%, 96%, 97%, 98%, or 99% identical to any of the nucleic acid sequences of SEQ ID NO: 12 to 145 (Table 2) or other nucleic acid described herein. In some aspects, the RNA molecule comprises an open reading frame transcribed from a DNA nucleic acid sequence that consists of any of the nucleic acid sequences of SEQ ID NO: 12 to 145 (Table 2) or other nucleic acid described herein.

In some aspects, the RNA molecule comprises an open reading frame comprising an RNA nucleic acid sequence that may be at least, at most, exactly, or between any two of 70%, 80%, 85%, 86%, 87%, 88%, 89%, 90%, 91%, 92%, 93%, 94%, 95%, 96%, 97%, 98%, or 99% identical to any of the nucleic acid sequences of SEQ ID NO: 146 to 279 (Table 3) or other nucleic acid described herein. In some aspects, the RNA molecule comprises an open reading frame comprising an RNA nucleic acid sequence that consists of any of the nucleic acid sequences of SEQ ID NO: 146 to 279 (Table 3) or other nucleic acid described herein. In some aspects, the RNA molecule comprises an ORF comprising a nucleic acid sequence of any of SEQ ID NO: 146 to 279 (Table 3), wherein all uridines have been replaced by N1-methylpseudouridine (designated as “ψ”).

### III. RNA Molecule

In some aspects, the RNA molecule described herein is a coding RNA molecule. Coding RNA includes a functional RNA molecule that may be translated into a peptide or polypeptide. In some aspects, the coding RNA molecule includes at least one open reading frame (ORF) coding for at least one peptide or polypeptide. An open reading frame comprises a sequence of codons that is translatable into a peptide or protein. The coding RNA molecule may include one (monocistronic), two (bicistronic) or more (multicistronic) OFRs, which may be a sequence of codons that is translatable into a polypeptide or protein of interest.

The coding RNA molecule may be a messenger RNA (mRNA) molecule, viral RNA molecule, or self-amplifying RNA molecule (saRNA, also referred to as a replicon). In some aspects, the RNA molecule is an mRNA. Preferably, the RNA molecule of the present disclosure is an mRNA. In some aspects, the RNA molecule is a saRNA. In some aspects, the saRNA molecule may be a coding RNA molecule.

The RNA molecule may encode one polypeptide of interest or more, such as an antigen or more than one antigen, e.g., two, three, four, five, six, seven, eight, nine, ten or more polypeptides. Alternatively, or in addition, one RNA molecule may also encode more than one polypeptide of interest, such as an antigen, e.g., a bicistronic, or tricistronic RNA molecule that encodes different or identical antigens.

The sequence of the RNA molecule may be codon optimized or deoptimized for expression in a desired host, such as a human cell. In some aspects, a gene of interest (e.g., an antigen) described herein is encoded by a coding sequence which is codon-optimized and/or the guanosine/cytidine (G/C) content of which is increased compared to wild type coding sequence. In some aspects, one or more sequence regions of the coding sequence are codon-optimized and/or increased in the G/C content compared to the corresponding sequence regions of the wild type coding sequence. In some aspects, codon-optimization and/or increasing the G/C content does not change the sequence of the encoded amino acid sequence.

The term “codon-optimized” is understood by those in the art to refer to alteration of codons in the coding region of a nucleic acid molecule to reflect the typical codon usage of a host organism without altering the amino acid sequence encoded by the nucleic acid molecule. Within the context of the present disclosure, in some aspects, coding regions are codon-optimized for optimal expression in a subject to be treated using an RNA polynucleotide described herein. Codon-optimization is based on the finding that the translation efficiency is also determined by a different frequency in the occurrence of tRNA molecules in cells. Thus, the sequence of RNA may be modified such that codons for which frequently occurring tRNA molecules are available are inserted in place of “rare codons.”

In some aspects, G/C content of a coding region (e.g., of a gene of interest sequence; open reading frame (ORF)) of an RNA is increased compared to the G/C content of the corresponding coding sequence of a wild type RNA encoding the gene of interest, wherein in some aspects, the amino acid sequence encoded by the RNA is not modified compared to the amino acid sequence encoded by the wild type RNA. This modification of the RNA sequence is based on the fact that the sequence of any RNA region to be translated is important for efficient translation of that mRNA. Sequences having an increased G (guanosine)/C (cytidine) content are more stable than sequences having an increased A (adenosine)/U (uridine) content. In respect to the fact that several codons code for one and the same amino acid (so-called degeneration of the genetic code), the most favorable codons for the stability may be determined (so-called alternative codon usage). Depending on the amino acid to be encoded by the RNA, there are various possibilities for modification of the RNA sequence, compared to its wild type sequence. In particular, codons which contain A and/or U nucleosides may be modified by substituting these codons by other codons, which code for the same amino acids but contain no A and/or U or contain a lower content of A and/or U nucleosides. Thus, in some aspects, G/C content of a coding region of an RNA described herein is increased by at least, at most, exactly, or between any two of 10%, 20%, 30%, 40%, 50%, 55%, or even more compared to the G/C content of a coding region of a wild type RNA. In some aspects, the coding region of the VZV RNA described herein comprises a G/C content of at least 50%, at least 55%, at least 60%, at least 65%, at least 70%, at least 75%, or about 80%. In some aspects, the coding region of the VZV RNA described herein comprises a G/C content of about 50% to 75%, about 55% to 70%, about 50% to 60%, about 60% to 70%, about 70% to 80%, about 50% to 55%, about 55% to 60%, about 60% to 65%, about 65% to 70%, about 70% to 75%, or about 75% to 80%. In some aspects, the coding region of the VZV RNA described herein comprises a G/C content of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, or about 75%. In some aspects, the coding region of the VZV RNA described herein comprises a G/C content of about 58%, about 66% or about 62%.

In some aspects, the RNA molecule includes from about 20 to about 100,000 nucleotides (e.g., from 30 to 50, from 30 to 100, from 30 to 250, from 30 to 500, from to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

In some aspects, the RNA molecule has at least, at most, exactly, or between any two of about 20, 40, 60, 80, 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, 9000, 9200, 9400, 9600, 9800, 10000, 10000, 12000, 14000, 16000, 18000, 20000, 22000, 24000, 26000, 28000, 30000, 32000, 34000, 36000, 38000, 40000, 42000, 44000, 46000, 48000, 50000, 52000, 54000, 56000, 58000, 60000, 62000, 64000, 66000, 68000, 70000, 72000, 74000, 76000, 78000, 80000, 82000, 84000, 86000, 88000, 90000, 92000, 94000, 96000, 98000, or 100000 nucleotides.

In some aspects, the RNA molecule includes at least 100 nucleotides. For example, in some aspects, the RNA has a length between 100 and 15,000 nucleotides; between 7,000 and 16,000 nucleotides; between 8,000 and 15,000 nucleotides; between 9,000 and 12,500 nucleotides; between 11,000 and 15,000 nucleotides; between 13,000 and 16,000 nucleotides; between 7,000 and 25,000 nucleotides. In some aspects, the RNA molecule has at least, at most, exactly, or between any two of about 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000, 4050, 4100, 4150, 4200, 4250, 4300, 4350, 4400, 4450, 4500, 4550, 4600, 4650, 4700, 4750, 4800, 4850, 4900, 4950, 5000, 5050, 5100, 5150, 5200, 5250, 5300, 5350, 5400, 5450, 5500, 5550, 5600, 5650, 5700, 5750, 5800, 5850, 5900, 5950, 6000, 6050, 6100, 6150, 6200, 6250, 6300, 6350, 6400, 6450, 6500, 6550, 6600, 6650, 6700, 6750, 6800, 6850, 6900, 6950, 7000, 7050, 7100, 7150, 7200, 7250, 7300, 7350, 7400, 7450, 7500, 7550, 7600, 7650, 7700, 7750, 7800, 7850, 7900, 7950, 8000, 8050, 8100, 8150, 8200, 8250, 8300, 8350, 8400, 8450, 8500, 8550, 8600, 8650, 8700, 8750, 8800, 8850, 8900, 8950, 9000, 9050, 9100, 9150, 9200, 9250, 9300, 9350, 9400, 9450, 9500, 9550, 9600, 9650, 9700, 9750, 9800, 9850, 9900, 9950, 10000, 10050, 10100, 10150, 10200, 10250, 10300, 10350, 10400, 10450, 10500, 10550, 10600, 10650, 10700, 10750, 10800, 10850, 10900, 10950, 11000, 11050, 11100, 11150, 11200, 11250, 11300, 11350, 11400, 11450, 11500, 11550, 11600, 11650, 11700, 11750, 11800, 11850, 11900, 11950, 12000, 12050, 12100, 12150, 12200, 12250, 12300, 12350, 12400, 12450, 12500, 12550, 12600, 12650, 12700, 12750, 12800, 12850, 12900, 12950, 13000, 13050, 13100, 13150, 13200, 13250, 13300, 13350, 13400, 13450, 13500, 13550, 13600, 13650, 13700, 13750, 13800, 13850, 13900, 13950, 14000, 14050, 14100, 14150, 14200, 14250, 14300, 14350, 14400, 14450, 14500, 14550, 14600, 14650, 14700, 14750, 14800, 14850, 14900, 14950, or 15000 nucleotides.

In some aspects of the present disclosure, an RNA is or comprises messenger RNA (mRNA) that relates to an RNA transcript which encodes a polypeptide. In some aspects, an RNA disclosed herein comprises: a 5′ cap comprising a 5′ cap disclosed herein; a 5′ untranslated region comprising a cap proximal sequence (5′ UTR), a sequence encoding a protein (e.g., a polypeptide); a 3′ untranslated region (3′ UTR); and/or a polyadenylate (Poly A) sequence.

In some aspects, an RNA disclosed herein comprises the following components in 5′ to 3′ orientation: a 5′ cap comprising a 5′ cap disclosed herein; a 5′ untranslated region comprising a cap proximal sequence (5′ UTR), a sequence encoding a protein (e.g., a polypeptide); a 3′ untranslated region (3′ UTR); and a Poly-A sequence.

A. Modified Nucleobases

In the present disclosure the RNA molecules may comprise modified nucleobases which may be incorporated into modified nucleosides and nucleotides. In some aspects, the RNA molecule may include one or more modified nucleotides. Naturally occurring nucleotide modifications are known in the art.

In some aspects, the RNA molecule may include a modified nucleotide. Non-limiting examples of modified nucleotides that may be included in the RNA molecule include pseudouridine, N1-methylpseudouridine, 5-methyluridine, 3-methyl-uridine, 5-methoxy-uridine, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), uridine 5-oxyacetic acid, uridine 5-oxyacetic acid methyl ester, 5-carboxymethyl-uridine, 1-carboxymethyl-pseudouridine, 5-carboxy hydroxymethyl-uridine, 5-carboxy hydroxy methyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 1-ethyl-pseudouridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-2-thio-uridine, 1-methyl-4-thio-pseudouridine, 4-thio-1-methyl-pseudouridine, 3-methyl-1-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine, 1-methyl-3-(3-amino-3-carboxypropyl)pseudouridine, 5-(isopentenylaminomethyl)uridine, 5-(isopentenylaminomethyl)-2-thio-uridine, a-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, any other modified uridine known in the art, or combinations thereof.

In some aspects of the present disclosure, modified nucleotides include any one of N1-methylpseudouridine or pseudouridine.

In some aspects, the RNA molecule comprises nucleotides that are N1-methylpseudouridine modified. In some aspects, the RNA molecule comprises nucleotides that are a pseudouridine modified.

In some aspects, an RNA comprises a modified nucleoside in place of at least one uridine. In some aspects, an RNA comprises a modified nucleoside in place of each uridine. In some aspects, the RNA molecule comprises a sequence having at least one uridine replaced by N1-methylpseudouridine. In some aspects, the RNA molecule comprises a sequence having all uridines replaced by N1-methylpseudouridine. N1-methylpseudouridine is designated in sequences as “ψ”. The term “uracil,” as used herein, describes one of the nucleobases that may occur in the nucleic acid of RNA. The term “uridine,” as used herein, describes one of the nucleosides that may occur in RNA. “Pseudouridine” is one example of a modified nucleoside that is an isomer of uridine, where the uracil is attached to the pentose ring via a carbon-carbon bond instead of a nitrogen-carbon glycosidic bond.

In some aspects, the RNA molecule comprises a nucleic acid sequence having at least one uridine replaced by N1-methylpseudouridine or pseudouridine. In some aspects, the RNA molecule comprises a nucleic acid sequence having at least, at most, exactly, or between any two of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of uridines replaced by N1-methylpseudouridine or pseudouridine. In some aspects, the RNA molecule comprises a nucleic acid sequence having all uridines replaced by N1-methylpseudouridine or pseudouridine.

Modifications that may be present in the RNA molecules further include, for example, m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); mil (1-methylinosine); m′Im (1,2′-O-dimethylinosine); m3C (3-methylcytidine); Cm (2T-O-methylcytidine); s2C (2-thiocytidine); ac4C (N4-acetylcytidine); f5C (5-formylcytosine); m5Cm (5,2-O-dimethylcytidine); ac4Cm (N4acetyl2TOmethylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-Omethyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-O-dimethyladenosine); rn62Am (N6,N6,O-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C1-C6)-alkyluracil, 5-methyluracil, 5-(C2-Ce)-alkenyluracil, 5-(C2-Ce)-alkynyluracil, 5-(hydroxymethyl)uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U.

In some aspects, the RNA molecule may include phosphoramidate, phosphorothioate, and/or methylphosphonate linkages.

The sequence of the RNA molecule may be modified if desired, for example to increase the efficacy of expression or replication of the RNA, or to provide additional stability or resistance to degradation. For example, the RNA sequence may be modified with respect to its codon usage, for example, to increase translation efficacy and half-life of the RNA.

In some aspects, the RNA molecule of the present disclosure comprises an open reading frame having at least one codon modified sequence. A codon modified sequence relates to coding sequences that differ in at least one codon (triplets of nucleotides coding for one amino acid) compared to the corresponding wild type coding sequence. A codon modified sequence may show improved resistance to degradation, improved stability, and/or improved translatability.

The sequence of the RNA molecule may be codon optimized or deoptimized for expression in a desired host, such as a human cell.

In some aspects, the RNA molecules may include one or more structural and/or chemical modifications or alterations which impart useful properties to the polynucleotide including, in some aspects, the lack of a substantial induction of the innate immune response of a cell into which the polynucleotide is introduced. As used herein, a “structural” feature or modification is one in which two or more linked nucleotides are inserted, deleted, duplicated, inverted or randomized in an RNA molecule without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In some aspects, the RNA molecule may include one or more modified nucleotides in addition to any 5′ cap structure. Naturally occurring nucleotide modifications are known in the art.

In some aspects, the RNA molecule does not include modified nucleotides, e.g., does not include modified nucleobases, and all of the nucleotides in the RNA molecule are conventional standard ribonucleotides A, U, G and C, with the exception of an optional 5′ cap that may include, for example, 7-methylguanosine, which is further described below. In some aspects, the RNA may include a 5′ cap comprising a 7′-methylguanosine, and the first 1, 2 or 3 5′ ribonucleotides may be methylated at the 2′ position of the ribose.

In some aspects, the RNA molecule described herein is a non-coding RNA molecule. A non-coding RNA (ncRNA) molecule includes a functional RNA molecule that is not translated into a peptide or polypeptide. Non-coding RNA molecules may include highly abundant and functionally important RNA molecules. In some aspects, the non-coding RNA is a functional mRNA molecule that is not translated into a peptide or polypeptide. The non-coding RNA may include modified nucleotides as described herein. Preferably, the RNA molecule is an mRNA

The RNA molecules of the present disclosure may be prepared by any method know in the art, including chemical synthesis and in vitro methods, such as RNA in vitro transcription. In some of the aspects, the RNA of the present disclosure is prepared using in vitro transcription.

In some aspects, the RNA molecule of the present disclosure is purified, e.g., such as by filtration that may occur via, e.g., ultrafiltration, diafiltration, or, e.g., tangential flow ultrafiltration/diafiltration.

In some aspects, the RNA molecule of the present disclosure is lyophilized to be temperature stable.

B. 5′ CAP

In some aspects, the RNA molecule described herein includes a 5′ cap which generally “caps” the 5′ end of the RNA and stabilizes the RNA molecule.

In some aspects, the 5′ cap moiety is a natural 5′ cap. A “natural 5′ cap” is defined as a cap that includes 7-methylguanosine connected to the 5′ end of an mRNA molecule through a 5′ to 5′ triphosphate linkage. In some aspects, a guanosine nucleoside included in a 5′ cap may be modified, for example, by methylation at one or more positions (e.g., at the 7-position) on a base (guanine), and/or by methylation at one or more positions of a ribose. In some aspects, a guanosine nucleoside included in a 5′ cap comprises a 3′O methylation at a ribose (3′OMeG). In some aspects, a guanosine nucleoside included in a 5′ cap comprises methylation at the 7-position of guanine (m7G). In some aspects, a guanosine nucleoside included in a 5′ cap comprises methylation at the 7-position of guanine and a 3′O methylation at a ribose (m7(3′OMeG)). The 5′ cap may be incorporated during RNA synthesis (e.g., co-transcriptional capping) or may be enzymatically engineered after RNA transcription (e.g., post-transcriptional capping). In some aspects, co-transcriptional capping with a cap disclosed herein improves the capping efficiency of an RNA compared to co-transcriptional capping with an appropriate reference comparator. In some aspects, improving capping efficiency may increase a translation efficiency and/or translation rate of an RNA, and/or increase expression of an encoded polypeptide. In some aspects, capping is performed after purification, e.g., tangential flow filtration, of the RNA molecule.

In some aspects, an RNA described herein comprises a 5′ cap or a 5′ cap analog, e.g., a Cap 0, a Cap 1 or a Cap 2. In some aspects, a provided RNA does not have uncapped 5′-triphosphates. In some aspects, the 5′ end of the RNA is capped with a modified ribonucleotide. In some aspects, the 5′ cap moiety is a 5′ cap analog. In some aspects, an RNA may be capped with a 5′ cap analog. Cap structures include, but are not limited to, 7mG(5′)ppp(5′)N,pN2p (Cap 0) and 7mG(5′)ppp(5′)N1mpNp (Cap 1). In some aspects, an RNA described herein comprises a Cap 0. Cap 0 is a N7-methyl guanosine connected to the 5′ nucleotide through a 5′ to 5′ triphosphate linkage, typically referred to as m7G cap or m7Gppp. In the cell, the Cap 0 structure is essential for efficient translation of the mRNA that carries the cap. An additional methylation on the 2′O position of the initiating nucleotide generates Cap 1, or referred to as m7GpppNm, wherein Nm denotes any nucleotide with a 2′O methylation. In some aspects, an RNA described herein comprises a Cap 1, e.g., as described herein. In some aspects, an RNA described herein comprises a Cap 2.

In some aspects, a Cap 0 structure comprises a guanosine nucleoside methylated at the 7-position of guanine (m7G). In some aspects, a Cap 0 structure is connected to an RNA via a 5′ to 5′-triphosphate linkage and is also referred to herein as m7Gppp or m7G(5′)ppp(5′). A 5′ cap may be methylated with the structure m7G (5′) ppp (5′) N (cap-0 structure) or a derivative thereof, wherein N is the terminal 5′ nucleotide of the nucleic acid carrying the 5′ cap, typically the 5′-end of an mRNA. An exemplary enzymatic reaction for capping may include use of Vaccinia Virus Capping Enzyme (VCE) that includes mRNA triphosphatase, guanylyl-transferase and guanine-7-methytransferase, which catalyzes the construction of N7-monomethylated Cap 0 structures. Cap 0 structure plays an important role in maintaining the stability and translational efficacy of the RNA molecule.

The 5′ cap of the RNA molecule may be further modified by a 2′-O-Methyltransferase which results in the generation of a Cap 1 structure (m7Gppp [m2′-O] N), which may further increase translation efficacy. In some aspects, a Cap 1 structure comprises a guanosine nucleoside methylated at the 7-position of guanine (m7G) and a 2′O methylated first nucleotide in an RNA (2′OmeN₁). In some aspects, a Cap 1 structure is connected to an RNA via a 5′- to 5′-triphosphate linkage and is also referred to herein as m7Gppp(2′OMeN₁) or m7G(5′)ppp(5′)(2′OMeN₁). In some aspects, N₁ is chosen from A, C, G, or U. In some aspects, N₁ is A. In some aspects, N₁ is C. In some aspects, N₁ is G. In some aspects, N₁ is U. In some aspects, a m7G(5′)ppp(5′)(2′OmeN₁) Cap 1 structure comprises a second nucleotide, N₂, which is a cap proximal nucleotide at position 2 and is chosen from A, G, C, or U (m7G(5′)ppp(5′)(2′OmeN₁)N₂). In some aspects, N₂ is A. In some aspects, N₂ is C. In some aspects, N₂ is G. In some aspects, N₂ is U.

In some aspects, a Cap 1 structure comprises a guanosine nucleoside methylated at the 7-position of guanine (m7G) and one or more additional modifications, e.g., methylation on a ribose, and a 2′O methylated first nucleotide in an RNA. In some aspects, a Cap 1 structure comprises a guanosine nucleoside methylated at the 7-position of guanine, a 3′O methylation at a ribose (m7(3′OMeG)), and a 2′O methylated first nucleotide in an RNA (2′OMeN₁). In some aspects, a Cap 1 structure is connected to an RNA via a 5′- to 5′-triphosphate linkage and is also referred to herein as m7(3′OMeG)ppp(2′OMeN₁) or m7(3′OMeG)(5′)ppp(5′)(2′OMeN₁). In some aspects, N₁ is chosen from A, C, G, or U. In some aspects, N₁ is A. In some aspects, N₁ is C. In some aspects, N₁ is G. In some aspects, N₁ is U. In some aspects, a m7(3′OMeG)(5′)ppp(5′)(2′OMeN₁) Cap 1 structure comprises a second nucleotide, N₂, which is a cap proximal nucleotide at position 2 and is chosen from A, G, C, or U (m7(3′OMeG)(5′)ppp(5′)(2′OmeN₁)N₂). In some aspects, N₂ is A. In some aspects, N₂ is C. In some aspects, N₂ is G. In some aspects, N₂ is U.

In some aspects, a second nucleotide in a Cap 1 structure may comprise one or more modifications, e.g., methylation. In some aspects, a Cap 1 structure comprising a second nucleotide comprising a 2′O methylation is a Cap 2 structure.

In some aspects, the RNA molecule may be enzymatically capped at the 5′ end using Vaccinia guanylyltransferase, guanosine triphosphate, and S-adenosyl-L-methionine to yield Cap 0 structure. An inverted 7-methylguanosine cap is added via a 5′ to 5′ triphosphate bridge. Alternatively, use of a 2′O-methyltransferase with Vaccinia guanylyltransferase yields the Cap 1 structure where in addition to the Cap 0 structure, the 2′OH group is methylated on the penultimate nucleotide. S-adenosyl-L-methionine (SAM) is a cofactor utilized as a methyl transfer reagent. Non-limiting examples of 5′ cap structures are those which, among other things, have enhanced binding of cap binding polypeptides, increased half-life, reduced susceptibility to 5′ endonucleases and/or reduced 5′ decapping, as compared to synthetic 5′ cap structures known in the art (or to a wild type, natural or physiological 5′ cap structure).

For example, recombinant Vaccinia Virus Capping Enzyme and recombinant 2′ O-methyltransferase enzyme may create a canonical 5′-5′-triphosphate linkage between the 5′-terminal nucleotide of an mRNA and a guanine cap nucleotide wherein the cap guanine includes an N7 methylation and the 5′-terminal nucleotide of the mRNA includes a 2′-O-methyl. Such a structure is termed the Cap 1 structure. This cap results in a higher translational-competency and cellular stability and a reduced activation of cellular pro-inflammatory cytokines, as compared, e.g., to other 5′ cap analog structures known in the art.

In some aspects, the 5′ terminal cap includes a cap analog, for example, a 5′ terminal cap may include a guanine analog. Exemplary guanine analogs include, but are not limited to, inosine, N1-methyl-guanosine, 2′-fluoro-guanosine, 7-deaza-guanosine, 8-oxo-guanosine, 2-amino-guanosine, LNA-guanosine, and 2-azido-guanosine.

In some aspects, the capping region may include a single cap or a series of nucleotides forming the cap. In this aspect the capping region may be from 1 to 10, e.g. 2-9, 3-8, 4-7, 1-5, 5-10, or at least 2, or 10 or fewer nucleotides in length. In this aspect the capping region is at least, at most, exactly, or between any two of 1, 2, 3, 4, 5, 6, 7, 8, 9, or 10 nucleotides in length. In some aspects, the cap is absent. In some aspects, the first and second operational regions may range from 3 to 40, e.g., 5-30, 10-20, 15, or at least 4, or 30 or fewer nucleotides in length and may comprise, in addition to a Start and/or Stop codon, one or more signal and/or restriction sequences. In some aspects, the first and second operational regions are at least, at most, exactly, or between any two of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length and may comprise, in addition to a Start and/or Stop codon, one or more signal and/or restriction sequences.

Further examples of 5′ cap structures include, but are not limited to, glyceryl, inverted deoxy abasic residue (moiety), 4′, 5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety.

In some aspects, the RNA molecule of the present disclosure comprises at least one 5′ cap structure. In some aspects, the RNA molecule of the present disclosure does not comprise a 5′ cap structure.

In one aspect, the 5′ capping structure comprises a modified 5′ Cap 1 structure (m⁷G⁺m^{3′}-5′-ppp-5′-Am). In one aspect, the 5′ capping structure comprises is (3′OMe)-m₂^{7,3-O}Gppp (m₁^{2′-O})ApG (TriLink BioTechnologies). This molecule is identical to the natural RNA cap structure in that it starts with a guanosine methylated at N7, and is linked by a 5′ to 5′ triphosphate linkage to the first coded nucleotide of the transcribed RNA (in this case, an adenosine). This guanosine is also methylated at the 3′ hydroxyl of the ribose to mitigate possible reverse incorporation of the cap molecule. The 2′ hydroxyl of the ribose on the adenosine is methylated, conferring a Cap1 structure.

C. Untranslated Regions (UTRS)

The 5′ UTR is a regulatory region situated at the 5′end of a protein open reading frame that is transcribed into mRNA but not translated into an amino acid sequence or to the corresponding region in an RNA polynucleotide, such as an mRNA molecule. An untranslated region (UTR) may be present 5′ (upstream) of an open reading frame (5′ UTR) and/or 3′ (downstream) of an open reading frame (3′ UTR).

In some aspects, the UTR is derived from an mRNA that is naturally abundant in a specific tissue (e.g., lymphoid tissue), to which the mRNA expression is targeted. In some aspects, the UTR increases protein synthesis. Without being bound by mechanism or theory, the UTR may increase protein synthesis by increasing the time that the mRNA remains in translating polysomes (message stability) and/or the rate at which ribosomes initiate translation on the message (message translation efficiency). Accordingly, the UTR sequence may prolong protein synthesis in a tissue-specific manner.

In some aspects, the 5′ UTR and the 3′ UTR sequences are computationally derived. In some aspects, the 5′ UTR and the 3′ UTRs are derived from a naturally abundant mRNA in a tissue. The tissue may be, for example, liver, a stem cell or lymphoid tissue. The lymphoid tissue may include, for example, any one of a lymphocyte (e.g., a B-lymphocyte, a helper T-lymphocyte, a cytotoxic T-lymphocyte, a regulatory T-lymphocyte, or a natural killer cell), a macrophage, a monocyte, a dendritic cell, a neutrophil, an eosinophil and a reticulocyte. In some aspects, the 5′ UTR and the 3′ UTR are derived from an alphavirus. In some aspects, the 5′ UTR and the 3′ UTR are from a wild type alphavirus.

i. 5′ UTRS

In some aspects, an RNA disclosed herein comprises a 5′ UTR. A 5′ UTR, if present, is located at the 5′ end and starts with the transcriptional start site upstream of the start codon of a protein encoding region. A 5′ UTR is downstream of the 5′ cap (if present), e.g. directly adjacent to the 5′ cap. The 5′ UTR may contain various regulatory elements, e.g., 5′ cap structure, stem-loop structure, and an internal ribosome entry site (IRES), which may play a role in the control of translation initiation.

In some aspects, a 5′ UTR disclosed herein comprises a cap proximal sequence, e.g., as disclosed herein. In some aspects, a cap proximal sequence comprises a sequence adjacent to a 5′ cap. In some aspects, a cap proximal sequence comprises nucleotides in positions +1, +2, +3, +4, and/or +5 of an RNA polynucleotide.

In some aspects, a Cap structure comprises one or more polynucleotides of a cap proximal sequence. In some aspects, a Cap structure comprises an m7 Guanosine cap and nucleotide +1 (N₁) of an RNA polynucleotide. In some aspects, a Cap structure comprises an m7 Guanosine cap and nucleotide +2 (N₂) of an RNA polynucleotide. In some aspects, a Cap structure comprises an m7 Guanosine cap and nucleotides +1 and +2 (N₁ and N₂) of an RNA polynucleotide.

Those skilled in the art, reading the present disclosure, will appreciate that, in some aspects, one or more residues of a cap proximal sequence (e.g., one or more of residues +1, +2, +3, +4, and/or +5) may be included in an RNA by virtue of having been included in a cap entity that (e.g., a Cap 1 structure, etc); alternatively, in some aspects, at least some of the residues in a cap proximal sequence may be enzymatically added (e.g., by a polymerase such as a T7 polymerase). For example, in certain exemplified aspects where a (m₂^{7,3′-O})Gppp(m^{2′-O})ApG cap is utilized, +1 and +2 residues are the (m₂^{7,3′-O}) A and G residues of the cap, and +3, +4, and +5 residues are added by polymerase (e.g., T7 polymerase).

In some aspects, a cap proximal sequence comprises N₁ and/or N₂ of a Cap structure, wherein N₁ and N₂ are any nucleotide, e.g., A, C, G or U. In some aspects, N₁ is A. In some aspects, N₁ is C. In some aspects, N₁ is G. In some aspects, N₁ is U. In some aspects, N₂ is A. In some aspects, N₂ is C. In some aspects, N₂ is G. In some aspects, N₂ is U. In some aspects, a cap proximal sequence comprises N₁ and N₂ of a Cap structure and N₃, N₄ and N₅, wherein N₁ to N₅ correspond to positions +1, +2, +3, +4, and/or +5 of an RNA polynucleotide. In some aspects, N₁, N₂, N₃, N₄, or N₅ are any nucleotide, e.g., A, C, G or U. In some aspects, N₁N₂ comprises any one of the following: AA, AC, AG, AU, CA, CC, CG, CU, GA, GC, GG, GU, UA, UC, UG, or UU. In some aspects, N₁N₂ comprises AG and N₃N₄N₅ comprises any one of the following: AAA, ACA, AGA, AUA, AAG, AGG, ACG, AUG, AAC, ACC, AGC, AUC, AAU, ACU, AGU, AUU, CAA, CCA, CGA, CUA, CAG, CGG, CCG, CUG, CAC, CCC, CGC, CUC, CAU, CCU, CGU, CUU, GAA, GCA, GGA, GUA, GAG, GGG, GCG, GUG, GAC, GCC, GGC, GUC, GAU, GCU, GGU, GUU, UAA, UCA, UGA, UUA, UAG, UGG, UCG, UUG, UAC, UCC, UGC, UUC, UAU, UCU, UGU, or UUU.

In some aspects, a cap proximal sequence comprises N₁ and N₂ of a Cap structure, and a sequence comprising: A₃A₄X₅ (SEQ ID NO: 307; wherein X₅ is A, G, C, or U), where N₁ and N₂ are each independently chosen from: A, C, G, or U. In some aspects, N₁ is A and N₂ is G. In some aspects, X₅ is chosen from A, C, G or U. In some aspects, X₅ is A. In some aspects, X₅ is C. In some aspects, X₅ is G. In some aspects, X₅ is U.

In some aspects, a cap proximal sequence comprises N₁ and N₂ of a Cap structure, and a sequence comprising: C₃A₄X₅ (SEQ ID NO: 308; wherein X₅ is A, G, C, or U), where N₁ and N₂ are each independently chosen from: A, C, G, or U. In some aspects, N₁ is A and N₂ is G. In some aspects, X₅ is chosen from A, C, G or U. In some aspects, X₅ is A. In some aspects, X₅ is C. In some aspects, X₅ is G. In some aspects, X₅ is U.

In some aspects, a cap proximal sequence comprises N₁ and N₂ of a Cap structure, and a sequence comprising X₃Y₄X₅ (SEQ ID NO: 309; wherein X₃ or X₅ are each independently chosen from A, G, C, or U; and Y₄ is not C). In some aspects, N₁ and N₂ are each independently chosen from: A, C, G, or U. In some aspects, N₁ is A and N₂ is G. In some aspects, X₃ and X₅ is each independently chosen from A, C, G or U. In some aspects, X₃ and/or X₅ is A. In some aspects, X₃ and/or X₅ is C. In some aspects, X₃ and/or X₅ is G. In some aspects, X₃ and/or X₅ is U. In some aspects, Y₄ is C. In other aspects, Y₄ is not C. In some aspects, Y₄ is A. In some aspects, Y₄ is G. In other aspects, Y₄ is not G. In some aspects, Y4 is U.

In some aspects, a cap proximal sequence comprises N₁ and N₂ of a Cap structure, and a sequence comprising A₃C₄A₅ (SEQ ID NO: 310). In some aspects, N₁ and N₂ are each independently chosen from: A, C, G, or U. In some aspects, N₁ is A and N₂ is G.

In some aspects, a cap proximal sequence comprises N₁ and N₂ of a Cap structure, and a sequence comprising A₃U₄G₅ (SEQ ID NO: 311). In some aspects, N₁ and N₂ are each independently chosen from: A, C, G, or U. In some aspects, N₁ is A and N₂ is G.

Exemplary 5′ UTRs include a human alpha globin (hAg) 5′UTR or a fragment thereof, a TEV 5′ UTR or a fragment thereof, a HSP705′ UTR or a fragment thereof, or a c-Jun 5′ UTR or a fragment thereof.

In some aspects, an RNA disclosed herein comprises a hAg 5′ UTR or a fragment thereof. In some aspects, an RNA disclosed herein comprises a hAg 5′ UTR having 99%, 98%, 97%, 96%, 95%, 90%, 85%, or 80% identity to a human alpha globin 5′ UTR provided in SEQ ID NO: 312. In some aspects, an RNA disclosed herein comprises a hAg 5′ UTR provided in SEQ ID NO: 312.

SEQ ID NO: 312
AGAAUAAACUAGUAUUCUUCUGGUCCCCACAGACUCAGAGAGAACCC

In some aspects, an RNA disclosed herein comprises a hAg 5′ UTR having 99%, 98%, 97%, 96%, 95%, 90%, 85%, or 80% identity to a human alpha globin 5′ UTR provided in SEQ ID NO: 313. In some aspects, an RNA disclosed herein comprises a hAg 5′ UTR provided in SEQ ID NO: 313.

SEQ ID NO: 313
AAACUAGUAUUCUUCUGGUCCCCACAGACUCAGAGAGAACCC

In one aspect, a DNA encoding a 5′ UTR disclosed herein comprises a sequence having at least, at most, exactly, or between any two of 99%, 98%, 97%, 96%, 95%, 90%, 85%, or 80% identity to SEQ ID NO: 280. In one aspect, the DNA encoding the 5′ UTR comprises a sequence of SEQ ID NO: 280. In one aspect, an RNA disclosed herein comprises a 5′ UTR comprising a sequence having at least, at most, exactly, or between any two of 99%, 98%, 97%, 96%, 95%, 90%, 85%, or 80% identity to a 5′ UTR provided in any of SEQ ID NO: 281 to 282 in which the transcribed 5′ cap structure is underlined. In one aspect, the 5′ UTR comprises a sequence of any of SEQ ID NO: 281 to 282, in which the transcribed 5′ cap structure is underlined.

(DNA)
SEQ ID NO: 280
AGAATAAACTAGTATTCTTCTGGTCCCCACAGACTCAGAGAGAACCCGC
CACC
(RNA)
SEQ ID NO: 281
AGAAUAAACUAGUAUUCUUCUGGUCCCCACAGACUCAGAGAGAACCCGC
CACC
(RNA)
SEQ ID NO: 282
AGAAΨAAACΨAGΨAΨΨCΨΨCΨGGΨCCCCACAGACΨCAGAGAGAACCCGC
CACC

ii. 3′ UTRS

In some aspects, an RNA disclosed herein comprises a 3′ UTR. A 3′ UTR, if present, is situated downstream of a protein coding sequence open reading frame, e.g., downstream of the termination codon of a protein-encoding region. A 3′ UTR is typically the part of an mRNA which is located between the protein coding sequence and the poly-A tail of the mRNA. Thus, in some aspects, the 3′ UTR is upstream of the poly-A sequence (if present), e.g. directly adjacent to the poly-A sequence. The 3′ UTR may be involved in regulatory processes including transcript cleavage, stability and polyadenylation, translation, and mRNA localization.

A 3′ UTR may also comprise elements, which are not encoded in the template, from which an RNA is transcribed, but which are added after transcription during maturation, e.g. a poly-A tail. A 3′ UTR of the mRNA is not translated into an amino acid sequence. In some aspects, an RNA disclosed herein comprises a 3′ UTR comprising an F element and/or an I element. In some aspects, a 3′ UTR or a proximal sequence thereto comprises a restriction site. In some aspects, a restriction site is a BamHI site. In some aspects, a restriction site is a XhoI site.

In some aspects, an RNA disclosed herein comprises a 3′ UTR having at least, at most, exactly, or between any two of 99%, 98%, 97%, 96%, 95%, 90%, 85%, or 80% identity to a 3′ UTR provided in SEQ ID NO: 314. In some aspects, an RNA disclosed herein comprises a 3′ UTR provided in SEQ ID NO: 314.

SEQ ID NO: 314
CUGGUACUGCAUGCACGCAAUGCUAGCUGCCCCUUUCCCGUCCUGGGUAC
CCCGAGUCUCCCCCGACCUCGGGUCCCAGGUAUGCUCCCACCUCCACCUG
CCCCACUCACCACCUCUGCUAGUUCCAGACACCUCCCAAGCACGCAGCAA
UGCAGCUCAAAACGCUUAGCCUAGCCACACCCCCACGGGAAACAGCAGUG
AUUAACCUUUAGCAAUAAACGAAAGUUUAACUAAGCUAUACUAACCCCAG
GGUUGGUCAAUUUCGUGCCAGCCACACC

In one aspect, a DNA encoding a 3′ UTR disclosed herein comprises a sequence having at least, at most, exactly, or between any two of 99%, 98%, 97%, 96%, 95%, 90%, 85%, or 80% identity to SEQ ID NO: 283. In one aspect, the DNA encoding the 5′ UTR comprises a sequence of SEQ ID NO: 283. In one aspect, an RNA disclosed herein comprises a 3′ UTR comprising a sequence having at least, at most, exactly, or between any two of 99%, 98%, 97%, 96%, 95%, 90%, 85%, or 80% identity to a 3′ UTR provided in any of SEQ ID NO: 284 to 285 and 317 to 318. In one aspect, the 3′ UTR comprises a sequence of any of SEQ ID NO: 284 to 285 and 317 to 318.

(DNA)
SEQ ID NO: 283
CTCGAGCTGGTACTGCATGCACGCAATGCTAGCTGCCCCTTTCCCGTCCT
GGGTACCCCGAGTCTCCCCCGACCTCGGGTCCCAGGTATGCTCCCACCTC
CACCTGCCCCACTCACCACCTCTGCTAGTTCCAGACACCTCCCAAGCACG
CAGCAATGCAGCTCAAAACGCTTAGCCTAGCCACACCCCCACGGGAAACA
GCAGTGATTAACCTTTAGCAATAAACGAAAGTTTAACTAAGCTATACTAA
CCCCAGGGTTGGTCAATTTCGTGCCAGCCACACCCTGGAGCTAGC
(RNA)
SEQ ID NO: 284
CUCGAGCUGGUACUGCAUGCACGCAAUGCUAGCUGCCCCUUUCCCGUCCU
GGGUACCCCGAGUCUCCCCCGACCUCGGGUCCCAGGUAUGCUCCCACCUC
CACCUGCCCCACUCACCACCUCUGCUAGUUCCAGACACCUCCCAAGCACG
CAGCAAUGCAGCUCAAAACGCUUAGCCUAGCCACACCCCCACGGGAAACA
GCAGUGAUUAACCUUUAGCAAUAAACGAAAGUUUAACUAAGCUAUACUAA
CCCCAGGGUUGGUCAAUUUCGUGCCAGCCACACCCUGGAGCUAGC
(RNA)
SEQ ID NO: 285
CΨCGAGCΨGGΨACΨGCAΨGCACGCAAΨGCΨAGCΨGCCCCΨΨΨCCCGΨCCΨ
GGGΨACCCCGAGΨCΨCCCCCGACCΨCGGGΨCCCAGGΨAΨGCΨCCCACCΨC
CACCΨGCCCCACΨCACCACCΨCΨGCΨAGΨΨCCAGACCΨCCCAAGCACGCA
GCAAΨGCAGCΨCAAAACGCΨΨAGCCΨAGCCACACCCCCACGGGAAACAGC
AGΨGAΨΨAACCΨΨΨAGCAAΨAAACGAAAGΨΨΨAACΨAAGCΨAΨACΨAACC
CCAGGGΨΨGGΨCAAΨΨΨCGΨGCCAGCCACACCCΨGGAGCΨAGC
(RNA)
SEQ ID NO: 317
CUCGAGCUGGUACUGCAUGCACGCAAUGCUAGCUGCCCCUUUCCCGUCCU
GGGUACCCCGAGUCUCCCCCGACCUCGGGUCCCAGGUAUGCUCCCACCUC
CACCUGCCCCACUCACCACCUCUGCUAGUUCCAGACACCUCCCAAGCACG
CAGCAAUGCAGCUCAAAACGCUUAGCCUAGCCACACCCCCACGGGAAACA
GCAGUGAUUAACCUUUAGCAAUAAACGAAAGUUUAACUAAGCUAUACUAA
CCCCAGGGUUGGUCAAUUUCGUGCCAGCCACACC
(RNA)
SEQ ID NO: 318
CΨCGAGCΨGGΨACΨGCAΨGCACGCAAΨGCΨAGCΨGCCCCΨΨΨCCCGΨCCΨ
GGGΨACCCCGAGΨCΨCCCCCGACCΨCGGGΨCCCAGGΨAΨGCΨCCCACCΨC
CACCΨGCCCCACΨCACCACCΨCΨGCΨAGΨΨCCAGACACCΨCCCAAGCACG
CAGCAAΨGCAGCΨCAAAACGCΨΨAGCCΨAGCCACACCCCCACGGGAAACA
GCAGΨGAΨΨAACCΨΨΨAGCAAΨAAACGAAAGΨΨΨAACΨAAGCΨAΨACΨAA
CCCCAGGGΨΨGGΨCAAΨΨΨCGΨGCCAGCCACACC

D. Open Reading Frame (ORF)

The 5′ and 3′ UTRs may be operably linked to an open reading frame (ORF), which may be a sequence of codons that is capable of being translated into a polypeptide of interest. An open reading frame may be a sequence of several DNA or RNA nucleotide triplets, which may be translated into a peptide or protein. An ORF may begin with a start codon, e.g., a combination of three subsequent nucleotides coding usually for the amino acid methionine (ATG or AUG), at its 5′ end and a subsequent region, which usually exhibits a length which is a multiple of 3 nucleotides. An open reading frame may terminate with at least one stop codon, including but not limited to TAA, TAG, TGA or UAA, UAG or UGA, or any combination thereof. In some aspects, an open reading frame may terminate with one, two, three, four or more stop codons, including but not limited to TAATAA (SEQ ID NO: 289), TAATAG (SEQ ID NO: 290), TAATGA (SEQ ID NO: 291), TAGTGA (SEQ ID NO: 292), TAGTAA (SEQ ID NO: 293), TAGTAG (SEQ ID NO: 294), TGATGA (SEQ ID NO: 295), TGATAG (SEQ ID NO: 296), TGATAA (SEQ ID NO: 297) or UAAUAA (SEQ ID NO: 298), UAAUAG (SEQ ID NO: 299), UAAUGA (SEQ ID NO: 300), UAGUGA (SEQ ID NO: 301), UAGUAA (SEQ ID NO: 302), UAGUAG (SEQ ID NO: 303), UGAUGA (SEQ ID NO: 304), UGAUAG (SEQ ID NO: 305), UGAUAA (SEQ ID NO: 306), or any combination thereof. An open reading frame may be isolated or it may be incorporated in a longer nucleic acid sequence, e.g. in a vector or an mRNA. An open reading frame may also be termed “(protein) coding region” or “coding sequence”.

As stated herein, the RNA molecule may include one (monocistronic), two (bicistronic) or more (multicistronic) open reading frames.

In some aspects, the ORF encodes a non-structural viral gene. In some aspects, the ORF further includes one or more subgenomic promoters. In some aspects, the RNA molecule includes a subgenomic promoter operably linked to the ORF. In some aspects, a first RNA molecule does not include an ORF encoding any polypeptide of interest, whereas a second RNA molecule includes an ORF encoding a polypeptide of interest. In some aspects, the first RNA molecule does not include a subgenomic promoter.

The present disclosure provides for an RNA molecule comprising at least one open reading frame encoding a varicella-zoster virus (VZV) polypeptide. In some aspects, an RNA molecule comprising at least one open reading frame encoding a VZV gE polypeptide.

E. Genes of Interest

The RNA molecules described herein may include a gene of interest. The gene of interest encodes a polypeptide of interest. Non-limiting examples of polypeptides of interest include, e.g., biologics, antibodies, vaccines, therapeutic polypeptides or peptides, cell penetrating peptides, secreted polypeptides, plasma membrane polypeptides, cytoplasmic or cytoskeletal polypeptides, intracellular membrane bound polypeptides, nuclear polypeptides, polypeptides associated with human disease, targeting moieties, those polypeptides encoded by the human genome for which no therapeutic indication has been identified but which nonetheless have utility in areas of research and discovery, or combinations thereof. The sequence for a particular gene of interest is readily identified by one of skill in the art using public and private databases, e.g., GENBANK®.

In some aspects, the RNA molecules include a coding region for a gene of interest. In some aspects, a gene of interest is or comprises an antigenic polypeptide or an immunogenic variant or an immunogenic fragment thereof. In some aspects, an antigenic polypeptide comprises one epitope from an antigen. In some aspects, an antigenic polypeptide comprises a plurality of distinct epitopes from an antigen. In some aspects, an antigenic polypeptide comprising a plurality of distinct epitopes from an antigen is polyepitopic. In some aspects, an antigenic polypeptide comprises: an antigenic polypeptide from an allergen, a viral antigenic polypeptide, a bacterial antigenic polypeptide, a fungal antigenic polypeptide, a parasitic antigenic polypeptide, an antigenic polypeptide from an infectious agent, an antigenic polypeptide from a pathogen, a tumor antigenic polypeptide, or a self-antigenic polypeptide.

The term “antigen” may refer to a substance, which is capable of being recognized by the immune system, e.g. by the adaptive immune system, and which is capable of eliciting an antigen-specific immune response, e.g. by formation of antibodies and/or antigen-specific T cells as part of an adaptive immune response. An antigen may be or may comprise a peptide or protein, which may be presented by the MHC to T-cells. An antigen may be the product of translation of a provided nucleic acid molecule, e.g. an RNA molecule comprising at least one coding sequence as described herein. In addition, fragments, variants and derivatives of an antigen, such as a peptide or a protein, comprising at least one epitope are understood as antigens.

In some aspects, an RNA encoding a gene of interest, e.g., an antigen, is expressed in cells of a subject treated to provide a gene of interest, e.g., an antigen. In some aspects, the RNA is transiently expressed in cells of the subject. In some aspects, expression of a gene of interest, e.g., an antigen, is at the cell surface. In some aspects, a gene of interest, e.g., an antigen, is expressed and presented in the context of MHC. In some aspects, expression of a gene of interest, e.g., an antigen, is into the extracellular space, e.g., the antigen is secreted.

In some aspects, the RNA molecules include a coding region for a gene of interest, e.g., an antigen. In some aspects, the RNA molecules include a coding region for a gene of interest, e.g., an antigen, that is derived from a pathogen associated with an infectious disease. In some aspects, the RNA molecules include a coding region for a gene of interest, e.g., an antigen, that is derived from varicella zoster virus (VZV).

In some aspects, the RNA molecule encodes a VZV gE protein or a fragment or a variant thereof. In some aspects, the RNA molecule encodes a VZV gE protein comprising the amino acid sequence according to any one of GENBANK® Accession No.: AAG32558.1, ABE03086.1, AAK01047.1, Q9J3M8.1, AEW88548.1, AGY33616.1, AEW89124.1, AIT53150.1, CAA25033.1, NP_040190.1, AKG56356.1, AEW89412.1, ABF21714.1, ABF21714.1, AAT07749.1, AEW88764.1, AAG48520.1, and/or AEW88980.1, the respective sequences of which are herein incorporated by reference. In some aspects, the RNA molecule encodes a VZV gE protein comprising the amino acid sequence according to GENBANK® Accession No. AH009994.2, the sequence of which is herein incorporated by reference.

In some aspects, an RNA polynucleotide described herein or a composition or medical preparation comprising the same comprises a nucleotide sequence disclosed herein. In some aspects, an RNA polynucleotide comprises a sequence having at least 80% identity to a nucleotide sequence disclosed herein. In some aspects, an RNA polynucleotide comprises a sequence encoding a polypeptide having at least 80% identity to a polypeptide sequence disclosed herein. In some aspects, an RNA polynucleotide described herein or a composition or medical preparation comprising the same is transcribed by a DNA template. In some aspects, a DNA template used to transcribe an RNA polynucleotide described herein comprises a sequence complementary to an RNA polynucleotide. In some aspects, a gene of interest described herein is encoded by an RNA polynucleotide described herein comprising a nucleotide sequence disclosed herein. In some aspects, an RNA polynucleotide encodes a polypeptide having at least 80% identity to a polypeptide sequence disclosed herein. In some aspects, a polypeptide described herein is encoded by an RNA polynucleotide transcribed by a DNA template comprising a sequence complementary to an RNA polynucleotide.

In some aspects, the RNA molecule encodes a VZV glycoprotein comprising the sequence of any one of SEQ ID NOs: 1-11, or a fragment or variant thereof.

In some aspects, the RNA molecule encodes a VZV glycoprotein synthesized from the nucleic acid sequence comprising any one of SEQ ID NOs: 12-145, or fragment or variant thereof.

F. Poly-A Tail

In some aspects, an RNA molecules disclosed herein comprise a poly-adenylate (poly-A) sequence, e.g., as described herein. In some aspects, a poly-A sequence is situated downstream of a 3′ UTR, e.g., adjacent to a 3′ UTR. A “poly-A tail” or “poly-A sequence” refers to a stretch of consecutive adenine residues, which may be attached to the 3′ end of the RNA molecule. Poly-A sequences are known to those of skill in the art and may follow the 3′ UTR in the RNA molecules described herein. The poly-A tail may increase the half-life of the RNA molecule.

RNA molecules disclosed herein may have a poly-A sequence attached to the free 3′-end of the RNA by a template-independent RNA polymerase after transcription or a poly-A sequence encoded by DNA and transcribed by a template-dependent RNA polymerase. In some aspects, a poly-A sequence is attached during RNA transcription, e.g., during preparation of in vitro transcribed RNA, based on a DNA template comprising repeated dT nucleotides (deoxythymidylate) in the strand complementary to the coding strand.

The DNA sequence encoding a poly-A sequence (coding strand) is referred to as poly-A cassette. In some aspects, the poly-A cassette present in the coding strand of DNA essentially consists of dA nucleotides, but is interrupted by a random sequence of the four nucleotides (dA, dC, dG, and dT). Such a random sequence may be at least, at most, exactly, or between any two of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. Such a cassette is disclosed in WO 2016/005324 A1, hereby incorporated by reference. Any poly-A cassette disclosed in WO 2016/005324 A1 may be used in the present invention. A poly-A cassette that essentially consists of dA nucleotides, but is interrupted by a random sequence having an equal distribution of the four nucleotides (dA, dC, dG, dT) and having a length of e.g., 5 to 50 nucleotides, shows, on DNA level, constant propagation of plasmid DNA in E. coli and is still associated, on RNA level, with the beneficial properties with respect to supporting RNA stability and translational efficiency is encompassed. In some aspects, the poly-A sequence contained in an RNA polynucleotide described herein essentially consists of adenosine nucleotides, but is interrupted by a random sequence of the four nucleotides (A, C, G, U). Such a random sequence may be at least, at most, exactly, or between any two of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some aspects, no nucleotides other than adenosine nucleotides flank a poly-A sequence at its 3′-end, e.g., the poly-A sequence, is not masked or followed at its 3′-end by a nucleotide other than adenosine.

In some aspects, the RNA molecule may further include an endonuclease recognition site sequence immediately downstream of the poly-A tail sequence. The RNA molecule may further include a poly-A polymerase recognition sequence (e.g. AAUAAA) near its 3′ end.

The poly-A sequence may be of any length. In some aspects, the poly-A tail may include 5 to 300 nucleotides in length. In some aspects, the RNA molecule includes a poly-A tail that comprises, essentially consists of, or consists of a sequence of about 25 to about 400 adenosine nucleotides, a sequence of about 50 to about 400 adenosine nucleotides, a sequence of about 50 to about 300 adenosine nucleotides, a sequence of about 50 to about 250 adenosine nucleotides, a sequence of about 60 to about 250 adenosine nucleotides, or a sequence of about 40 to about 100 adenosine nucleotides. In some aspects, the poly-A tail comprises, essentially consists of, or consists of at least, at most, exactly, or between any two of 5, 10, 15, 20, 25, 30, 35, 40, 45, 50, 55, 60, 65, 70, 75, 80, 85, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 adenosine nucleotides. In this context, “essentially consists of” means that most nucleotides in the poly-A sequence, typically at least 75%, at least 80%, at least 85%, at least 90%, at least 95%, at least 96%, at least 97%, at least 98%, or at least 99% by number of nucleotides in the poly-A sequence are adenosine nucleotides, but permits that remaining nucleotides are nucleotides other than adenosine nucleotides, such as uridine, guanosine, or cytosine. In this context, “consists of” means that all nucleotides in the poly-A sequence, e.g., 100% by number of nucleotides in the poly-A sequence, are adenosine nucleotides.

In some aspects, the RNA molecule includes a poly-A tail that includes a sequence of greater than 30 adenosine nucleotides. In some aspects, the RNA molecule includes a poly-A tail that includes about 40 adenosine nucleotides. In some aspects, the RNA molecule includes a poly-A tail that includes about 80 adenosine nucleotides. In some aspects, the 3′ poly-A tail has a stretch of at least 10 consecutive adenosine residues and at most 300 consecutive adenosine residues. In some specific aspects, the RNA molecule includes about 40 consecutive adenosine residues. In some aspects, the RNA molecule includes about 80 consecutive adenosine residues. Poly-A tails may play key regulatory roles in enhancing translation efficiency and regulating the efficiency of mRNA quality control and degradation. Short sequences or hyperpolyadenylation may signal for RNA degradation. Some designs include a poly-A tails of about 40 adenosine nucleotides, about adenosine nucleotides.

In some aspects, a poly-A tail may be located within an RNA molecule or other nucleic acid molecule, such as, e.g., in a vector, for example, in a vector serving as template for the generation of an RNA, e.g. an mRNA, e.g., by transcription of the vector. In some aspects, the RNA molecule may not include a poly-A tail.

In one aspect, a DNA encoding a poly-A tail disclosed herein comprises a sequence having at least, at most, exactly, or between any two of 99%, 98%, 97%, 96%, 95%, 90%, 85%, or 80% identity to SEQ ID NO: 286. In one aspect, the DNA encoding the poly-A tail comprises a sequence of SEQ ID NO: 286. In one aspect, an RNA disclosed herein comprises a poly-A tail comprising a sequence having at least, at most, exactly, or between any two of 99%, 98%, 97%, 96%, 95%, 90%, 85%, or 80% identity to any of SEQ ID NO: 287 to 288 and 315 to 316. In one aspect, the poly-A tail comprises a sequence of any of SEQ ID NO: 287 to 288+/−2 adenosine (A) nucleotides. In one aspect, the poly-A tail comprises a sequence of any of SEQ ID NO: 287 to 288+/−1 adenosine (A) nucleotides. In one aspect, the poly-A tail comprises a sequence of any of SEQ ID NO: 287 to 288. In one aspect, the poly-A tail comprises a sequence of any of SEQ ID NO: 315 to 316+/−2 adenosine (A) nucleotides. In one aspect, the poly-A tail comprises a sequence of any of SEQ ID NO: 315 to 316+/−1 adenosine (A) nucleotides. In one aspects, the poly-A tail comprises a sequence of any of SEQ ID NO: 315 to 316.

(DNA)
SEQ ID NO: 286
AAAAAAAAAAAAAAAAAAAAAAAAAAAAAAGCATATGACTAAAAAAAAA
AAAAAAAAAAAAAAAAAAAAAAAAAAAAAAAAAAAAAAAAAAAAAAAAA
AAAAAAAAAAAA
(RNA)
SEQ ID NO: 287
AAAAAAAAAAAAAAAAAAAAAAAAAAAAAAGCAUAUGACUAAAAAAAAA
AAAAAAAAAAAAAAAAAAAAAAAAAAAAAAAAAAAAAAAAAAAAAAAAA
AAAAAAAAAAAA
(RNA)
SEQ ID NO: 288
AAAAAAAAAAAAAAAAAAAAAAAAAAAAAAGCAΨAΨGACΨAAAAAAAAA
AAAAAAAAAAAAAAAAAAAAAAAAAAAAAAAAAAAAAAAAAAAAAAAAA
AAAAAAAAAAAA
(RNA)
SEQ ID NO: 315
AAAAAAAAAAAAAAAAAAAAAAAAAAAAAAGCAUAUGACUAAAAAAAAA
AAAAAAAAAAAAAAAAAAAAAAAAAAAAAAAAAAAAAAAAAAAAAAAAA
AAAAAAAAAAA
(RNA)
SEQ ID NO: 316
AAAAAAAAAAAAAAAAAAAAAAAAAAAAAAGCAΨAΨGACΨAAAAAAAAA
AAAAAAAAAAAAAAAAAAAAAAAAAAAAAAAAAAAAAAAAAAAAAAAAA
AAAAAAAAAAA

G. Self-Amplifying RNA (SARNA)

In some aspects, the RNA molecule may be an saRNA. “Self-amplifying RNA,” “self-amplifying RNA,” and “replicon” refer to RNA with the ability to replicate itself. Self-amplifying RNA molecules may be produced by using replication elements derived from, e.g. alphaviruses, and substituting the structural viral polypeptides with a nucleotide sequence encoding a polypeptide of interest. A self-amplifying RNA molecule is typically a positive-strand molecule that may be directly translated after delivery to a cell, and this translation provides an RNA-dependent RNA polymerase which then produces both antisense and sense transcripts from the delivered RNA. The delivered RNA leads to the production of multiple daughter RNA molecules. These daughter RNA molecules, as well as collinear subgenomic transcripts, may be translated themselves to provide in situ expression of an encoded gene of interest, e.g., a viral antigen, or may be transcribed to provide further transcripts with the same sense as the delivered RNA which are translated to provide in situ expression of the antigen. The overall result of this sequence of transcriptions is an amplification in the number of the introduced saRNA molecules and so the encoded gene of interest, e.g., a viral antigen, becomes a major polypeptide product of the cells.

In some aspects, the self-amplifying RNA includes at least one or more genes including any one of viral replicases, viral proteases, viral helicases and other nonstructural viral proteins, or combination thereof. In some aspects, the self-amplifying RNA may also include 5′- and 3′-end tractive replication sequences, and optionally a heterologous sequence that encodes a desired amino acid sequence (e.g., an antigen of interest). A subgenomic promoter that directs expression of the heterologous sequence may be included in the self-amplifying RNA. Optionally, the heterologous sequence (e.g., an antigen of interest) may be fused in frame to other coding regions in the self-amplifying RNA and/or may be under the control of an internal ribosome entry site (IRES).

In some aspects, a self-amplifying RNA molecule described herein encodes (i) an RNA-dependent RNA polymerase that may transcribe RNA from the self-amplifying RNA molecule and (ii) a polypeptide of interest, e.g., a viral antigen. In some aspects, the polymerase may be an alphavirus replicase, e.g., including any one of alphavirus protein nsP1, nsP2, nsP3, nsP4, and any combination thereof.

In some aspects, the self-amplifying RNA molecule may have two open reading frames. The first (5′) open reading frame may encode a replicase; the second (3′) open reading frame may encode a polypeptide comprising an antigen of interest. In some aspects the RNA may have additional (e.g., downstream) open reading frames, e.g., to encode further antigens or to encode accessory polypeptides.

In some aspects, the saRNA molecule further includes (1) an alphavirus 5′ replication recognition sequence, and (2) an alphavirus 3′ replication recognition sequence. In some aspects, the 5′ sequence of the self-amplifying RNA molecule is selected to ensure compatibility with the encoded replicase.

In some aspects, the self-amplifying RNA molecule may encode a single polypeptide antigen or, optionally, two or more of polypeptide antigens linked together in a way that each of the sequences retains its identity (e.g., linked in series) when expressed as an amino acid sequence. The polypeptides generated from the self-amplifying RNA may then be produced as a fusion polypeptide or engineered in such a manner to result in separate polypeptide or peptide sequences.

In some aspects, the self-amplifying RNA described herein may encode one or more polypeptide antigens that include a range of epitopes. In some aspects, the self-amplifying RNA described herein may encode epitopes capable of eliciting either a helper T-cell response or a cytotoxic T-cell response or both.

### IV. RNA Transcription

In some aspects, the RNA disclosed herein is produced by in vitro transcription or chemical synthesis. In the context of the present disclosure, the term “transcription” relates to a process, wherein the genetic code in a DNA sequence is transcribed into RNA. Subsequently, the RNA may be translated into peptide or protein.

According to the present disclosure, “transcription” comprises “in vitro transcription” or “IVT,” which refers to the process whereby transcription occurs in vitro in a non-cellular system to produce a synthetic RNA product for use in various applications, including, e.g., production of protein or polypeptides. Cloning vectors may be applied for the generation of transcripts. These cloning vectors are generally designated as transcription vectors and are according to the present invention encompassed by the term “vector.” According to specific aspects, the RNA used is in vitro transcribed RNA (IVT-RNA) and may be obtained by in vitro transcription of an appropriate DNA template. The promoter for controlling transcription may be any promoter for any RNA polymerase. Particular examples of RNA polymerases are the T7, T3, and SP6 RNA polymerases. Preferably, the in vitro transcription according to the invention is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

Synthetic IVT RNA products may be translated in vitro or introduced directly into cells, where they may be translated. With respect to RNA, the term “expression” or “translation” relates to the process in the ribosomes of a cell by which a strand of mRNA directs the assembly of a sequence of amino acids to make a peptide or protein. Such synthetic RNA products include, e.g., but are not limited to mRNA molecules, saRNA molecules, antisense RNA molecules, shRNA molecules, long non-coding RNA molecules, ribozymes, aptamers, guide RNA molecules (e.g., for CRISPR), ribosomal RNA molecules, small nuclear RNA molecules, small nucleolar RNA molecules, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

In some aspects, an mRNA is produced by in vitro transcription using a DNA template where DNA refers to a nucleic acid that contains deoxyribonucleotides. In some aspects, an RNA disclosed herein is in vitro transcribed RNA (IVT-RNA) and may be obtained by in vitro transcription of an appropriate DNA template. The promoter for controlling transcription may be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In some aspects, starting material for IVT may include linearized DNA template, nucleotides, RNase inhibitor, pyrophosphatase, and/or T7 RNA polymerase. In some aspects, the IVT process is conducted in a bioreactor. The bioreactor may comprise a mixer. In some aspects, nucleotides may be added into the bioreactor throughout the IVT process.

In some aspects, one or more post-IVT agents are added into the IVT mixture comprising RNA in the bioreactor after the IVT process. Exemplary post-IVT agents may include DNAse I configured to digest the linearized DNA template, and proteinase K configured to digest DNAse I and T7 RNA polymerase. In some aspects, the post-IVT agents are incubated with the mixture in the bioreactor after IVT. In some aspects, the bioreactor may contain at least, at most, exactly, or between any two of 60, 70, 80, 90, 100, 110, 120, 130, 140, 150,160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 or more liters IVT mixture. The IVT mixture may have an RNA concentration at least, at most, exactly, or between any two of 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 7.0, 8.0, 9.0, 10, 11, 12, 13, 14, 15, 16, 17, 18, 19, 20, 30, 40, 50, 60, 70, 80, 90, and 100 mg/mL or more RNA.

In some aspects, the IVT mixture may include residual spermidine, residual DNA, residual proteins, peptides, HEPES, EDTA, ammonium sulfate, cations (e.g., Mg2+, Na+, Ca2+), RNA fragments, residual nucleotides, free phosphates, or any combinations thereof.

In some aspects, at least a portion of the IVT mixture is filtered. The IVT mixture may be filtered via ultrafiltration and/or diafiltration to remove at least some impurities from the IVT mixture and/or to change buffer solution for the at least a portion of IVT mixture to produce a concentrated RNA solution as a retentate.

In some aspects, both “ultrafiltration” and “diafiltration” refer to a membrane filtration process. Ultrafiltration typically uses membranes having pore sizes of at least, at most, exactly, or between any two of 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, and 0.1 μm. In some aspects, ultrafiltration membranes are typically classified by molecular weight cutoff (MWCO) rather than pore size. For example, the MWCO may be at least, at most, exactly, or between any two of 30 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390 kDa, 400 kDa, 500 kDa, 600 kDa, 700 kDa, 800 kDa, 900 kDa, 1000 kDa, 2000 kDa, 3000 kDa, 4000 kDa, 5000 kDa, 6000 kDa, 7000 kDa, 8000 kDa, 9000 kDa, and 10000 kDa. A skilled artisan will understand that filtration membranes may be of different suitable materials, including, e.g., polymeric, cellulose, ceramic, etc., depending upon the application. In some aspects, membrane filtration may be more desirable for large volume purification process.

In some aspects, ultrafiltration and diafiltration of the IVT mixture for purifying RNA may include (1) Direct Flow Filtration (DFF), also known as “dead-end” filtration, that applies a feed stream perpendicular to the membrane face and attempts to pass 100% of the fluid through the membrane, and/or (2) Tangential Flow Filtration (TFF), also known as crossflow filtration, where a feed stream passes parallel to the membrane face as one portion passes through the membrane (permeate) while the remainder (retentate) is retained and/or recirculated back to the feed tank.

In some aspects, the filtering of the IVT mixture is conducted via TFF that comprises an ultrafiltration step, a first diafiltration step, and a second diafiltration step. In some aspects, the first diafiltration step is conducted in the presence of ammonium sulfate. The first diafiltration step may be configured to remove a majority of impurities from the IVT mixture. In some aspects, the second diafiltration step is conducted without ammonium sulfate. The second diafiltration step may be configured to transfer the RNA into a DS buffer formulation.

A filtration membrane with an appropriate MWCO may be selected for the ultrafiltration in the TFF process. The MWCO of a TFF membrane determines which solutes may pass through the membrane into the filtrate and which are retained in the retentate. The MWCO of a TFF membrane may be selected such that substantially all of the solutes of interest (e.g., desired synthesized RNA species) remains in the retentate, whereas undesired components (e.g., excess ribonucleotides, small nucleic acid fragments such as digested or hydrolyzed DNA template, peptide fragments such as digested proteins and/or other impurities) pass into the filtrate. In some aspects, the retentate comprising desired synthesized RNA species may be re-circulated to a feed reservoir to be re-filtered in additional cycles. In some aspects, a TFF membrane may have a MWCO equal to at least, at most, exactly, or between any two of 30 kDa, 40 kDa, 50 kDa, 60 kDa, 70 kDa, 80 kDa, 90 kDa, or more. In some aspects, a TFF membrane may have a MWCO equal to at least, at most, exactly, or between any two of 100 kDa, 150 kDa, 200 kDa, 250 kDa, 300 kDa, 350 kDa, 400 kDa, or more. In some aspects, a TFF membrane may have a MWCO of about 250-350 kDa. In some aspects, a TFF membrane (e.g., a cellulose-based membrane) may have a MWCO of about 30-300 kDa; in some aspects about 50-300 kDa, about 100-300 kDa, or about 200-300 kDa.

Diafiltration may be performed either discontinuously, or alternatively, continuously. For example, in continuous diafiltration, a diafiltration solution may be added to a sample feed reservoir at the same rate as filtrate is generated. In this way, the volume in the sample reservoir remains constant but small molecules (e.g., salts, solvents, etc.) that may freely permeate through a membrane are removed. Using solvent removal as an example, each additional diafiltration volume (DV) reduces the solvent concentration further. In discontinuous diafiltration, a solution is first diluted and then concentrated back to the starting volume. This process is then repeated until the desired concentration of small molecules (e.g. salts, solvents, etc.) remaining in the reservoir is reached. Each additional diafiltration volume (DV) reduces the small molecule (e.g., solvent) concentration further. Continuous diafiltration typically requires a minimum volume for a given reduction of molecules to be filtered. Discontinuous diafiltration, on the other hand, permits fast changes of the retentate condition, such as pH, salt content, and the like. In some aspects, the first diafiltration step is conducted with diavolumes equal to at least, at most, exactly, or between any two of 2, 3, 4, 5, 6, 7, 8, 9, 10, or more. In some aspects, the second diafiltration step is conducted with diavolumes equal to at least, at most, exactly, or between any two of 5, 6, 7, 8, 9, 10, 11, 12, 13, 14, 15, 16, 17, 18, 19, 20, or more. In some aspects, the first diafiltration step is conducted with 5 diavolumes, and second diafiltration step is conducted with 10 diavolumes.

In some aspects, for the ultrafiltration and/or diafiltration, the IVT mixture is filtered at a rate equal to at least, at most, exactly, or between any two of 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 600, 700, 800, 900, or 1000 L/m2 of filter area per hour, or more. The concentrated RNA solution may comprise at least, at most, exactly, or between any two of 2.0, 2.1, 2.2, 2.3, 2.4, or 2.5 mg/mL single stranded RNA.

The bioburden of the concentrated RNA solution via filtration to obtain an RNA product solution may also be reduced, in some aspects. The filtration for reducing bioburden may be conducted using one or more filters. The one or more filters may include a filter with a pore size of at least, at most, exactly, or between any two of 0.2 μm, 0.45 μm, 0.65 μm, 0.8 μm, or any other pore size configured to remove bioburdens.

As one example, reducing the bioburden may include draining a retentate tank containing retentate obtained from the ultrafiltration and/or diafiltration to obtain the retentate. Reducing the bioburden may include flushing a filtration system for ultrafiltration and/or diafiltration using a wash buffer solution to obtain a wash pool solution comprising residue RNA remaining in the filtration system. The retentate may be filtered to obtain a filtered retentate. The wash pool solution may be filtered using a first 0.2 μm filter to obtain a filtered wash pool solution. The retentate may be filtered using the first 0.2 μm filter or another 0.2 μm filter.

The filtered wash pool solution and the filtered retentate may be combined to form a combined pool solution. The combined pool solution may be filtered using a second 0.2 μm filter to obtain a filtered combined pool solution, which is further filtered using a third 0.2 μm filter to produce an RNA product solution.

### V. RNA Encapsulation

The RNA in an RNA product solution may be encapsulated, and the RNA solution may further comprise at least one encapsulating agent. In one aspect, the encapsulating agent comprises a lipid, a lipid nanoparticle (LNP), lipoplexes, polymeric particles, polyplexes, and monolithic delivery systems, and a combination thereof.

In one aspect, the encapsulating agent is a lipid, and produced is lipid nanoparticle (LNP)-encapsulated RNA. Without intending to be bound by any theory, it is believed that the cationic or cationically ionizable lipid or lipid-like material and/or the cationic polymer combine together with the nucleic acid to form aggregates, and this aggregation results in colloidally stable particles. A lipid may be a naturally occurring lipid or a synthetic lipid. However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof. A lipid is a substance that is insoluble in water and extractable with an organic solvent. Compounds other than those specifically described herein are understood by one of skill in the art as lipids, and are encompassed by the compositions and methods of the present disclosure. A lipid component and a non-lipid may be attached to one another, either covalently or non-covalently.

In some aspects, LNPs may be designed to protect RNA molecules (e.g., saRNA, mRNA) from extracellular RNases and/or may be engineered for systemic delivery of the RNA to target cells. In some aspects, such LNPs may be particularly useful to deliver RNA molecules (e.g., saRNA, mRNA) when RNA molecules are intravenously administered to a subject in need thereof. In some aspects, such LNPs may be particularly useful to deliver RNA molecules (e.g., saRNA, mRNA) when RNA molecules are intramuscularly administered to a subject in need thereof.

In one aspect, the RNA in the RNA solution is at a concentration of <1 mg/mL. In another aspect, the RNA is at a concentration of at least about 0.05 mg/mL. In another aspect, the RNA is at a concentration of at least about 0.5 mg/mL. In another aspect, the RNA is at a concentration of at least about 1 mg/mL. In another aspect, the RNA concentration is from about 0.05 mg/mL to about 0.5 mg/mL. In another aspect, the RNA is at a concentration of at least 10 mg/mL. In another aspect, the RNA is at a concentration of at least 50 mg/mL. In some aspects, the RNA is at a concentration of at least, at most, exactly, or between any two of about 0.05 mg/mL, 0.5 mg/mL, 1 mg/mL, 10 mg/mL, 50 mg/mL, 75 mg/mL, 100 mg/mL, 150 mg/mL, 200 mg/mL, 250 mg/mL, 300 mg/mL, 400 mg/mL, or more.

The present disclosure provides for an RNA solution and lipid preparation mixture or compositions thereof comprising at least one RNA encoding, e.g., an antigen (e.g., a VZV polypeptide) complexed with, encapsulated in, and/or formulated with one or more lipids, and forming lipid nanoparticles (LNPs), liposomes, lipoplexes and/or nanoliposomes. In some aspects, the composition comprises a lipid nanoparticle.

A lipid nanoparticle or LNP refers to particles of any morphology generated when a cationic lipid and optionally one or more further lipids are combined, e.g. in an aqueous environment and/or in the presence of RNA. In some aspects, lipid nanoparticles are included in a formulation that may be used to deliver an active agent or therapeutic agent, such as a nucleic acid (e.g., mRNA) to a target site of interest (e.g., cell, tissue, organ, tumor, and the like). In some aspects, the lipid nanoparticles of the present disclosure comprise a nucleic acid. Such lipid nanoparticles typically comprise a cationic lipid and one or more excipients, e.g., one or more neutral lipids, charged lipids, steroids, polymer conjugated lipids, or combinations thereof. In some aspects, the active agent or therapeutic agent, such as a nucleic acid (e.g., mRNA), may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response. The nucleic acid (e.g., mRNA) or a portion thereof may also be associated and complexed with the lipid nanoparticle. A lipid nanoparticle may comprise any lipid capable of forming a particle to which the nucleic acids are attached, or in which the one or more nucleic acids are encapsulated.

In some aspects, provided RNA molecules (e.g., saRNA, mRNA) may be formulated with LNPs. In some aspects, the lipid nanoparticles may have a mean diameter of about 1 to 500 nm. In some aspects, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or at least, at most, exactly, or between any two of 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321). Here, “mean diameter,” “diameter,” or “size” for particles is used synonymously with this value of the Z-average.

LNPs described herein may exhibit a polydispersity index less than about 0.5, less than about 0.4, less than about 0.3, or about 0.2 or less. By way of example, the LNPs may exhibit a polydispersity index of at least, at most, exactly, or between any two of 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5. The polydispersity index is, in some aspects, calculated based on dynamic light scattering measurements by the so-called cumulant analysis as mentioned in the definition of the “average diameter.” Under certain prerequisites, it may be taken as a measure of the size distribution of an ensemble of nanoparticles.

In certain aspects, nucleic acids (e.g., RNA molecules), when present in provided LNPs, are resistant in aqueous solution to degradation with a nuclease. In some aspects, LNPs are liver-targeting lipid nanoparticles. In some aspects, LNPs are cationic lipid nanoparticles comprising one or more cationic lipids (e.g., ones described herein). In some aspects, cationic LNPs may comprise at least one cationic lipid, at least one polymer conjugated lipid, and at least one helper lipid (e.g., at least one neutral lipid).

In certain aspects, the RNA solution and lipid preparation mixture or compositions thereof may have, have at least, or have at least, at most, exactly, or between any two of about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of a particular lipid, lipid type, or non-lipid component such as lipid-like materials and/or cationic polymers or an adjuvant, antigen, peptide, polypeptide, sugar, nucleic acid or other material disclosed herein or as would be known to one of skill in the art.

LNPs described herein may be prepared using a wide range of methods that may involve obtaining a colloid from at least one cationic or cationically ionizable lipid or lipid-like material and/or at least one cationic polymer and mixing the colloid with nucleic acid to obtain nucleic acid particles. The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media). In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in some aspects, formed as follows: an ethanol solution comprising lipids, such as cationic lipids and additional lipids, is injected into an aqueous solution under stirring. In some aspects, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

In some aspects, LNP-encapsulated RNA may be produced by rapid mixing of an RNA solution described herein (e.g., the RNA product solution) and a lipid preparation described herein (comprising, e.g., at least one cationic lipid and optionally one or more other lipid components, in an organic solvent) under conditions such that a sudden change in solubility of lipid component(s) is triggered, which drives the lipids towards self-assembly in the form of LNPs. In some aspects, suitable buffering agents comprise tris, histidine, citrate, acetate, phosphate, or succinate. The pH of a liquid formulation relates to the pKa of the encapsulating agent (e.g. cationic lipid). The pH of the acidifying buffer may be at least half a pH scale less than the pKa of the encapsulating agent (e.g. cationic lipid), and the pH of the final buffer may be at least half a pH scale greater than the pKa of the encapsulating agent (e.g. cationic lipid). In some aspects, properties of a cationic lipid are chosen such that nascent formation of particles occurs by association with an oppositely charged backbone of a nucleic acid (e.g., RNA). In this way, particles are formed around the nucleic acid, which, for example, in some aspects, may result in much higher encapsulation efficiency than it is achieved in the absence of interactions between nucleic acids and at least one of the lipid components.

In certain aspects, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

Some aspects described herein relate to compositions, methods and uses involving more than one, e.g., 2, 3, 4, 5, 6 or even more nucleic acid species such as RNA species. In an LNP formulation, it is possible that each nucleic acid species is separately formulated as an individual LNP formulation. In that case, each individual LNP formulation will comprise one nucleic acid species. The individual LNP formulations may be present as separate entities, e.g. in separate containers. Such formulations are obtainable by providing each nucleic acid species separately (typically each in the form of a nucleic acid-containing solution) together with suitable cationic or cationically ionizable lipids or lipid-like materials and cationic polymers that allow the formation of LNPs. Respective particles will contain exclusively the specific nucleic acid species that is being provided when the particles are formed (individual particulate formulations).

In some aspects, a composition such as a pharmaceutical composition comprises more than one individual LNP formulation. Respective pharmaceutical compositions are referred to as mixed LNP formulations. Mixed LNP formulations according to the invention are obtainable by forming, separately, individual LNP formulations, as described above, followed by a step of mixing of the individual LNP formulations. By the step of mixing, a formulation comprising a mixed population of nucleic acid-containing LNPs is obtainable. Individual LNP populations may be together in one container, comprising a mixed population of individual LNP formulations.

Alternatively, it is possible that different nucleic acid species are formulated together as a combined LNP formulation. Such formulations are obtainable by providing a combined formulation (typically combined solution) of different RNA species together with suitable cationic or cationically ionizable lipids or lipid-like materials and cationic polymers that allow the formation of LNPs. As opposed to a mixed LNP formulation, a combined LNP formulation will typically comprise LNPs that comprise more than one RNA species. In a combined LNP composition, different RNA species are typically present together in a single particle.

A. Cationic Polymeric Materials

Given their high degree of chemical flexibility, polymeric materials are commonly used for nanoparticle-based delivery. Typically, cationic materials are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic materials useful in some aspects herein. In addition, some investigators have synthesized polymeric materials specifically for nucleic acid delivery. Poly(P-amino esters), in particular, have gained widespread use in nucleic acid delivery owing to their ease of synthesis and biodegradability. In some aspects, such synthetic materials may be suitable for use as cationic materials herein.

A “polymeric material,” as used herein, is given its ordinary meaning, e.g., a molecular structure comprising one or more repeat units (monomers), connected by covalent bonds. In some aspects, such repeat units may all be identical; alternatively, in some cases, there may be more than one type of repeat unit present within the polymeric material. In some cases, a polymeric material is biologically derived, e.g., a biopolymer such as a protein. In some cases, additional moieties may also be present in the polymeric material, for example targeting moieties such as those described herein.

Those skilled in the art are aware that, when more than one type of repeat unit is present within a polymer (or polymeric moiety), then the polymer (or polymeric moiety) is said to be a “copolymer.” In some aspects, a polymer (or polymeric moiety) utilized in accordance with the present disclosure may be a copolymer. Repeat units forming the copolymer may be arranged in any fashion. For example, in some aspects, repeat units may be arranged in a random order; alternatively or additionally, in some aspects, repeat units may be arranged in an alternating order, or as a “block” copolymer, e.g., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

In certain aspects, a polymeric material for use in accordance with the present disclosure is biocompatible. Biocompatible materials are those that typically do not result in significant cell death at moderate concentrations. In certain aspects, a biocompatible material is biodegradable, e.g., is able to degrade, chemically and/or biologically, within a physiological environment, such as within the body. In certain aspects, a polymeric material may be or comprise protamine or polyalkyleneimine, in particular protamine.

As those skilled in the art are aware term “protamine” is often used to refer to any of various strongly basic proteins of relatively low molecular weight that are rich in arginine and are found associated especially with DNA in place of somatic histones in the sperm cells of various animals (as fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some aspects, the term “protamine” as used herein is refers to a protamine amino acid sequence obtained or derived from natural or biological sources, including fragments thereof and/or multimeric forms of said amino acid sequence or fragment thereof, as well as (synthesized) polypeptides which are artificial and specifically designed for specific purposes and cannot be isolated from native or biological sources.

In some aspects, a polyalkyleneimine comprises polyethylenimine and/or polypropylenimine. In some aspects, the polyalkyleneimine is polyethyleneimine (PEI). In some aspects, the polyalkyleneimine is a linear polyalkyleneimine, e.g., linear polyethyleneimine (PEI).

Cationic materials (e.g., polymeric materials, including polycationic polymers) contemplated for use herein include those which are able to electrostatically bind nucleic acid. In some aspects, cationic polymeric materials contemplated for use herein include any cationic polymeric materials with which nucleic acid may be associated, e.g. by forming complexes with the nucleic acid or forming vesicles in which the nucleic acid is enclosed or encapsulated.

In some aspects, particles described herein may comprise polymers other than cationic polymers, e.g., non-cationic polymeric materials and/or anionic polymeric materials. Collectively, anionic and neutral polymeric materials are referred to herein as non-cationic polymeric materials.

B. Lipids & Lipid-Like Materials

The terms “lipid” and “lipid-like material” are used herein to refer to molecules which comprise one or more hydrophobic moieties or groups and optionally also one or more hydrophilic moieties or groups. According to the disclosure, lipids and lipid-like materials may be cationic, anionic or neutral. Neutral lipids or lipid-like materials exist in an uncharged or neutral zwitterionic form at a selected pH.

The term “lipid” refers to a group of organic compounds that are characterized by being insoluble in water but soluble in many organic solvents. Generally, lipids may be divided into eight categories: fatty acids and their derivatives (including tri-, di-, monoglycerides, and phospholipids), glycerolipids, glycerophospholipids, sphingolipids, saccharolipids, polyketides, sterol lipids as well as sterol-containing metabolites such as cholesterol, and prenol lipids. Examples of fatty acids include, but are not limited to, fatty esters and fatty amides. Examples of glycerolipids include, but are not limited to, glycosylglycerols and glycerophospholipids (e.g., phosphatidylcholine, phosphatidylethanolamine, phosphatidylserine). Examples of sphingolipids include, but are not limited to, ceramides phosphosphingolipids (e.g., sphingomyelins, phosphocholine), and glycosphingolipids (e.g., cerebrosides, gangliosides). Examples of sterol lipids include, but are not limited to, cholesterol and its derivatives and tocopherol and its derivatives.

The term “lipid-like material,” “lipid-like compound,” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

In some aspects, the RNA solution and lipid preparation mixture or compositions thereof may comprise cationic lipids, neutral lipids, cholesterol, and/or polymer (e.g., polyethylene glycol) conjugated lipids which form lipid nanoparticles that encompass the RNA molecules. Therefore, in some aspects, the LNP may comprise a cationic lipid and one or more excipients, e.g., one or more neutral lipids, charged lipids, steroids or steroid analogs (e.g., cholesterol), polymer conjugated lipids (e.g. PEG-lipid), or combinations thereof. In some aspects, the LNPs encompass, or encapsulate, the nucleic acid molecules.

i. Cationic Lipids

Cationic or cationically ionizable lipids or lipid-like materials refer to a lipid or lipid-like material capable of being positively charged and able to electrostatically bind nucleic acid. As used herein, a “cationic lipid” or “cationic lipid-like material” refers to a lipid or lipid like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge. Exemplary cationic lipids include one or more amine group(s) which bear the positive charge. Cationic lipids may encapsulate negatively charged RNA.

In some aspects, cationic lipids are ionizable such that they may exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “cationically ionizable” lipids or lipid-like materials are comprised by the term “cationic lipid” or “cationic lipid-like material” unless contradicted by the circumstances.

In some aspects, a cationic lipid may comprise from about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle. In some aspects, a cationic lipid may be at least, at most, exactly, or between any two of 10 mol %, 20 mol %, 30 mol %, 40 mol %, 50 mol %, 60 mol %, 70 mol %, 80 mol %, 90 mol %, or 100 mol %, or any range or value derivable therein, of the total lipid present in the particle.

Examples of cationic lipids include, but are not limited to: ((4-hydroxybutyl)azanediyl)bis(hexane-6,1-diyl)bis(2-hexyldecanoate); 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-I-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-I-(cis,cis-9,12-oc-tadecadienoxy)propane (CLinDMA), 2-[5′-(cholest-5-en-3-beta-oxy)-3′-oxapentoxy)-3-dimethyl-I-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DM A), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-I-propanaminium bromide (GAP-DMRIE), N-(2-Aminoethyl)-N,N-dimethyl-2,3-bis(tetradecyloxy)-1-propanaminium bromide (bAE-DMRIE), N-(4-carboxybenzyl)-N,N-dimethyl-2,3-bis(oleoyloxy)propan-1-aminium (DOBAQ), 2-({8-[(3b)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-ammonium bromide (DLRIE), N-(2-aminoethyl)-N,N-dimethyl-2,3-bis(tetradecyloxy)propan-1-aminium bromide (DMORIE), di((Z)-non-2-en-I-yl) 8,8′-((((2(dimethylamino)ethyl)thio)carbonyl)azanediyl)dioctanoate (ATX), N,N-dimethyl-2,3-bis(dodecyloxy)propan-1-amine (DLDMA), N,N-dimethyl-2,3-bis(tetradecyloxy)propan-1-amine (DMDMA), Di((Z)-non-2-en-I-yl)-9-((4-(dimethylaminobutanoyl)oxy)heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino)propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid 02-200); or heptadecan-9-yl 8-((2-hydroxyethyl) (6-oxo-6-(undecyloxy)hexyl) amino) octanoate (SM-102).

In some aspects, the lipid nanoparticles comprise one or more cationic lipids. In one aspect, the lipid nanoparticles comprise (4-hydroxybutyl)azanediyl)bis(hexane-6,1-diyl)bis(2-hexyldecanoate) (ALC-0315), having the formula:

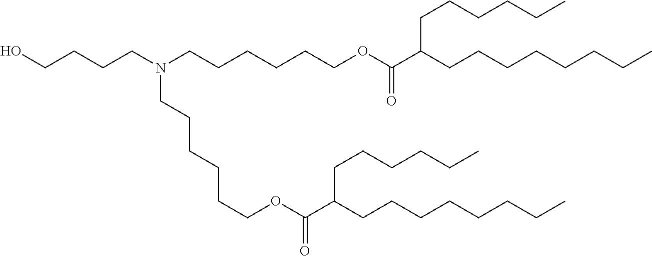 [SMILES: CCCCCCCCC(CCCCCC)C(=O)OCCCCCCN(CCCCO)CCCCCCOC(=O)C(CCCCCC)CCCCCCCC]

Cationic lipids are disclosed in, e.g., U.S. Pat. No. 10,166,298, the full disclosures of which are herein incorporated by reference in their entirety for all purposes. Representative cationic lipids include:

| No. | Structure |
|---|---|
| 1 | 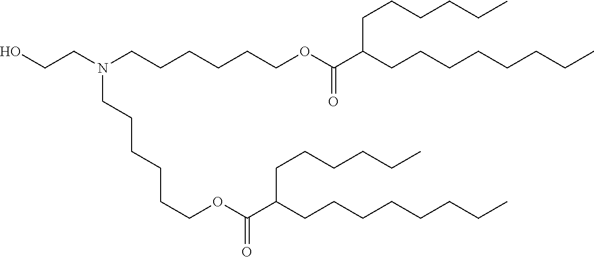 [SMILES: CCCCCCCCC(CCCCCC)C(=O)OCCCCCCN(CCO)CCCCCCOC(=O)C(CCCCCC)CCCCCCCC] |
| 2 | 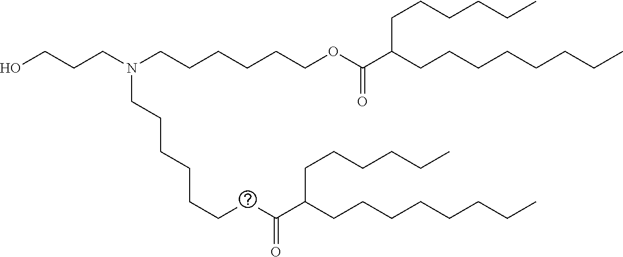 [SMILES: CCCCCCCCC(CCCCCC)C(=O)CCCCCCCN(CCCO)CCCCCCOC(=O)C(CCCCCC)CCCCCCCC] |
| 3 | 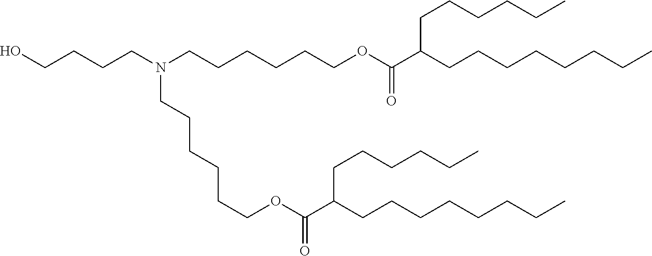 [SMILES: CCCCCCCCC(CCCCCC)C(=O)OCCCCCCN(CCCCO)CCCCCCOC(=O)C(CCCCCC)CCCCCCCC] |
| 4 | 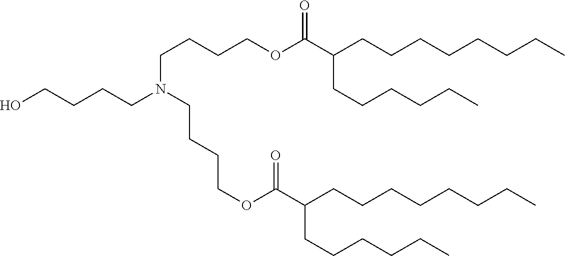 [SMILES: CCCCCCCCC(CCCCCC)C(=O)OCCCCN(CCCCO)CCCCOC(=O)C(CCCCCC)CCCCCCCC] |
| 5 | 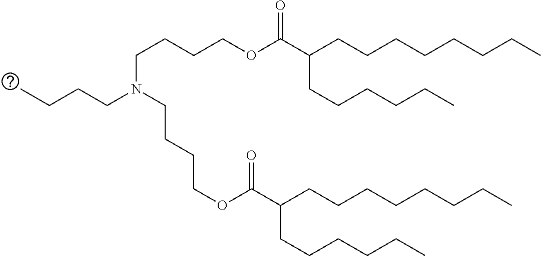 [SMILES: CCCCCCCCC(CCCCCC)C(=O)OCCCCN(CCCC)CCCCOC(=O)C(CCCCCC)CCCCCCCC] |
| 6 | 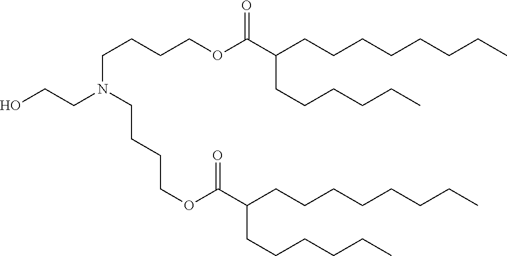 [SMILES: CCCCCCCCC(CCCCCC)C(=O)OCCCCN(CCO)CCCCOC(=O)C(CCCCCC)CCCCCCCC] |
| 7 | 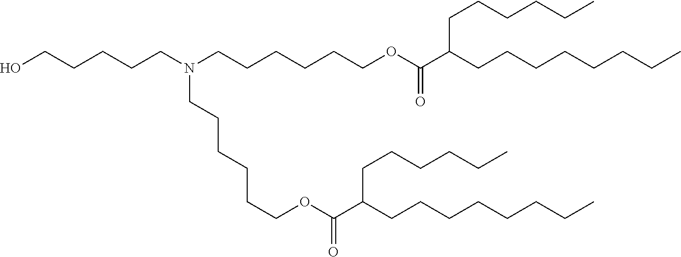 [SMILES: CCCCCCCCC(CCCCCC)C(=O)OCCCCCCN(CCCCCO)CCCCCCOC(=O)C(CCCCCC)CCCCCCCC] |
| 8 | 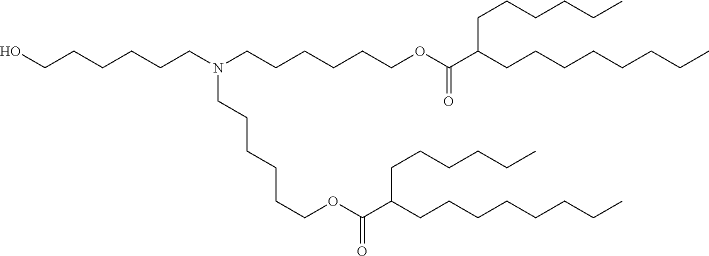 [SMILES: CCCCCCCCC(CCCCCC)C(=O)OCCCCCCN(CCCCCCO)CCCCCCOC(=O)C(CCCCCC)CCCCCCCC] |
| 9 | 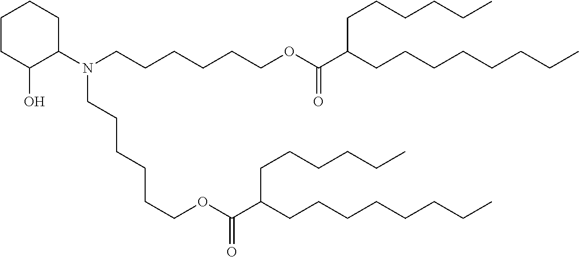 [SMILES: CCCCCCCCC(CCCCCC)C(=O)OCCCCCCN(CCCCCCOC(=O)C(CCCCCC)CCCCCCCC)C1CCCCC1O] |
| 10 | 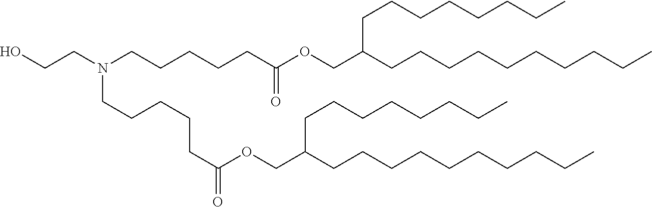 [SMILES: CCCCCCCCCCC(CCCCCCCC)COC(=O)CCCCCN(CCO)CCCCCC(=O)OCC(CCCCCCCC)CCCCCCCCCC] |
| 11 | 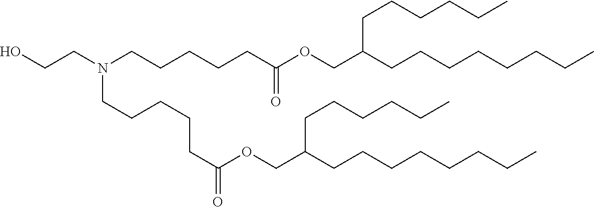 [SMILES: CCCCCCCCC(CCCCCC)COC(=O)CCCCCN(CCO)CCCCCC(=O)OCC(CCCCCC)CCCCCCCC] |
| 12 | 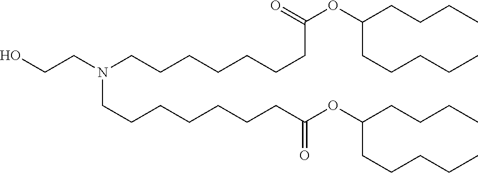 [SMILES: CCCCCC(CCCC)OC(=O)CCCCCCCN(CCO)CCCCCCCC(=O)OC(CCCC)CCCCC] |
| 13 | 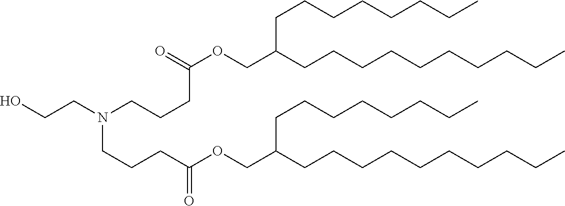 [SMILES: CCCCCCCCCCC(CCCCCCCC)COC(=O)CCCN(CCO)CCCC(=O)OCC(CCCCCCCC)CCCCCCCCCC] |
| 14 | 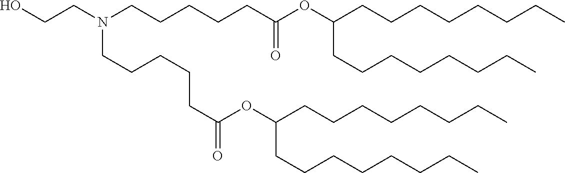 [SMILES: CCCCCCCCC(CCCCCCCC)OC(=O)CCCCCN(CCO)CCCCCC(=O)OC(CCCCCCCC)CCCCCCCC] |
| 15 | 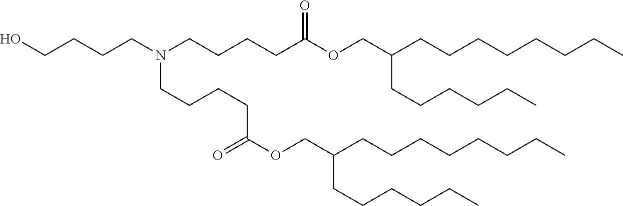 [SMILES: CCCCCCCCC(CCCCCC)COC(=O)CCCCN(CCCCO)CCCCC(=O)OCC(CCCCCC)CCCCCCCC] |
| 16 | 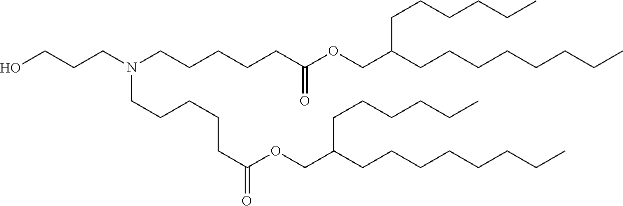 [SMILES: CCCCCCCCC(CCCCCC)COC(=O)CCCCCN(CCCO)CCCCCC(=O)OCC(CCCCCC)CCCCCCCC] |
| 17 | 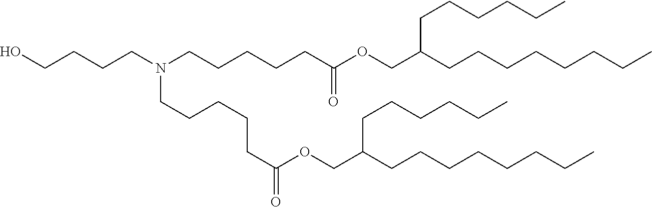 [SMILES: CCCCCCCCC(CCCCCC)COC(=O)CCCCCN(CCCCO)CCCCCC(=O)OCC(CCCCCC)CCCCCCCC] |
| 18 | 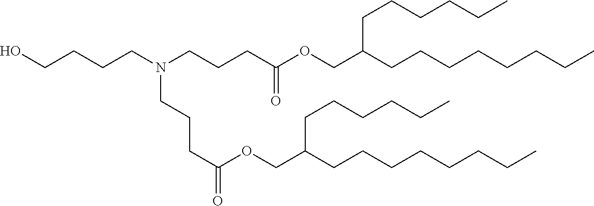 [SMILES: CCCCCCCCC(CCCCCC)COC(=O)CCCN(CCCCO)CCCC(=O)OCC(CCCCCC)CCCCCCCC] |
| 19 | 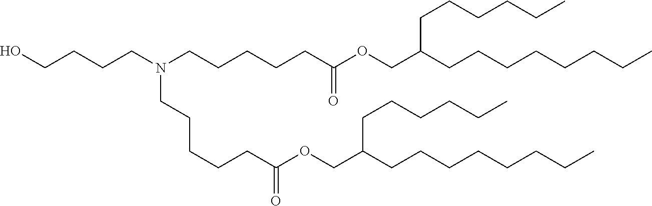 [SMILES: CCCCCCCCC(CCCCCC)COC(=O)CCCCCN(CCCCO)CCCCCC(=O)OCC(CCCCCC)CCCCCCCC] |
| 20 | 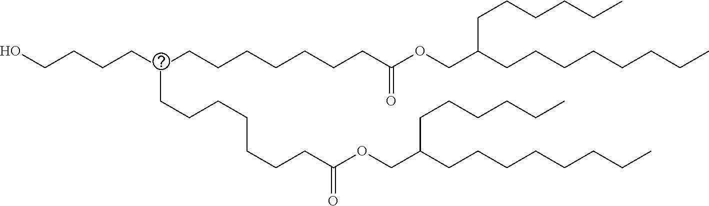 [SMILES: CCCCCCCCC(CCCCCC)COC(=O)CCCCCCCC(CCCCO)CCCCCCCC(=O)OCC(CCCCCC)CCCCCCCC] |
| 21 | 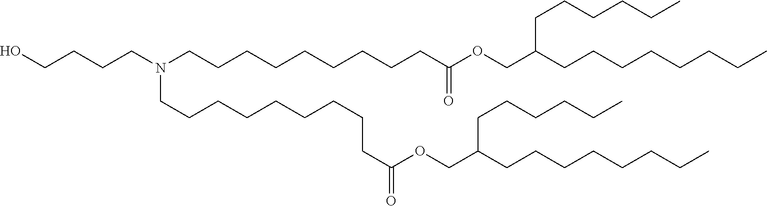 [SMILES: CCCCCCCCC(CCCCCC)COC(=O)CCCCCCCCCN(CCCCO)CCCCCCCCCC(=O)OCC(CCCCCC)CCCCCCCC] |
| 22 | 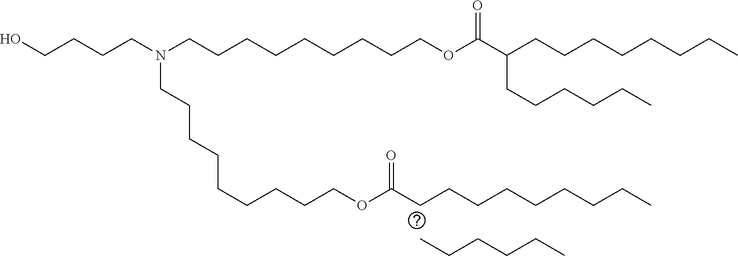 |
| 23 | 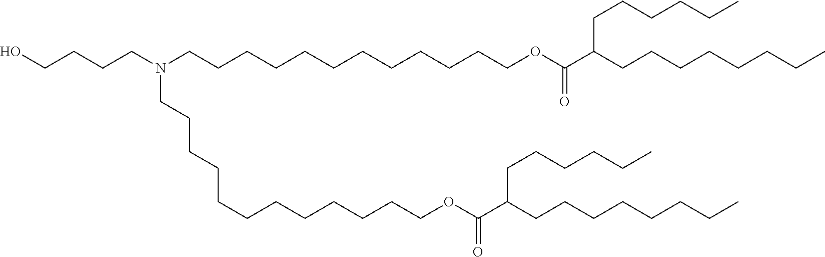 [SMILES: CCCCCCCCC(CCCCCC)C(=O)OCCCCCCCCCCCCN(CCCCO)CCCCCCCCCCCCOC(=O)C(CCCCCC)CCCCCCCC] |
| 24 | 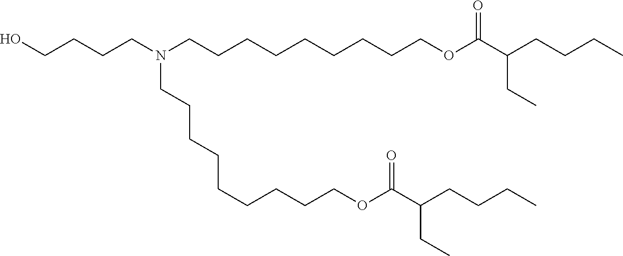 [SMILES: CCCCC(CC)C(=O)OCCCCCCCCCN(CCCCO)CCCCCCCCCOC(=O)C(CC)CCCC] |
| 25 | 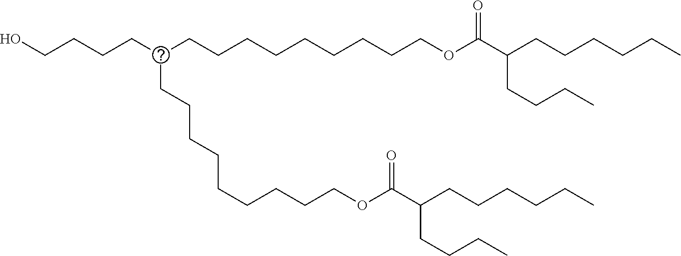 [SMILES: CCCCCCC(CCCC)C(=O)OCCCCCCCCCC(CCCCO)CCCCCCCCCOC(=O)C(CCCC)CCCCCC] |
| 26 | 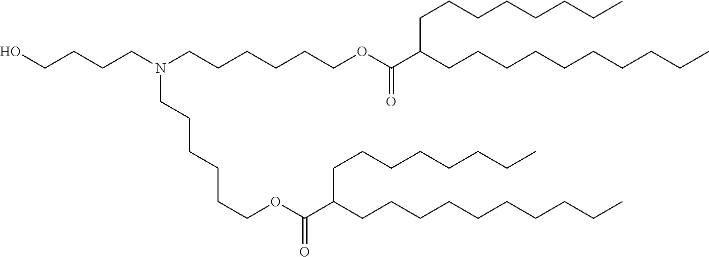 [SMILES: CCCCCCCCCCC(CCCCCCCC)C(=O)OCCCCCCN(CCCCO)CCCCCCOC(=O)C(CCCCCCCC)CCCCCCCCCC] |
| 27 | 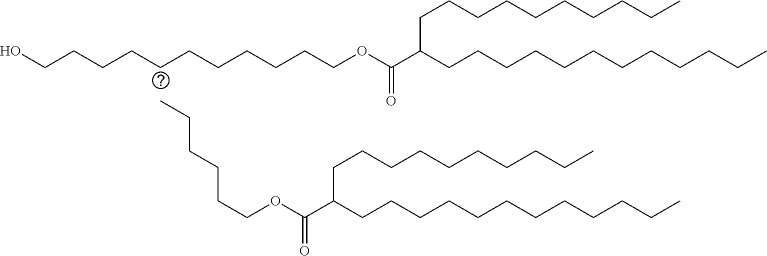 |
| 28 | 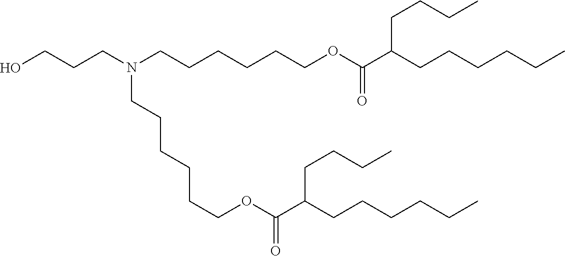 [SMILES: CCCCCCC(CCCC)C(=O)OCCCCCCN(CCCO)CCCCCCOC(=O)C(CCCC)CCCCCC] |
| 29 | 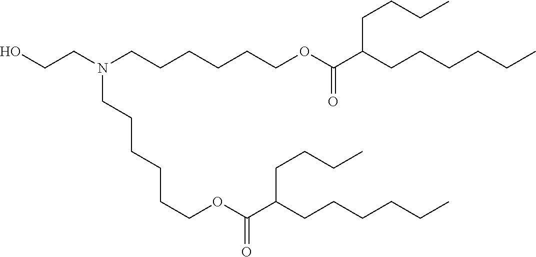 [SMILES: CCCCCCC(CCCC)C(=O)OCCCCCCN(CCO)CCCCCCOC(=O)C(CCCC)CCCCCC] |
| 30 | 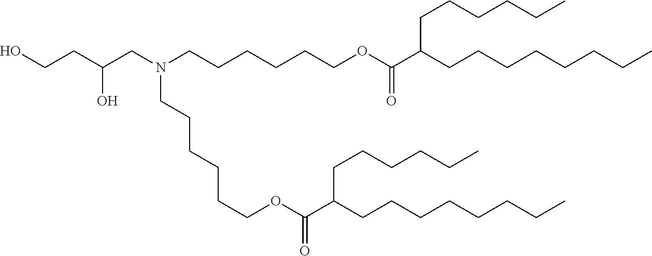 [SMILES: CCCCCCCCC(CCCCCC)C(=O)OCCCCCCN(CCCCCCOC(=O)C(CCCCCC)CCCCCCCC)CC(O)CCO] |
| 31 | 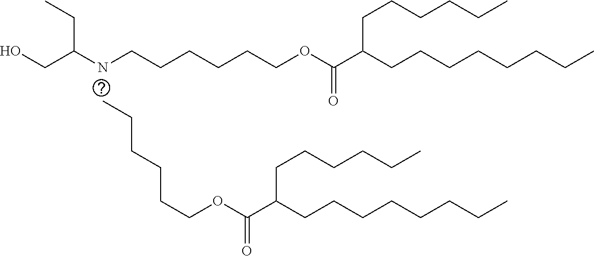 |
| 32 | 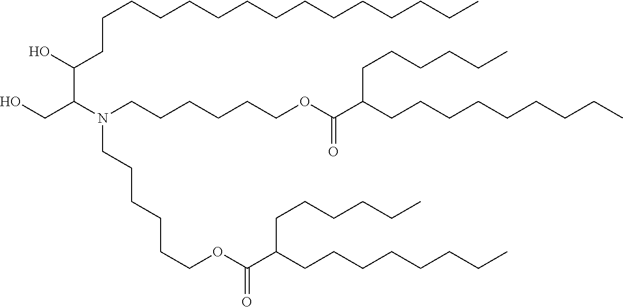 [SMILES: CCCCCCCCCCCCCCCC(O)C(CO)N(CCCCCCOC(=O)C(CCCCCC)CCCCCCCC)CCCCCCOC(=O)C(CCCCCC)CCCCCCCCC] |
| 33 | 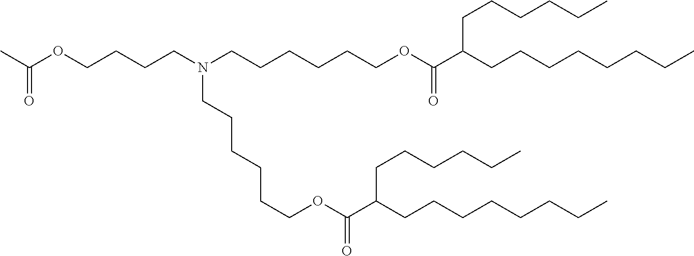 [SMILES: CCCCCCCCC(CCCCCC)C(=O)OCCCCCCN(CCCCCCOC(=O)C(CCCCCC)CCCCCCCC)CCCCOC(C)=O] |
| 34 | 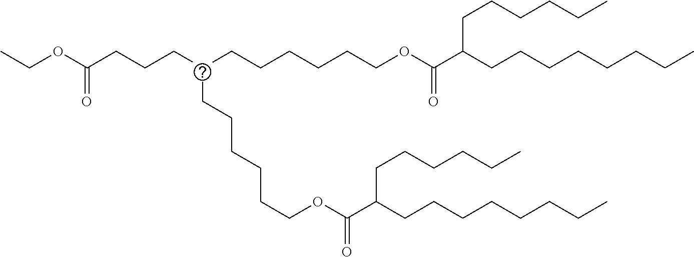 [SMILES: CCCCCCCCC(CCCCCC)C(=O)OCCCCCCC(CCCCCCOC(=O)C(CCCCCC)CCCCCCCC)CCCC(=O)OCC] |
| 35 | 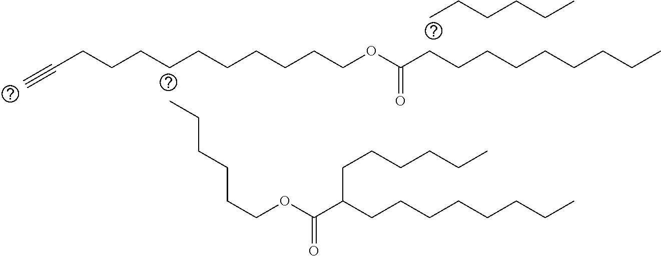 |
| 36 | 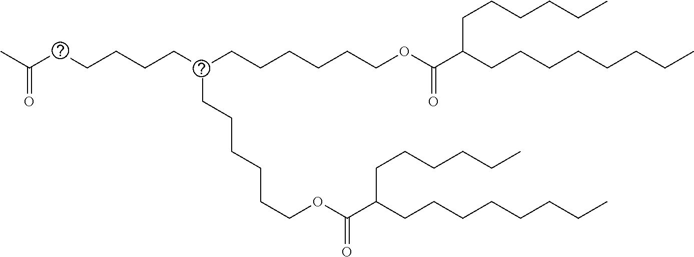 [SMILES: CCCCCCCCC(CCCCCC)C(=O)OCCCCCCC(CCCCCCOC(=O)C(CCCCCC)CCCCCCCC)CCCCCC(C)=O] |

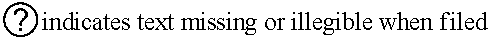

In some aspects, the RNA-LNPs comprise a cationic lipid, a RNA molecule as described herein and one or more of neutral lipids, steroids, pegylated lipids, or combinations thereof. If more than one cationic lipid is incorporated within the LNP, such percentages apply to the combined cationic lipids. In one aspect, the cationic lipid is present in the LNP in an amount such as at least, at most, exactly, or between any two of about 40, 41, 42, 43, 44, 45, 46, 47, 48, 49, 50, 51, 52, 53, 54, 55, 56, 57, 58, 59 or 60 mole percent, respectively.

In some aspects of the disclosure the LNP comprises a combination or mixture of any the lipids described above.

ii. Polymer Conjugated Lipid

In some aspects, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), 2-[(polyethylene glycol)-2000]-N,N-ditetradecylacetamide, and the like.

In certain aspects, the LNP comprises an additional, stabilizing-lipid which is a polyethylene glycol-lipid (pegylated lipid). A polymer conjugated lipid (e.g. PEG-lipid) refers to a molecule comprising both a lipid portion and a polymer portion. An example of a polymer conjugated lipid is a PEG-lipid. A PEG-lipid refers to a molecule comprising both a lipid portion and a polyethylene glycol portion. PEG-lipids include, but are not limited to, PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one aspect, the polyethylene glycol-lipid is N-[(methoxy polyethylene glycol)2000)carbamyl]-1,2-dimyristyloxlpropyl-3-amine (PEG-c-DMA). In one aspect, the polyethylene glycol-lipid is PEG-2000-DMG. In one aspect, the polyethylene glycol-lipid is PEG-c-DOMG). In other aspects, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-((o-methoxy(polyethoxy)ethyl)butanedioate (PEG-S-DMG), a PEGylated ceramide (PEG-cer), or a PEG dialkoxypropylcarbamate such as co-methoxy(polyethoxy)ethyl-N-(2,3di(tetradecanoxy)propyl)carbamate or 2,3-di(tetradecanoxy)propyl-N-(u>-methoxy(polyethoxy)ethyl)carbamate. PEG-lipids are disclosed in, e.g., U.S. Pat. No. 9,737,619, the full disclosures of which are herein incorporated by reference in their entirety for all purposes.

In some aspects, the lipid nanoparticles comprise a polymer conjugated lipid. In one aspect, the lipid nanoparticle comprises 2-[(polyethylene glycol)-2000]-N,N-ditetradecylacetamide (ALC-0159), having the formula:

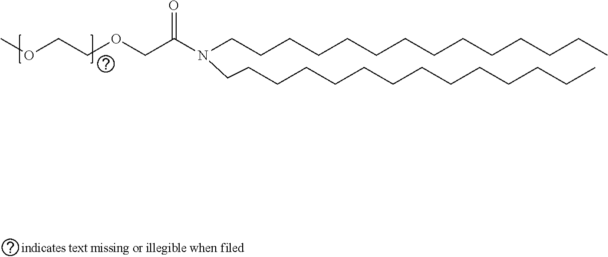

In various aspects, the molar ratio of the cationic lipid to the pegylated lipid ranges from about 100:1 to about 20:1, e.g., from about 20:1, 25:1, 30:1, 35:1, 40:1, 45:1, 50:1, 55:1, 60:1, 65:1, 70:1, 75:1, 80:1, 85:1, 90:1, 95:1, or 100:1, or any range or value derivable therein.

In certain aspects, the PEG-lipid is present in the LNP in an amount from about 1 to about 10 mole percent (mol %) (e.g., at least, at most, exactly, or between any two of 1, 2, 3, 4, 5, 6, 7, 8, 9, or 10 mol %), relative to the total lipid content of the nanoparticle.

iii. Additional Lipids

In certain aspects, the LNP comprises one or more additional lipids or lipid-like materials that stabilize the formation of particles during their formation. Suitable stabilizing or structural lipids include non-cationic lipids, e.g., neutral lipids and anionic lipids. Without being bound by any theory, optimizing the formulation of LNPs by addition of other hydrophobic moieties, such as cholesterol and lipids, in addition to an ionizable/cationic lipid or lipid-like material may enhance particle stability and efficacy of nucleic acid delivery.

As used herein, an “anionic lipid” refers to any lipid that is negatively charged at a selected pH. The term “neutral lipid” refers to any one of a number of lipid species that exist in either an uncharged or neutral zwitterionic form at physiological pH. In some aspects, additional lipids comprise one of the following neutral lipid components: (1) a phospholipid, (2) cholesterol or a derivative thereof; or (3) a mixture of a phospholipid and cholesterol or a derivative thereof.

Representative neutral lipids include phosphatidylcholines, phosphatidylethanolamines, phosphatidylglycerols, phosphatidic acids, phosphatidylserines, ceramides, sphingomyelins, dihydro-sphingomyelins, cephalins, and cerebrosides. Exemplary phospholipids include, for example, phosphatidylcholines, e.g., diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), dioleoylphosphatidylglycerol (DOPG), dipalmitoylphosphatidylglycerol (DPPG), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), I-oleoyl-2-cholesterylhemisuccinoyl-sn-glycero-3-phosphocholine (OChemsPC), and 1-hexadecyl-sn-glycero-3-phosphocholine (C16 Lyso PC), and phosphatidylethanolamines, e.g., diacylphosphatidylethanolamines, such as dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-Icarboxylate (DOPE-mal), dipalmitoyl phosphatidyl ethanolamine (DPPE), dimyristoylphosphoethanolamine (DMPE), dilauroyl-phosphatidylethanolamine (DLPE), distearoyl-phosphatidylethanolamine (DSPE), iphytanoyl-phosphatidylethanolamine (DpyPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one aspect, the neutral lipid is 1,2-distearoyl-sn-glycero-3phosphocholine (DSPC), having the formula:

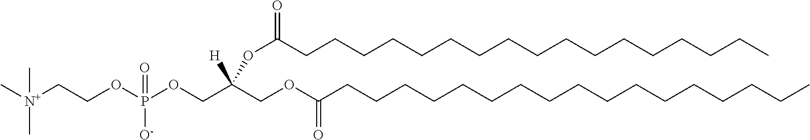 [SMILES: CCCCCCCCCCCCCCCCCC(=O)OC[C@H](COP(=O)([O-])OCC[N+](C)(C)C)OC(=O)CCCCCCCCCCCCCCCCC]

In some aspects, the LNPs comprise a neutral lipid, and the neutral lipid comprises one or more of DSPC, DPPC, DMPC, DOPC, POPC, DOPE, or SM.

In various aspects, the LNPs further comprise a steroid or steroid analogue. A “steroid” is a compound comprising the following carbon skeleton:

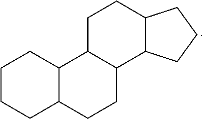 [SMILES: C1CCC2C(C1)CCC1C3CCCC3CCC21]

In certain aspects, the steroid or steroid analogue is cholesterol. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof. In one aspect, the cholesterol has the formula:

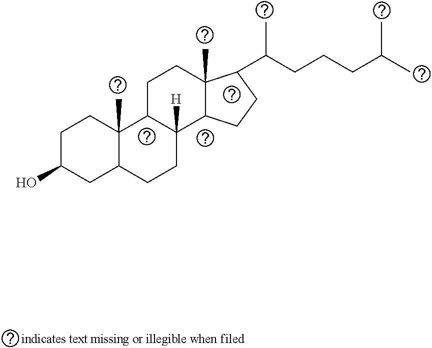

Without being bound by any theory, the amount of the at least one cationic lipid compared to the amount of the at least one additional lipid may affect important nucleic acid particle characteristics, such as charge, particle size, stability, tissue selectivity, and bioactivity of the nucleic acid. Accordingly, in some aspects, the molar ratio of the cationic lipid to the neutral lipid ranges from about 2:1 to about 8:1, or from about 10:0 to about 1:9, about 4:1 to about 1:2, or about 3:1 to about 1:1.

In some aspects, the non-cationic lipid, e.g., neutral lipid (e.g., one or more phospholipids and/or cholesterol), may comprise from about 0 mol % to about 90 mol %, from about 0 mol % to about 80 mol %, from about 0 mol % to about 70 mol %, from about 0 mol % to about 60 mol %, or from about 0 mol % to about 50 mol %, of the total lipid present in the particle. In some aspects, the non-cationic lipid, e.g., neutral lipid (e.g., one or more phospholipids and/or cholesterol), may be at least, at most, exactly, or between any two of 0 mol %, 10 mol %, 20 mol %, 30 mol %, 40 mol %, 50 mol %, 60 mol %, 70 mol %, 80 mol %, or 90 mol % of the total lipid present in the particle.

### VI. Characterization and Analysis of RNA Molecule

The RNA molecule described herein may be analyzed and characterized using various methods. Analysis may be performed before or after capping. Alternatively, analysis may be performed before or after poly-A capture-based affinity purification. In another aspect, analysis may be performed before or after additional purification steps, e.g., anion exchange chromatography and the like. For example, RNA template quality may be determined using Bioanalyzer chip based electrophoresis system. In other aspects, RNA template purity is analyzed using analytical reverse phase HPLC respectively. Capping efficiency may be analyzed using, e.g., total nuclease digestion followed by MS/MS quantitation of the dinucleotide cap species vs. uncapped GTP species. In vitro efficacy may be analyzed by, e.g., transfecting RNA molecule into a human cell line. Protein expression of the polypeptide of interest may be quantified using methods such as ELISA or flow cytometry. Immunogenicity may be analyzed by, e.g., transfecting RNA molecules into cell lines that indicate innate immune stimulation, e.g., PBMCs. Cytokine induction may be analyzed using, e.g., methods such as ELISA to quantify a cytokine, e.g., Interferon-α. Biodistribution may be analyzed, e.g. by bioluminescence measurements.

In some aspects, an RNA polynucleotide disclosed herein is characterized in that, when assessed in an organism administered a composition or medical preparation comprising an RNA polynucleotide, elevated expression of a gene of interest (e.g., an antigen); increased duration of expression (e.g., prolonged expression) of a gene of interest (e.g., an antigen); elevated expression and increased duration of expression (e.g., prolonged expression) of a gene of interest (e.g., an antigen); decreased interaction with IFIT1 of an RNA polynucleotide; increased translation of an RNA polynucleotide; is observed relative to an appropriate reference.

In some aspects, a reference comprises an organism administered an otherwise similar RNA polynucleotide without a m7(3′OMeG)(5′)ppp(5′)(2′OMeAi)pG2 cap. In some aspects, a reference comprises an organism administered an otherwise similar RNA polynucleotide without a cap proximal sequence disclosed herein. In some aspects, a reference comprises an organism administered an otherwise similar RNA polynucleotide with a self-hybridizing sequence.

In some aspects, elevated expression is determined at least 24 hours, at least 48 hours at least 72 hours, at least 96 hours, or at least 120 hours after administration of a composition or medical preparation comprising an RNA polynucleotide. In some aspects, elevated expression is determined at least 24 hours after administration of a composition or medical preparation comprising an RNA polynucleotide. In some aspects, elevated expression is determined at least 48 hours after administration of a composition or medical preparation comprising an RNA polynucleotide. In some aspects, elevated expression is determined at least 72 hours after administration of a composition or medical preparation comprising an RNA polynucleotide. In some aspects, elevated expression is determined at least 96 hours after administration of a composition or medical preparation comprising an RNA polynucleotide. In some aspects, elevated expression is determined at least 120 hours after administration of a composition or medical preparation comprising an RNA polynucleotide.

In some aspects, elevated expression is determined at about 24-120 hours after administration of a composition or medical preparation comprising an RNA polynucleotide. In some aspects, elevated expression is determined at about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide.

In some aspects, elevated expression of a gene of interest (e.g., an antigen) is at least 2-fold to at least 10-fold. In some aspects, elevated expression of a gene of interest (e.g., an antigen) is at least 2-fold. In some aspects, elevated expression of a gene of interest (e.g., an antigen) is at least 3-fold. In some aspects, elevated expression of a gene of interest (e.g., an antigen) is at least 4-fold. In some aspects, elevated expression of a gene of interest (e.g., an antigen) is at least 6-fold. In some aspects, elevated expression of a gene of interest (e.g., an antigen) is at least 8-fold. In some aspects, elevated expression of a gene of interest (e.g., an antigen) is at least 10-fold.

In some aspects, elevated expression of a gene of interest (e.g., an antigen) is about 2-fold to about 50-fold. In some aspects, elevated expression of a gene of interest (e.g., an antigen) is about 2-fold to about 45-fold, about 2-fold to about 40-fold, about 2-fold to about 30-fold, about 2-fold to about 25-fold, about 2-fold to about 20-fold, about 2-fold to about 15-fold, about 2-fold to about 10-fold, about 2-fold to about 8-fold, about 2-fold to about 5-fold, about 5-fold to about 50-fold, about 10-fold to about 50-fold, about 15-fold to about 50-fold, about 20-fold to about 50-fold, about 25-fold to about 50-fold, about 30-fold to about 50-fold, about 40-fold to about 50-fold, or about 45-fold to about 50-fold. In some aspects, elevated expression of a gene of interest (e.g., an antigen) is at least, at most, exactly, or between any two of 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold, or any range or value derivable therein.

In some aspects, elevated expression (e.g., increased duration of expression) of a gene of interest (e.g., an antigen) persists for at least, at most, exactly, or between any two of 24 hours, 48 hours, 72 hours, 96 hours, or 120 hours after administration of a composition or a medical preparation comprising an RNA polynucleotide. In some aspects, elevated expression of a gene of interest (e.g., an antigen) persists for at least 24 hours after administration. In some aspects, elevated expression of a gene of interest (e.g., an antigen) persists for at least 48 hours after administration. In some aspects, elevated expression of a gene of interest (e.g., an antigen) persists for at least 72 hours after administration. In some aspects, elevated expression of a gene of interest (e.g., an antigen) persists for at least 96 hours after administration. In some aspects, elevated expression of a gene of interest (e.g., an antigen) persists for at least 120 hours after administration of a composition or medical preparation comprising an RNA polynucleotide.

In some aspects, elevated expression of a gene of interest (e.g., an antigen) persists for about 24-120 hours after administration of a composition or medical preparation comprising an RNA polynucleotide. In some aspects, elevated expression persists for about 24-110 hours, about 24-100 hours, about 24-90 hours, about 24-80 hours, about 24-70 hours, about 24-60 hours, about 24-50 hours, about 24-40 hours, about 24-30 hours, about 30-120 hours, about 40-120 hours, about 50-120 hours, about 60-120 hours, about 70-120 hours, about 80-120 hours, about 90-120 hours, about 100-120 hours, or about 110-120 hours after administration of a composition or medical preparation comprising an RNA polynucleotide. In some aspects, elevated expression of a gene of interest (e.g., an antigen) persists for at least, at most, exactly, or between any two of 24 hours, 36 hours, 48 hours, 60 hours, 72 hours, 84 hours, 96 hours, 108 hours, or 120 hours, or any range or value derivable therein.

### VII. Immune Response and Assays

As discussed herein, the disclosure concerns evoking or inducing an immune response in a subject against a VZV protein, e.g., a wild type or variant VZV glycoprotein. In one aspect, the immune response may protect against or treat a subject having, suspected of having, or at risk of developing an infection or related disease, particularly those related to VZV. One use of the immunogenic compositions of the disclosure is to prevent VZV infections by inoculating or vaccination of a subject.

A. Immunoassays

The present disclosure includes the implementation of serological assays to evaluate whether and to what extent an immune response is induced or evoked by compositions of the disclosure. There are many types of immunoassays that may be implemented. Immunoassays encompassed by the present disclosure include, but are not limited to, those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

Immunoassays generally are binding assays. In some aspects, the immunoassays are the various types of enzyme linked immunosorbent assays (ELISAs) and radioimmunoassays (RIA) known in the art. Immunohistochemical detection using tissue sections is also particularly useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non-specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non-specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

B. Diagnosis of VZV Infection

The present disclosure contemplates the use of VZV polypeptides, proteins, and/or peptides in a variety of ways, including the detection of the presence of VZV to diagnose an infection. In accordance with the disclosure, a method of detecting the presence of infections involves the steps of obtaining a sample suspected of being infected by one or more VZV strains, such as a sample taken from an individual, for example, from one's blood, saliva, tissues, bone, muscle, cartilage, or skin. Following isolation of the sample, diagnostic assays utilizing the polypeptides, proteins, and/or peptides of the present disclosure may be carried out to detect the presence of VZV, and such assay techniques for determining such presence in a sample are well known to those skilled in the art and include methods such as radioimmunoassay, western blot analysis and ELISA assays.

In general, in accordance with the disclosure, a method of diagnosing an infection is contemplated wherein a sample suspected of being infected with VZV has added to it the polypeptide, protein, or peptide, in accordance with the present disclosure, and VZV is indicated by antibody binding to the polypeptides, proteins, and/or peptides, or polypeptides, proteins, and/or peptides binding to the antibodies in the sample.

Accordingly, RNA molecules encoding VZV polypeptides, proteins, and/or peptides in accordance with the disclosure may be used for to treat, prevent, or reduce the severity of illness from infection due to VZV infection (e.g., active or passive immunization) or for use as research tools.

Any of the above described polypeptides, proteins, and/or peptides may be labeled directly with a detectable label for identification and quantification of VZV. Labels for use in immunoassays are generally known to those skilled in the art and include enzymes, radioisotopes, and fluorescent, luminescent and chromogenic substances, including colored particles such as colloidal gold or latex beads. Suitable immunoassays include enzyme-linked immunosorbent assays (ELISA).

C. Protective Immunity

In some aspects of the disclosure, RNA molecules encoding VZV polypeptides, RNA-LNPs and compositions thereof, confer protective immunity to a subject. Protective immunity refers to a body's ability to mount a specific immune response that protects the subject from developing a particular disease or condition that involves the agent against which there is an immune response. An immunogenically effective amount is capable of conferring protective immunity to the subject.

As used herein the phrase “immune response” or its equivalent “immunological response” refers to the development of a humoral (antibody mediated), cellular (mediated by antigen-specific T cells or their secretion products) or both humoral and cellular response directed against an antigen. Such a response may be an active response or a passive response. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject from the production of antibodies in response to the presence of an of an antigen, e.g. a VZV polypeptide encoded by a RNA molecule of the present disclosure.

As used herein “passive immunity” includes, but is not limited to, administration of activated immune effectors including cellular mediators or protein mediators (e.g., monoclonal and/or polyclonal antibodies) of an immune response. A monoclonal or polyclonal antibody composition may be used in passive immunization to treat, prevent, or reduce the severity of illness caused by infection by organisms that carry the antigen recognized by the antibody. An antibody composition may include antibodies that bind to a variety of antigens that may in turn be associated with various organisms. The antibody component may be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as viruses, including but not limited to VZV.

Passive immunity may be imparted to a patient or subject by administering to the patient immunoglobulins (Ig) and/or other immune factors obtained from a donor or other non-patient source having a known immunoreactivity. In other aspects, an immunogenic composition of the present disclosure may be administered to a subject who then acts as a source or donor for globulin, produced in response to challenge with the immunogenic composition (“hyperimmune globulin”), that contains antibodies directed against a VZV or other organism. A subject thus treated would donate plasma from which hyperimmune globulin would then be obtained, via conventional plasma-fractionation methodology, and administered to another subject in order to impart resistance against or to treat VZV infection.

For purposes of this specification and the accompanying claims the terms “epitope” and “antigenic determinant” are used interchangeably to refer to a site on an antigen to which B and/or T cells respond or recognize. B-cell epitopes may be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996). Antibodies that recognize the same epitope may be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope may be identified by in vitro assays that measure antigen-dependent proliferation, as determined by ³H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response may be determined by proliferation assays (CD4 (+) T cells) or CTL (cytotoxic T lymphocyte) assays. The relative contributions of humoral and cellular responses to the protective or therapeutic effect of an immunogenic composition may be distinguished by separately isolating IgG and T-cells from an immunized syngeneic animal and measuring protective or therapeutic effect in a second subject.

As used herein, the terms “antibody” or “immunoglobulin” are used interchangeably and refer to any of several classes of structurally related proteins that function as part of the immune response of an animal or recipient, which proteins include IgG, IgD, IgE, IgA, IgM and related proteins. Under normal physiological conditions antibodies are found in plasma and other body fluids and in the membrane of certain cells and are produced by lymphocytes of the type denoted B cells or their functional equivalent.

As used herein the terms “immunogenic agent” or “immunogen” or “antigen” are used interchangeably to describe a molecule capable of inducing an immunological response against itself on administration to a recipient, either alone, in conjunction with an adjuvant, or presented on a display vehicle.

### VIII. Compositions

In some aspects, an RNA molecules and/or RNA-LNPs disclosed herein may be administered in a pharmaceutical composition or a medicament and may be administered in the form of any suitable pharmaceutical composition. In some aspects, a pharmaceutical composition is for therapeutic or prophylactic treatments. In one aspect, the disclosure relates to a composition for administration to a host. In some aspects, the host is a human. In other aspects, the host is a non-human.

In some aspects, an RNA molecules and/or RNA-LNPs disclosed herein may be administered in a pharmaceutical composition which may be formulated into preparations in solid, semi-solid, liquid, lyophilized, frozen, or gaseous forms. In some aspects, an RNA molecule and/or RNA-LNPs disclosed herein may be administered in a pharmaceutical composition which may comprise a pharmaceutically acceptable carrier and may optionally comprise one or more adjuvants, stabilizers, salts, buffers, preservatives, and optionally other therapeutic agents. In some aspects, a pharmaceutical composition disclosed herein comprises one or more pharmaceutically acceptable carriers, diluents and/or excipients. In some aspects, pharmaceutical compositions do not include an adjuvant (e.g., they are adjuvant free).

Suitable preservatives for use in a pharmaceutical compositions of the present disclosure include, without limitation, benzalkonium chloride, chlorobutanol, paraben and thimerosal. The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol saline and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some aspects, the pharmaceutical composition of the present disclosure includes sodium chloride.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents may be selected with regard to the intended route of administration and standard pharmaceutical practice.

In some aspects, the composition comprises an RNA molecule comprising an open reading frame encoding an immunogenic polypeptide. In some aspects, the immunogenic polypeptide comprises a VZV antigen. In some aspects, the VZV antigen is a VZV polypeptide. In some aspects, the VZV polypeptide is a VZV glycoprotein (e.g. gK, gN, gC, gB, gH, gM, gL gI, and gE) or a fragment or a variant thereof. In some aspects, the RNA molecule encodes a VZV gK polypeptide, the RNA molecule encodes a VZV gN polypeptide, the RNA molecule encodes a VZV gC polypeptide, the RNA molecule encodes a VZV gB polypeptide, the RNA molecule encodes a VZV gH polypeptide, the RNA molecule encodes a VZV gM polypeptide, the RNA molecule encodes a VZV gL polypeptide, the RNA molecule encodes a VZV gI polypeptide, and/or the RNA molecule encodes a VZV gE polypeptide. In one aspect, the RNA molecule encodes a VZV gE polypeptide. In some aspects, the VZV polypeptide comprises two or more (e.g., 2, 3, 4, 5, 6, 7, 8, 9, or more) VZV polypeptides.

In some aspects, the composition comprises an RNA molecule comprising an open reading frame encoding a full-length VZV polypeptide. In some aspects, the encoded immunogenic polypeptide is a truncated VZV polypeptide. In some aspects, the encoded immunogenic polypeptide is a variant of a VZV polypeptide. In some aspects, the encoded immunogenic polypeptide is a fragment of a VZV polypeptide.

A. Immunogenic Compositions Including LNPS

In some aspects, a pharmaceutical composition comprises an RNA molecule (e.g., polynucleotide) disclosed herein formulated with a lipid-based delivery system. Thus, some aspects, the composition includes a lipid-based delivery system (e.g., LNPs) (e.g., a lipid-based vaccine), which delivers a nucleic acid molecule to the interior of a cell, where it may then replicate, inhibit protein expression of interest, and/or express the encoded polypeptide of interest. The delivery system may have adjuvant effects which enhance the immunogenicity of an encoded antigen. In some aspects, the composition comprises at least one RNA molecule encoding a VZV polypeptide complexed with, encapsulated in, and/or formulated with one or more lipids, and forming lipid nanoparticles (LNPs), liposomes, lipoplexes and/or nanoliposomes. In some aspects, the composition comprises a lipid nanoparticle. Thus, in certain aspects, the present disclosure concerns compositions comprising one or more lipids associated with a nucleic acid or a polypeptide/peptide (e.g., VZV RNA-LNPs).

The immunogenic composition including a lipid-based delivery system may further include one or more salts and/or one or more pharmaceutically acceptable surfactants, preservatives, carriers, diluents, and/or excipients, in some cases. In some aspects, the immunogenic composition including a lipid-based delivery system further include a pharmaceutically acceptable vehicle. In some aspects, each of a buffer, stabilizing agent, and optionally a salt, may be included in the immunogenic composition including a lipid-based delivery system. In other aspects, any one or more of a buffer, stabilizing agent, salt, surfactant, preservative, and excipient may be excluded from the immunogenic composition including a lipid-based delivery system.

In a further aspect, the immunogenic composition including a lipid-based delivery system further comprises a stabilizing agent. In some aspects, the stabilizing agent comprises sucrose, mannose, sorbitol, raffinose, trehalose, mannitol, inositol, sodium chloride, arginine, lactose, hydroxyethyl starch, dextran, polyvinylpyrolidone, glycine, or a combination thereof. In some aspects, the stabilizing agent is a disaccharide, or sugar. In one aspect, the stabilizing agent is sucrose. In another aspect, the stabilizing agent is trehalose. In a further aspect, the stabilizing agent is a combination of sucrose and trehalose. In some aspects, the total concentration of the stabilizing agent(s) in the composition is about 5% to about 10% w/v. For example, the total concentration of the stabilizing agent may be equal to at least, at most, exactly, or between any two of 1%, 2%, 3%, 4%, 5%, 6%, 7%, 8%, 9%, 10%, 11%, 12%, 13%, 14%, 15%, 16%, 17%, 18%, 19%, or 20% w/v or any range or value derivable therein. In some aspects, the stabilizing agent concentration includes, but is not limited to, a concentration of about 10 mg/mL to about 400 mg/mL, about 100 mg/mL to about 200 mg/mL, about 100 mg/mL to about 150 mg/mL, about 100 mg/mL to about 140 mg/mL, about 100 mg/mL to about 130 mg/mL, about 100 mg/mL to about 120 mg/mL, about 100 mg/mL to about 110 mg/mL, or about 100 mg/mL to about 105 mg/mL. In some aspects, the concentration of the stabilizing agent is equal to at least, at most, exactly, or between any two of 10 mg/mL, 20 mg/mL, 50 mg/mL, 100 mg/mL, 101 mg/mL, 102 mg/mL, 103 mg/mL, 104 mg/mL, 105 mg/mL, 106 mg/mL, 107 mg/mL, 108 mg/mL, 109 mg/mL, 110 mg/mL, 150 mg/mL, 200 mg/mL, 300 mg/mL, 400 mg/mL, or more.

In a further aspect, the mass amount of the stabilizing agent and the mass amount of the RNA are in a specific ratio. In one aspect, the ratio of the mass amount of the stabilizing agent and the RNA is no greater than 5000. In another aspect, the ratio of the mass amount of the stabilizing agent and the RNA is no greater than 2000. In another aspect, the ratio of the mass amount of the stabilizing agent and the RNA is no greater than 1000. In another aspect, the ratio of the mass amount of the stabilizing agent and the RNA is no greater than 500. In another aspect, the ratio of the mass amount of the stabilizing agent and the RNA is no greater than 100. In another aspect, the ratio of the mass amount of the stabilizing agent and the pharmaceutical substance is no greater than 50. In another aspect, the ratio of the mass amount of the stabilizing agent and the RNA is no greater than 10. In another aspect, the ratio of the mass amount of the stabilizing agent and the RNA is no greater than 1. In another aspect, the ratio of the mass amount of the stabilizing agent and the RNA is no greater than 0.5. In another aspect, the ratio of the mass amount of the stabilizing agent and the RNA is no greater than 0.1. In another aspect, the stabilizing agent and RNA comprise a mass ratio of about 200-2000 of the stabilizing agent:1 of the RNA.

In some aspects, the immunogenic composition including a lipid-based delivery system further comprises a buffer. Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, Tris hydrochloride (HCl), amino-sulfonate buffers (e.g., HEPES), magnesium hydroxide, aluminum hydroxide, alginic acid, pyrogen-free water, isotonic saline, Ringer's solution, ethyl alcohol, and/or combinations thereof. In some aspects, the buffer is a HEPES buffer, a Tris buffer, or a PBS buffer. In one aspect, the buffer is Tris buffer. In another aspect, the buffer is a HEPES buffer. In a further aspect, the buffer is a PBS buffer. For example, the buffer concentration may be equal to at least, at most, exactly, or between any two of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM, or any range or value derivable therein. The buffer may be at a neutral pH, pH 6.5 to 8.5, pH 7.0 to pH 8.0, or pH 7.2 to pH 7.6. For example, the buffer may be at least, at most, exactly, or between any two of pH 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5, or any range or value derivable therein. In specific aspects, the buffer is at pH 7.4.

In some aspects, the immunogenic composition including a lipid-based delivery system may further comprise a salt. Examples of salts include but not limited to sodium salts and/or potassium salts. In one aspect, the salt is a sodium salt. In a specific aspect, the sodium salt is sodium chloride. In one aspect, the salt is a potassium salt. In some aspects, the potassium salt comprises potassium chloride. The concentration of the salts in the composition may be about 70 mM to about 140 mM. For example, the salt concentration may be equal to at least, at most, exactly, or between any two of 50 mM, 60 mM, 70 mM, 80 mM, 90 mM, 100 mM, 120 mM, 130 mM, 140 mM, 150 mM, 160 mM, 170 mM, 180 mM, 190 mM, or 200 mM.

In some aspects, the salt concentration includes, but is not limited to, a concentration of about 1 mg/mL to about 100 mg/mL, about 1 mg/mL to about 50 mg/mL, about 1 mg/mL to about 40 mg/mL, about 1 mg/mL to about 30 mg/mL, about 1 mg/mL to about 20 mg/mL, about 1 mg/mL to about 10 mg/mL, or about 1 mg/mL to about 15 mg/mL. In some aspects, the concentration of the salt is equal to at least, at most, exactly, or between any two of 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, or more. The salt may be at a neutral pH, pH 6.5 to 8.5, pH 7.0 to pH 8.0, or pH 7.2 to pH 7.6. For example, the salt may be at a pH equal to at least, at most, exactly, or between any two of 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, or 8.5.

In some aspects, the immunogenic composition including a lipid-based delivery system further comprises a surfactant, a preservative, any other excipient, or a combination thereof. As used herein, “any other excipient” includes, but is not limited to, antioxidants, glutathione, EDTA, methionine, desferal, antioxidants, metal scavengers, or free radical scavengers. In one aspect, the surfactant, preservative, excipient or combination thereof is sterile water for injection (sWFI), bacteriostatic water for injection (BWFI), saline, dextrose solution, polysorbates, poloxamers, Triton, divalent cations, Ringer's lactate, amino acids, sugars, polyols, polymers, or cyclodextrins.

Examples of excipients, which refer to ingredients in the immunogenic compositions that are not active ingredients, include but are not limited to carriers, binders, diluents, lubricants, thickeners, surface active agents, preservatives, stabilizers, emulsifiers, buffers, flavoring agents, disintegrants, coatings, plasticizers, compression agents, wet granulation agents, or colorants. Preservatives for use in the compositions disclosed herein include but are not limited to benzalkonium chloride, chlorobutanol, paraben and thimerosal. As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. Diluents, or diluting or thinning agents, include but are not limited to ethanol, glycerol, water, sugars such as lactose, sucrose, mannitol, and sorbitol, and starches derived from wheat, corn rice, and potato, and celluloses such as microcrystalline cellulose. The amount of diluent in the composition may range from about 10% to about 90% by weight of the total composition, about 25% to about 75%, about 30% to about 60% by weight, or about 12% to about 60%.

The pH and exact concentration of the various components in the immunogenic composition including a lipid-based delivery system are adjusted according to well-known parameters. The use of such media and agents for pharmaceutical active substances is well known in the art. Except insofar as any conventional media or agent is incompatible with the active ingredients, its use in immunogenic, prophylactic and/or therapeutic compositions is contemplated.

In one aspect, a pharmaceutical composition comprises a VZV RNA molecule encoding a VZV polypeptide as disclosed herein that is complexed with, encapsulated in, and/or formulated with one or more lipids to form VZV RNA-LNPs. In some aspects, the VZV RNA-LNP composition is a liquid. In some aspects, the VZV RNA-LNP composition is frozen. In some aspects, the VZV RNA-LNP composition is lyophilized. In some aspects, a VZV RNA-LNP composition comprises a VZV RNA polynucleotide molecule encoding a VZV polypeptide as disclosed herein, encapsulated in LNPs with a lipid composition of a cationic lipid, a PEGylated lipid (i.e. PEG-lipid), and one or more structural lipids (e.g., a neutral lipid).

In some aspects, a VZV RNA-LNP composition comprises an cationic lipid. The cationic lipid may comprise any one or more cationic lipids disclosed herein. In specific aspects, the cationic lipid comprises ((4-hydroxybutyl)azanediyl)bis(hexane-6,1-diyl)bis(2-hexyldecanoate) (ALC-0315). In some aspects, the cationic lipid (e.g., ALC-0315) is included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the cationic lipid (e.g., ALC-0315) is included in the composition at a concentration of at least, at most, between any two of, or exactly 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 mg/mL. In some aspects, the cationic lipid (e.g., ALC-0315) is included in the composition at a concentration of at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the cationic lipid (e.g., ALC-0315) is included in the composition at a concentration of between 0.4 and 0.5, between 0.5 and 0.6, between 0.6 and 0.7, between 0.7 and 0.8, between 0.8 and 0.9, or between 0.9 and 1. In some aspects, the cationic lipid (e.g., ALC-0315) is included in the composition at a concentration of between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95, or between 0.95 and 1 mg/mL.

In specific aspects, the cationic lipid (e.g., ALC-0315) is included in the composition at a concentration of 0.8 to 0.95 mg/mL. In specific aspects, the cationic lipid (e.g., ALC-0315) is included in the composition at a concentration of about 0.8 to 0.9 mg/mL. In specific aspects, the cationic lipid (e.g., ALC-0315) is included in the composition at a concentration of about 0.85 to 0.9 mg/mL. In specific aspects, the cationic lipid (e.g., ALC-0315) is included in the composition at a concentration of about 0.8, 0.81, 0.82, 0.83, 0.84, 0.85, 0.86, 0.87, 0.88, 0.89, 0.9, 0.91, 0.92, 0.93, 0.94, or 0.95 mg/mL. In specific aspects, the cationic lipid (e.g., ALC-0315) is included in the composition at a concentration of about 0.86 mg/mL. Concentrations for lyophilized compositions are determined post-reconstitution.

In some aspects, a VZV RNA-LNP composition further comprises a PEGylated lipid (i.e., PEG-lipid). The PEGylated lipid may comprise any one or more PEGylated lipids disclosed herein. In specific aspects, the PEGylated lipid comprises 2-[(polyethylene glycol)-2000]-N,N-ditetradecylacetamide (ALC-0159). In some aspects, the PEGylated lipid (e.g., ALC-0159) is included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the PEGylated lipid (e.g., ALC-0159) is included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the PEGylated lipid (e.g., ALC-0159) is included in the composition at a concentration of at least 0.01, at least 0.05, at least 0.1, at least 0.15, at least 0.2, at least 0.25 mg/mL, at least 0.3 mg/mL, at least 0.35 mg/mL, at least 0.4 mg/mL, at least 0.45 mg/mL or at least 0.5 mg/mL. In some aspects, the PEGylated lipid (e.g., ALC-0159) is included in the composition at a concentration of between 0.01 and 0.05, between 0.05 and 0.1, between 0.1 and 0.15, between 0.15 and 0.2, or between 0.2 and 0.25 mg/mL.

In specific aspects, the PEGylated lipid (e.g., ALC-0159) is included in the composition at a concentration of about 0.05 to 0.15 mg/mL. In specific aspects, the PEGylated lipid (e.g., ALC-0159) is included in the composition at a concentration of about 0.10 to 0.15 mg/mL. In specific aspects, the PEGylated lipid (e.g., ALC-0159) is included in the composition at a concentration of about 0.05, 0.06, 0.07, 0.08, 0.09, 0.1, 0.11, 0.12, 0.13, 0.14 or 0.15 mg/mL. In specific aspects, the PEGylated lipid (e.g., ALC-0159) is included in the composition at a concentration of about 0.11 mg/mL Concentrations for lyophilized compositions are determined post-reconstitution.

In some aspects, a VZV RNA-LNP composition further comprises one or more structural lipids. The one or more structural lipids may comprise any one or more structural lipids disclosed herein. In specific aspects, the one or more structural lipids comprise a neutral lipid and a steroid or steroid analog. In specific aspects, the one or more structural lipids comprise 1,2-Distearoyl-sn-glycero-3-phosphocholine (DSPC) and cholesterol. In some aspects, the one or more structural lipids (e.g., DSPC and cholesterol) are included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. In some aspects, the one or more structural lipids (e.g., DSPC and cholesterol) are included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the one or more structural lipids (e.g., DSPC and cholesterol) are included in the composition at a concentration of at least 0.05, at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the one or more structural lipids (e.g., DSPC and cholesterol) are included in the composition at a concentration of between 0.05 and 0.1, between 0.1 and 0.15, between 0.15 and 0.2, between 0.2 and 0.25, between 0.25 and 0.3, between 0.3 and 0.35, between 0.35 and 0.4, between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95 or between 0.95 and 1 mg/mL.

In specific aspects, the one or more structural lipids include DSPC, and the DSPC is included in the composition at a concentration of about 0.1 to 0.25 mg/mL. In specific aspects, the one or more structural lipids include DSPC, and the DSPC is included in the composition at a concentration of about 0.15 to 0.25 mg/mL. In specific aspects, the one or more structural lipids include DSPC, and the DSPC is included in the composition at a concentration of about 0.1, 0.11, 0.12, 0.13, 0.14, 0.15, 0.16, 0.17, 0.18, 0.19, 0.2, 0.21, 0.22, 0.23, 0.24 or 0.25 mg/mL. In specific aspects, the DSPC is included in the composition at a concentration of about 0.19 mg/mL.

In specific aspects, the one or more structural lipids include cholesterol, and the cholesterol is included in the composition at a concentration of about 0.3 to 0.45 mg/mL. In specific aspects, the one or more structural lipids include cholesterol, and the cholesterol is included in the composition at a concentration of about 0.3 to 0.4. In specific aspects, the one or more structural lipids include cholesterol, and the cholesterol is included in the composition at a concentration of about 0.35 to 0.45. In specific aspects, the one or more structural lipids include cholesterol, and the cholesterol is included in the composition at a concentration of about 0.30, 0.31, 0.32, 0.33, 0.34, 0.35, 0.36, 0.37, 0.38, 0.39, 0.40, 0.41, 0.42, 0.43, 0.44, or 0.45 mg/mL. In specific aspects, the cholesterol is included in the composition at a concentration of about 0.37 mg/mL. Concentrations for lyophilized compositions are determined post-reconstitution.

In some aspects, the VZV RNA-LNP composition further comprises one or more buffers and stabilizing agents, and optionally, salt diluents. Thus, in some aspects, the VZV RNA-LNP composition comprises an cationic lipid, a PEGylated lipid, one or more structural lipids, one or more buffers, a stabilizing agent, and optionally, a salt diluent.

In some aspects, a VZV RNA-LNP composition comprises one or more buffers. The one or more buffers may comprise any one or more buffering agents disclosed herein. In specific aspects, the composition comprises a Tris buffer comprising at least a first buffer and a second buffer. In some aspects, the first buffer is tromethamine. In some aspects, the second buffer is Tris hydrochloride (HCl). In some aspects, the first buffer and second buffer of the Tris buffer (e.g., tromethamine and Tris HCl) are included in the composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, 1.99, or 2 ng/μg/mg per mL. Concentrations for lyophilized compositions are determined post-reconstitution.

In some aspects, the VZV RNA-LNP composition is a liquid composition comprising a Tris buffer. In some aspects, the Tris buffer comprises a first buffer. In some aspects, the first buffer is tromethamine. In some aspects, the first buffer (e.g., tromethamine) is included in the liquid composition at a concentration of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the first buffer (e.g., tromethamine) is included in the liquid composition at a concentration of at least 0.1, at least 0.05, at least 0.1, at least 0.15, at least 0.2, at least 0.25, at least 0.3, at least 0.35, at least 0.4, at least 0.45, at least 0.5, at least 0.55, at least 0.6, at least 0.65, at least 0.7, at least 0.75, at least 0.8, at least 0.85, at least 0.9, at least 0.95 or at least 1 mg/mL. In some aspects, the first buffer (e.g., tromethamine) is included in the liquid composition at a concentration of between 0.05 and 0.15, between 0.15 and 0.25, between 0.25 and 0.35, between 0.35 and 0.45, between 0.45 and 0.55, between 0.55 and 0.65, between 0.65 and 0.75, between 0.75 and 0.85, or between 0.85 and 0.95. In some aspects, the first buffer (e.g., tromethamine) is included in the liquid composition at a concentration of between 0.05 and 0.1, between 0.1 and 0.15, between 0.15 and 0.2, between 0.2 and 0.25, between 0.25 and 0.3, between 0.3 and 0.35, between 0.35 and 0.4, between 0.4 and 0.45, between 0.45 and 0.5, between 0.5 and 0.55, between 0.55 and 0.6, between 0.6 and 0.65, between 0.65 and 0.7, between 0.7 and 0.75, between 0.75 and 0.8, between 0.8 and 0.85, between 0.85 and 0.9, between 0.9 and 0.95 or between 0.95 and 1 mg/mL.

In specific aspects, the first buffer (e.g., tromethamine) is included in the liquid composition at a concentration of about 0.1 to 0.3 mg/mL. In specific aspects, the first buffer (e.g., tromethamine) is included in the liquid composition at a concentration of about 0.15 to 0.25 mg/mL. In specific aspects, the first buffer (e.g., tromethamine) is included in the liquid composition at a concentration of about 0.1, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29 or 0.3 mg/mL. In specific aspects, the first buffer (e.g., tromethamine) is included in the liquid composition at a concentration of about 0.20 mg/mL.

In some aspects, the VZV RNA-LNP composition is a liquid composition comprising a Tris buffer comprising a second buffer. In some aspects, the second buffer comprises Tris HCl. In some aspects, the second buffer (e.g., Tris HCl) is included in the liquid composition at a concentration of at least, at most, between any two of, or exactly 0.5, 0.55, 1, 1.01, 1.02, 1.03, 1.04, 1.05, 1.06, 1.07, 1.08, 1.09, 1.1, 1.11, 1.12, 1.13, 1.14, 1.15, 1.16, 1.17, 1.18, 1.19, 1.2, 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or 1.5 mg/mL. In some aspects, the second buffer (e.g., Tris HCl) is included in the liquid composition at a concentration of at least 0.5, at least 0.55, at least 0.6, at least 0.65, at least 0.7, at least 0.75, at least 0.8, at least 0.85, at least 0.9, at least 0.95, at least 1, at least 1.05, at least 1.10, at least 1.15, at least 1.20, at least 1.25, at least 1.30, at least 1.35, at least 1.40, at least 1.45, or at least 1.50 mg/mL. In some aspects, the second buffer (e.g., Tris HCl) is included in the liquid composition at a concentration of between 0.5 and 0.6, between 0.6 and 0.7, between 0.7 and 0.8, between 0.8 and 0.9, between 0.9 and 1, between 1 and 1.10, between 1.10 and 1.20, between 1.20 and 1.30, between 1.30 and 1.40, or between 1.40 and 1.50 mg/mL.

In specific aspects, the second buffer (e.g., Tris HCl) is included in the liquid composition at a concentration of about 1.25 to 1.40 mg/mL. In specific aspects, the second buffer (e.g., Tris HCl) is included in the liquid composition at a concentration of about 1.30 to 1.40 mg/mL. In specific aspects, the second buffer (e.g., Tris HCl) is included in the liquid composition at a concentration of about 1.25, 1.26, 1.27, 1.28, 1.29, 1.30, 1.31, 1.32, 1.33, 1.34, or 1.35, 1.36, 1.37, 1.38, 1.39, or 1.40 mg/mL. In specific aspects, the second buffer (e.g., Tris HCl) is included in the liquid composition at a concentration of about 1.32 mg/mL.

In some aspects, the VZV RNA-LNP composition is a lyophilized composition comprising a Tris buffer. In some aspects, the Tris buffer comprises a first buffer. In some aspects, the first buffer is tromethamine. In some aspects, the first buffer (e.g., tromethamine) is included in the lyophilized composition at a concentration, after reconstitution, of at least, at most, between any two of, or exactly 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 mg/mL. In some aspects, the first buffer (e.g., tromethamine) is included in the lyophilized composition at a concentration, after reconstitution, of at least 0.01, of at least 0.05, of at least 0.1, of at least 0.15, of at least 0.2, of at least 0.25, of at least 0.3, of at least 0.35, of at least 0.4, of at least 0.45, or of at least 0.5 mg/mL. In some aspects, the first buffer (e.g., tromethamine (Tris base)) is included in the lyophilized composition at a concentration, after reconstitution, of between 0.01 and 0.05, between 0.05 and 0.1, between 0.1 and 0.15, between 0.15 and 0.2, between 0.2 and 0.25 mg/mL, between 0.25 and 0.3 mg/mL, between 0.3 and 0.35 mg/mL, between 0.35 and 0.4 mg/mL, between 0.4 and 0.45 mg/mL, or between 0.45 and 0.5 mg/mL.

In specific aspects, the first buffer (e.g., tromethamine) is included in the lyophilized composition at a concentration, after reconstitution, of about 0.01 and 0.15 mg/mL. In specific aspects, the first buffer (e.g., tromethamine) is included in the lyophilized composition at a concentration, after reconstitution, of about 0.01 and 0.10 mg/mL. In specific aspects, the first buffer (e.g., tromethamine) is included in the lyophilized composition at a concentration, after reconstitution, of about 0.05 and 0.15 mg/mL. In specific aspects, the first buffer (e.g., tromethamine) is included in the lyophilized composition at a concentration, after reconstitution, of about 0.01, 0.02, 0.03, 0.04, 0.05, 0.06, 0.07, 0.08, 0.09, 0.1, 0.11, 0.12, 0.13, 0.14, or 0.15 mg/mL. In specific aspects, the first buffer (e.g., tromethamine) is included in the lyophilized composition at a concentration, after reconstitution, of about 0.09 mg/mL.

In some aspects, the VZV RNA-LNP composition is a lyophilized composition comprising a Tris buffer comprising a second buffer. In some aspects, the second buffer comprises Tris HCl. In some aspects, the second buffer (e.g., Tris HCl) is included in the lyophilized composition at a concentration, after reconstitution, of at least, at most, between any two of, or exactly 0.1, 0.15, 0.2, 0.25, 0.3, 0.35, 0.4, 0.45, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, or 1 mg/mL. In some aspects, the second buffer (e.g., Tris HCl) is included in the lyophilized composition at a concentration, after reconstitution, of at least 0.1, at least 0.2, at least 0.3, at least 0.4, at least 0.5, at least 0.6, at least 0.7, at least 0.8, at least 0.9, or at least 1 mg/mL. In some aspects, the second buffer (e.g., Tris HCl) is included in the lyophilized composition at a concentration, after reconstitution, of between 0.1 and 0.2, between 0.2 and 0.3, between 0.3 and 0.4, between 0.4 and 0.5, between 0.5 and 0.6, between 0.6 and 0.7, between 0.7 and 0.8, between 0.8 and 0.9, or between 0.9 and 1 mg/mL.

In specific aspects, the second buffer (e.g., Tris HCl) is included in the lyophilized composition at a concentration, after reconstitution, of about 0.5 and 0.65 mg/mL. In specific aspects, the second buffer (e.g., Tris HCl) is included in the lyophilized composition at a concentration, after reconstitution, of about 0.5 and 0.6 mg/mL. In specific aspects, the second buffer (e.g., Tris HCl) is included in the lyophilized composition at a concentration, after reconstitution, of about 0.55 and 0.65 mg/mL. In specific aspects, the second buffer (e.g., Tris HCl) is included in the lyophilized composition at a concentration, after reconstitution, of about 0.5, 0.51, 0.52, 0.53, 0.54, 0.55, 0.56, 0.57, 0.58, 0.59, 0.6, 0.61, 0.62, 0.63, 0.64, or 0.65 mg/mL. In specific aspects, the second buffer (e.g., Tris HCl) is included in the lyophilized composition at a concentration, after reconstitution, of about 0.57 mg/mL.

In some aspects, a VZV RNA-LNP composition comprises a stabilizing agent. The stabilizing agent may comprise any one or more stabilizing agents disclosed herein. In some aspects, the stabilizing agent also functions as a cryoprotectant. In specific aspects, the stabilizing agent comprises sucrose. In some aspects, the stabilizing agent (e.g., sucrose) is included in the composition at a concentration of at least, at most, between any two of, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 ng/μg/mg per mL.

In some aspects, the VZV RNA-LNP composition is a liquid composition, and the stabilizing agent (e.g., sucrose) is included in the liquid composition at a concentration of at least, at most, between any two of, or exactly 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 mg/mL. In some aspects, the stabilizing agent (e.g., sucrose) is included in the liquid composition at a concentration of at least 70, at least 75, at least 80, at least 85, at least 90, at least 95, at least 100, at least 105, at least 110, at least 115, at least 120, at least 125 or at least 130 mg/mL. In some aspects, the stabilizing agent (e.g., sucrose) is included in the liquid composition at a concentration of between 70 and 80, between 80 and 90, between 90 and 100, between 100 and 110, between 110 and 120, or between 120 and 130 mg/mL.

In specific aspects, the stabilizing agent (e.g., sucrose) is included in the liquid composition at a concentration of about 95 to 110 mg/mL. In specific aspects, the stabilizing agent (e.g., sucrose) is included in the liquid composition at a concentration of about 95 to 105 mg/mL. In specific aspects, the stabilizing agent (e.g., sucrose) is included in the liquid composition at a concentration of about 100 to 110 mg/mL. In specific aspects, the stabilizing agent (e.g., sucrose) is included in the liquid composition at a concentration of about 95, 96, 97, 98, 99, 100, 101, 102, 103, 104, 105, 106, 107, 108, 109, or 110 mg/mL. In specific aspects, the stabilizing agent (e.g., sucrose) is included in the liquid composition at a concentration of about 103 mg/mL.

In some aspects, the VZV RNA-LNP composition is a lyophilized composition, and the stabilizing agent (e.g., sucrose) is included in the lyophilized composition at a concentration, after reconstitution, of at least, at most, between any two of, or exactly 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 mg/mL. In some aspects, the stabilizing agent (e.g., sucrose) is included in the lyophilized composition at a concentration, after reconstitution, of at least 20, at least 25, at least 30, at least 35, at least 40, at least 45, at least 50, at least 55, at least 60, at least 65, at least 70, at least 75 or at least 80 mg/mL. In some aspects, the stabilizing agent (e.g., sucrose) is included in the lyophilized composition at a concentration, after reconstitution, of between 20 to 30, between 30 to 40, between 40 to 50, between 50 to 60, between 60 to 70 or between 70 to 80 mg/mL.

In specific aspects, the stabilizing agent (e.g., sucrose) is included in the lyophilized composition at a concentration, after reconstitution, of about 35 to 50 mg/mL. In specific aspects, the stabilizing agent (e.g., sucrose) is included in the lyophilized composition at a concentration, after reconstitution, of about 35 to 45 mg/mL. In specific aspects, the stabilizing agent (e.g., sucrose) is included in the lyophilized composition at a concentration, after reconstitution, of about 40 to 50 mg/mL. In specific aspects, the stabilizing agent (e.g., sucrose) is included in the lyophilized composition at a concentration, after reconstitution, of about 35, 36, 37, 38, 39, 40, 41, 42, 43, 44, 45, 46, 47, 48, 49 or 50 mg/mL. In specific aspects, the stabilizing agent (e.g., sucrose) is included in the lyophilized composition at a concentration, after reconstitution, of about 44/mL.

In some aspects, lyophilized compositions are reconstituted in a suitable carrier or diluent. The carrier or diluent may comprise any one or more carriers or diluents disclosed herein. In specific aspects, the carrier or diluent comprises a salt diluent, such as sodium chloride (NaCl) (e.g., saline, e.g., physiological or normal saline). The sodium chloride may comprise 0.9% sodium chloride for injection. In some aspects, the lyophilized compositions are reconstituted in at least, at most, between any two of, or exactly 0.1, 0.2, 0.3, 0.4, 0.5, 0.6, 0.7, 0.8, 0.9, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1 mL of saline. In some aspects, the lyophilized compositions are reconstituted in at least 0.1, at least 0.2, at least 0.3, at least 0.4, at least 0.5, at least 0.6, at least 0.7, at least 0.8, at least 0.9, or at least 1 mL of sodium chloride.

In specific aspects, the lyophilized compositions are reconstituted in about 0.6 to 0.75 mL of sodium chloride/saline. In specific aspects, the lyophilized compositions are reconstituted in about 0.65 to 0.75 mL of sodium chloride/saline. In specific aspects, the lyophilized compositions are reconstituted in about 0.6, 0.61, 0.62, 0.63, 0.64, 0.65, 0.66, 0.67, 0.68, 0.69, 0.7, 0.71, 0.72, 0.73, 0.74 or 0.75 mL of sodium chloride/saline.

In some aspects, the salt diluent (e.g., NaCl) is included in the lyophilized composition at a concentration, after reconstitution, of at least, at most, between any two of, or exactly 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, or 50 ng/μg/mg per mL. In some aspects, the salt diluent (e.g., NaCl) is included in the lyophilized composition at a concentration, after reconstitution, of in at least, at most, between any two of, or exactly 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or 20 mg/mL. In some aspects, the salt diluent (e.g., NaCl) is included in the lyophilized composition at a concentration, after reconstitution, of at least 1, at least 2, at least 3, at least 4, at least 5, at least 6, at least 7, at least 8, at least 9, at least 10, at least 11, at least 12, at least 13, at least 14, at least 15, or at least 20 mg/mL.

In specific aspects, the salt diluent (e.g., NaCl) is included in the lyophilized composition at a concentration, after reconstitution, of between about 5 and 15 mg/mL. In some aspects, the salt diluent (e.g., NaCl) is included in the lyophilized composition at a concentration, after reconstitution, of between about 5 and 10 mg/mL. In specific aspects, the salt diluent (e.g., NaCl) is included in the lyophilized composition at a concentration, after reconstitution, of about 5, 6, 7, 8, 9, 10, 11, 12, 13, 14, or 15 mg/mL. In specific aspects, the salt diluent (e.g., NaCl) is included in the lyophilized composition at a concentration, after reconstitution, of about 9 mg/mL.

The pH of the VZV RNA-LNP composition may be at least, at most, exactly, or between any two of pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5, or any range or value derivable therein. In some aspects, the VZV RNA-LNP composition is at a pH of at least 6.5, at least 7.0, at least 7.5, at least 8.0, or at least 8.5. In specific aspects, the VZV RNA-LNP composition is at a pH between 6.0 and 7.5, between 6.5 and 7.5, between 7.0 and 8.0, between and 7.5 and 8.5. In specific aspects, the VZV RNA-LNP composition is between 7.0 and 8.0. In specific aspects, the VZV RNA-LNP composition is at pH 7.0, 7.1, 7.2, 7.3, 7.4, 7.5, 7.6, 7.7, 7.8, 7.9 or 8.0. In specific aspects, the VZV RNA-LNP composition is at about pH 7.4. In some aspects, sodium hydroxide buffer may be used for a buffer pH adjustment.

In specific aspects, a VZV RNA-LNP composition comprises a VZV RNA polynucleotide encoding a VZV polypeptide as disclosed herein, encapsulated in LNPs with a lipid composition of an cationic lipid at a concentration of about 0.8 to 0.95 mg/mL, a PEGylated lipid at a concentration of about 0.05 to 0.15 mg/mL, a first structural lipid at a concentration of about 0.1 to 0.25 mg/mL, and a second structural lipid at a concentration of about 0.3 to 0.45 mg/mL.

In specific aspects, a VZV RNA-LNP composition comprises a VZV RNA polynucleotide encoding a VZV polypeptide as disclosed herein, encapsulated in LNPs with a lipid composition of ALC-0315 at a concentration of about 0.8 to 0.95 mg/mL, ALC-0159 at a concentration of about 0.05 to 0.15 mg/mL, DSPC at a concentration of about 0.1 to 0.25 mg/mL, and cholesterol at a concentration of about 0.3 to 0.45 mg/mL.

In specific aspects, the VZV RNA-LNP composition is a liquid VZV RNA-LNP composition, and the liquid VZV RNA-LNP composition further comprises a buffer composition comprising a first buffer at a concentration of about 0.15 to 0.3 mg/mL, a second buffer at a concentration of about 1.25 to 1.4 mg/mL, and a stabilizing agent at a concentration of about 95 to 110 mg/mL. In specific aspects, the VZV RNA-LNP composition is a liquid VZV RNA-LNP composition, and the liquid VZV RNA-LNP composition further comprises a Tris buffer composition comprising tromethamine at a concentration of about 0.1 to 0.3 mg/mL, Tris HCl at a concentration of about 1.25 to 1.4 mg/mL, and sucrose at a concentration of about 95 to 110 mg/mL.

Thus, in specific aspects, a liquid VZV RNA-LNP composition comprises an cationic lipid at a concentration of about 0.8 to 0.95 mg/mL, a PEGylated lipid at a concentration of about 0.05 to 0.15 mg/mL, a first structural lipid at a concentration of about 0.1 to 0.25 mg/mL, a second structural lipid at a concentration of about 0.3 to 0.45 mg/mL, and further comprises a first buffer at a concentration of about 0.1 to 0.3 mg/mL, a second buffer at a concentration of about 1.25 to 1.4 mg/mL, and a stabilizing agent at a concentration of about 95 to 110 mg/mL.

Thus, in specific aspects, a liquid VZV RNA-LNP composition comprises ALC-0315 at a concentration of about 0.8 to 0.95 mg/mL, ALC-0159 at a concentration of about 0.05 to 0.15 mg/mL, DSPC at a concentration of about 0.1 to 0.25 mg/mL, cholesterol at a concentration of about 0.3 to 0.45 mg/mL, and further comprises a Tris buffer composition comprising tromethamine at a concentration of about 0.1 to 0.3 mg/mL, Tris HCl at a concentration of about 1.25 to 1.4 mg/mL, and sucrose at a concentration of about 95 to 110 mg/mL.

In specific aspects, the VZV RNA-LNP composition is a lyophilized VZV RNA-LNP composition, and the lyophilized VZV RNA-LNP composition further comprises (after reconstitution) a first buffer at a concentration of about 0.01 and 0.15 mg/mL, a second buffer at a concentration of about 0.5 and 0.65 mg/mL, a stabilizing agent at a concentration of about 35 to 50 mg/m L, and a salt diluent at a concentration of between about 5 and 15 mg/mL.

In specific aspects, the VZV RNA-LNP composition is a lyophilized VZV RNA-LNP composition, and the lyophilized VZV RNA-LNP composition further comprises (after reconstitution) a Tris buffer composition comprising tromethamine at a concentration of about 0.01 and 0.15 mg/mL, Tris HCl at a concentration of about 0.5 and 0.65 mg/mL, sucrose at a concentration of about 35 to 50 mg/mL, and sodium chloride (NaCl) at a concentration of about 5 to 15 mg/mL.

Thus, in specific aspects, a lyophilized VZV RNA-LNP composition comprises (after reconstitution) a cationic lipid at a concentration of about 0.8 to 0.95 mg/mL, a PEGylated lipid at a concentration of about 0.05 to 0.15 mg/mL, a first structural lipid at a concentration of about 0.1 to 0.25 mg/mL, a second structural lipid at a concentration of about 0.3 to 0.45 mg/mL, and further comprises a first buffer at a concentration of about 0.01 and 0.15 mg/mL, a second buffer at a concentration of about 0.5 and 0.65 mg/mL, a stabilizing agent at a concentration of about 35 to 50 mg/mL, and a salt diluent at a concentration of about 5 to 15 mg/mL. In specific aspects, the lyophilized compositions are reconstituted in 0.6 to 0.75 mL of the salt diluent.

Thus, in some aspects, a lyophilized VZV RNA-LNP composition comprises (after reconstitution) ALC-0315 at a concentration of about 0.8 to 0.95 mg/mL, ALC-0159 at a concentration of about 0.05 to 0.15 mg/mL, DSPC at a concentration of about 0.1 to 0.25 mg/mL, cholesterol at a concentration of about 0.3 to 0.45 mg/mL, and further comprises tromethamine at a concentration of about 0.01 and 0.15 mg/mL, Tris HCl at a concentration of about 0.5 and 0.65 mg/mL, sucrose at a concentration of about 35 to 50 mg/mL, and NaCl at a concentration of about 5 to 15 mg/mL. In specific aspects, the lyophilized compositions are reconstituted in 0.6 to 0.75 mL of NaCl (saline).

Concentrations in the lyophilized VZV RNA-LNP composition above are determined post-reconstitution.

In some aspects, a VZV RNA-LNP composition (pre-lyophilization) comprises a cationic lipid at a concentration of about 1.0 to 3.0 mg/mL, a PEGylated lipid at a concentration of about 0.10 to 0.35 mg/mL, a first structural lipid at a concentration of about 0.4 to 0.55 mg/mL, a second structural lipid at a concentration of about 0.85 to 1.0 mg/mL, and further comprises a first buffer at a concentration of about 0.1 and 0.3 mg/mL, a second buffer at a concentration of about 1.25 and 1.40 mg/mL, a stabilizing agent at a concentration of about 95 to 110 mg/mL.

Thus, in some aspects, a VZV RNA-LNP composition (pre-lyophilization) comprises ALC-0315 at a concentration of about 1.0 to 3.0 mg/mL, ALC-0159 at a concentration of about 0.10 to 0.35 mg/mL, DSPC at a concentration of about 0.4 to 0.55 mg/mL, cholesterol at a concentration of about 0.85 to 1.0 mg/mL, and further comprises tromethamine at a concentration of about 0.1 and 0.3 mg/mL, Tris HCl at a concentration of about 1.25 and 1.40 mg/mL, sucrose at a concentration of about 95 to 110 mg/mL.

The VZV RNA-LNP compositions further comprise VZV RNA described herein encapsulated in LNPs, see section D. ADMINISTRATION.

In specific aspects, a VZV RNA-LNP composition is a liquid VZV RNA-LNP composition comprising a VZV RNA polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, encapsulated in LNPs with a lipid composition of an cationic lipid at a concentration of about 0.8 to 0.95 mg/mL, a PEGylated lipid at a concentration of about 0.05 to 0.15 mg/mL, a first structural lipid at a concentration of about 0.1 to 0.25 mg/mL, and a second structural lipid at a concentration of about 0.3 to 0.45 mg/mL, and further comprising a buffer composition comprising a first buffer at a concentration of about 0.15 to 0.3 mg/mL, a second buffer at a concentration of about 1.25 to 1.4 mg/mL, and a stabilizing agent at a concentration of about 95 to 110 mg/mL.

In specific aspects, a liquid VZV RNA-LNP composition comprises a VZV RNA polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, and more preferably about 0.06 mg/mL, encapsulated in LNPs with a lipid composition of ALC-0315 at a concentration of about 0.8 to 0.95 mg/mL, ALC-0159 at a concentration of about 0.05 to 0.15 mg/mL, DSPC at a concentration of about 0.1 to 0.25 mg/mL, and cholesterol at a concentration of about 0.3 to 0.45 mg/mL, and further comprising a Tris buffer composition comprising tromethamine at a concentration of about 0.1 to 0.3 mg/mL, Tris HCl at a concentration of about 1.25 to 1.4 mg/mL, and sucrose at a concentration of about 95 to 110 mg/mL.

In specific aspects, the VZV RNA-LNP composition is a lyophilized VZV RNA-LNP composition comprising a VZV RNA polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, encapsulated in LNPs with a lipid composition of a cationic lipid at a concentration of about 0.8 to 0.95 mg/mL, a PEGylated lipid at a concentration of about 0.05 to 0.15 mg/mL, a first structural lipid at a concentration of about 0.1 to 0.25 mg/mL, and a second structural lipid at a concentration of about 0.3 to 0.45 mg/mL, and further comprising a first buffer at a concentration of about 0.01 and 0.15 mg/mL, a second buffer at a concentration of about 0.5 and 0.65 mg/mL, a stabilizing agent at a concentration of about 35 to 50 mg/mL, and a salt diluent at a concentration of 5 to 15 mg/mL. In specific aspects, the lyophilized compositions are reconstituted in 0.6 to 0.75 mL of the salt diluent. Concentrations in the lyophilized VZV RNA-LNP composition are determined post-reconstitution.

In specific aspects, a lyophilized VZV RNA-LNP composition comprises a VZV RNA polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, and more preferably about 0.06 mg/mL, encapsulated in LNPs with a lipid composition of ALC-0315 at a concentration of about 0.8 to 0.95 mg/mL, ALC-0159 at a concentration of about 0.05 to 0.15 mg/mL, DSPC at a concentration of about 0.1 to 0.25 mg/mL, and cholesterol at a concentration of about 0.3 to 0.45 mg/mL, and further comprising tromethamine at a concentration of about 0.01 and 0.15 mg/mL, Tris HCl at a concentration of about 0.5 and 0.65 mg/mL, sucrose at a concentration of about 35 to 50 mg/mL, and NaCl at a concentration of about 5 to 15 mg/mL. In specific aspects, the lyophilized compositions are reconstituted in 0.6 to 0.75 mL of the NaCl diluent (saline). Concentrations in the lyophilized VZV RNA-LNP composition are determined post-reconstitution.

In some aspects, a VZV RNA-LNP composition (pre-lyophilization) comprises a VZV RNA polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, encapsulated in LNPs with a lipid composition of a cationic lipid at a concentration of about 1.0 to 3.0 mg/mL, a PEGylated lipid at a concentration of about 0.10 to 0.35 mg/mL, a first structural lipid at a concentration of about 0.4 to 0.55 mg/mL, a second structural lipid at a concentration of about 0.85 to 1.0 mg/mL, and further comprises a first buffer at a concentration of about 0.1 and 0.3 mg/mL, a second buffer at a concentration of about 1.25 and 1.40 mg/mL, a stabilizing agent at a concentration of about 95 to 110 mg/mL.

Thus, in some aspects, a VZV RNA-LNP composition (pre-lyophilization) comprises a VZV RNA polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, and more preferably 0.15 mg/mL, encapsulated in LNPs with a lipid composition of comprises ALC-0315 at a concentration of about 1.0 to 3.0 mg/mL, ALC-0159 at a concentration of about 0.10 to 0.35 mg/mL, DSPC at a concentration of about 0.4 to 0.55 mg/mL, cholesterol at a concentration of about 0.85 to 1.0 mg/mL, and further comprises tromethamine at a concentration of about 0.1 and 0.3 mg/mL, Tris HCl at a concentration of about 1.25 and 1.40 mg/mL, sucrose at a concentration of about 95 to 110 mg/mL.

In some aspects, the liquid RNA-LNP immunogenic composition comprises a RNA molecule/polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, encapsulated in a LNP, and further comprising about 5 to 15 mM Tris buffer, 200 to 400 mM sucrose at a pH of about 7.0 to 8.0.

In some aspects, the liquid RNA-LNP immunogenic composition comprises a RNA molecule/polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, and more preferably about 0.06 mg/mL, encapsulated in a LNP, and further comprising about 10 mM Tris buffer, 300 mM sucrose at a pH of about 7.4.

In some aspects, the RNA-LNP immunogenic composition (pre-lyophilized) comprises a RNA molecule/polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, encapsulated in a LNP, and further comprising about 5 to 15 mM Tris buffer, 200 to 400 mM sucrose at a pH of about 7.0 to 8.0, and reconstituted with 0.9% sodium chloride diluent.

In some aspects, the RNA-LNP immunogenic composition (pre-lyophilized) comprises a RNA molecule/polynucleotide encoding a VZV polypeptide as disclosed herein at a concentration of at least, at most, exactly, or between any two of 0.01, 0.15, 0.30, 0.45, 0.60, 0.75, or 0.90 mg/mL, preferably about 0.01 to 0.09 mg/mL, and more preferably 0.15 mg/mL, encapsulated in a LNP, and further comprising about 10 mM Tris buffer, 300 mM sucrose at a pH of about 7.4, and reconstituted with 0.9% sodium chloride diluent.

B. Vaccines

In some aspects, a pharmaceutical composition described herein is an immunogenic composition for inducing an immune response. For example, in some aspects, an immunogenic composition is a vaccine. In some aspects, the compositions described herein include at least one isolated nucleic acid or polypeptide molecule as described herein. In specific aspects, the immunogenic compositions comprise nucleic acids, and the immunogenic compositions are nucleic acid vaccines. In some aspects, the immunogenic compositions comprise RNA (e.g. mRNA, saRNA), and vaccines are RNA vaccines. In other aspects, the immunogenic compositions comprise DNA, and vaccines are DNA vaccines. In yet other aspects, the immunogenic compositions comprise a polypeptide, and vaccines are polypeptide vaccines. Conditions and/or diseases that may be treated with the nucleic acid and/or peptide or polypeptide compositions include, but are not limited to, those caused and/or impacted by infection, cancer, rare diseases, and other diseases or conditions caused by overproduction, underproduction, or improper production of protein or nucleic acids.

In some aspects, the composition is substantially free of one or more impurities or contaminants and, for instance, includes nucleic acid or polypeptide molecules that are equal to at least, at most, exactly, or between any two of 90%, 91%, 92%, 93%, 94%, 95%, 96%, 97%, 98%, or 99% pure; at least 98% pure, or at least 99% pure.

The present disclosure includes methods for preventing, treating or ameliorating an infection, disease or condition in a subject, including administering to a subject an effective amount of an RNA molecule that includes at least one open reading frame encoding a polypeptide or composition described herein. As such, the disclosure contemplates vaccines for use in both active and passive immunization aspects. Immunogenic compositions, proposed to be suitable for use as a vaccine, may be prepared from RNA molecules encoding polypeptide(s), such as VZV glycoproteins. In certain aspects, immunogenic compositions are lyophilized for more ready formulation into a desired vehicle.

The preparation of vaccines that contain nucleic acid and/or peptide or polypeptide as active ingredients is generally well understood in the art, as exemplified by U.S. Pat. Nos. 4,608,251; 4,601,903; 4,599,231; 4,599,230; 4,596,792; and 4,578,770, all of which are incorporated herein by reference. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific aspects, vaccines are formulated with a combination of substances, as described in U.S. Pat. Nos. 6,793,923 and 6,733,754, which are incorporated herein by reference.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%. In some aspects, suppositories may be formed from mixtures containing the active ingredient in the range of about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient.

The polypeptide-encoding nucleic acid constructs and polypeptides may be formulated into a vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms of active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application within a solid physiologically acceptable base or in a physiologically acceptable dispersion, parenterally, by injection and the like. The dosage of the vaccine will depend on the route of administration and will vary according to the size and health of the subject.

In certain aspects, it will be desirable to have one administration of the vaccine. In some aspects, it will be desirable to have multiple administrations of the vaccine, e.g., 2, 3, 4, 5, 6 or more administrations. The vaccinations may be at 1, 2, 3, 4, 5, 6, 7, 8, to 5, 6, 7, 8, 9, 10, 11, 12 twelve week intervals, including all ranges there between. In some aspects, vaccinations may be at 1, 2, 3, 4, 5, 6, 7, 8, 9, 10, 11, 12 month intervals, including all ranges there between. Periodic boosters at intervals of 1-5 years may be desirable to maintain protective levels of the antibodies.

i. Carriers

A pharmaceutically acceptable carrier may include the liquid or non-liquid basis of a composition. If a composition is provided in liquid form, the carrier may be water, such as pyrogen-free water, isotonic saline or buffered (aqueous) solutions, e.g. phosphate, citrate buffered solutions. Water or a buffer, such as an aqueous buffer, may be used, containing a sodium salt, a calcium salt, and and/or a potassium salt. The sodium, calcium and/or potassium salts may occur in the form of their halogenides, e.g. chlorides, iodides, or bromides, in the form of their hydroxides, carbonates, hydrogen carbonates, or sulfates, etc. Examples of sodium salts include, but are not limited to, NaCl, NaI, NaBr, Na₂CO₃, NaHCO₃, Na₂SO₄, Na₂HPO₄, Na₂HPO₄·₂H₂O, examples of potassium salts include, but are not limited to, KCl, KI, KBr, K₂CO₃, KHCO₃, K₂SO₄, KH₂PO₄, and examples of calcium salts include, but are not limited to, CaCl₂, CaI₂, CaBr₂, CaCO₃, CaSO₄, Ca(OH)₂. Examples of further carriers may include sugars, such as, for example, lactose, glucose, trehalose and sucrose; starches, such as, for example, corn starch or potato starch; dextrose; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulfate; vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid. Examples of further carriers may include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

ii. Adjuvants

Suitable adjuvants include all acceptable immunostimulatory compounds, such as cytokines, toxins, or synthetic compositions. A number of adjuvants may be used to enhance an antibody response. Adjuvants include, but are not limited to, oil-in-water emulsions, water-in-oil emulsions, mineral salts, polynucleotides, and natural substances. Specific adjuvants that may be used include Freund's adjuvant, oil such as MONTANIDE® ISA51, IL1, IL2, IL3, IL4, IL5, IL6, IL7, IL8, IL9, IL10, IL12, alpha-interferon, PTNGg, GM-CSF, GMCSP, BCG, LT-a, aluminum salts, such as aluminum hydroxide or other aluminum compound, MDP compounds, such as thur-MDP and nor-MDP, CGP (MTP-PE), lipid A, monophosphoryl lipid A (MPL), lipopeptides (e.g., Pam3Cys). RIBI, which contains three components extracted from bacteria, MPL, trehalose dimycolate (TDM), and cell wall skeleton (CWS) in a 2% squalene/Tween 80 emulsion. MHC antigens may even be used.

Various methods of achieving adjuvant affect for the vaccine includes use of agents such as aluminum hydroxide or phosphate (alum), commonly used as about 0.05 to about 0.1% solution in phosphate buffered saline, admixture with synthetic polymers of sugars (CARBOPOL®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin; mixture with bacterial cells (e.g., C. parvum), endotoxins or lipopolysaccharide components of Gram-negative bacteria; emulsion in physiologically acceptable oil vehicles (e.g., mannide mono-oleate (Aracel A)); or emulsion with a 20% solution of a perfluorocarbon (FLUOSOL-DA®) used as a block substitute may also be employed to produce an adjuvant effect.

In addition to adjuvants, it may be desirable to co-administer biologic response modifiers (BRM) to enhance immune responses. BRMs have been shown to upregulate T cell immunity or downregulate suppresser cell activity. Such BRMs include, but are not limited to, Cimetidine (CIM; 1200 mg/d) (Smith/Kline, PA); or low-dose Cyclophosphamide (CYP; 300 mg/m²) (Johnson/Mead, NJ) and cytokines such as γ-interferon, IL-2, or IL-12 or genes encoding proteins involved in immune helper functions, such as B-7.

C. Combination Therapy

The compositions and related methods of the present disclosure, particularly administration of a RNA molecule encoding a VZV polypeptide, may also be used in combination with the administration of traditional therapies. These include, but are not limited to, the administration of antiviral therapies such as acyclovir, valacyclovir, and famciclovir, or various combinations of antivirals. Also included are the administration of one or more therapies to treat one or more symptoms of VZV infection, including, but not limited to, steroids including corticosteroids, anti-inflammatories including acetaminophen or ibuprofen, pain-relief agents, creams or lotions to relieve itching, cool compresses, or various combinations thereof.

In one aspect, it is contemplated that a vaccine and/or therapy is used in conjunction with antiviral treatment. Alternatively, the therapy may precede or follow the other agent treatment by intervals ranging from minutes to weeks. In aspects where the other agents and/or vaccines are administered separately, one would generally ensure that a significant period of time did not expire between the time of each delivery, such that the agent and immunogenic composition would still be able to exert an advantageously combined effect on the subject. In such aspects, it is contemplated that one may administer both modalities within about 12-24 h of each other or within about 6-12 h of each other. In some situations, it may be desirable to extend the time period for administration significantly, where several days (2, 3, 4, 5, 6 or 7) to several weeks (1, 2, 3, 4, 5, 6, 7 or 8) lapse between the respective administrations.

Various combinations may be employed, for example antiviral therapy “A” and immunogenic polypeptide given as part of an immune therapy regime “B”:

| | | | | | | | |
|---|---|---|---|---|---|---|---|
| A/B/A | B/A/B | B/B/A | A/A/B | A/B/B | B/A/A | A/B/B/B | B/A/B/B |
| B/B/B/A | B/B/A/B | A/A/B/B | A/B/A/B | A/B/B/A | B/B/A/A | | |
| B/A/B/A | B/A/A/B | A/A/A/B | B/A/A/A | A/B/A/A | A/A/B/A | | |

Administration of the immunogenic compositions of the present disclosure to a patient/subject will follow general protocols for the administration of such compounds, taking into account the toxicity, if any, of the VZV RNA vaccine composition, or other compositions described herein. It is expected that the treatment cycles would be repeated as necessary. It also is contemplated that various standard therapies, such as hydration, may be applied in combination with the described therapy.

D. Administration

Administration of the compositions described herein may be carried out via any of the accepted modes of administration of agents for serving similar utilities. In some aspects, a pharmaceutical composition described herein may be administered intravenously, intraarterially, subcutaneously, intradermally or intramuscularly. In specific aspects, the VZV RNA molecules and/or RNA-LNP compositions are administered intramuscularly. In certain aspects, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In one aspect, the pharmaceutical composition is formulated for intramuscular administration. In another aspect, the pharmaceutical composition is formulated for systemic administration, e.g., for intravenous administration.

Pharmaceutical compositions may be formulated into preparations in solid, semi-solid, liquid, lyophilized, frozen, or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection, or infusion techniques. Pharmaceutical compositions described herein are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound in aerosol form may hold a plurality of dosage units. The composition to be administered will, in any event, contain a therapeutically and/or prophylactically effective amount of a compound within the scope of this disclosure, or a pharmaceutically acceptable salt thereof, for treatment of a disease or condition of interest in accordance with the teachings described herein.

A pharmaceutical composition within the scope of this disclosure may be in the form of a solid or liquid and may be frozen or lyophilized. In one aspect, the carrier(s) are particulate, so that the compositions are, for example, in tablet or powder form. The carrier(s) may be liquid, with the compositions being, for example, an oral syrup, injectable liquid, or an aerosol, which is useful in, for example, inhalatory administration. In some aspects, when intended for oral administration, the pharmaceutical composition is in either solid or liquid form, where semi-solid, semi-liquid, suspension, and gel forms are included within the forms considered herein as either solid or liquid. As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present or exclude: binders such as carboxymethylcellulose, ethyl cellulose, microcrystalline cellulose, gum tragacanth, or gelatin; excipients such as starch, lactose, or dextrins; disintegrating agents such as alginic acid, sodium alginate, PRIMOJEL®, corn starch and the like; lubricants such as magnesium stearate or STEROTEX®; glidants such as colloidal silicon dioxide; sweetening agents such as sucrose or saccharin; a flavoring agent such as peppermint, methyl salicylate, or orange flavoring; and a coloring agent. When the pharmaceutical composition is in the form of a capsule, for example, a gelatin capsule, it may contain, in addition to materials of the above type, a liquid carrier such as polyethylene glycol or oil. The pharmaceutical composition may be in the form of a liquid, for example, an elixir, syrup, solution, emulsion or suspension. The liquid may be for oral administration or for delivery by injection, as two examples. In some aspects, when intended for oral administration, compositions contain, in addition to the present compounds, one or more of a sweetening agent, preservatives, dye/colorant, and flavor enhancer. In a composition intended to be administered by injection, one or more of a surfactant, preservative, wetting agent, dispersing agent, suspending agent, buffer, stabilizer, and isotonic agent may be included or excluded.

A liquid pharmaceutical composition, whether they be solutions, suspensions, or other like form, may include or exclude one or more of the following adjuvants: sterile diluents such as water for injection, saline solution, e.g., physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, or phosphates; and agents for the adjustment of tonicity such as sodium chloride or dextrose; agents to act as cryoprotectants such as sucrose or trehalose. The parenteral preparation may be enclosed in ampoules, disposable syringes, or multiple dose vials made of glass or plastic. In one aspect, physiological saline is the adjuvant. In one aspect, an injectable pharmaceutical composition is sterile. A liquid pharmaceutical composition intended for either parenteral or oral administration should contain an amount of a compound such that a suitable dosage will be obtained.

The pharmaceutical compositions may be prepared by methodology well known in the pharmaceutical art. For example, a pharmaceutical composition intended to be administered by injection may be prepared by combining the nucleic acid or polypeptide with sterile, distilled water or other carrier so as to form a solution. A surfactant may be added to facilitate the formation of a homogeneous solution or suspension. Surfactants are compounds that non-covalently interact with a compound consistent with the teachings herein so as to facilitate dissolution or homogeneous suspension of the compound in the aqueous delivery system.

The pharmaceutical compositions according to the present disclosure, or their pharmaceutically acceptable salts, are generally applied in a “therapeutically effective amount” or a “prophylactically effective amount” and in “a pharmaceutically acceptable preparation.” The term “pharmaceutically acceptable” refers to the non-toxicity of a material which does not interact with the action of the active component of the pharmaceutical composition. The terms “therapeutically effective amount” and “prophylactically effective amount” refer to the amount which achieves a desired reaction or a desired effect alone or together with further doses. In the case of the treatment of a particular disease, in one aspect, the desired reaction relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition.

The compositions within the scope of the disclosure are administered in a therapeutically and/or prophylactically effective amount, which will vary depending upon a variety of factors including the activity of the specific therapeutic and/or prophylactic agent employed; the metabolic stability and length of action of the therapeutic and/or prophylactic agent; the individual parameters of the patient, including the age, body weight, general health, gender, and diet of the patient; the mode, time, and/or duration of administration; the rate of excretion; the drug combination; the severity of the particular disorder or condition; and the subject undergoing therapy. Accordingly, the doses administered of the compositions described herein may depend on various of such parameters. In the case that a reaction in a patient is insufficient with an initial dose, higher doses (or effectively higher doses achieved by a different, more localized route of administration) may be used. In some aspects, compositions (e.g., VZV RNA-LNP compositions) may be administered at dosage levels sufficient to deliver 0.0001 ng/μg/mg per kg to 100 ng/μg/mg per kg, 0.001 ng/μg/mg per kg to 0.05 ng/μg/mg per kg, 0.005 ng/μg/mg per kg to 0.05 ng/μg/mg per kg, 0.001 ng/μg/mg per kg to 0.005 ng/μg/mg per kg, 0.05 ng/μg/mg per kg to 0.5 ng/μg/mg per kg, 0.01 ng/μg/mg per kg to 50 ng/μg/mg per kg, 0.1 ng/μg/mg per kg to 40 ng/μg/mg per kg, 0.5 ng/μg/mg per kg to 30 ng/μg/mg per kg, 0.01 ng/μg/mg per kg to 10 ng/μg/mg per kg, 0.1 ng/μg/mg per kg to 10 ng/μg/mg per kg, or 1 ng/μg/mg per kg to 25 ng/μg/mg per kg, of subject body weight per day, one or more times a day, per week, per month, etc. to obtain the desired therapeutic, diagnostic, prophylactic, or imaging effect (see e.g., the range of unit doses described in International Publication No. WO2013/078199, herein incorporated by reference in its entirety). In some aspects, compositions (e.g., VZV RNA-LNP compositions) may be administered at dosage levels sufficient to deliver at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg per kg, of subject body weight per day, one or more times a day, per week, per month, etc. to obtain the desired therapeutic, diagnostic, prophylactic, or imaging effect.

In some aspects, compositions (e.g., VZV RNA-LNP compositions) may be administered at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg per day, one or more times a day, per week, per month, etc. to obtain the desired therapeutic, diagnostic, prophylactic, or imaging effect.

In specific aspects, compositions (e.g., VZV RNA-LNP compositions) may be administered at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/mL VZV RNA encapsulated in LNP.

In exemplary aspects, compositions (e.g., VZV RNA-LNP compositions) may be administered at dose levels of at least, at most, exactly, or between any two of 0.01, 0.15, 0.30, 0.45, 0.60, 0.75, or 0.90 mg/mL VZV RNA encapsulated in LNP. In exemplary aspects, compositions (e.g., VZV RNA-LNP compositions) may be administered at dose levels of at least, at most, exactly, or between any two of 0.01, 0.15, 0.30, 0.45, 0.60, 0.75, or 0.90 mg VZV RNA encapsulated in LNP.

In specific aspects, compositions (e.g., VZV RNA-LNP compositions) may be administered at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μg/mL VZV RNA encapsulated in LNP.

In exemplary aspects, compositions (e.g., VZV RNA-LNP compositions) may be administered at dose levels of at least, at most, exactly, or between any two of 1, 15, 30, 45, 60, 75, 90, 100 or higher μg/mL VZV RNA encapsulated in LNP. In exemplary aspects, compositions (e.g., VZV RNA-LNP compositions) may be administered at dose levels of at least, at most, exactly, or between any two of 1, 15, 30, 45, 60, 75, 90, 100 or higher μg VZV RNA encapsulated in LNP.

The desired dosage may be delivered multiple times a day (e.g., 1, 2, 3, 4, 5, or more times a day), every other day, every third day, every week, every two weeks, every three weeks, every four weeks, every 2 months, every three months, every 6 months, etc. In certain aspects, the desired dosage may be delivered using a single-dose administration. In certain aspects, the desired dosage may be delivered using multiple administrations (e.g., two, three, four, five, six, seven, eight, nine, ten, eleven, twelve, thirteen, fourteen, or more administrations). When multiple administrations are employed, split dosing regimens may be used. The time of administration between the initial administration of the composition and a subsequent administration of the composition may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

In some aspects, compositions (e.g., VZV RNA-LNP compositions) may be administered in a single dose. In some aspects, compositions (e.g., VZV RNA-LNP compositions) may be administered twice (e.g., Day 0 and about Day 7, Day 0 and about Day 14, Day 0 and about Day 21, Day 0 and about Day 28, Day 0 and about Day 60, Day 0 and about Day 90, Day 0 and about Day 120, Day 0 and about Day 150, Day 0 and about Day 180, Day 0 and about 1 month later, Day 0 and about 2 months later, Day 0 and about 3 months later, Day 0 and about 6 months later, Day 0 and about 9 months later, Day 0 and about 12 months later, Day 0 and about 18 months later, Day 0 and about 2 years later, Day 0 and about 5 years later, or Day 0 and about 10 years later), with each administration at a total dose of or at dosage levels sufficient to deliver a total dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg VZV RNA encapsulated in LNP. Higher and lower dosages and frequency of administration are encompassed by the present disclosure. For example, compositions (e.g., VZV RNA-LNP compositions) may be administered three or four times. Periodic boosters at intervals of 1-5 years may be desirable to maintain protective levels of the antibodies.

In some aspects, the compositions (e.g., VZV RNA-LNP compositions) are administered to a subject as a single dose of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg of VZV RNA encapsulated in LNP. In some aspects, the compositions (e.g., VZV RNA-LNP compositions) are administered the subject as a single dose of at least, at most, exactly, or between any two of 1 μg, 15 μg, 30 μg, 45 μg, 60 μg, 75 μg, 90 μg, 100 μg or higher of VZV RNA encapsulated in LNP.

In some aspects, the compositions (e.g., VZV RNA-LNP compositions) are administered to a subject as two doses of at least, at most, exactly, or between any two of 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 ng/μg/mg of VZV RNA encapsulated in LNP. In some aspects, the compositions (e.g., VZV RNA-LNP compositions) are administered the subject as two doses of at least, at most, exactly, or between any two of 1 μg, 15 μg, 30 μg, 45 μg, 60 μg, 75 μg, 90 μg, 100 μg or higher of VZV RNA encapsulated in LNP.

In specific aspects, compositions (e.g., VZV RNA-LNP compositions) may be administered twice (e.g., Day 0 and Day 28, Day 0 and Day 60, Day 0 and Day 180, Day 0 and 2 months later, Day 0 and 6 months later), with each administration at a total dose of or at dosage levels sufficient to deliver a total dose of at least, at most, exactly, or between any two of 1 μg, 15 μg, 30 μg, 45 μg, 60 μg, 75 μg, 90 μg, 100 μg or higher VZV RNA encapsulated in LNP.

### IX. Methods of Use

Provided herein are compositions (e.g., pharmaceutical compositions comprising VZV RNA molecules and/or VZV RNA-LNPs), methods, kits and reagents for prevention and/or treatment of VZV in humans and other mammals.

VZV RNA compositions (e.g., VZV RNA-LNP compositions) may be used as therapeutic or prophylactic agents. They may be used in medicine to prevent and/or treat infectious disease. In exemplary aspects, the VZV RNA compositions are used to provide prophylactic protection from varicella or herpes zoster. Varicella is an acute infectious disease caused by VZV. The VZV vaccines of the present disclosure may be used to prevent and/or treat VZV (shingles or herpes zoster) and may be particularly useful for prevention and/or treatment of immunocompromised and elderly patients to prevent or to reduce the severity and/or duration of herpes zoster.

In some aspects, the VZV RNA compositions (e.g., VZV RNA-LNP compositions) of the disclosure are administered to a subject (e.g., a mammalian subject, such as a human subject), and the RNA polynucleotides are translated in vivo to produce an antigenic polypeptide.

In some aspects, the VZV RNA compositions of the disclosure may be used to prime immune effector cells, for example, to activate peripheral blood mononuclear cells (PBMCs) ex vivo, which are then infused (re-infused) into a subject.

In some aspects, after administration of a VZV RNA molecule described herein, e.g., formulated as RNA-LNPs, at least a portion of the RNA is delivered to a target cell. In some aspects, at least a portion of the RNA is delivered to the cytosol of the target cell. In some aspects, the RNA is translated by the target cell to produce the polypeptide or protein it encodes. In some aspects, the target cell is a spleen cell. In some aspects, the target cell is an antigen presenting cell such as a professional antigen presenting cell in the spleen. In some aspects, the target cell is a dendritic cell or macrophage. RNA molecules such as RNA-LNPs described herein may be used for delivering RNA to such target cell. Accordingly, the present disclosure also relates to a method for delivering RNA to a target cell in a subject comprising the administration of the RNA-particles described herein to the subject.

In some aspects, the RNA is delivered to the cytosol of the target cell. In some aspects, the RNA is translated by the target cell to produce the polypeptide or protein encoded by the RNA. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, may be referred to as encoding the protein or other product of that gene or cDNA.

In some aspects, nucleic acid compositions described herein, e.g., compositions comprising a VZV RNA-LNP are characterized by (e.g., when administered to a subject) sustained expression of an encoded polypeptide. For example, in some aspects, such compositions are characterized in that, when administered to a human, they achieve detectable polypeptide expression in a biological sample (e.g., serum) from such human and, in some aspects, such expression persists for a period of time that is at least at least 36 hours or longer, including, e.g., at least 48 hours, at least 60 hours, at least 72 hours, at least 96 hours, at least 120 hours, at least 148 hours, or longer.

In some aspects, the disclosure relates to a method of inducing an immune response against VZV in a subject. The method includes administering to the subject an effective amount of an RNA molecule, RNA-LNP and/or composition as described herein.

In another aspect, the disclosure relates to a method of vaccinating a subject. The method includes administering to the subject in need thereof an effective amount of an RNA molecule, RNA-LNP and/or composition described herein.

In another aspect, the disclosure relates to a method of treating or preventing an infectious disease. The method includes administering to the subject an effective amount of an RNA molecule RNA-LNP and/or composition as described herein.

In another aspect, the disclosure relates to a method of treating or preventing or reducing the severity of a VZV infection and/or illness caused by VZV. The method includes administering to the subject an effective amount of an RNA molecule, RNA-LNP and/or composition as described herein.

In another aspect, the disclosure relates to a method of treating or preventing or reducing the severity of an infectious disease in a subject by, for example, inducing an immune response to an infectious disease in the subject. In some aspects, the method includes administering a priming composition that includes an effective amount of an RNA molecule, RNA-LNP and/or composition described herein, and administering a booster composition including an effective amount of an RNA molecule, RNA-LNP and/or composition. In some aspects, the composition elicits an immune response including an antibody response. In some aspects, the composition elicits an immune response including a T cell response.

In another aspect, the disclosure relates to a method of treating or preventing or reducing the severity of a VZV infection and/or illness caused by VZV in a subject by, for example, inducing an immune response to VZV in the subject. In some aspects, the method includes administering a priming composition that includes an effective amount of an RNA molecule, RNA-LNP and/or composition described herein, and administering a booster composition including an effective amount of an RNA molecule RNA-LNP and/or composition as described herein. In some aspects, the composition elicits an immune response including an antibody response. In some aspects, the composition elicits an immune response including a T cell response.

The methods disclosed herein may involve administering to the subject a VZV RNA-LNP composition comprising at least one VZV RNA molecule having an open reading frame encoding at least one VZV antigenic polypeptide, thereby inducing in the subject an immune response specific to VZV antigenic polypeptide, wherein anti-antigenic polypeptide antibody titer in the subject is increased following vaccination relative to anti-antigenic polypeptide antibody titer in a subject vaccinated with a prophylactically effective dose (e.g., a therapeutically effective dose that prevents infection with the virus at a clinically acceptable level) of a traditional vaccine against the VZV. An “anti-antigenic polypeptide antibody” is a serum antibody the binds specifically to the antigenic polypeptide. In some aspects, the anti-antigenic polypeptide antibody titer in the subject is increased at least, at most, between any two of, or exactly 1, 2, 3, 4, 5, 6, 7, 8, 9, or 10 log following administration of the VZV RNA-LNP composition relative to anti-antigenic polypeptide antibody titer in a subject administered a prophylactically effective dose of a traditional composition against VZV.

The methods disclosed herein may involve administering to the subject a VZV RNA-LNP composition comprising at least one VZV RNA molecule having an open reading frame encoding at least one VZV antigenic polypeptide, thereby inducing in the subject an immune response specific to VZV antigenic polypeptide, wherein the immune response in the subject is equivalent to an immune response in a subject administered with a traditional composition against the VZV at least, at most, in between any two of, or exactly 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or 100 times the dosage level relative to the RNA composition.

In some aspects, the RNA molecule, RNA-LNP and/or composition is used as a vaccine. In some aspects, the RNA molecule, RNA-LNP and/or composition may be used in various therapeutic or prophylactic methods for preventing, treating or ameliorating of herpes zoster or shingles, or a disorder related to herpes zoster or shingles.

In some aspects, the RNA molecule, RNA-LNP and/or composition may be used in various therapeutic or prophylactic methods for preventing, treating or ameliorating of post herpetic neuralgia.

VZV RNA compositions may be administered prophylactically or therapeutically to healthy subjects or early in infection during the incubation phase or during active infection after onset of symptoms. In some aspects, the subject is immunocompetent.

In some aspects, the subject is immunocompromised.

In some aspects, the RNA molecule, RNA-LNP and/or composition is administered in a single dose. In some aspects, a second, third or fourth dose may be given. In some aspects, the RNA molecule, RNA-LNP and/or composition is administered in multiple doses.

In some aspects, the RNA molecule, RNA-LNP and/or composition is administered intramuscularly (IM) or intradermally (ID).

The present disclosure further provides a kit comprising the RNA molecule, RNA-LNP, and/or composition.

In some aspects, the RNA molecule, RNA-LNP and/or composition described herein is administered to a subject that is less than about 1 years old, or about 1 years old to about 10 years old, or about 10 years old to about 20 years old, or about 20 years old to about 50 years old, or about 60 years old to about 70 years old, or older.

In some aspects the subject is at least, at most, exactly, or between any two of less than 1 year of age, greater than 1 year of age, greater than 5 years of age, greater than 10 years of age, greater than 20 years of age, greater than 30 years of age, greater than 40 years of age, greater than 50 years of age, greater than 60 years of age, greater than 70 years of age, or older. In some aspects, the subject is greater than 50 years of age.

In some aspects the subject is at least, at most, exactly, or between any two of about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older. In some aspects, the subject may be about 50 years of age or older.

In some aspects the subject is at least, at most, exactly, or between any two of 1 year of age or older, 5 years of age or older, 10 years of age or older, 20 years of age or older, 30 years of age or older, 40 years of age or older, 50 years of age or older, 60 years of age or older, 70 years of age or older, or older. In some aspects the subject may be 50 years of age or older.

### X. Clinical Studies

The VZV RNA-LNP vaccines of the present disclosure comprise nucleoside-modified mRNA encoding glycoprotein E (gE) from VZV (modified RNA; modRNA). The VZV RNA-LNP vaccines may comprise RNA comprising a single-stranded, 5′-capped and polyadenylated modified RNA that is translated after entering the cell. The RNA comprises an open reading frame (ORF) that encodes variations of the VZV gE. For example, the RNA molecule may comprise gE_WT CO2 (RNA encodes the full length gE protein which is localized in the plasma membrane and Golgi), gE ms5 CO1 (RNA encodes a truncated gE protein in the C-terminal which is localized mainly in the plasma membrane) and/or gE ms6 CO2 (RNA encodes for the ectodomain of the gE protein which gets secreted). Further, as described herein, the RNA may comprise structural elements, such as untranslated regions (UTRs), optimized for high efficacy of the RNA. The VZV RNA-LNPs may comprise RNA as provided in Table 5 of Example 1 disclosed herein. The VZV RNA-LNPs may comprise RNA as provided in Tables 1 to 3 of Example 7 disclosed herein. The RNA may also comprise a substitution of 1-methyl-pseudouridine for uridine to decrease recognition of the vaccine RNA by innate immune sensors, such as toll-like receptors (TLRs) 7 and 8, resulting in decreased innate immune activation and increased protein translation.

The RNA molecules described herein are formulated/encapsulated into lipid nanoparticles (LNPs) to enable delivery of the RNA into host cells after intramuscular (IM) injection. The LNP formulation may comprise two functional lipids, ALC-0315 and ALC-0159, and two structural lipids, DSPC (1,2-distearoyl-sn-glycero-3-phosphocholine) and cholesterol. The potency of RNA vaccines is optimized by LNP encapsulation, which protects the RNA from degradation by extracellular RNases and facilitates delivery in the cell. After IM injection of VZV RNA-LNP vaccines, the LNPs are taken up by the cells, and the RNA is released into the cytosol. In the cytosol, the RNA is translated, and the encoded viral antigen is produced.

The Examples herein demonstrate the VZV RNA-LNP vaccines of the present disclosure are immunogenic in mice and induce both humoral and cell mediated immune responses in mice.

Clinical studies of the present disclosure evaluate the safety, tolerability, and immunogenicity of VZV RNA-LNP vaccines against VZV. For example, the VZV RNA-LNPs vaccines may be indicated for active immunization for the prevention of shingles disease caused by VZV for adults (e.g., ≥45, ≥50, ≥55, ≥60, ≥70 . . . etc. years of age or 50 through 69 years of age). VZV RNA-LNP vaccines may be administered in different dose level(s), dose formulation, number of doses and dosing schedules, as described herein, including but not limited to:
As a single-dose schedule or a two-dose schedule (e.g., Day 0 and about 2 months after or Day 0 and about 6 months after)
At different dose levels (e.g., about 15 μg, about 30 μg, about 60 μg, about 90 μg, about 100 μg or higher per administration)
At different formulations (non-lyophilized and/or lyophilized)

The VZV RNA-LNPs may be presented as a liquid or lyophilized formulation. Administration of the VZV RNA-LNP vaccines may be dosed in the range of about 15 μg, about 30 μg, about 60 μg, about 90 μg, about 100 μg or higher per dose with an injection volume of about 0.25 to 1 mL (e.g., about 0.25, 0.5, 1 mL). Dilution with sterile 0.9% sodium chloride (normal saline) may be required.

The objectives of VZV RNA-LNP clinical studies may include, but are not limited to:
To describe the safety and tolerability profile of VZV RNA-LNP vaccines administered at selected dose levels and schedules in participants.
To describe the immune responses elicited by SHINGRIX® and VZV RNA-LNP vaccines administered at selected dose levels and schedules in participants.

### Examples

Below are examples of specific aspects for carrying out the present disclosure. The following examples are included to demonstrate aspects of the disclosure. The examples are offered for illustrative purposes only and are not intended to limit the scope of the present disclosure in any way. It should be appreciated by those of skill in the art that the techniques disclosed in the examples which follow represent techniques discovered by the inventor to function well in the practice of the disclosure. However, those of skill in the art should, in light of the present disclosure, appreciate that many changes may be made in the specific aspects which are disclosed and still obtain a like or similar result without departing from the spirit and scope of the disclosure. Efforts have been made to ensure accuracy with respect to numbers used (e.g., amounts, temperatures, etc.), but some experimental error and deviation should, of course, be allowed for.

### Example 1. Generation of VZV RNA Constructs

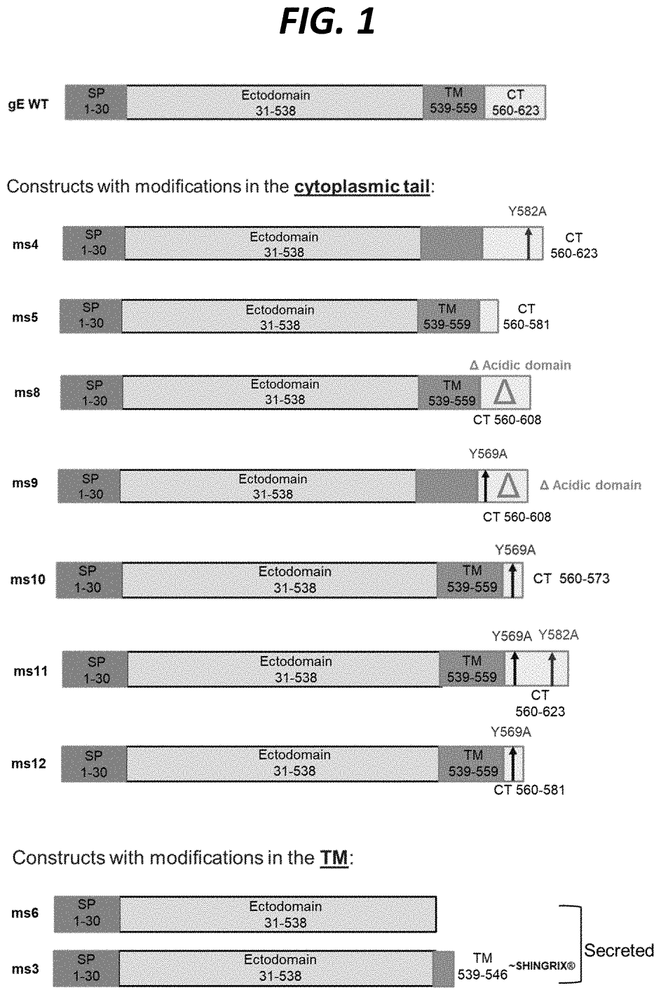

RNA constructs generated herein encode VZV gE wild-type (WT) and gE variant proteins having cytoplasmic tail (CT) and/or transmembrane (TM) domain modifications. FIG. 1 and Table 4 show WT gE proteins (gE WT), variant gE proteins having cytoplasmic tail modifications (ms4, ms5, ms8, ms9, ms10, ms11, and ms12), and variant gE proteins having TM modifications (ms3 and ms6).

TABLE 4
VZV gE proteins and description

| VZV Protein | VZV Protein Description |
|---|---|
| gE WT | Full length WT VZV gE (623aa) |
| SEQ ID NO: 1 | |
| ms3 | VZV gE with deletion of aa 547-623 (partial TM and full CT deletion) |
| SEQ ID NO: 2 | (546aa), protein component of SHINGRIX ®, *SECRETED |
| ms4 | Full length VZV gE with substitution Y582A in YAGL (aa 582-585) |
| SEQ ID NO: 3 | endocytosis recycling signal to prevent gE endocytosis (623aa) |
| ms5 | VZV gE with deletion of aa 582-623 (partial TM deletion) (581 aa) |
| SEQ ID NO: 4 | |
| ms6 | VZV gE with deletion of aa 539-623 (full TM and CT deletion) (538 |
| SEQ ID NO: 5 | aa), *SECRETED |
| ms8 | VZV gE with deletion of the acidic domain (aa 588-602) in the CT (608 |
| SEQ ID NO: 6 | aa) |
| ms9 | VZV gE with substitution Y569A in AYRV (aa 568-571) domain that |
| SEQ ID NO: 7 | targets gE to the trans-Golgi network and deletion of the acidic domain |
| | aa 588-602 in the CT (608aa) |
| ms10 | VZV gE with substitution Y569A and deleted aa 574-623 (partial CT |
| SEQ ID NO: 8 | deletion) (573 aa) |
| ms11 | Full length VZV gE with substitutions Y569A (TGN) and Y582A (623 |
| SEQ ID NO: 9 | aa) |
| ms12 | VZV gE with substitution Y569A and deletion of aa 582-623 (partial |
| SEQ ID NO: 10 | TM deletion) (581 aa) |

DNA sequences encoding VZV proteins were prepared and utilized for in vitro transcription reactions to generate RNA. In vitro transcription of RNA is known in the art and is described herein. DNA templates were cloned into a plasmid vector with backbone sequence elements (T7 promoter, 5′ and 3′ UTR, poly-A tail for improved RNA stability and translational efficiency. The DNA was purified, spectrophotometrically quantified and in vitro-transcribed by T7 RNA polymerase in the presence of a trinucleotide cap1 analogue ((m₂^{7,3′-O})Gppp(m^{2′-O})ApG) (TriLink) and with N1-methylpseudouridine (φ) replacing uridine (modified RNA; modRNA).

The VZV RNA was generated from codon-optimized (CO) DNA for stabilization and superior protein expression. As used herein, CO1 indicates about 58% G/C content, CO2 indicates about 66% G/C content, and CO3 indicates about 62% G/C content. Table 5 shows RNA constructs of the present disclosure, and corresponding sequences, comprising a 5′ UTR, an open reading frame encoding a varicella-zoster virus (VZV) polypeptide, a 3′ UTR and a poly-A tail.

TABLE 5
VZV gE RNA constructs/molecules

| | | | | | VZV gE |
|---|---|---|---|---|---|
| RNA | 5′ UTR | VZV [ORF] | 3′ UTR | Poly-A tail* | Protein/DNA |
| Construct | SEQ ID NO | SEQ ID NO | SEQ ID NO | SEQ ID NO | SEQ ID NO |
| gE WT | 281 or 312 | 146 | 284 or 317 | 287 or 315 | 1/12 |
| gEWT CO1 | 281 or 312 | 147 | 284 or 317 | 287 or 315 | 1/13 |
| gEWT CO2 | 281 or 312 | 148 | 284 or 317 | 287 or 315 | 1/14 |
| ms3 CO1 | 281 or 312 | 149 | 284 or 317 | 287 or 315 | 2/15 |
| ms3 CO2 | 281 or 312 | 150 | 284 or 317 | 287 or 315 | 2/16 |
| ms4 CO1 | 281 or 312 | 151 | 284 or 317 | 287 or 315 | 3/17 |
| ms4 CO2 | 281 or 312 | 152 | 284 or 317 | 287 or 315 | 3/18 |
| ms5 CO1 | 281 or 312 | 153 | 284 or 317 | 287 or 315 | 4/19 |
| ms5 CO2 | 281 or 312 | 154 | 284 or 317 | 287 or 315 | 4/20 |
| ms5 CO2 v2 | 281 or 312 | 155 | 284 or 317 | 287 or 315 | 4/21 |
| ms6 CO1 | 281 or 312 | 156 | 284 or 317 | 287 or 315 | 5/22 |
| ms6 CO2 | 281 or 312 | 157 | 284 or 317 | 287 or 315 | 5/23 |
| ms8 CO1 | 281 or 312 | 158 | 284 or 317 | 287 or 315 | 6/24 |
| ms9 CO1 | 281 or 312 | 159 | 284 or 317 | 287 or 315 | 7/25 |
| ms9 CO2 | 281 or 312 | 160 | 284 or 317 | 287 or 315 | 7/26 |
| ms10 CO1 | 281 or 312 | 161 | 284 or 317 | 287 or 315 | 8/27 |
| ms10 CO2 | 281 or 312 | 162 | 284 or 317 | 287 or 315 | 8/28 |
| ms10 CO3 | 281 or 312 | 163 | 284 or 317 | 287 or 315 | 8/29 |
| ms11 CO1 | 281 or 312 | 164 | 284 or 317 | 287 or 315 | 9/30 |
| ms11 CO2 | 281 or 312 | 165 | 284 or 317 | 287 or 315 | 9/31 |
| ms12 CO1 | 281 or 312 | 166 | 284 or 317 | 287 or 315 | 10/32 |
| ms12 CO2 | 281 or 312 | 167 | 284 or 317 | 287 or 315 | 10/33 |
| gE_P1_IRES_CA | 281 or 312 | 168 | 284 or 317 | 287 or 315 | 11/34 |
| gE_P2 | 281 or 312 | 169 | 284 or 317 | 287 or 315 | 1/35 |
| gE_P3 | 281 or 312 | 170 | 284 or 317 | 287 or 315 | 1/36 |
| gE_P4 | 281 or 312 | 171 | 284 or 317 | 287 or 315 | 1/37 |
| gE_P6 | 281 or 312 | 172 | 284 or 317 | 287 or 315 | 1/38 |
| gE_P7 | 281 or 312 | 173 | 284 or 317 | 287 or 315 | 1/39 |
| gE EB1 | 281 or 312 | 174 | 284 or 317 | 287 or 315 | 1/40 |
| gE MM_1 to | 281 or 312 | 175 to 238 | 284 or 317 | 287 or 315 | 1/41 to 104 |
| gE MM_64 | | | | | |
| gE FO_D15_1_EB | 281 or 312 | 239 | 284 or 317 | 287 or 315 | 1/105 |
| gE FO_D15_1 to | 281 or 312 | 240 to 254 | 284 or 317 | 287 or 315 | 1/106 to 120 |
| gE FO_D15_15 | | | | | |
| gE FO_D13_1 to | 281 or 312 | 255 to 267 | 284 or 317 | 287 or 315 | 1/121 to 133 |
| gE FO_D13_13 | | | | | |
| gE FO_D12_1 to | 281 or 312 | 268 to 279 | 284 or 317 | 287 or 315 | 1/134 to 145 |
| gE FO_D12_12 | | | | | |

*Poly-A tail length may contain +2/−2 A or +1/−1 A.

VZV RNA constructs/molecules and RNA-LNPs evaluated in in vitro and in vivo experiments described herein in the Examples comprise modified RNA (modRNA) comprising an RNA sequence having all uridines replaced by N1-methylpseudouridine (ψ).

### Example 2. VZV gE Expression and Subcellular Location (Vero Cells)

Expression of VZV RNA constructs were tested in transfected Vero cells, a kidney epithelial cell culture line derived from African green monkeys. Seeded Vero cells were transfected for 24 hours at 37° C., 5% CO₂ with 10 ng, 25 ng, or 50 ng of RNA constructs (Table 5) using MESSENGERMAX™ in accordance with the manufacturer's instructions. Cells were washed three times with PBS+Ca/Mg, and fixed in 4% paraformaldehyde (PFA) for 20 minutes at 25° C. Cells were washed twice with 3% bovine serum albumin (BSA) in PBS+Ca/Mg. Primary and secondary staining antibodies were diluted in 0.1% saponin in goat serum. Cells were stained with a 1:1000 dilution of primary antibody for 1 hour at 37° C., washed three times with PBS+Ca/Mg, and incubated with secondary antibody (1:500) and CELLMASK™ (1:140,000) for 45 minutes at 37° C. Cells were then stained with Dapi (1:15,000-1:20,000) for 15 minutes at 25° C., and washed three times with PBS+Ca/Mg. Cells were analyzed for VZV gE expression and subcellular localization using an Opera PHENIX® Plus High-Content Screening System at 10× or 63× magnification.

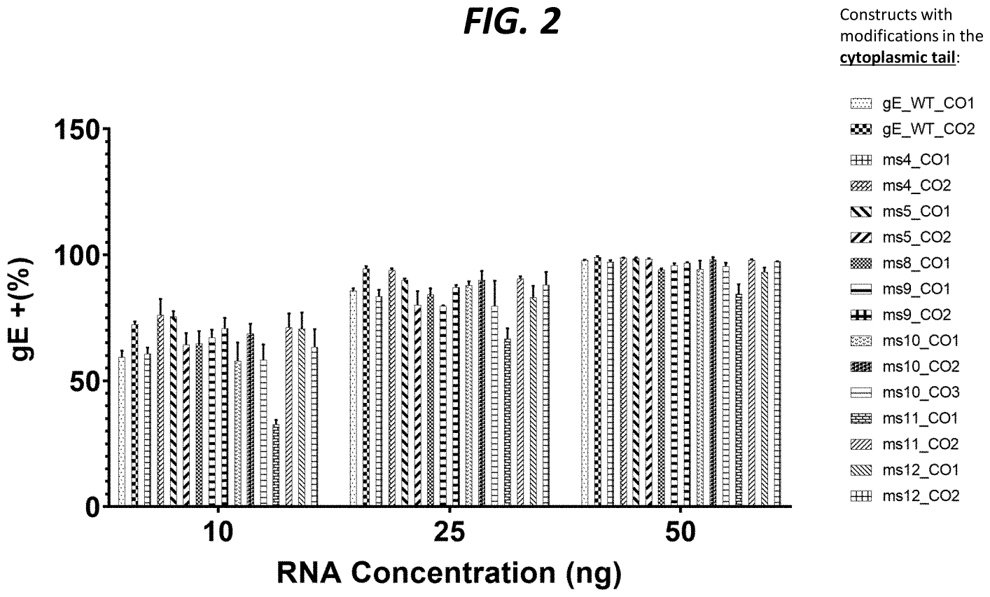

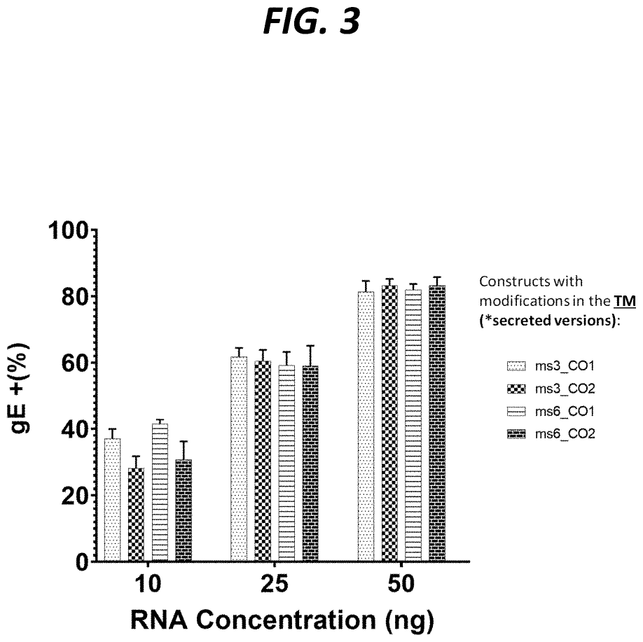

Imaging analyses reveal an RNA dose-dependent increase in the percentage of VZV gE⁺ transfected Vero cells, with almost 100% of cells expressing VZV gE at the 50 ng dose among cytoplasmic tail mutants (FIG. 2), and about 80% of cells expressing VZV gE at the 50 ng dose among the secreted mutants (FIG. 3). Since the secreted mutants are mainly transported outside of the cell, lower levels are detected inside.

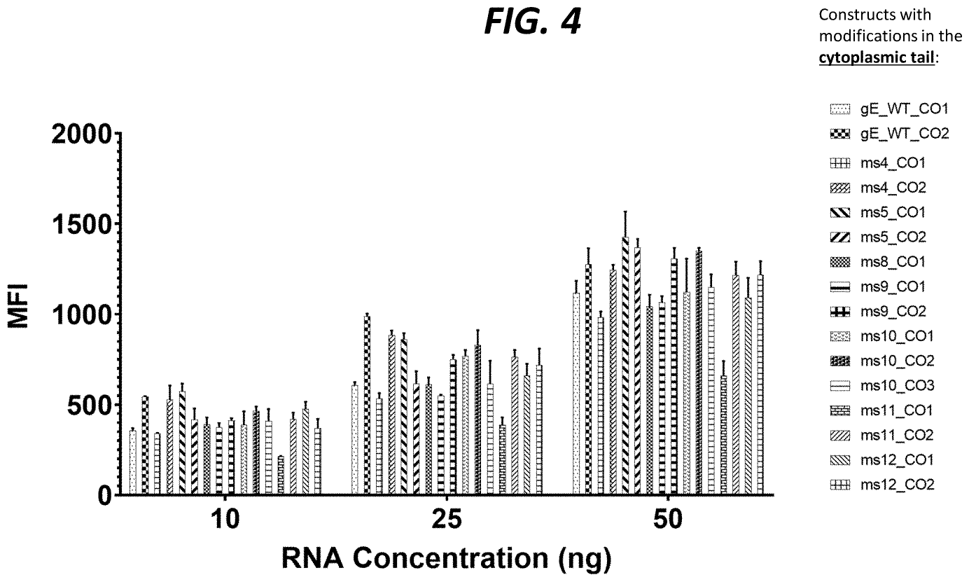

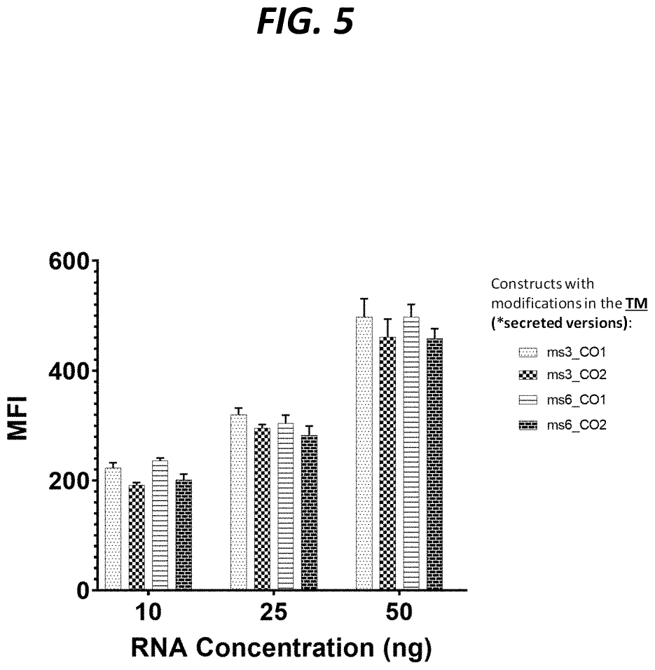

The mean fluorescence intensity (MFI) of each transfected RNA construct reveals a dose-dependent increase in VZV gE expression among cytoplasmic tail mutants (FIG. 4) and secreted mutants (FIG. 5), as well as a positive correlation between higher G/C content (CO2) and higher VZV gE expression levels among cytoplasmic tail mutants (FIG. 4).

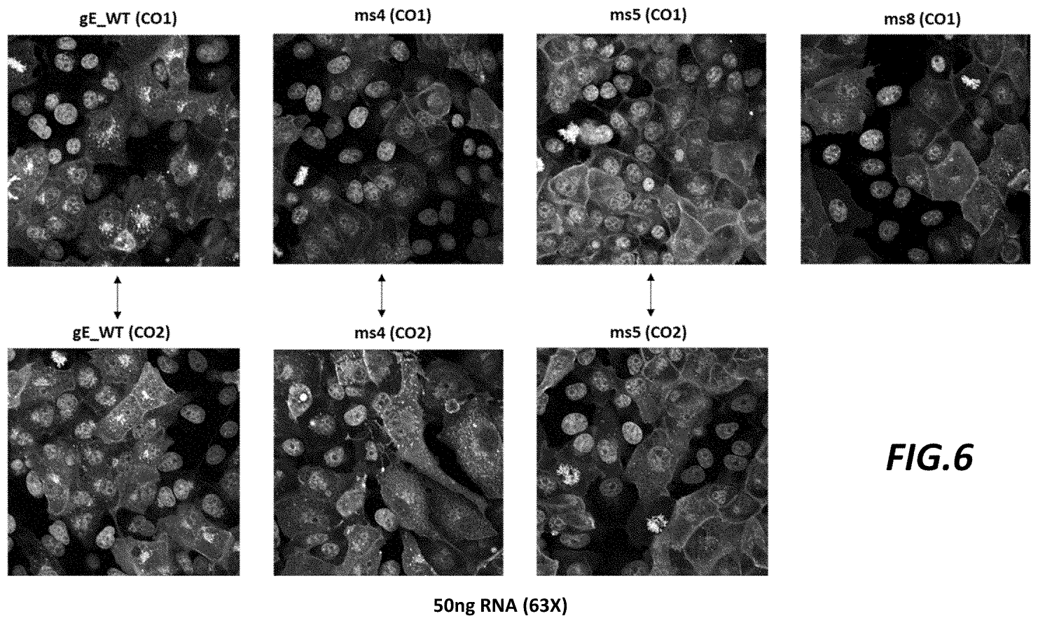

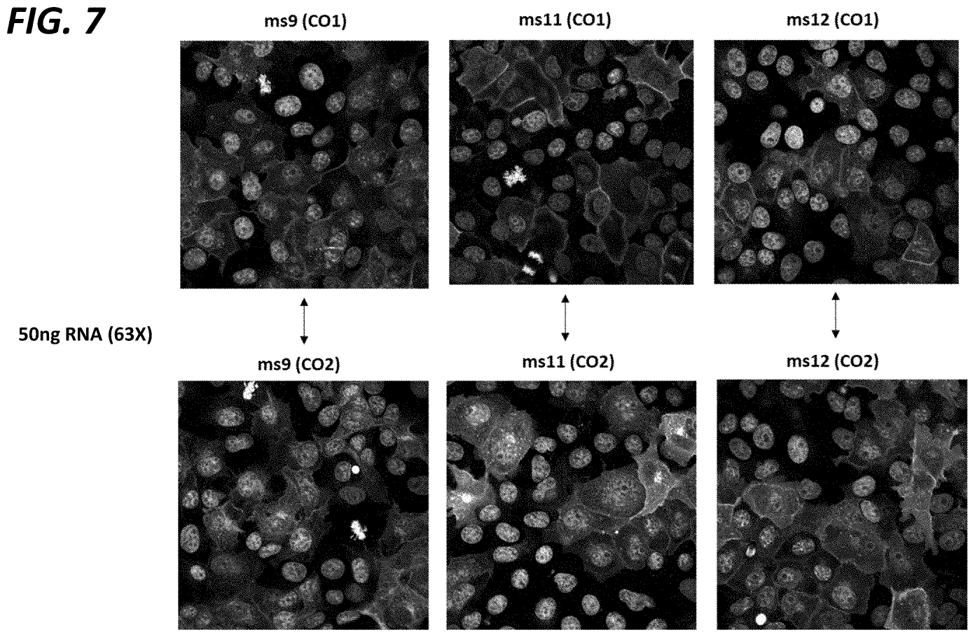

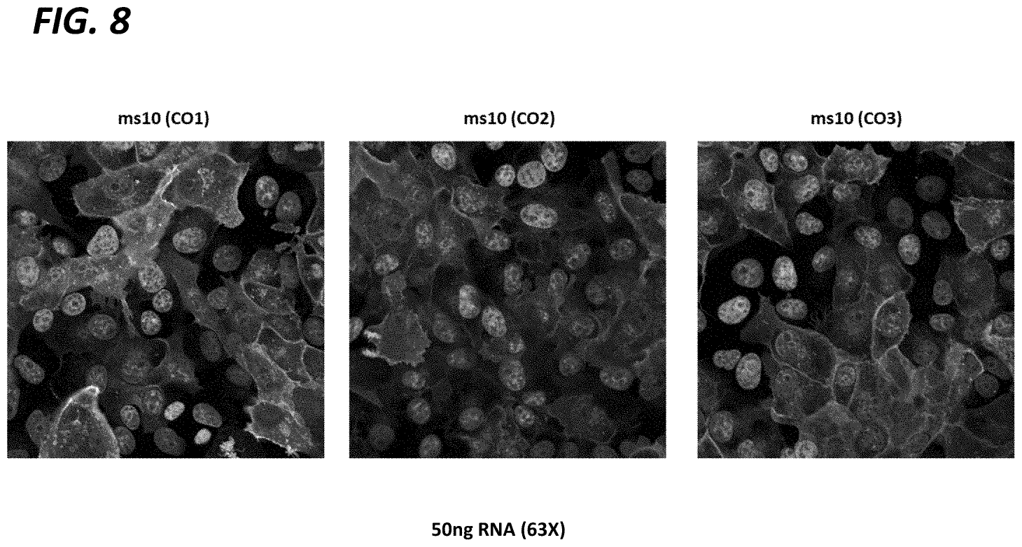

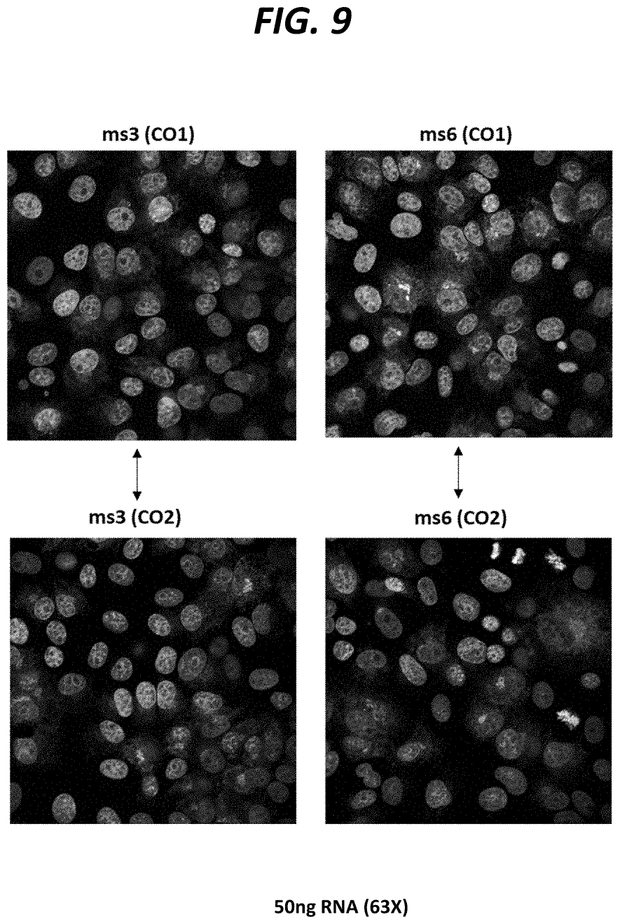

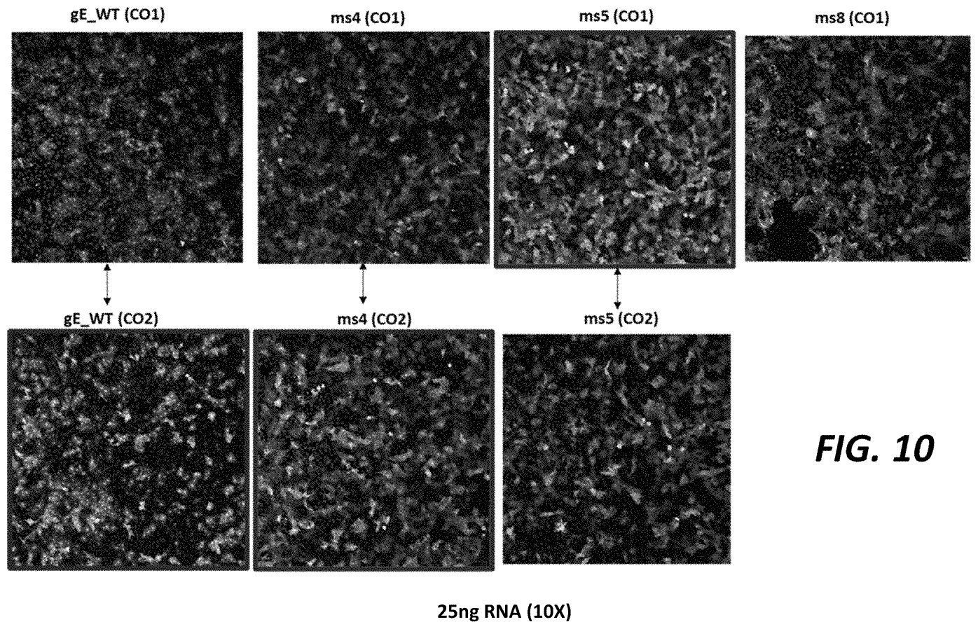

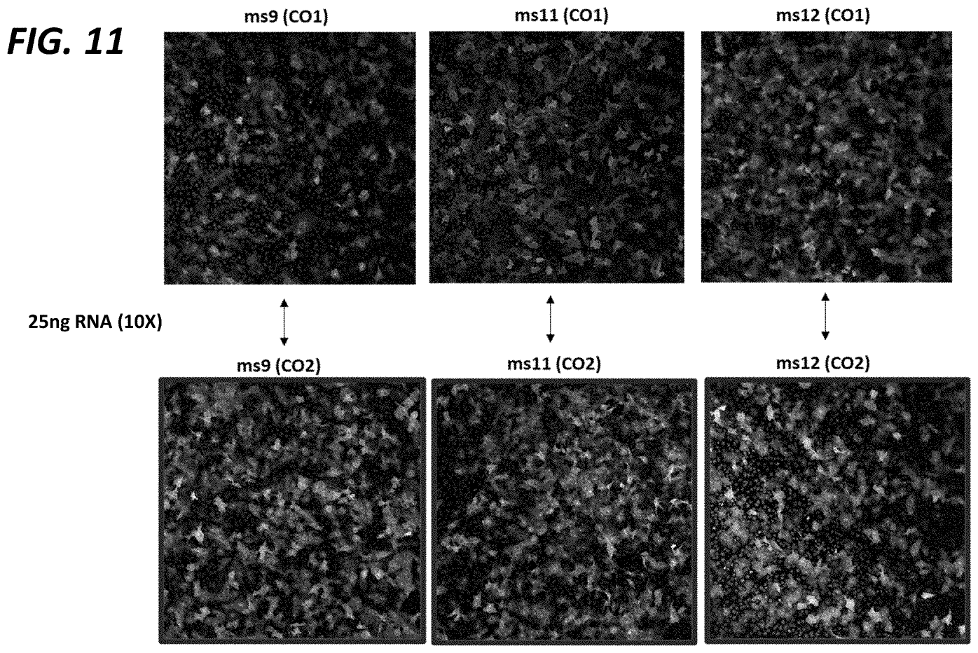

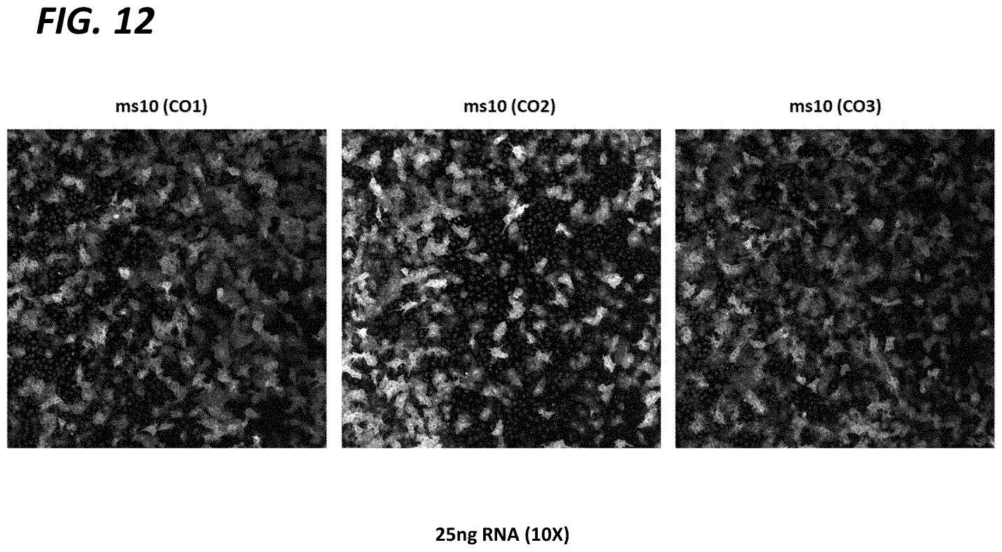

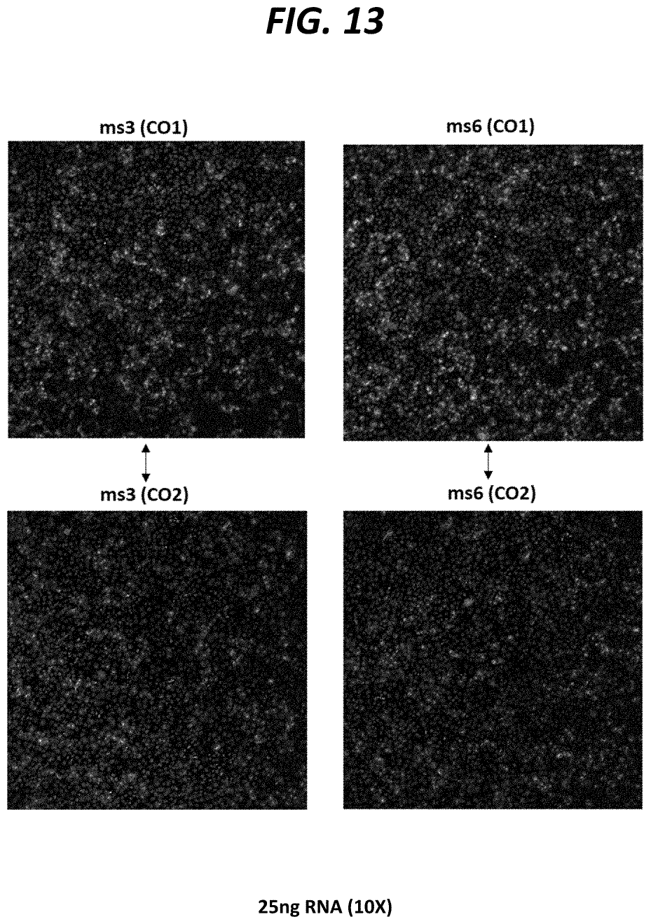

Imaging analyses reveals the subcellular localization of VZV gE in Vero cells transfected with the various RNA constructs. Localization of VZV gE in gE_WT-transfected cells occurred within cellular membranes and the trans-Golgi network (TGN; FIG. 6, 63× magnification; FIG. 10, 10× magnification). Cytoplasmic tail mutants (ms4, ms5, ms8, ms9, ms10, ms11, ms12) displayed VZV gE localization preferentially within cellular membranes (FIGS. 6 to 8, 63× magnification; FIGS. 10 to 12, 10× magnification). Secreted mutants (ms3, ms6) displayed VZV gE localization within the culture supernatant (FIG. 9, 63× magnification; FIG. 13, 10× magnification).

### Example 3. Preparation of VZV gE modRNA Formulated in LNP

Purified RNA (as described in Table 5) was formulated/encapsulated into lipid nanoparticles (RNA-LNPs) using an ethanolic lipid mixture of ionizable cationic lipid and transferred into an aqueous buffer system via diafiltration to yield a lipid nanoparticle composition, as described herein. The RNA-LNP comprises a VZV RNA molecule, a cationic lipid, ((4-hydroxybutyl)azanediyl)bis(hexane-6,1-diyl)bis(2-hexyldecanoate)), a PEGylated lipid, 2-[(polyethylene glycol)-2000]-N,N-ditetradecylacetamide and two structural lipids (1,2-distearoyl-sn-glycero-3-phosphocholine (DSPC]) and cholesterol), see Table 6.

TABLE 6
Lipid formulation

| | Molecular | | |
|---|---|---|---|
| | Weight | | |
| Lipid | [Da] | Molecular Formula | Chemical name and structure |
| Cationic | 766 | C₄₈H₉₅NO₅ | (4-hydroxybutyDazanediyl)bishesane-6.1- |
| Lipid | | | diyl)bis(2-hexyldecanoate) |
| ALC-0315 | | | 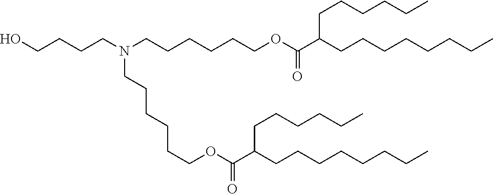 [SMILES: CCCCCCCCC(CCCCCC)C(=O)OCCCCCCN(CCCCO)CCCCCCOC(=O)C(CCCCCC)CCCCCCCC] |
| PEG-Lipid | About | C₃₀H₆₀NO(C₂H₄O)ₙOCH₃ | 2-((polyethylene alycol)-2000]-N,N- |
| ALC-0159 | 2400- | n = 45-50 | ditetradecyclacetamide |
| | 2600 | | 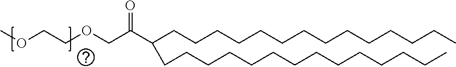 |
| DSPC | 790 | C₃₀H₈₈NO₈P | 12-Distearoyl-sn-glycero-3-phosphocholine |
| | | | 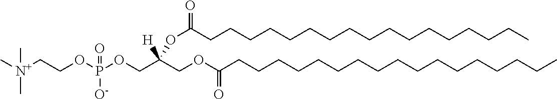 [SMILES: CCCCCCCCCCCCCCCCCC(=O)OC[C@H](COP(=O)([O-])OCC[N+](C)(C)C)OC(=O)CCCCCCCCCCCCCCCCC] |
| Cholesterol | 387 | C₂₇H₄₆O | 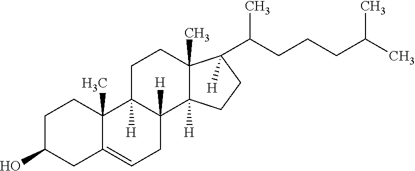 |

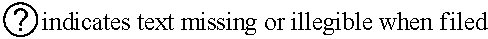

### Example 4: In Vitro Expression (HEK 293T Cells)

In vitro expression (IVE) analyses were conducted to assess the potency of VZV RNA-LNP vaccines. RNA constructs were formulated into LNPs as previously described in Example 3, and a range of input VZV RNA-LNP quantities was transfected into human embryonic kidney (HEK) 293T cells for 24 hours at 37° C., 5% CO₂. Transfected cells were washed with DPBS, detached from plate wells using ACCUTASE®, and rinsed with cold PBS. Cells were subjected to live/dead staining, fixation, and permeabilization prior to FACS staining. Fixed and permeabilized cells were incubated with an anti-gE primary antibody (1:1000) for 45 minutes at 2-8° C., washed twice with BD PERM/WASH™ buffer and incubated with a goat anti-human kappa-PE secondary antibody (1:1000) for 30 minutes at 2-8° C. Cells were washed twice with BD PERM/WASH™ buffer, resuspended in 1×FACS buffer, and analyzed on a BD LSRII to determine the proportion of cells expressing VZV gE for each VZV RNA-LNP input. A titration curve was created whereby input RNA quantities were plotted against the percentage of gE-expressing cells to determine the EC50 of each VZV RNA-LNP vaccine.

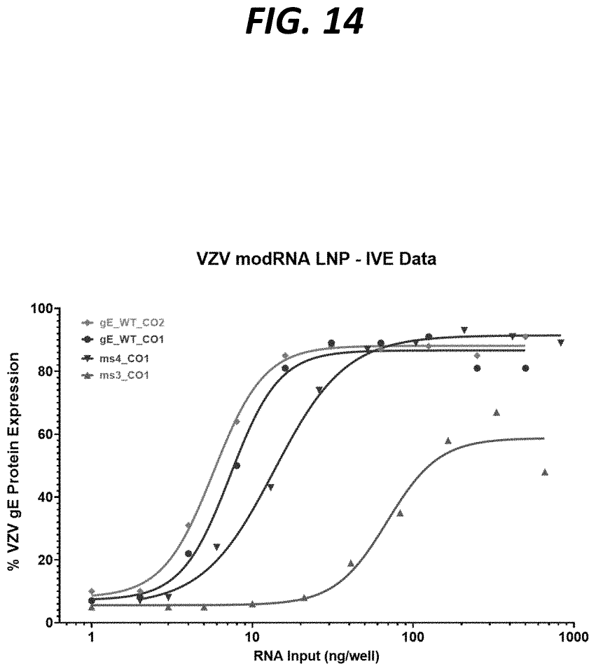

The data in Table 7 and FIG. 14 shows positive correlation between VZV RNA-LNP vaccine potency (e.g., lower EC50) and higher MFI. Comparing the MFI of VZV RNA-LNP (293T cell) and VZV gE RNA (Vero cells; see Example 2) shows that both VZV RNA-LNP and VZV gE RNA share similar MFI trends (Table 7), wherein the potency and MFI of gE_WT CO2>gE_WT CO1>ms4 CO1>ms3 CO1.

TABLE 7
In vitro expression

| | VZV RNA-LNPs | | |
|---|---|---|---|
| | (293T cells) | | VZV RNA |
| | EC50 | | (Vero cells) |
| RNA construct | (ng/well) | MFI | MFI |
| gE_WT CO2 | 6 | 2442 | 1274 |
| gE_WT CO1 | 8 | 2057 | 1116 |
| ms4 CO1 | 15 | 1713 | 981 |
| *ms3 CO1 | 231 | 663 | 497 |

*secreted

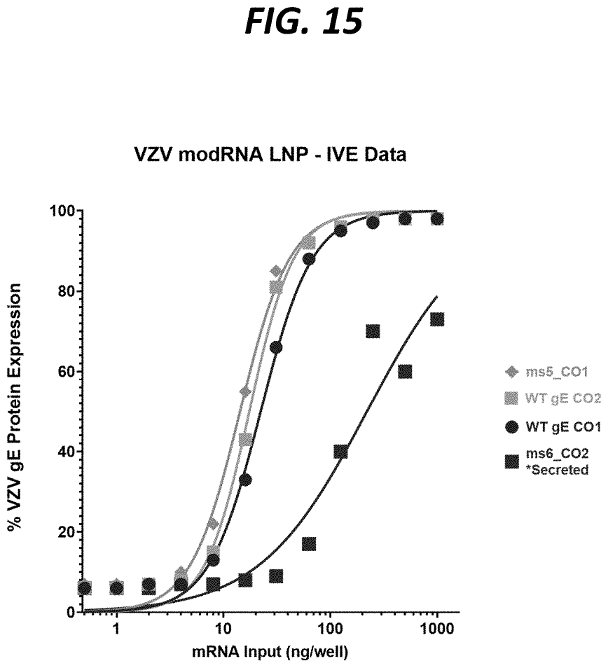

These trends were also observed for the data in Table 8 and FIG. 15, wherein the potency and MFI of ms5 CO1>gE_WT CO2>gE_WT CO1>ms6 CO2.

TABLE 8
In vitro expression

| | VZV RNA-LNPs | | |
|---|---|---|---|
| | (293T cells) | | VZV RNA |
| | EC50 | | (Vero cells) |
| RNA construct | (ng/well) | MFI | MFI |
| ms5_CO1 | 14 | 543 | 1368 |
| gE_WT CO2 | 17 | 452 | 1274 |
| gE_WT CO1 | 22 | 337 | 1116 |
| *ms6 CO2 | 220 | 162 | 458 |

*secreted

### Example 5. Immune Responses (In Vivo Experiments)

VZV RNA-LNP vaccines were tested for their ability to induce IgG and T cell responses in Balb/c mice. 15 mice per group were immunized in accordance with the schedule and specifications in Table 9. Briefly, 15 mice per group were immunized intramuscularly (IM) on Day 0 and boosted IM on Day 28. Mice immunized with SHINGRIX® received 1, 2.5, or 5 μg, corresponding to 1/50, 1/20, and 1/10 of the human clinical dose (50 μg), respectively; mice immunized with gE_WT CO1 received 0.5 or 1 μg; mice immunized with ms3 CO1 received 0.5 μg; mice immunized with ms4 CO1 received 0.5 μg; mice immunized with gE_WT CO2 received 0.5 μg; mice immunized with lyophilized gE_WT CO1 received 0.5 or 1 μg. Saline was administered on Days 0 and 28 to a negative control group. Serum was collected from mice on Days 28 and 42 for characterization of VZV gE-binding IgG levels (LUMINEX® analyses). Spleens were harvested, and sera collected, from five mice in each group on Day 35 for cellular analyses.

TABLE 9
Administration schedule of SHINGRIX® and VZV RNA-LNPs

| | | | Dose | | Dose Vol/ | Admin | Bleed |
|---|---|---|---|---|---|---|---|
| # | Mice | Vaccine | (μg) | | Route | (Day) | (Day) |
| 1 | 15 | Saline | — | | 50 μl/IM | 0, 28 | 28, 34*, 42 |
| 2 | 15 | SHINGRIX® | 5 | μg | 50 μl/IM | 0, 28 | 28, 34*, 42 |
| 3 | 15 | SHINGRIX® | 2.5 | μg | 50 μl/IM | 0, 28 | 28, 34*, 42 |
| 4 | 15 | SHINGRIX® | 1 | μg | 50 μl/IM | 0, 28 | 28, 34*, 42 |
| 5 | 15 | gE_WT CO1 | 0.5 | μg | 50 μl/IM | 0, 28 | 28, 34*, 42 |
| 6 | 15 | gE_WT CO1 | 1 | μg | 50 μl/IM | 0, 28 | 28, 34*, 42 |
| 7 | 15 | ms3 CO1 | 0.5 | μg | 50 μl/IM | 0, 28 | 28, 34*, 42 |
| 8 | 15 | ms4 CO1 | 0.5 | μg | 50 μl/IM | 0, 28 | 28, 34*, 42 |
| 9 | 15 | gE_WT CO2 | 0.5 | μg | 50 μl/IM | 0, 28 | 28, 34*, 42 |
| 10 | 15 | gE_WT CO1 | 0.5 | μg | 50 μl/IM | 0, 28 | 28, 34*, 42 |
| | | lyophilized | | | | | |
| 11 | 15 | gE_WT CO1 | 1 | μg | 50 μl/IM | 0, 28 | 28, 34*, 42 |
| | | lyophilized | | | | | |

*Euthanize 5 mice at day 34 for spleens (T cells)

### IgG Antibodies Titers

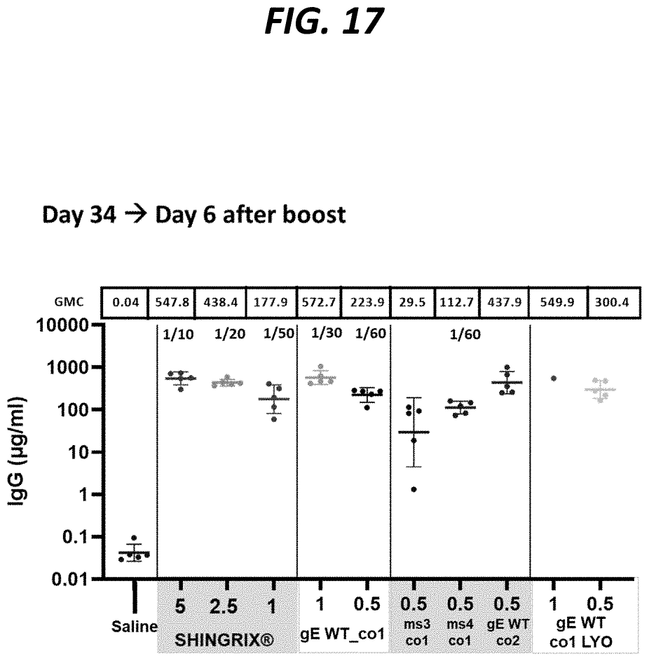

Serum IgG levels were determined for each mouse sample using the LUMINEX® platform. Briefly, diluted sera were incubated in the dark in the presence of blocked, recombinant gE protein-bound single-plex microspheres (MAGPLEX® Microspheres, region 79) for 20±4 hours at 2-8° C., 300 rpm. A secondary antibody solution (R-phycoerythrin Conjugated AffiniPure F(ab)′2 Fragment Goat Anti-Mouse IgG F(ab)′2 Fragment Specific) was prepared at 1:250 in LXA-16 buffer. Plates were washed three times in LXA-20 (EIA-7), and secondary antibody was applied to each well of the assay plate. Plates were covered with aluminum sealers and incubated for 2 hours±15 minutes at 25° C., 300 rpm. Plates were washed three times with LXA-20 (EIA-7) buffer, and 100 ul LXA-20 was applied to each well. Plates were covered with aluminum sealers and incubated at 25° C., 300 rpm for a minimum of 4 minutes, or up to 2 hours. Plates were analyzed on a BIO-PLEX® Reader. IgG titers for Day, 28, 34 and 42 are show in Table 10 and FIGS. 17-19.

TABLE 10
Geometric Mean Titers (GMCs) on Days 28, 34 and 42

| | | | Day 28 | Day 34 | Day 42 |
|---|---|---|---|---|---|
| | Dose | | (GMC) | (GMC) | (GMC) |
| Saline | — | | 0.03 | 0.04 | 0.09 |
| SHINGRIX ® | 5 | μg | 37.74 | 547.8 | 1260.76 |
| | 2.5 | μg | 6.77 | 438.4 | 959.78 |
| | 1 | μg | 2.33 | 177.9 | 498.79 |
| gE_WT CO1 | 1 | μg | 33.78 | 572.7 | 1630.85 |
| | 0.5 | μg | 7.69 | 223.9 | 918.43 |
| ms3 CO1 | 0.5 | μg | 3.98 | 29.5 | 370.11 |
| ms4 CO1 | 0.5 | μg | 3.55 | 112.7 | 598.57 |
| gE_WT CO2 | 0.5 | μg | 12.90 | 437.9 | 524.39 |
| gE_WT CO1 Iyo | 1 | μg | 27.03 | 549.9 | 1979.67 |
| | 0.5 | μg | 7.33 | 300.4 | 1166.40 |

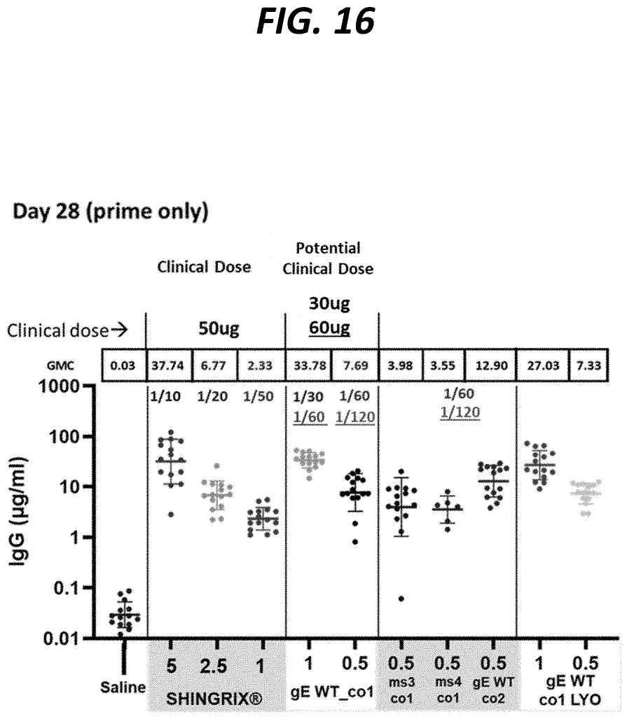

As shown in FIG. 16, IgG titers on Day 28 (prime only/prior to receiving the boost) reveal a dose-dependent change in geometric mean concentration (GMC) in mice receiving SHINGRIX®, gE_WT CO1, and lyophilized gE_WT CO1. The GMC of gE_WT CO1 (7.69) and gE_WT CO2 (12.90) administered at 0.5 μg (1/60 of a potential clinical dose of 30 μg) are notably higher than the GMC of SHINGRIX® (2.33) at administered at 1 μg (1/50 its human clinical dose of 50 μg) (FIG. 16). Potential clinical doses for VZV RNA-LNPs include, but are not limited to, 15 μg, 30 μg, 60 μg, 90 μg, 100 μg or higher. Accordingly, the GMC of gE_WT CO1 (33.78) administered at 1 μg (1/60 of a potential clinical dose of 60 μg) is higher than the GMC of SHINGRIX® (2.33) administered at 1 μg (1/50 its human clinical dose of 50 μg).

As shown in FIG. 17, IgG titers on Day 34 (six days after receiving the boost on Day 28) similarly reveal a dose-dependent change in GMC in mice receiving SHINGRIX®, gE_WT CO1, and lyophilized gE_WT CO1 (FIG. 17). Higher IgG levels were observed for VZV RNA-LNP vaccines compared to SHINGRIX® (in particular, GMC of 223.9 for gE_WT CO1 and 437.9 for gE_WT CO2 at 1/60 of a potential clinical dose of 30 μg vs. GMC of 177.9 for SHINGRIX® at 1/50 its human clinical dose (FIG. 17)).

A positive correlation was observed between higher G/C content and higher IgG titers when comparing gE_WT CO1 (about 58% G/C) and gE_WT CO2 (about 66% G/C), see FIGS. 16 and 17.

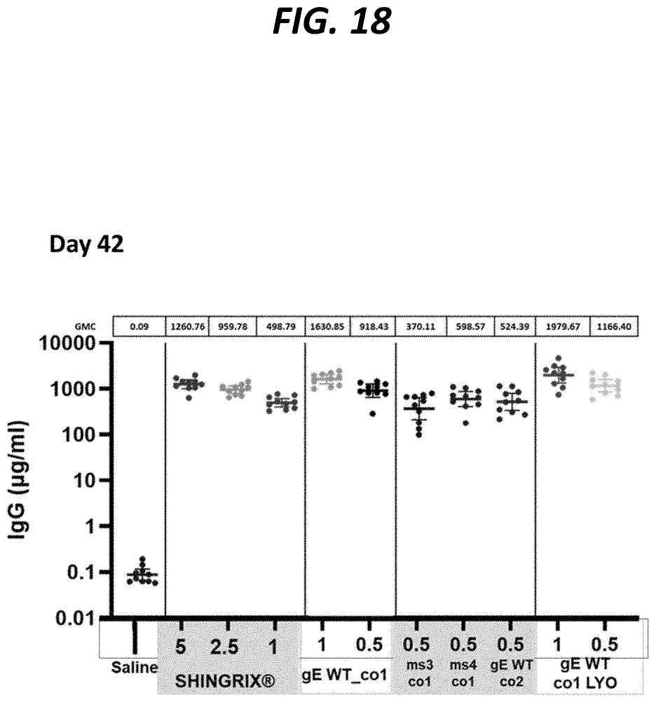

As shown in FIG. 18, IgG titers on Day 42 further reveal a dose-dependent change in geometric mean concentration (GMC). Higher IgG levels were observed for gE_WT CO1 (GMC of 918.43) and gE_WT CO2 (GMC of 524.39) compared to SHINGRIX® (GMC of 498.79).

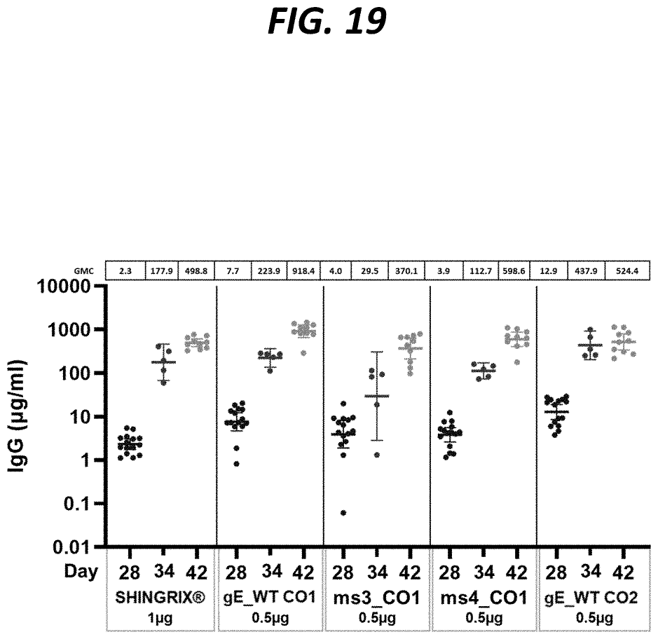

FIG. 19 shows compiles the IgG levels of SHINGRIX® (1 μg) and RNA-LNP vaccines (0.5 μg) observed at Days 28 (prime only), 34 (six days after boost) and 42.

Comparable IgG titers were observed for the non-lyophilized gE_WT CO1 and lyophilized gE_WT CO1 Lyo.

### Cell-Meditated Immunity (T Cell Responses)

Splenocytes were harvested from Balb/c mice on Day 34, (34 days after immunization, 6 days after boost) to assess gE-specific T cell responses induced. An Intracellular Cytokine Staining (ICS) assay was used to detect the presence of cytokines within CD4⁺ or CD8⁺ T cells following antigen peptide stimulation. ICS assay can detect multiple cytokines, including IFN-γ, produced in both CD4⁺ and CD8⁺ T cells following antigen peptide stimulation. During the ex vivo stimulation of splenocytes, reagents to block protein secretion are added to retain the synthesized cytokine to allow their detection by intracellular staining. Following stimulation, cells are stained for surface and intracellular markers to identify T cell types (CD3⁺ cells for CD4 and CD8 T cells), activation markers (CD154/CD40L) and cytokines. CD4⁺ T cells expressing IFN-γ, IL-2, TNFα and CD40L, and CD8⁺ T cells expressing IFN-γ were assessed to evaluate gE-specific T cells.

2×10⁶ splenocytes were stimulated with a 2 μg/mL gE peptide pool mix, a mix of 10 ng/ml phorbol myristate acetate (PMA) and 1 μg/mL ionomycin (positive control), or DMSO (negative control). BD GOLGISTOP™ and BD GOLGIPLUG™ were added to block protein secretion. Following incubation for 6 hours at 37° C., cells were stained for viability (10 min at 25° C.) and extracellular markers with directly labelled antibodies (20 min at 25° C.). Cells were fixed and permeabilized with BD CYTOFIX/CYTOPERM™ solution. Intracellular staining for cytokines (IFN-γ, IL-2, TNFα) and activation markers (CD154/CD40L) was performed in BD CYTOFIX/CYTOPERM™ solution (30 min at 25° C.). Cells were washed, resuspended in 2% FBS/PBS buffer and acquired on an LSRFORTESSA™. Data were analyzed by FlowJo 10.7.1. Results shown are background (media-DMSO) subtracted.

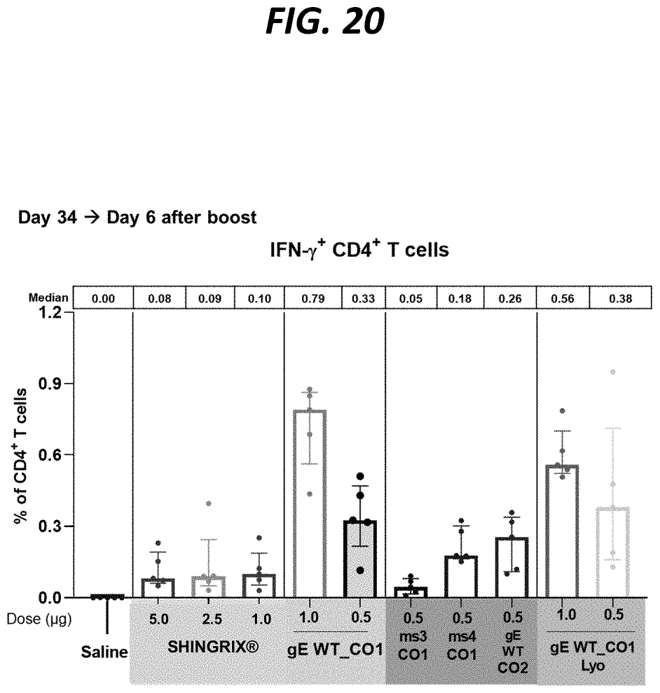

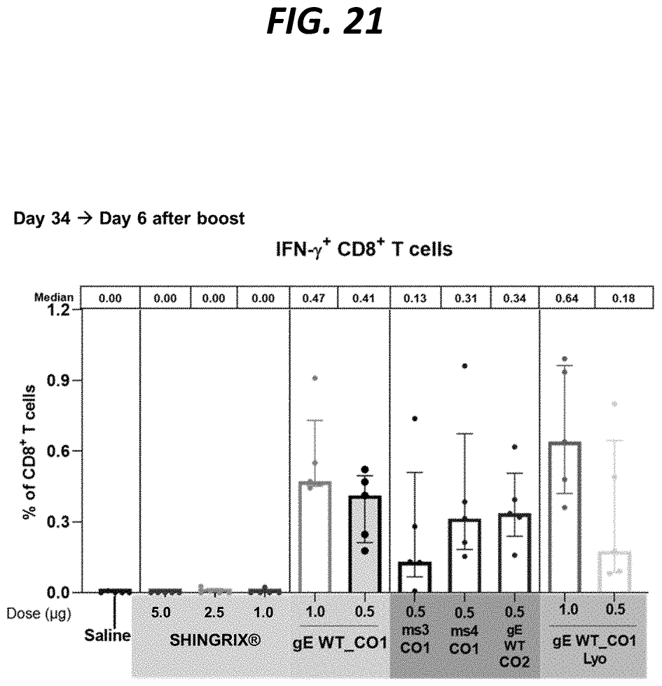

As shown in FIG. 20, examination of CD4⁺ IFN-γ⁺ (Th1) T cells revealed a strong, dose-dependent response in groups receiving VZV RNA-LNP vaccines (including lyophilized vaccine), and minimal response in mice receiving SHINGRIX®. As shown in FIG. 21, examination of CD8⁺ IFN-γ⁺ T cells revealed a strong, but variable, response in groups receiving VZV RNA-LNP vaccines, and an undetectable response in mice receiving SHINGRIX®.

### Example 6. Immune Responses—LAV-Experienced Mice (In Vivo Experiments)

VZV RNA-LNP vaccines were tested for their ability to induce IgG and T cell responses in C57BL/6 mice. 15 mice per group were immunized in accordance with the schedule and specification in Table 11. Briefly, 10 or 15 mice per group were primed subcutaneously (SQ) on Day 0 with a live-attenuated varicella (LAV) vaccine (VARIVAX®, Merck) using a full human dose per mouse (1350 pfu), immunized intramuscularly (IM) on Day 35 day and boosted on Day 63. The “infection” with a LAV vaccine mimics exposure to VZV that humans receive when infected with VZV as children and develop chickenpox.

Mice immunized with SHINGRIX® received 1, 2.5, or 5 μg, corresponding to 1/50, 1/20, and 1/10 of the human clinical dose, respectively; mice immunized with gE_WT CO2 received 0.5 or 1 μg VZV RNA-LNP; mice immunized with ms5 CO1 received 0.5 or 1 ug VZV RNA-LNP; mice immunized with ms6 CO2 received 0.5 or 1 ug VZV RNA-LNP; and mice immunized with lyophilized gE_WT CO2 received 0.5 or 1 ug VZV RNA-LNP. Saline was administered on Day 35 and 63 to a negative control group. Sera was collected from mice on Day 35, 63 and 76 for characterization of VZV gE-binding IgG levels (LUMINEX® analyses). Spleens were harvested on Day 48 (13 days post dose 1 for selected groups) and Day 76 for cellular analysis (T cell and B cell responses).

TABLE 11
Administration schedule of SHINGRIX® and VZV RNA-LNPs

| | | | | | | | | Spleen |
|---|---|---|---|---|---|---|---|---|
| | | | | | | | | collection |
| | | | Dose | | Dose Vol/ | Admin | Bleed | D48*/D76* |
| # | Mice | Vaccine | (μg) | | Route | (Day) | (Day) | (n = 5) |
| 1 | 15 | Saline | — | | 50 μl/IM | 0**, | 35, 48*, | Yes/Yes |
| | | | | | | 35, 63 | 63, 76* | |
| 2 | 15 | SHINGRIX® | 5 | μg | 50 μl/IM | 0**, | 35, 48*, | Yes/Yes |
| | | | | | | 35, 63 | 63, 76* | |
| 3 | 10 | SHINGRIX® | 2.5 | μg | 50 μl/IM | 0**, | 35, 63, | No/Yes |
| | | | | | | 35, 63 | 76* | |
| 4 | 15 | SHINGRIX® | 1 | μg | 50 μl/IM | 0**, | 35, 48*, | Yes/Yes |
| | | | | | | 35, 63 | 63, 76* | |
| 5 | 15 | gE_WT CO2 | 1 | μg | 50 μl/IM | 0** | 35, 48*, | Yes/Yes |
| | | | | | | 35, 63 | 63, 76* | |
| 6 | 15 | gE_WT CO2 | 0.5 | μg | 50 μl/IM | 0** | 35, 48*, | Yes/Yes |
| | | | | | | 35, 63 | 63, 76* | |
| 7 | 15 | ms5 CO1 | 1 | μg | 50 μl/IM | 0** | 35, 48*, | Yes/Yes |
| | | | | | | 35, 63 | 63, 76* | |
| 8 | 10 | ms5 CO1 | 0.5 | μg | 50 μl/IM | 0** | 35, 63, | No/No |
| | | | | | | 35, 63 | 76 | |
| 9 | 15 | ms6 CO2 | 1 | μg | 50 μl/IM | 0** | 35, 48*, | Yes/Yes |
| | | | | | | 35, 63 | 63, 76* | |
| 10 | 10 | ms6 CO2 | 0.5 | μg | 50 μl/IM | 0** | 35, 63, | No/No |
| | | | | | | 35, 63 | 76 | |
| 11 | 15 | gE_WT CO2 lyophilized | 1 | μg | 50 μl/IM | 0** | 35, 48*, | Yes/Yes |
| | | | | | | 35, 63 | 63,76* | |
| 12 | 10 | gE_WT CO2 lyophilized | 0.5 | μg | 50 μl/IM | 0** | 35, 63, | No/Yes |
| | | | | | | 35, 63 | 76 | |

* Spleen collection, Day 48: 8 groups, 40 mice; Day 76: 10 groups, 50 mice
^{**} VARIVAX® (LAV) administered at a full clinical dose (1350 pfu) SQ on Day 0 in a total volume of 0.5 mL

### IgG Antibodies Titers

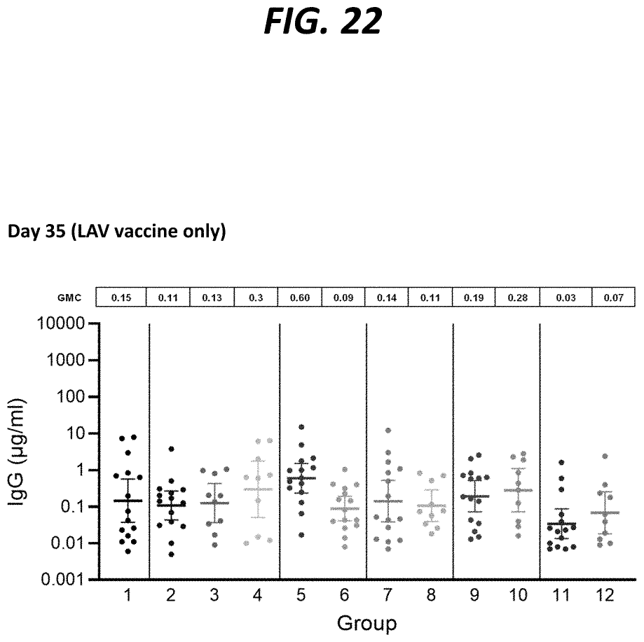

Serum IgG levels were determined for each mouse sample using the LUMINEX® platform (described herein). IgG titers for Day 35, 63 (1 month post dose 1) and 76 (13 days pose dose 2) are show in Table 12 and FIGS. 22-24. Each data point in the figures is a result from an individual animal; each horizontal lines represents the geometric mean IgG concentration (μg/ml) and whiskers represent the 95% confidence interval.

TABLE 12
Geometric Mean Titers (GMCs) on Days 35, 63 and 76

| | | | | Day 35 | Day 63 | Day 76 |
|---|---|---|---|---|---|---|
| | # | Dose | | (GMC) | (GMC) | (GMC) |
| Saline | 1 | — | | 0.15 | 0.17 | 0.17 |
| SHINGRIX ® | 2 | 5 | μg | 0.11 | 101 | 1148 |
| | 3 | 2.5 | μg | 0.13 | 115 | 1649 |
| | 4 | 1 | μg | 0.3 | 94 | 887 |
| gE_WT CO2 | 5 | 1 | μg | 0.60 | 112 | 695 |
| | 6 | 0.5 | μg | 0.09 | 33 | 316 |
| ms5 CO1 | 7 | 1 | μg | 0.14 | 76 | 530 |
| | 8 | 0.5 | μg | 0.11 | 41 | 556 |
| ms6 CO2 | 9 | 1 | μg | 0.19 | 58 | 723 |
| | 10 | 0.5 | μg | 0.28 | 50 | 464 |
| gE_WT CO2 Iyo | 11 | 1 | μg | 0.03 | 69 | 917 |
| | 12 | 0.5 | μg | 0.07 | 56 | 460 |

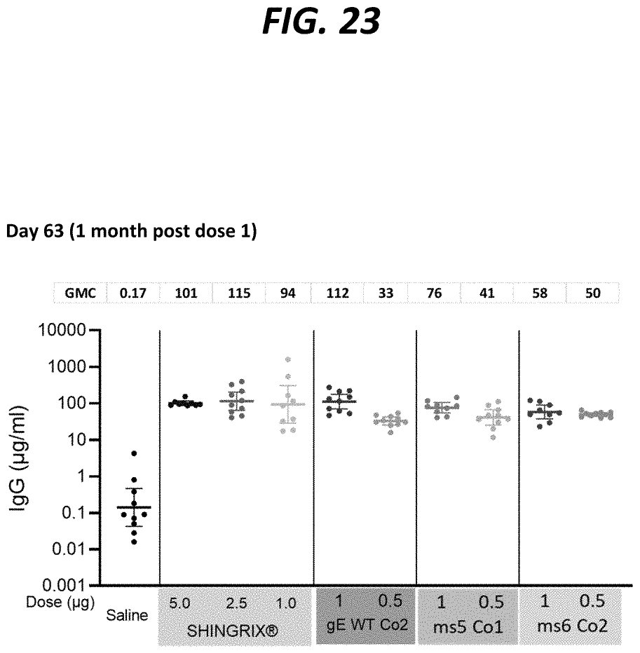

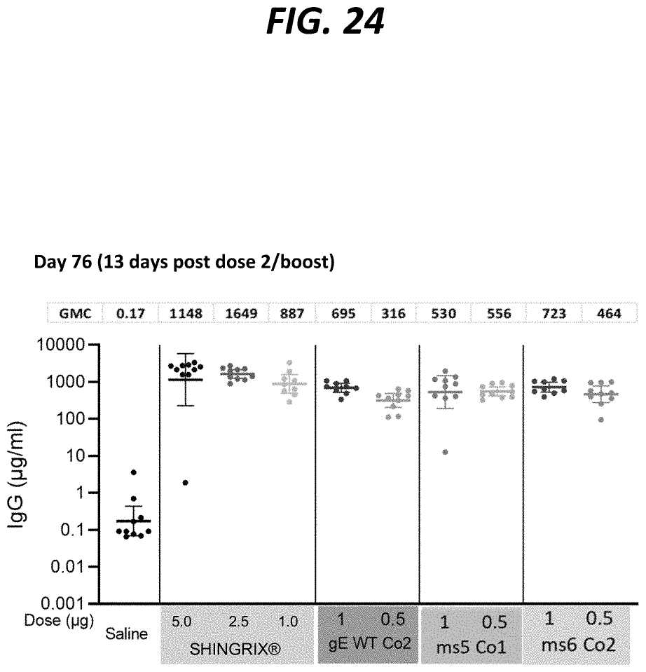

As expected prior to immunization, FIG. 22 shows low levels of IgG titers on Day 35 after SQ prime with LAV vaccine (VARIVAX®) on Day 0. As shown in FIG. 23, IgG titers significantly increased in a dose-dependent response on Day 63 (1 month post dose 1) in LAV-experienced mice receiving VZV RNA-LNP vaccines or SHINGRIX®. FIG. 24 shows a further increase in IgG titers in a dose-dependent response on Day 76 (13 days post dose 2/boost).

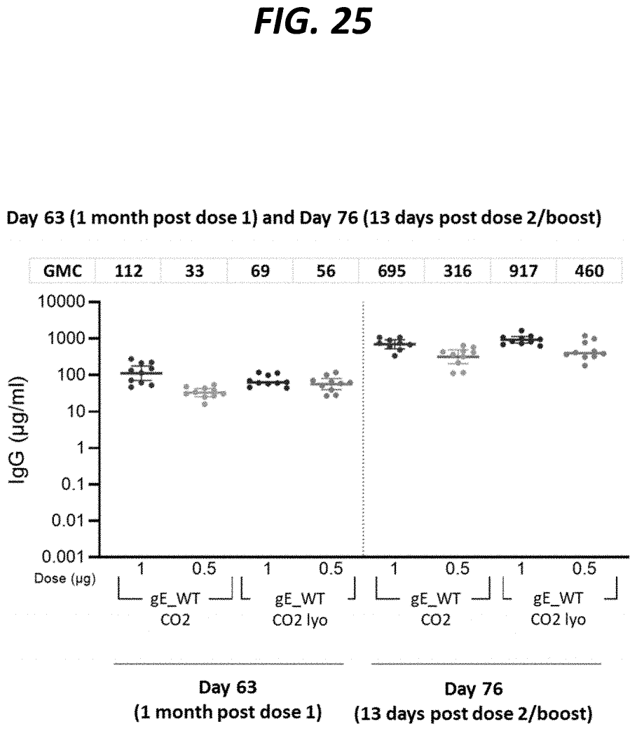

The effect of lyophilization on immunogenicity was evaluated. As shown in FIG. 25, comparable IgG titers were observed for the non-lyophilized gE_WT CO2 and lyophilized gE_WT CO2 at both Day 63 (1 month post dose 1) and Day 76 (13 days post dose 2/boost).

### Cell-Meditated Immunity (T Cell and B Cell Responses)

Vaccine-induced T cell response was measured following ex vivo stimulation of splenocytes with gE peptide pool (2 μg/mL of each peptide) by ELISpot and Intracellular Cytokine Staining (ICS) assay (described herein). For ELISpot, the cytokine IFN-γ secreted by activated T cells were captured by an anti-IFN-γ antibody coated onto the polyvinylidene fluoride (PVDF) membrane of the well bottom on a microplate. The captured IFN-γ was developed into a spot by another non-competing biotinylated anti-IFN-γ secondary antibody followed by an enzymatic color reaction using streptavidin-alkaline phosphatase (ALP) conjugate and the substrate solution, nitro-blue tetrazolium and 5-bromo-4-chloro-3′-indolyphosphate (BCIP/NBT-plus) that yielded a dark purple precipitate or spot. T cell IFN-γ response was measured using Mabtech Mouse IFN-γ ELISpot PLUS kit (ALP) and expressed as spot forming cells (SFC) per million cells. The ICS assay measured IFN-γ-expressing cells within CD4⁺ and CD8⁺ T cells expressed as percentage of IFN-γ⁺ cells within CD4⁺ and CD8⁺ T cells.

Vaccine-induced B cell response was evaluated by measuring the frequencies of VZV gE-specific B cells in the spleen. Wild type gE protein ectodomain with streptag was coupled to streptavidin (SA)-fluorochromes PE and APC, both from BioLegend in the ratio of 2:1 in separate tubes for 1 hour at room temperature (RT) at 20× of desired staining concentration (10 μg/mL for each protein). Each of the SA-fluorochrome-coupled spike proteins were pooled and diluted to 1× using flow cytometry (FC) buffer (2% FBS/PBS) to generate B-cell probes to identify gE-specific B cells. Single cell suspensions of splenocytes (5×10⁶ cells per well) were first washed in PBS and stained with eFluor 506 Fixable Viability dye for 10 minutes at RT to identify live from dead cells. Following washes in FC buffer, cells were incubated with 50 μL/well of 1×B-cell probe for 30 to 45 minutes and then washed to remove unbound probes. Cells were surface stained with a cocktail of flow cytometry antibodies (CD19, IgD, IgM, IgD, All from BioLegend) to identify B cell surface phenotypes. Following washing, cells were fixed using BD Fixation buffer and suspended in FC buffer. Cells were acquired on LSR Fortessa and data analyzed by FlowJo (10.7.1). The results of gE-specific B cells were expressed as the percentage of IgG-expressing B cells.

### Day 48

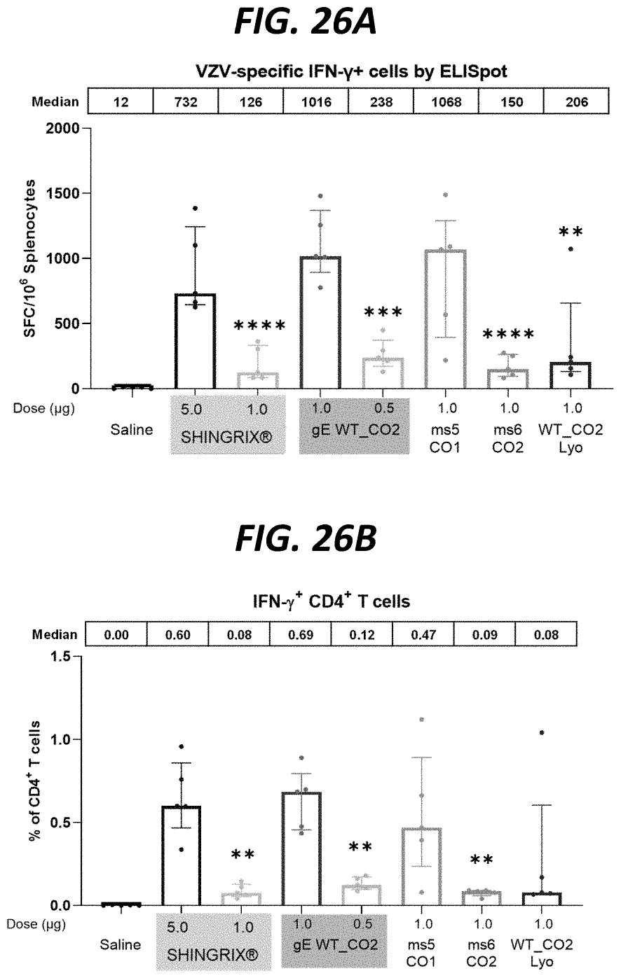

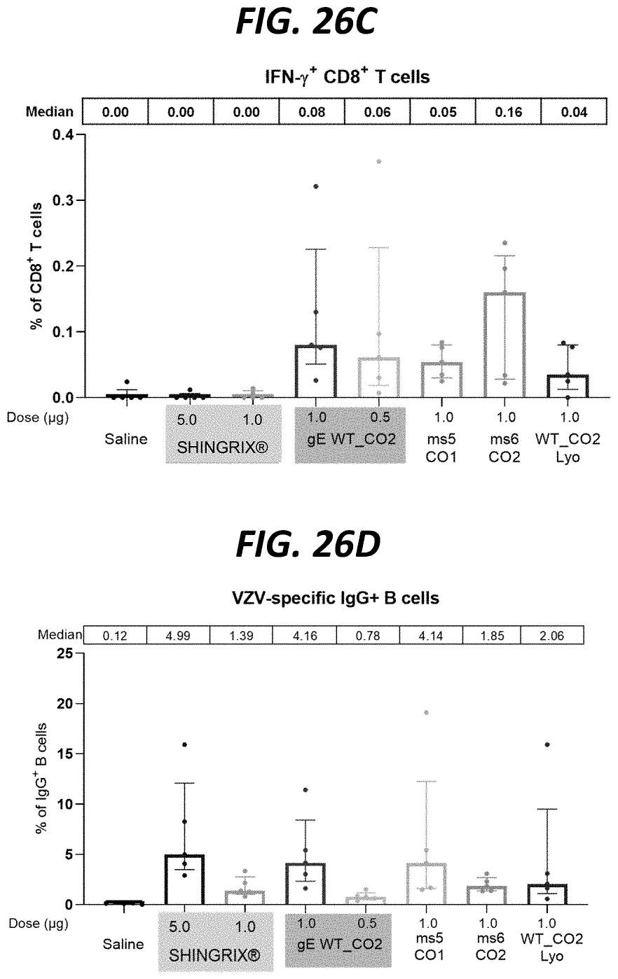

Splenocytes were harvested from C57BL/6 mice on Day 48 (13 days post dose 1) to assess gE-specific cellular immune responses induced after a single vaccine dose, see Table 13 and FIGS. 26A-26D. LAV-experienced mice were immunized IM at Day 35 with SHINGRIX® or VZV RNA-LNP vaccine, and spleens collected on Day 48 (13 days post dose 1).

TABLE 13
Cellular immune responses on Day 48 (13 days post dose 1)

| | | | gE-specific | IFN-γ⁺ CD4⁺ | IFN-γ⁺ CD8⁺ | gE-specific |
|---|---|---|---|---|---|---|
| | Dose | | IFN-γ⁺cells | T cell | T cell | IgG⁺ B cells |
| Saline | — | | 12 | 0.00 | 0.00 | 0.12 |
| SHINGRIX ® | 5 | μg | 732 | 0.60 | 0.00 | 4.99 |
| | 1 | μg | 126 | 0.08 | 0.00 | 1.39 |
| gE_WT CO2 | 1 | μg | 1016 | 0.69 | 0.08 | 4.16 |
| | 0.5 | μg | 238 | 0.12 | 0.06 | 0.78 |
| ms5 CO1 | 1 | μg | 1068 | 0.47 | 0.05 | 4.14 |
| ms6 CO2 | 1 | μg | 150 | 0.09 | 0.16 | 1.85 |
| gE_WT CO2 Iyo | 1 | μg | 206 | 0.08 | 0.04 | 2.06 |

FIG. 26A shows LAV-experienced mice elicited a strong gE-specific IFN-γ (T-cell) response in a dose-dependent manner, as measured by IFN-γ ELISpot, after a single vaccine dose. As shown in FIG. 26B, ICS assay results revealed a similar strong, dose-dependent gE-specific IFN-γ⁺ CD4⁺ T cell response induced by the vaccines as demonstrated by ELISpot. FIG. 26C shows a gE-specific IFN-γ⁺ CD8⁺ T cell response was only induced by VZV RNA-LNP vaccines (gE_WT CO2, ms5 CO1, ms6 CO2, gE_WT CCO2 lyo) but not SHINGRIX®, demonstrating the unique immune response induced by VZV-RNA-LNP vaccines. Overall, at Day 48 (13 days post dose 1), the T cell response induced by gE_WT CO2 at 1 μg (1/60 of a potential clinical dose of 60 μg) was significantly higher than the T cell response induced by SHINGRIX® at 1 μg (1/50 of its human dose).

B cell response was evaluated in splenocytes by measuring the frequency of gE-specific IgG⁺ B cells by flow cytometry. As shown in FIG. 26D, the frequency of gE-specific IgG⁺ B cells revealed that the B cell response induced by the vaccines is similar to the gE-specific IFN-γ⁺ CD4⁺ T-cell response.

### Day 76

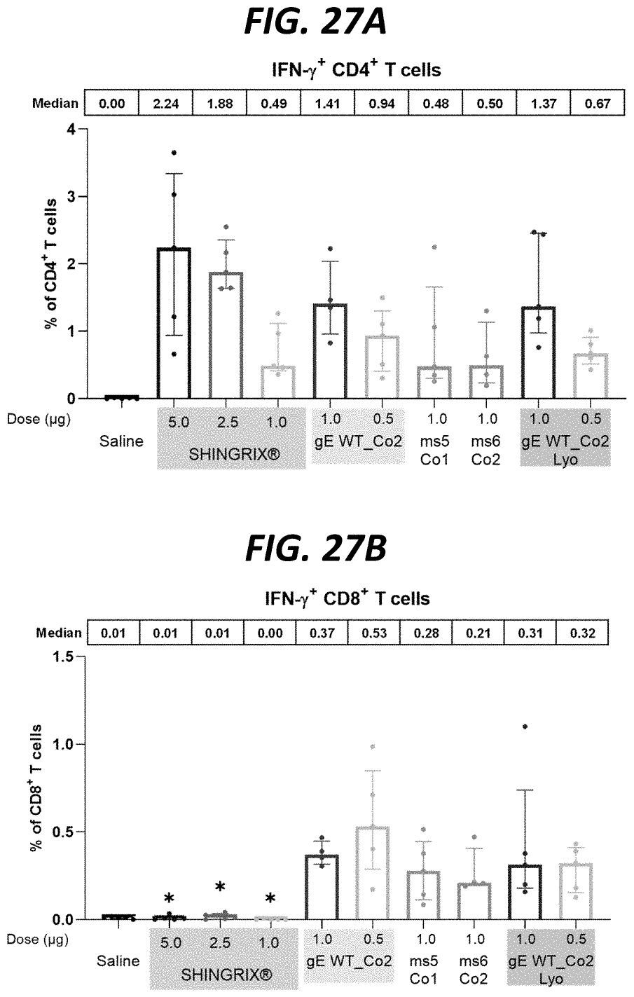

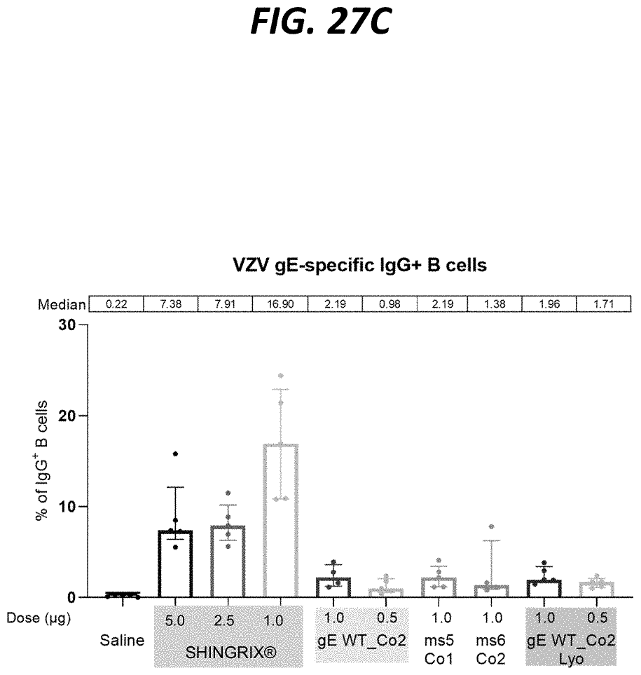

Splenocytes were harvested from C57BL/6 mice on Day 76 (13 days post dose 2/boost) to assess gE-specific cellular immune responses induced after a second/boost vaccine dose, see Table 14 and FIGS. 27A-27C. LAV-experienced mice were immunized IM at Day 35 and day 63 with SHINGRIX® or VZV RNA-LNP vaccine, and spleens collected on day 76 (13 days dost post 2/boost).

TABLE 14
Cellular immune responses on Day 76 (13 days post dose 2/boost)

| | | | IFN-γ⁺ CD4⁺ | IFN-γ⁺ CD8⁺ | gE-specific |
|---|---|---|---|---|---|
| | Dose | | T cell | T cell | IgG⁺ B cells |
| Saline | — | | 0.00 | 0.01 | 0.22 |
| SHINGRIX ® | 5 | μg | 2.24 | 0.01 | 7.38 |
| | 2.5 | μg | 1.88 | 0.01 | 7.91 |
| | 1 | μg | 0.49 | 0.00 | 16.90 |
| gE_WT CO2 | 1 | μg | 1.41 | 0.37 | 2.19 |
| | 0.5 | μg | 0.94 | 0.53 | 0.98 |
| ms5 CO1 | 1 | μg | 0.48 | 0.28 | 2.19 |
| ms6 CO2 | 1 | μg | 0.50 | 0.21 | 1.38 |
| gE_WT CO2 Iyo | 1 | μg | 0.08 | 0.31 | 1.96 |
| | 0.5 | μg | 0.67 | 0.32 | 1.71 |

FIG. 27A shows the second/boost vaccine dose significantly increased the gE-specific CD4⁺ T cell response in a dose-dependent manner, as measured by ICS assay. As shown in FIG. 27B, a robust increase in gE-specific IFN-γ⁺ CD8⁺ T-cell response was only induced by VZV RNA-LNP vaccines (gE_WT CO2, ms5 CO1, ms6 CO2, gE_WT 0002 lyo) but not SHINGRIX®, confirming the unique immune response induced by VZV-RNA-LNP vaccines. Overall, at Day 76 (13 days post dose 2/boost), the T cell response induced by gE_WT CO2 at 1 μg (1/60 of a potential clinical dose of 60 μg) was higher than the T cell response induced by SHINGRIX® at 1 μg (1/50 of its human dose).

B cell response was evaluated in splenocytes by measuring the frequency of gE-specific IgG⁺ B cells by flow cytometry and are shown in FIG. 270.

### Example 7. VZV Antigens

Sequences of the VZV anti gens/polypeptides, VZV DNA and VZV RNA of the present invention are provided in Tables 1 to 3. The sequences may comprise any stop codon, including but not limited to the stop codons provided in the Tables.

Lengthy table referenced here
US20230233671A1-20230727-T00001
Please refer to the end of the specification for access instructions.

Lengthy table referenced here
US20230233671A1-20230727-T00002
Please refer to the end of the specification for access instructions.

Lengthy table referenced here
US20230233671A1-20230727-T00003
Please refer to the end of the specification for access instructions.

The following paragraphs describe additional aspects of the disclosure:
1. An RNA molecule comprising at least one open reading frame encoding a varicella-zoster virus (VZV) polypeptide.
2. The RNA molecule of paragraph 1, wherein the VZV polypeptide is a VZV glycoprotein.
3. The RNA molecule of paragraph 2, wherein the VZV glycoprotein is selected from VZV gK, gN, gC, gB, gH, gM, gL gI and gE.
4. The RNA molecule of paragraph 3, wherein the VZV glycoprotein is VZV gE.
5. The RNA molecule of any one of paragraphs 1 to 4, wherein the VZV polypeptide is a full-length, truncated, fragment or variant thereof.
6. The RNA molecule of any one of paragraphs 1 to 5, wherein the VZV polypeptide comprises at least one mutation.
7. The RNA molecule of any one of paragraphs 1 to 6, wherein the VZV polypeptide comprises an amino acid of Table 1, including but not limited to any of SEQ ID NO: 1 to 11.
8. The RNA molecule of any one of paragraphs 1 to 7, wherein the VZV polypeptide has at least 90%, 95, 96%, 97%, 98% or 99% identity to the amino acid sequence selected from SEQ ID NO: 1 to 11.
9. The RNA molecule of any one of paragraphs 1 to 8, wherein the VZV polypeptide comprises an amino acid sequence selected from SEQ ID NO: 1 to 11.
10. The RNA molecule of any one of paragraphs 1 to 9, wherein the open reading frame is transcribed from a nucleic acid sequence of Table 2, including by not limited to any of SEQ ID NO: 12 to 145.
11. The RNA molecule of any one of paragraphs 1 to 10, wherein the open reading frame is transcribed from a nucleic acid sequence having at least 90%, 95, 96%, 97%, 98% or 99% identity to the sequence of any of SEQ ID NO: 12 to 145.
12. The RNA molecule of any one of paragraphs 1 to 11, wherein the open reading frame comprises a nucleic acid sequence of Table 3, including but not limited to any of SEQ ID NO: 146 to 279.
13. The RNA molecule of any one of paragraphs 1 to 12, wherein the open reading frame comprises a nucleic acid sequence having at least 90%, 95, 96%, 97%, 98% or 99% identity to the sequence of any of SEQ ID NO: 146 to 279.
14. The RNA molecule of any one of paragraphs 1 to 13, wherein the open reading frame comprises a nucleic acid sequence of SEQ ID NO: 146 (gE WT).
15. The RNA molecule of any one of paragraphs 1 to 14, wherein the open reading frame comprises a nucleic acid sequence of SEQ ID NO: 147 (gE WT CO1).
16. The RNA molecule of any one of paragraphs 1 to 15, wherein the open reading frame comprises a nucleic acid sequence of SEQ ID NO: 148 (gE WT CO2).
17. The RNA molecule of any one of paragraphs 1 to 16, wherein the open reading frame comprises a nucleic acid sequence of SEQ ID NO: 149 (ms3 CO1).
18. The RNA molecule of any one of paragraphs 1 to 17, wherein the open reading frame comprises a nucleic acid sequence of SEQ ID NO: 150 (ms3 CO2).
19. The RNA molecule of any one of paragraphs 1 to 18, wherein the open reading frame comprises a nucleic acid sequence of SEQ ID NO: 151 (ms4 CO1).
20. The RNA molecule of any one of paragraphs 1 to 19, wherein the open reading frame comprises a nucleic acid sequence of SEQ ID NO: 152 (ms4 CO2).
21. The RNA molecule of any one of paragraphs 1 to 20, wherein the open reading frame comprises a nucleic acid sequence of SEQ ID NO: 153 (ms5 CO1).
22. The RNA molecule of any one of paragraphs 1 to 21, wherein the open reading frame comprises a nucleic acid sequence of SEQ ID NO: 154 (ms5 CO2).
23. The RNA molecule of any one of paragraphs 1 to 22, wherein the open reading frame comprises a nucleic acid sequence of SEQ ID NO: 155 (ms5 CO2 v2).
24. The RNA molecule of any one of paragraphs 1 to 23, wherein the open reading frame comprises a nucleic acid sequence of SEQ ID NO: 156 (ms6 CO1).
25. The RNA molecule of any one of paragraphs 1 to 24, wherein the open reading frame comprises a nucleic acid sequence of SEQ ID NO: 157 (ms6 CO2).
26. The RNA molecule of any one of paragraphs 1 to 25, wherein the open reading frame comprises a nucleic acid sequence of SEQ ID NO: 158 (ms8 CO1).
27. The RNA molecule of any one of paragraphs 1 to 26, wherein the open reading frame comprises a nucleic acid sequence of SEQ ID NO: 159 (ms9 CO1).
28. The RNA molecule of any one of paragraphs 1 to 27, wherein the open reading frame comprises a nucleic acid sequence of SEQ ID NO: 160 (ms9 CO2).
29. The RNA molecule of any one of paragraphs 1 to 28, wherein the open reading frame comprises a nucleic acid sequence of SEQ ID NO: 161 (ms10 CO1).
30. The RNA molecule of any one of paragraphs 1 to 29, wherein the open reading frame comprises a nucleic acid sequence of SEQ ID NO: 162 (ms10 CO2).
31. The RNA molecule of any one of paragraphs 1 to 30, wherein the open reading frame comprises a nucleic acid sequence of SEQ ID NO: 163 (ms10 CO3).
32. The RNA molecule of any one of paragraphs 1 to 31, wherein the open reading frame comprises a nucleic acid sequence of SEQ ID NO: 164 (ms11 CO1).
33. The RNA molecule of any one of paragraphs 1 to 32, wherein the open reading frame comprises a nucleic acid sequence of SEQ ID NO: 165 (ms11 CO2).
34. The RNA molecule of any one of paragraphs 1 to 33, wherein the open reading frame comprises a nucleic acid sequence of SEQ ID NO: 166 (ms12 CO1).
35. The RNA molecule of any one of paragraphs 1 to 34, wherein the open reading frame comprises a nucleic acid sequence of SEQ ID NO: 167 (ms12 CO2).
36. The RNA molecule of any one of paragraphs 1 to 35, wherein each uridine is replaced by N1-methylpseudouridine (ψ).
37. The RNA molecule of any one of paragraphs 1 to 36, further comprising a 5′ untranslated region (5′ UTR).
38. The RNA molecule of paragraph 37, wherein the 5′ UTR comprises a sequence selected from any of SEQ ID NO: 281 and 312 to 313.
39. The RNA molecule any one of paragraphs 1 to 38, further comprising a 3′ untranslated region (3′ UTR).
40. The composition of paragraph 39, wherein the 3′ UTR comprises a sequence selected from any of SEQ ID NO: 284, 314 and 317.
41. The RNA molecule of any one of paragraphs 1 to 40, wherein the RNA molecule comprises a 5′ cap moiety.
42. The RNA molecule of paragraph 41, wherein the RNA molecule comprises a 5′ cap moiety comprising (3′OMe)-m₂^{7,3-O}Gppp (m₁^{2′-O})ApG.
43. The RNA molecule of any one of paragraphs 1 to 42, further comprising a 3′ poly-A tail.
44. The RNA molecule of paragraph 43, wherein the poly-A tail comprises a sequence selected from any of SEQ ID NO: 287 and 315 comprising +/−1 or +/−2 adenosine (A).
45. The RNA molecule of paragraph 43, wherein the poly-A tail comprises a sequence of SEQ ID NO: 287 comprising +/−1 or +/−2 adenosine (A).
46. The RNA molecule of paragraph 43, wherein the poly-A tail comprises a sequence of SEQ ID NO: 315 comprising +/−1 or +/−2 adenosine (A).
47. The RNA molecule of any one of paragraphs 1 to 46, wherein the RNA molecule comprises a 5′ UTR and 3′ UTR.
48. The RNA molecule of any one of paragraphs 1 to 47, wherein the RNA molecule comprises a 5′ cap, 5′ UTR, and 3′ UTR.
49. The RNA molecule of any one of paragraphs 1 to 48, wherein the RNA molecule comprises a 5′ cap, 5′ UTR, 3′ UTR, and poly-A tail.
50. The RNA molecule of any of paragraphs 1 to 49, comprising a 5′ UTR comprising the sequence of SEQ ID NO: 281 or 312, an open reading frame comprising the sequence of any of SEQ ID NO: 146 to 279 and a 3′ UTR comprising the sequence of SEQ ID NO: 284 or 317.
51. The RNA molecule of any of paragraphs 1 to 50, comprising a 5′ UTR comprising the sequence of any of SEQ ID NO: 28, 312 and 313, an open reading frame comprising the sequence of any of SEQ ID NO: 146 to 279, a 3′ UTR comprising the sequence of any of SEQ ID NO: 284, 314 and 317, and a poly-A tail comprising a sequences of any of SEQ ID NO: 287 and 315.
52. The RNA molecule of any one of paragraphs 1 to 51, wherein the open reading frame was generated from codon-optimized DNA.
53. The RNA molecule of any one of paragraphs 1 to 52, wherein the open reading frame comprises a G/C content of at least 55%, at least 60%, at least 65%, at least 70%, at least 75%, about 50% to 75%, about 55% to 70%, about 58%, about 66% or about 62%.
54. The RNA molecule of any one of paragraphs 1 to 53, wherein the encoded VZV polypeptide localizes in the cellular membrane, localizes in the Golgi and/or is anchored in the membrane and is secreted.
55. The RNA molecule of any of paragraphs 1 to 54, wherein the RNA molecule comprises stabilized RNA.
56. The RNA molecule of any one of paragraphs 1 to 55, wherein the RNA comprises at least one modified nucleotide.
57. The RNA molecule of paragraph 56, wherein the modified nucleotide is pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine or 2′-O-methyl uridine.
58. The RNA molecule of paragraph 56 or 57, wherein the modified nucleotide is N1-methylpseudouridine (ψ).
59. The RNA molecule of any one of paragraphs 1 to 58, wherein each uridine is replaced by N1-methylpseudouridine (ψ).
60. The RNA molecule of any one of paragraphs 1 to 59, wherein the RNA is mRNA or self-replicating RNA.
61. The RNA molecule of paragraph 60, wherein the RNA is a mRNA.
62. A composition comprising the RNA molecule of any one of paragraphs 1 to 61, wherein the RNA molecule is formulated in a lipid nanoparticle (LNP).
63. The composition of paragraph 62, wherein the lipid nanoparticle comprises at least one of a cationic lipid, a PEGylated lipid, and at least a first and second structural lipid.
64. The composition of paragraph 62 or 63, wherein the lipid nanoparticle comprises a cationic lipid.
65. The composition of paragraph 64, wherein the cationic lipid is (4-hydroxybutyl)azanediyl)bis(hexane-6,1-diyl)bis(2-hexyldecanoate) (ALC-0315).
66. The composition of any one of paragraphs 61 to 64, wherein the lipid nanoparticle comprises a PEGylated lipid.
67. The composition of any one of paragraphs 63 to 66, wherein the PEGylated lipid is PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols, 2-[(polyethylene glycol)-2000]-N,N-ditetradecylacetamide, glycol-lipids including PEG-c-DOMG, PEG-c-DMA, PEG-s-DMG,N-[(methoxy polyethylene glycol)2000)carbamyl]-1,2-dimyristyloxlpropyl-3-amine (PEG-c-DMA), and PEG-2000-DMG, PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-((o-methoxy(polyethoxy)ethyl)butanedioate (PEG-S-DMG), a PEGylated ceramide (PEG-cer), or a PEG dialkoxypropylcarbamate such as co-methoxy(polyethoxy)ethyl-N-(2,3di(tetradecanoxy)propyl)carbamate or 2,3-di(tetradecanoxy)propyl-N-(u>-methoxy(polyethoxy)ethyl)carbamate.
68. The composition of any one of paragraphs 63 to 67, wherein the PEGylated lipid is 2-[(polyethylene glycol)-2000]-N,N-ditetradecylacetamide (ALC-0159).
69. The composition of any one of paragraphs 63 to 68, wherein the first structural lipid is a neutral lipid.
70. The composition of paragraph 69, wherein the neutral lipid is 1,2-distearoyl-sn-glycero-3-phosphocholine (DSPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1 carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), or 1,2-dielaidoyl-sn-glycero-3-phophoethanolamine (transDOPE).
71. The composition of paragraph 69 or 70, wherein the neutral lipid is 1,2-distearoyl-sn-glycero-3-phosphocholine (DSPC).
72. The composition of any one of paragraphs 63 to 71, wherein the second structural lipid is a steroid or steroid analog.
73. The composition of paragraph 72, wherein the steroid or steroid analog is cholesterol.
74. The composition of any one of paragraphs 62 to 73, wherein lipid nanoparticle has a mean diameter of about 1 to about 500 nm.
75. The composition of any one of paragraphs 62 to 74, comprising an RNA molecule at a concentration of about 0.01 to 0.09 mg/mL formulated in a lipid nanoparticle (LNP) comprising a cationic lipid at a concentration of about 0.8 to 0.95 mg/mL, a PEGylated lipid at a concentration of about 0.05 to 0.15 mg/mL, a neutral lipid at a concentration of about 0.1 to 0.25 mg/mL and a steroid or steroid analog at a concentration of about 0.3 to 0.45 mg/mL.
76. The composition of any one of paragraphs 62 to 75, comprising an RNA molecule at a concentration of about 0.01 to 0.09 mg/mL formulated in a lipid nanoparticle (LNP) comprising (4-hydroxybutyl)azanediyl)bis(hexane-6,1-diyl)bis(2-hexyldecanoate) (ALC-0315) at a concentration of about 0.8 to 0.95 mg/mL, 2-[(polyethylene glycol)-2000]-N,N-ditetradecylacetamide (ALC-0159) at a concentration of about 0.05 to 0.15 mg/mL, 1,2-distearoyl-sn-glycero-3-phosphocholine (DSPC) at a concentration of about 0.1 to 0.25 mg/mL and cholesterol at a concentration of about 0.3 to 0.45 mg/mL.
77. The composition of any one of paragraphs 62 to 76, comprising an RNA molecule at a concentration of about 0.06 mg/mL formulated in a lipid nanoparticle (LNP).
78. The composition of any one of paragraphs 62 to 77, further comprising at least one of a buffer, a stabilizing agent, salt, surfactant, preservative, excipient, or adjuvant.
79. The composition of any one of paragraphs 62 to 78, further comprising at least a buffer and a stabilizing agent, and optionally, a salt diluent.
80. The composition of paragraph 79 or 79, wherein the buffer is a Tris buffer.
81. The composition of paragraph 80, wherein the Tris buffer comprises tromethamine and Tris hydrochloride (HCl).
82. The composition of paragraph 81, wherein the tromethamine is at a concentration of about 0.1 to 0.3 mg/mL or about 0.01 to 0.15 mg/mL.
83. The composition of paragraph 81 or 82, wherein and the Tris HCl is at a concentration of about 1.25 to 1.40 mg/mL or about 0.5 to 0.65 mg/mL.
84. The composition of any one of paragraphs 78 to 83, wherein the stabilizing agent is sucrose.
85. The composition of paragraph 84, wherein the sucrose is at a concentration of about 95 to 110 mg/mL or about 35 to 50 mg/mL.
86. The composition of any one of paragraphs 78 to 85, wherein the salt diluent for reconstitution is sodium chloride.
87. The composition of paragraph 86, wherein the sodium chloride is at a concentration of about 5 to 15 mg/mL.
88. The composition of any one of paragraphs 62 to 87, wherein the composition is a liquid or lyophilized.
89. The composition of paragraph 62, comprising an RNA molecule at a concentration of about 0.01 to 0.09 mg/mL formulated in a lipid nanoparticle (LNP) comprising a cationic lipid at a concentration of about 0.8 to 0.95 mg/mL, a PEGylated lipid at a concentration of about 0.05 to 0.15 mg/mL, a neutral lipid at a concentration of about 0.1 to 0.25 mg/mL and a steroid or steroid analog at a concentration of about 0.3 to 0.45 mg/mL, and further comprising a Tris buffer comprising tromethamine at a concentration of about 0.1 to 0.3 mg/mL and Tris hydrochloride (HCl) at a concentration of about 1.25 to 1.40 mg/mL, and sucrose at a concentration of about 95 to 110 mg/mL, wherein the composition is a liquid composition.
90. The composition of paragraph 62, comprising an RNA molecule at a concentration of about 0.01 to 0.09 mg/mL formulated in a lipid nanoparticle (LNP) comprising a cationic lipid at a concentration of about 0.8 to 0.95 mg/mL, a PEGylated lipid at a concentration of about 0.05 to 0.15 mg/mL, a neutral lipid at a concentration of about 0.1 to 0.25 mg/mL and a steroid or steroid analog at a concentration of about 0.3 to 0.45 mg/mL, and further comprising a Tris buffer comprising tromethamine at a concentration of about 0.01 to 0.15 mg/mL and Tris hydrochloride (HCl) at a concentration of about 0.5 to 0.65 mg/mL, sucrose at a concentration of about 35 to 50 mg/mL.
91. The composition of paragraph 90, wherein the composition is reconstituted with sodium chloride at a concentration of about 5 to 15 mg/mL.
92. The composition of paragraph 91, wherein the composition is reconstituted with about 0.6 to 0.75 mL sodium chloride.
93. The composition of paragraph 62, further comprising about 5 to 15 mM Tris buffer, 200 to 400 mM sucrose at a pH of about 7.0 to 8.0, and optionally, 0.9% sodium chloride diluent to reconstitute.
94. A method of inducing an immune response against VZV in a subject, comprising administering to the subject an effective amount of the RNA molecule and/or composition of any one of paragraphs 1 to 93.
95. A method of preventing, treating or ameliorating an infection, disease or condition in a subject, comprising administering to a subject an effective amount of the RNA molecule and/or composition of any one of paragraphs 1 to 93.
96. The method of paragraph 95, wherein the infection, disease or condition is associated with VZV.
97. The method of paragraph 95 or 96, wherein the infection, disease or condition is herpes zoster (shingles).
98. The method of paragraph 95 or 96, wherein the infection, disease or condition is postherpetic neuralgia.
99. Use of the RNA molecule and/or composition of any one of paragraphs 1 to 93 in the manufacture of a medicament for use in inducing an immune response against VZV in a subject.
100. Use of the RNA molecule or composition of any one of paragraphs 1 to 93 in the manufacture of a medicament for use in preventing, treating or ameliorating an infection, disease or condition in a subject.
101. The use of paragraph 100, wherein the infection, disease or condition is associated with VZV.
102. The use of paragraph 100 or 101, wherein the infection, disease or condition is herpes zoster (shingles).
103. The use of paragraph 100 or 101, wherein the infection, disease or condition is postherpetic neuralgia.
104. The method or use of any one of paragraphs 94 to 103, wherein the subject is less than about 1 year of age, about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older.
105. The method or use of any one of paragraphs 94 to 104, wherein the subject the subject is about 50 years of age or older.
106. The method or use of any one of paragraphs 94 to 105, wherein the subject is immunocompetent.
107. The method or use of any one of paragraphs 94 to 105, wherein the subject is immunocompromised.
108. The method or use of any one of paragraphs 94 to 107, wherein the RNA molecule and/or composition is administered as a vaccine.
109. The method or use of any one of paragraphs 94 to 108, wherein the RNA molecule and/or composition is administered by intradermal or intramuscular injection.
110. The method or use of any one of paragraphs 94 to 109, wherein the subject is administered a single dose, two doses, three doses or more doses of the RNA molecule and/or composition.
111. The method or use of any one of paragraphs 94 to 110, wherein the subject is administered a single dose of the RNA molecule and/or composition.
112. The method or use of any one of paragraphs 94 to 110, wherein the subject is administered two doses of the RNA molecule and/or composition.
113. The method or use of any one of paragraphs 94 to 110, wherein the subject is administered two doses of the RNA molecule and/or composition on Day 0 and about 2 months later.
114. The method or use of any one of paragraphs 94 to 110, wherein the subject is administered two doses of the RNA molecule and/or composition on Day 0 and about 6 months later.
115. The method or use of any one of paragraphs 94 to 114, wherein the subject is administered at least one booster dose of the RNA molecule and/or composition.
116. The method or use of any one of paragraphs 94 to 115, wherein the subject is administered a dose of at least about 15 μg, at least about 30 μg, at least about 60 μg, at least about 90 μg, at least about 100 μg or higher RNA molecule and/or composition per administration.
117. The method or use of any one of paragraphs 94 to 116, wherein the subject is administered an injection with a volume of about 0.25 to 1 mL, including but not limited to, about 0.25, 0.5, 1 mL.

All of the methods disclosed and claimed herein can be made and executed without undue experimentation in light of the present disclosure. While the compositions and methods of this disclosure have been described in terms of certain aspects, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

The contents of all cited references (including literature references, issued patents, published patent applications, and GENBANK® Accession numbers as cited throughout this application) recited in the application, to the extent that they provide exemplary procedural or other details supplementary to those set forth herein, are hereby specifically and expressly incorporated by reference. When definitions of terms in documents that are incorporated by reference herein conflict with those used herein, the definitions used herein govern

LENGTHY TABLES
The patent application contains a lengthy table section. A copy of the table is available in electronic form from the USPTO web site (https://seqdata.uspto.gov/?pageRequest=docDetail&DocID=US20230233671A1). An electronic copy of the table will also be available from the USPTO upon request and payment of the fee set forth in 37 CFR 1.19(b)(3).

What is claimed is:

1. An RNA molecule comprising at least one open reading frame encoding a varicella-zoster virus (VZV) glycoprotein E (gE) polypeptide.

2. The RNA molecule of claim 1, wherein the VZV polypeptide is a full-length, truncated, fragment or variant thereof.

3. The RNA molecule of claim 1, wherein the VZV polypeptide comprises at least one mutation.

4. The RNA molecule of claim 1, wherein the VZV polypeptide has at least 90%, 95, 96%, 97%, 98% or 99% identity to any one of the amino acid sequences selected from SEQ ID NO: 1 to 11.

5. The RNA molecule of claim 1, wherein the open reading frame is transcribed from a nucleic acid sequence having at least 90%, 95, 96%, 97%, 98% or 99% identity to any one of the sequences selected from SEQ ID NO: 12 to 145.

6. The RNA molecule of claim 1, wherein the open reading frame comprises a nucleic acid sequence having at least 90%, 95, 96%, 97%, 98% or 99% identity to any one of the sequences selected from SEQ ID NO: 146 to 279.

7. The RNA molecule of claim 1, wherein the open reading frame comprises a nucleic acid sequence selected from any one of SEQ ID NO: 146 to 279.

8. The RNA molecule of claim 1, further comprising a 5′ untranslated region (5′ UTR).

9. The RNA molecule of claim 8, wherein the 5′ UTR comprises a sequence selected from any one of SEQ ID NO: 281, 312 or 313.

10. The RNA molecule of claim 1, further comprising a 3′ untranslated region (3′ UTR).

11. The RNA molecule of claim 10, wherein the 3′ UTR comprises the sequence selected from any one of SEQ ID NO: 284, 314 or 317.

12. The RNA molecule of claim 1, wherein the RNA molecule further comprises a 5′ cap moiety and/or a 3′ poly-A tail.

13. The RNA molecule of claim 12, wherein the poly-A tail comprises a sequence selected from any one of SEQ ID NO: 287 or 315.

14. The RNA molecule of claim 1, wherein the open reading frame comprises a G/C content of at least 55%, at least 60%, at least 65%, at least 70%, at least 75%, about 50% to 75%, or about 55% to 70%.

15. The RNA molecule of claim 1, wherein the encoded VZV polypeptide localizes in the cellular membrane, localizes in the Golgi and/or is secreted.

16. The RNA molecule of claim 1, wherein the RNA comprises at least one modified nucleotide.

17. The RNA molecule of claim 1, wherein each uridine is replaced by N1-methylpseudouridine (ψ).

18. The RNA molecule of claim 1, wherein the RNA is a mRNA.

19. A composition comprising the RNA molecule of claim 1, wherein the RNA molecule is formulated in a lipid nanoparticle (LNP).

20. The composition of claim 19, wherein the lipid nanoparticle comprises at least one of a cationic lipid, a PEGylated lipid, a neutral lipid, and a steroid or steroid analog.

21. The composition of claim 20, wherein the cationic lipid is (4-hydroxybutyl)azanediyl)bis(hexane-6,1-diyl)bis(2-hexyldecanoate) (ALC-0315).

22. The composition of claim 20, wherein the PEGylated lipid is PEG-modified phosphatidylethanolamine, PEG-modified phosphatidic acid, PEG-modified ceramides (e.g. PEG-CerC14 or PEG-CerC20), PEG-modified dialkylamines, PEG-modified diacylglycerols, PEG-modified dialkylglycerols, 2-[(polyethylene glycol)-2000]-N,N-ditetradecylacetamide (ALC-0159), glycol-lipids including PEG-c-DOMG, PEG-c-DMA, PEG-s-DMG,N-[(methoxy polyethylene glycol)2000)carbamyl]-1,2-dimyristyloxlpropyl-3-amine (PEG-c-DMA), and PEG-2000-DMG, PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-((o-methoxy(polyethoxy)ethyl)butanedioate (PEG-S-DMG), a PEGylated ceramide (PEG-cer), or a PEG dialkoxypropylcarbamate such as co-methoxy(polyethoxy)ethyl-N-(2,3di(tetradecanoxy)propyl)carbamate or 2,3-di(tetradecanoxy)propyl-N-(u>-methoxy(polyethoxy)ethyl)carbamate.

23. The composition of claim 20, wherein the neutral lipid is 1,2-distearoyl-sn-glycero-3-phosphocholine (DSPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1 carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), or 1,2-dielaidoyl-sn-glycero-3-phophoethanolamine (transDOPE).

24. The composition of claim 20, wherein the steroid or steroid analog is cholesterol.

25. A method of inducing an immune response against VZV in a subject, comprising administering to the subject an effective amount of the RNA molecule of claim 1.

26. A method of preventing, treating or ameliorating an infection, disease or condition associated with VZV in a subject, comprising administering to a subject an effective amount of the RNA molecule of claim 1.

27. The method of claim 26, wherein the infection, disease or condition is herpes zoster (shingles) or postherpetic neuralgia.

28. The method of claim 25, wherein the subject is less than about 1 year of age, about 1 year of age or older, about 5 years of age or older, about 10 years of age or older, about 20 years of age or older, about 30 years of age or older, about 40 years of age or older, about 50 years of age or older, about 60 years of age or older, about 70 years of age or older, or older.

29. The method of claim 25, wherein the RNA molecule is administered as a vaccine.

30. The method of claim 25, wherein the subject is administered a single dose, two doses, three doses or more, and optionally, a booster dose of the RNA molecule.

31. A method of inducing an immune response against VZV in a subject, comprising administering to the subject an effective amount of the composition of claim 19.

32. A method of preventing, treating or ameliorating an infection, disease or condition associated with VZV in a subject, comprising administering to a subject an effective amount of the composition of claim 19.
